(19) United States
(12) Patent Application Publication
(10) Pub. No.: US 2024/0115554 A1
(43) Pub. Date: Apr. 11, 2024

(54) METHODS FOR TREATING OBSTRUCTIVE HYPERTROPHIC CARDIOMYOPATHY

(71) Applicant: Cytokinetics, Incorporated, South San Francisco, CA (US)

(72) Inventors: Fady MALIK, Burlingame, CA (US); Stuart KUPFER, Highland Park, IL (US); Daniel Louis JACOBY, Madison, CT (US); Stephen B. HEITNER, Portland, OR (US); Chiara MELLONI, South San Francisco, CA (US); Lisa MENG, South San Francisco, CA (US); Qi WOHLTMAN, Belmont, CA (US)

(21) Appl. No.: 18/365,038

(22) Filed: Aug. 3, 2023

Related U.S. Application Data
(60) Provisional application No. 63/524,559, filed on Jun. 30, 2023.
(60) Provisional application No. 63/485,215, filed on Feb. 15, 2023.
(60) Provisional application No. 63/483,882, filed on Feb. 8, 2023.
(60) Provisional application No. 63/427,067, filed on Nov. 21, 2022.
(60) Provisional application No. 63/377,279, filed on Sep. 27, 2022.
(60) Provisional application No. 63/405,310, filed on Sep. 9, 2022.
(60) Provisional application No. 63/370,435, filed on Aug. 4, 2022.

(51) Int. Cl. A61K 31/4245; A61K 45/06; A61P 9/04

(52) U.S. Cl. CPC A61K 31/4245; A61K 45/06; A61P 9/04

(57) ABSTRACT

Methods for treating symptomatic obstructive hypertrophic cardiomyopathy (oHCM) are described herein. The treatment methods include the administration of a cardiac myosin inhibitor (CK-3773274, also referred to as CK-274 or aficamten) and may include titrating an administrated daily dose based on one or more components of an echocardiogram. The daily dose may be increased, maintained, decreased, or terminated, based on the echocardiogram. The methods may be useful for treating patients who have been recently diagnosed with oHCM, patients who are treatment naïve for oHCM, or patients who have previously received SOC medical therapy for oHCM and have discontinued said therapy prior to administration of aficamten, such as patients with chronic oHCM.

### CROSS-REFERENCE TO RELATED APPLICATIONS

This application claims priority to U.S. Provisional Application No. 63/370,435, filed Aug. 4, 2022; U.S. Provisional Application No. 63/405,310, filed Sep. 9, 2022; U.S. Provisional Application No. 63/377,279, filed Sep. 27, 2022; U.S. Provisional Application No. 63/427,067, filed Nov. 21, 2022; U.S. Provisional Application No. 63/483,882, filed Feb. 8, 2023; U.S. Provisional Application No. 63/485,215, filed Feb. 15, 2023; and U.S. Provisional Application No. 63/524,559, filed Jun. 30, 2023; the contents of which are hereby incorporated by reference in their entirety for all purposes.

### FIELD

The disclosure herein relates to the treatment of obstructive hypertrophic cardiomyopathy, and compounds and compositions that may be used for treating obstructive hypertrophic cardiomyopathy.

### BACKGROUND

Hypertrophic cardiomyopathy (HCM) is a disease in which the heart muscle (myocardium) becomes abnormally thick (hypertrophied). The thickening of cardiac muscle leads to the inside of the left ventricle becoming smaller and stiffer, and thus the ventricle becomes less able to relax and fill with blood. Patients with obstructive hypertrophic cardiomyopathy may therefore suffer from abnormalities in diastology and mitral regurgitation (MR). This ultimately limits the heart's pumping function, resulting in symptoms including chest pain, dizziness, shortness of breath, or fainting during physical activity. A subset of patients with HCM are at high risk of progressive disease which can lead to atrial fibrillation, stroke and death due to arrhythmias Adverse cardiac remodeling in oHCM is a known risk factor for progression toward arrhythmias and heart failure. Thus, there is a need for therapies that address this condition.

The treatments currently used for oHCM (e.g., beta-blockers, calcium channel blockers, and disopyramide) were developed for other medical conditions and subsequently applied to oHCM due to their underlying negative inotropic characteristics. While beta-blockers have been shown to reduce symptom burden and improve hemodynamics, they do not improve exercise capacity (Dybro 2021) and can be associated with poor tolerability. There is a need for treatments that improve exercise capacity, compared with a beta blocker, in both recently diagnosed and chronic symptomatic oHCM patients.

### BRIEF SUMMARY

Methods and compositions for treating symptomatic obstructive hypertrophic cardiomyopathy (oHCM), comprising administering a cardiac myosin inhibitor aficamten, or a pharmaceutically acceptable salt thereof, are described herein. In some embodiments, the present disclosure provides methods for improving exercise capacity in a patient, e.g., a patient suffering from oHCM, comprising administering or delivering to the patient aficamten, or a pharmaceutically acceptable salt thereof as described herein. In some embodiments, the present disclosure provides methods for reducing symptom burden, improving hemodynamics, and improving exercise capacity in a patient, e.g., a patient suffering from oHCM, comprising administering or delivering to the patient aficamten, or a pharmaceutically acceptable salt thereof as described herein. The methods disclosed herein may be useful for treating patients who have been recently diagnosed with oHCM (diagnosed within 12 months prior to administering aficamten, or a pharmaceutically acceptable salt thereof). Patients who have been recently diagnosed with oHCM may include patients that are treatment naïve, and patients who have been treated or are being treated with a standard of care (SOC) medical therapy for oHCM. The methods disclosed herein may be useful for treating patients who are treatment naïve (not yet treated for oHCM). The methods disclosed herein may be useful for treating patients with chronic oHCM. Patients with chronic oHCM include patients with a history of oHCM >12 months who are currently being treated with SOC therapy for oHCM, or who have received SOC therapy for oHCM within 12 months prior to administering aficamten, or a pharmaceutically acceptable salt thereof. SOC therapies include treatment of oHCM comprising administration of a beta-blocker, a calcium channel blocker, or disopyramide.

In some implementations of the methods disclosed herein, the methods comprise administering aficamten, or a pharmaceutically acceptable salt thereof, as a monotherapy for oHCM. It is to be understood that administering a monotherapy for oHCM indicates that the patient only receives one therapy (e.g., aficamten, or a pharmaceutically acceptable salt thereof) for the treatment of oHCM; however, it is to be understood that the patient may also receive other therapy or therapies for the treatment of other diseases. As described herein, “other therapy or therapies for the treatment of other diseases” excludes SOC therapies for oHCM (e.g., one or more of a beta-blocker, a calcium channel blocker, or disopyramide). As further described herein, the daily dose of aficamten may be titrated based on the results of an echocardiogram.

In some embodiments of the methods disclosed herein, the patient has one or more of the following prior to administering aficamten, or a pharmaceutically acceptable salt thereof: LVEF ≥60%; resting LVOT-G ≥30 mm Hg; post-Valsalva LVOT-G ≥50 mmHg; NYHA Class II or Class III. In some embodiments, a patient has pVO₂ <80%. In some embodiments, a patient has a predicted pVO₂ <80%. In some embodiments, a patent has a resting LVOT-G ≥30 mmHg, post-Valsalva peak LVOT-G ≥50 mmHg, is characterized as being in NYHA functional class II or III, and has a pVO₂ <80% (measured or predicted).

In some implementations of the methods disclosed herein, administration with aficamten, or a pharmaceutically acceptable salt thereof, results in an improvement in one or more of: exercise capacity; heart function as measured by improvement in NYHA Functional Classification; resting LVOT-G; post-Valsalva LVOT-G; health status as measured by Kansas City Cardiomyopathy Questionnaire Clinical Summary Score (KCCQ-CSS); structural remodeling as measured by reduction in one or more of mean left ventricular mass index (LVMI) and left atrial volume index (LAVI); N-terminal prohormone of brain natriuretic peptide (NT-proBNP) levels; high sensitivity cardiac troponin I (hs-cTnI) levels; diastolic function as measured by reduction in lateral E/e′; interventricular septal thickness remodeling, as measured by change in interventricular septal thickness (IVST); CPET parameters selected from: ventilatory efficiency/carbon dioxide production (VE/VCO₂ slope), circulatory power (VO2×systolic blood pressure), ventilatory anaerobic threshold (VAT), total workload (watts), and heart rate response; and health status and health-related quality of life as measured by patient-reported outcome (PRO) questionnaires EuroQol 5 dimension 5-level instrument (EQ-5D-5L), Clinical Global Impression (CGI), Patient Global Impression of Change (PGI-C), and Seattle Angina Questionnaire-7 (SAQ-7). In some implementations of the methods disclosed herein, administration with aficamten, or a pharmaceutically acceptable salt thereof, results in an improvement in one or more of: exercise capacity; heart function as measured by improvement in NYHA Functional Classification; resting LVOT-G; post-Valsalva LVOT-G; health status as measured by Kansas City Cardiomyopathy Questionnaire Clinical Summary Score (KCCQ-CSS); structural remodeling as measured by reduction in one or more of mean left ventricular mass index (LVMI) and left atrial volume index (LAVI); N-terminal prohormone of brain natriuretic peptide (NT-proBNP) levels; high sensitivity cardiac troponin I (hs-cTnI) levels; diastolic function as measured by reduction in lateral E/e′; interventricular septal thickness remodeling, as measured by change in interventricular septal thickness (IVST); CPET parameters selected from: ventilatory efficiency/carbon dioxide production (VE/VCO₂ slope), circulatory power (VO2×systolic blood pressure), ventilatory anaerobic threshold (VAT), total workload (watts), and heart rate response; and health status and health-related quality of life as measured by patient-reported outcome (PRO) questionnaires EuroQol 5 dimension 5-level instrument (EQ-5D-5L), Clinical Global Impression (CGI), Patient Global Impression of Change (PGI-C), Seattle Angina Questionnaire-7 (SAQ-7), and Short-Form Survey (SF-36). In some embodiments, the one or more improved results are improved as compared to treatment with a SOC therapy (e.g., metoprolol). In some embodiments administration with aficamten, or a pharmaceutically acceptable salt thereof, results in an improvement in HCM symptoms, such as reductions of chest pain, dizziness, shortness of breath, fainting during physical activity, tiredness, low energy, limitations in physical activity.

A method of treating obstructive hypertrophic cardiomyopathy (oHCM) in a patient in need thereof, can include administering to the patient a therapeutically effective amount of aficamten, or a pharmaceutically acceptable salt thereof, wherein the therapeutically effective amount of aficamten, or a pharmaceutically acceptable salt thereof, is selected by titrating a daily dose of aficamten, or a pharmaceutically acceptable salt thereof, administered to the patient. In some embodiments, the dose is titrated once during a course of treatment. In some embodiments, the dose is titrated two or more times during a course of treatment. The daily dose may be administered to a patient at a constant amount for about two weeks before the daily dose amount is titrated.

In some implementations of the above methods, aficamten, or a pharmaceutically acceptable salt thereof is administered at a daily dose of about 5 mg to about 20 mg. In some embodiments, the daily dose is about 5 mg. In some embodiments, the daily dose is about 10 mg. In some embodiments, the daily dose is about 15 mg. In some embodiments, the daily dose is about 20 mg. For example, as described herein, in some embodiments, patients begin with 5 mg once daily; at about weeks 2, 4, and 6 patients receive an echocardiogram to determine if they will be up-titrated to doses of 10, 15 or 20 mg; and dose escalation will occur if a patient has a post-Valsalva LVOT-G ≥30 mmHg and a biplane left ventricular ejection fraction (LVEF) ≥55%, and patients who do not meet escalation criteria will continue to receive their current dose or may be down-titrated if their LVEF is below 50%. See, e.g., Table 26 for increasing, maintaining, reducing or discontinuing doses. As appreciated by those skilled in the art and described herein, unless indicated otherwise, amount, e.g., in a dose, is of the amount of aficamten free base or the corresponding amount of aficamten free base when a non-free base form, e.g., a pharmaceutically acceptable salt, is administered.

In some implementations, the daily dose is administered as a single dose each day. In some implementations, the daily dose is administered in 2 divided doses.

In some embodiments, a method of treating obstructive hypertrophic cardiomyopathy (oHCM) in a patient in need thereof, comprises: administering to the patient a first daily dose of aficamten, or a pharmaceutically acceptable salt thereof, for a first time period; and based on one or more components of a first echocardiogram for the patient acquired after the first time period, administering to the patient a second daily dose of aficamten, or a pharmaceutically acceptable salt thereof, for a second time period or terminating the administering of aficamten, or a pharmaceutically acceptable salt thereof, to the patient. The method may include selecting the second daily dose of aficamten, or a pharmaceutically acceptable salt thereof, based on the one or more components of the first echocardiogram. In some embodiments, the one or more components of the first echocardiogram comprises a biplane LVEF, or a biplane LVEF and a post-Valsalva LVOT-G. In some embodiments, the one or more components of the first echocardiogram comprises a biplane LVEF. In some embodiments, the one or more components of the first echocardiogram comprises a biplane LVEF and a post-Valsalva LVOT-G. In some embodiments of the methods described herein, the patient undergoes two or more echocardiograms within the first time period, and the second daily dose is selected based on the combined results of two or more echocardiograms obtained within the first time period.

In some implementations of the above methods, the one or more components of the first echocardiogram comprises a biplane LVEF, and the second daily dose of aficamten, or a pharmaceutically acceptable salt thereof, is lower than the first daily dose of aficamten, or a pharmaceutically acceptable salt thereof, when the biplane LVEF of the first echocardiogram is below a predetermined biplane LVEF threshold. For example, the predetermined biplane LVEF threshold may be 50%.

In some implementations of the above methods, the one or more components of the first echocardiogram comprises a biplane LVEF, and the administering of aficamten, or a pharmaceutically acceptable salt thereof, to the patient is terminated when the biplane LVEF of the first echocardiogram is below a predetermined biplane LVEF threshold. For example, the predetermined biplane LVEF threshold may be 50%.

In some implementations of the above methods, the one or more components of the first echocardiogram comprises a biplane LVEF, and wherein the second daily dose of aficamten, or a pharmaceutically acceptable salt thereof, is the same as the first daily dose of aficamten, or a pharmaceutically acceptable salt thereof, when the biplane LVEF of the first echocardiogram is at or above the predetermined biplane LVEF threshold and below a second predetermined biplane LVEF threshold. In some embodiments, the predetermined biplane LVEF threshold is 50%, and the second predetermined biplane LVEF threshold is 55%.

In some implementations of the above methods, the one or more components of the first echocardiogram comprises a biplane LVEF and a post-Valsalva LVOT-G, and wherein the second daily dose of aficamten, or a pharmaceutically acceptable salt thereof, is the same as the first daily dose of aficamten, or a pharmaceutically acceptable salt thereof, when the biplane LVEF of the first echocardiogram is at or above a second predetermined biplane LVEF threshold, and the post-Valsalva LVOT-G of the first echocardiogram is below a predetermined post-Valsalva LVOT-G threshold. In some embodiments, the second predetermined biplane LVEF threshold is 55%, and the predetermined post-Valsalva LVOT-G threshold is 30 mmHg.

In some implementations of the above method, the one or more components of the first echocardiogram comprises a biplane LVEF and a post-Valsalva LVOT-G, and wherein the second daily dose of aficamten, or a pharmaceutically acceptable salt thereof, is greater than the first daily dose of aficamten, or a pharmaceutically acceptable salt thereof, when the biplane LVEF is at or above the second predetermined biplane LVEF threshold and the post-Valsalva LVOT-G is at or above the predetermined post-Valsalva LVOT-G threshold. In some embodiments, the predetermined biplane LVEF threshold is 55%, and the post-Valsalva LVOT-G threshold is 30 mmHg.

In some implementations of the above methods, the first daily dose of aficamten, or a pharmaceutically acceptable salt thereof, is about 5 mg of aficamten. In some embodiments, the second daily dose of aficamten, or a pharmaceutically acceptable salt thereof, is about 5 mg or about 10 mg of aficamten. In some implementations of the above method, the first daily dose of aficamten, or a pharmaceutically acceptable salt thereof, is about 5 mg of aficamten and the second is about 10 mg of aficamten.

In some implementations of the above methods, the method further comprises measuring the one or more components of the first echocardiogram.

In some implementations of the above methods, first time period is about 2 weeks. In some embodiments, the second time period is about 2 weeks. For example, as described herein, in some embodiments, a provided method comprises:
administering to a patient a first daily dose of aficamten, or a pharmaceutically acceptable salt thereof, for a first time period, wherein the first daily dose is about 5 mg of aficamten and the first time period is about 2 weeks;
assessing post-Valsalva LVOT-G and LVEF of the patient (e.g., using echocardiogram) after the first time period; and
if the patient has a post-Valsalva LVOT-G ≥30 mmHg and LVEF ≥55%, administering to the patient a second daily dose of aficamten, or a pharmaceutically acceptable salt thereof, wherein the second daily dose is about 10 mg of aficamten; and if the patient has a post-Valsalva LVOT-G <30 mmHg or LVEF <55%, administering to the patient a second daily dose of aficamten, or a pharmaceutically acceptable salt thereof, wherein the second daily dose is about 5 mg of aficamten.
   As described herein, in some embodiments, a second daily dose is administered for a second time period (e.g., about 2 weeks) before a patient is assessed for post-Valsalva LVOT-G and LVEF.

In some implementations of the above methods, the second daily dose of aficamten, or a pharmaceutically acceptable salt thereof, is administered to the patient for the second time period, and the method further comprises, based on one or more components of a second echocardiogram for the patient acquired after the second time period and the second daily dose of aficamten, or a pharmaceutically acceptable salt thereof, administering to the patient a third daily dose of aficamten, or a pharmaceutically acceptable salt thereof for a third time period or terminating the administering of aficamten, or a pharmaceutically acceptable salt thereof to the patient. In some embodiments, the method comprises selecting the third daily dose of aficamten, or a pharmaceutically acceptable salt thereof, based on the one or more components of the second echocardiogram and the second daily dose. In some embodiments, the one or more components of the second echocardiogram comprises a biplane LVEF or a post-Valsalva LVOT-G. In some embodiments, the one or more components of the second echocardiogram comprises a biplane LVEF. In some embodiments, the one or more components of the second echocardiogram comprises a biplane LVEF and a post-Valsalva LVOT-G. In some embodiments of the methods described herein, the patient undergoes two or more echocardiograms within the second time period, and the third daily dose is selected based on the combined results of two or more echocardiograms obtained within the second time period.

In some embodiments of the above methods, the one or more components of the second echocardiogram comprises a biplane LVEF, and the third daily dose of aficamten, or a pharmaceutically acceptable salt thereof, is lower than the second daily dose of aficamten, or a pharmaceutically acceptable salt thereof, or the administering of aficamten, or a pharmaceutically acceptable salt thereof, to the patient is terminated, when the biplane LVEF of the second echocardiogram is below the predetermined biplane LVEF threshold. In some embodiments, the predetermined biplane LVEF threshold is 50%.

In some embodiments of the above methods, the administering of aficamten, or a pharmaceutically acceptable salt thereof, to the patient is terminated when the biplane LVEF of the second echocardiogram is below the predetermined biplane LVEF threshold and the second daily dose of aficamten, or a pharmaceutically acceptable salt thereof is the same as the first daily dose of aficamten or lower. In some embodiments, the predetermined biplane LVEF threshold is 50%.

In some embodiments of the above methods, the third daily dose of aficamten, or a pharmaceutically acceptable salt thereof, is lower than the second daily dose of aficamten, or a pharmaceutically acceptable salt thereof, when the second daily dose of aficamten, or a pharmaceutically acceptable salt thereof, is higher than the first daily dose of aficamten, or a pharmaceutically acceptable salt thereof, and the biplane LVEF of the second echocardiogram is below the predetermined biplane LVEF threshold. In some embodiments, the predetermined biplane LVEF threshold is 50%. In some embodiments, the third daily dose of aficamten, or a pharmaceutically acceptable salt thereof, is the same as the first daily dose of aficamten, or a pharmaceutically acceptable salt thereof.

In some embodiments of the above methods, the one or more components of the second echocardiogram comprises a biplane LVEF, and wherein the third daily dose of aficamten, or a pharmaceutically acceptable salt thereof, is the same as the second daily dose of aficamten, or a pharmaceutically acceptable salt thereof, when the biplane LVEF is at or above the predetermined biplane LVEF threshold and below the second predetermined biplane LVEF threshold. In some embodiments, the predetermined biplane LVEF threshold is 50%, the second predetermined biplane LVEF threshold is 55%, and the second predetermined post-Valsalva LVOT-G threshold is 30 mmHg.

In some embodiments of the above methods, the one or more components of the second echocardiogram comprises a biplane LVEF and a post-Valsalva LVOT-G, and wherein the third daily dose of aficamten, or a pharmaceutically acceptable salt thereof, is the same as the second daily dose of aficamten, or a pharmaceutically acceptable salt thereof, when the biplane LVEF is at or above the second predetermined biplane LVEF threshold and the post-Valsalva LVOT-G is below the predetermined post-Valsalva LVOT-G threshold. In some embodiments, the second predetermined biplane LVEF threshold is 55%, and the predetermined post-Valsalva LVOT-G threshold is 30 mmHg.

In some embodiments of the above methods, the one or more components of the second echocardiogram comprises a biplane LVEF and a post-Valsalva LVOT-G, and wherein the third daily dose of aficamten, or a pharmaceutically acceptable salt thereof, is greater than the second daily dose of aficamten, or a pharmaceutically acceptable salt thereof, when the biplane LVEF of the second echocardiogram is above the second predetermined biplane LVEF threshold and the post-Valsalva LVOT-G of the second echocardiogram is at or above the predetermined post-Valsalva LVOT-G threshold. In some embodiments, the second predetermined biplane LVEF threshold is 55%, and the predetermined post-Valsalva LVOT-G threshold is 30 mmHg.

In some embodiments of the above method, the first daily dose of aficamten, or a pharmaceutically acceptable salt thereof, is about 5 mg of aficamten, the second daily dose of aficamten, or a pharmaceutically acceptable salt thereof, is about 5 mg or about 10 mg of aficamten, and the third daily dose of aficamten, or a pharmaceutically acceptable salt thereof, is about 5 mg, about 10 mg, or about 15 mg of aficamten. For example, as described herein, in some embodiments, a provided method comprises:
administering to the patient a daily dose of aficamten, or a pharmaceutically acceptable salt thereof (may be referred to as a second daily dose), for a time period (may be referred to as a second time period), wherein the daily dose is about 10 mg of aficamten and the time period is about 2 week;
assessing post-Valsalva LVOT-G and LVEF of the patient (e.g., using echocardiogram) after the time period; and
if the patient has a post-Valsalva LVOT-G ≥30 mmHg and LVEF ≥55%, administering to the patient a daily dose of aficamten, or a pharmaceutically acceptable salt thereof (may be referred to as a third daily dose), wherein the daily dose is about 15 mg of aficamten; and if the patient has a post-Valsalva LVOT-G <30 mmHg or LVEF <55%, administering to the patient a daily dose of aficamten, or a pharmaceutically acceptable salt thereof (may be referred to as a third daily dose), wherein the daily dose is about 10 mg of aficamten (e.g., LVEF ≥50%) or about 5 mg of aficamten (e.g., LVEF <50% but ≥40%), or discontinuing administration for a period of time if LVEF is <40%.
   For example, in some embodiments, a provided method comprises:
administering to a patient a first daily dose of aficamten, or a pharmaceutically acceptable salt thereof, for a first time period, wherein the first daily dose is about 5 mg of aficamten and the first time period is about 2 weeks;
assessing post-Valsalva LVOT-G and LVEF of the patient (e.g., using echocardiogram) after the first time period, wherein the patient has a post-Valsalva LVOT-G ≥30 mmHg and LVEF ≥55%;
administering to the patient a second daily dose of aficamten, or a pharmaceutically acceptable salt thereof, for a second time period, wherein the second daily dose is about 10 mg of aficamten and second time period is about 2 week;
assessing post-Valsalva LVOT-G and LVEF of the patient (e.g., using echocardiogram) after the second time period; and
if the patient has a post-Valsalva LVOT-G ≥30 mmHg and LVEF ≥55%, administering to the patient a third daily dose of aficamten, or a pharmaceutically acceptable salt thereof, wherein the third daily dose is about 15 mg of aficamten; and if the patient has a post-Valsalva LVOT-G <30 mmHg or LVEF <55%, administering to the patient a third daily dose of aficamten, or a pharmaceutically acceptable salt thereof, wherein the third daily dose is about 10 mg of aficamten (e.g., LVEF ≥50%) or about 5 mg of aficamten (e.g., LVEF <50% but ≥40%), or discontinuing administration for a period of time if LVEF is <40%. As described herein, in some embodiments, a third daily dose is administered for a third time period (e.g., about 2 weeks) before a patient is assessed for post-Valsalva LVOT-G and LVEF.

In some embodiments of the above methods, the method further comprises measuring the one or more components of the second echocardiogram.

In some embodiments of the above methods, the third time period is about 2 weeks.

In some embodiments of the above methods, the third daily dose of aficamten, or a pharmaceutically acceptable salt thereof, is administered to the patient for the third time period, the method further comprising, based on one or more components of a third echocardiogram for the patient acquired after the third time period and the third daily dose of aficamten, or a pharmaceutically acceptable salt thereof, administering to the patient a fourth daily dose of aficamten, or a pharmaceutically acceptable salt thereof, for a fourth time period or terminating the administering of aficamten, or a pharmaceutically acceptable salt thereof, to the patient. In some embodiments, the method further comprises selecting the fourth daily dose of aficamten, or a pharmaceutically acceptable salt thereof, based on the one or more components of the third echocardiogram and the third daily dose. In some embodiments, the one or more components of the third echocardiogram comprises a biplane LVEF or a post-Valsalva LVOT-G. In some embodiments, the one or more components of the third echocardiogram comprises a biplane LVEF. In some embodiments, the one or more components of the third echocardiogram comprises a biplane LVEF and a post-Valsalva LVOT-G. In some embodiments of the methods described herein, the patient undergoes two or more echocardiograms within the third time period, and the fourth daily dose is selected based on the combined results of two or more echocardiograms obtained within the third time period.

In some embodiments of the above methods, the one or more components of the third echocardiogram comprises a biplane LVEF, and the fourth daily dose of aficamten, or a pharmaceutically acceptable salt thereof, is lower than the third daily dose of aficamten, or a pharmaceutically acceptable salt thereof, or the administering of aficamten, or a pharmaceutically acceptable salt thereof, to the patient is terminated, when the biplane LVEF of the third echocardiogram is below the predetermined biplane LVEF threshold. In some embodiments, the administering of aficamten, or a pharmaceutically acceptable salt thereof, to the patient is terminated when the biplane LVEF of the third echocardiogram is below the predetermined biplane LVEF threshold and the third daily dose of aficamten, or a pharmaceutically acceptable salt thereof, is the same as the first daily dose of aficamten, or a pharmaceutically acceptable salt thereof. In some embodiments, the predetermined biplane LVEF threshold is 50%.

In some embodiments of the above methods, the one or more components of the third echocardiogram comprises a biplane LVEF, and wherein the fourth daily dose of aficamten, or a pharmaceutically acceptable salt thereof, is the same as the third daily dose of aficamten, or a pharmaceutically acceptable salt thereof, when the biplane LVEF is at or above the predetermined biplane LVEF threshold and below the second predetermined biplane LVEF threshold. In some embodiments, the predetermined biplane LVEF threshold is 50%, the second predetermined biplane LVEF threshold is 55%.

In some embodiments of the above methods, the one or more components of the third echocardiogram comprises a biplane LVEF and a post-Valsalva LVOT-G, and wherein the fourth daily dose of aficamten, or a pharmaceutically acceptable salt thereof, is the same as the third daily dose of aficamten, or a pharmaceutically acceptable salt thereof, when the biplane LVEF is at or above the second predetermined biplane LVEF threshold and the post-Valsalva LVOT-G is below the predetermined post-Valsalva LVOT-G threshold. In some embodiments, the second predetermined biplane LVEF threshold is 55%, and the second predetermined post-Valsalva LVOT-G threshold is 30 mmHg.

In some embodiments of the above methods, the one or more components of the third echocardiogram comprises a biplane LVEF and a post-Valsalva LVOT-G, and wherein the fourth daily dose of aficamten, or a pharmaceutically acceptable salt thereof, is greater than the third daily dose of aficamten, or a pharmaceutically acceptable salt thereof, when the biplane LVEF of the third echocardiogram is above the second predetermined biplane LVEF threshold and the post-Valsalva LVOT-G of the third echocardiogram is at or above the predetermined post-Valsalva LVOT-G threshold. In some embodiments, the second predetermined biplane LVEF threshold is 55%, and the second predetermined post-Valsalva LVOT-G threshold is 30 mmHg.

In some embodiments of the above methods, the first daily dose of aficamten, or a pharmaceutically acceptable salt thereof, is about 5 mg of aficamten, the second daily dose of aficamten, or a pharmaceutically acceptable salt thereof, is about 5 mg or about 10 mg of aficamten, the third daily dose of aficamten, or a pharmaceutically acceptable salt thereof, is about 5 mg, about 10 mg, or about 15 mg of aficamten, and the fourth daily dose of aficamten, or a pharmaceutically acceptable salt thereof, is about 5 mg, about 10 mg, about 15 mg, or about 20 mg of aficamten. For example, as described herein, in some embodiments, a provided method comprises:
administering to the patient a daily dose of aficamten, or a pharmaceutically acceptable salt thereof (may be referred to as a third daily dose), for a time period (may be referred to as a third time period), wherein the daily dose is about 15 mg of aficamten and the time period is about 2 week;
assessing post-Valsalva LVOT-G and LVEF of the patient (e.g., using echocardiogram) after the time period; and
if the patient has a post-Valsalva LVOT-G ≥30 mmHg and LVEF ≥55%, administering to the patient a daily dose of aficamten, or a pharmaceutically acceptable salt thereof (may be referred to as a fourth daily dose), wherein the daily dose is about 20 mg of aficamten; and if the patient has a post-Valsalva LVOT-G <30 mmHg or LVEF <55%, administering to the patient a daily dose of aficamten, or a pharmaceutically acceptable salt thereof (may be referred to as a fourth daily dose), wherein the daily dose is about 15 mg of aficamten (e.g., LVEF ≥50%) or about 10 mg of aficamten (e.g., LVEF <50% but ≥40%), or discontinuing administration for a period of time if LVEF is <40%.
   For example, in some embodiments, a provided method comprises:
administering to a patient a first daily dose of aficamten, or a pharmaceutically acceptable salt thereof, for a first time period, wherein the first daily dose is about 5 mg of aficamten and the first time period is about 2 weeks;
assessing post-Valsalva LVOT-G and LVEF of the patient (e.g., using echocardiogram) after the first time period, wherein the patient has a post-Valsalva LVOT-G ≥30 mmHg and LVEF ≥55%;
administering to the patient a second daily dose of aficamten, or a pharmaceutically acceptable salt thereof, for a second time period, wherein the second daily dose is about 10 mg of aficamten and second time period is about 2 week;
assessing post-Valsalva LVOT-G and LVEF of the patient (e.g., using echocardiogram) after the second time period; and
administering to the patient a third daily dose of aficamten, or a pharmaceutically acceptable salt thereof, for a third time period, wherein the third daily dose is about 15 mg of aficamten and third time period is about 2 week;
assessing post-Valsalva LVOT-G and LVEF of the patient (e.g., using echocardiogram) after the third time period; and
if the patient has a post-Valsalva LVOT-G ≥30 mmHg and LVEF ≥55%, administering to the patient a fourth daily dose of aficamten, or a pharmaceutically acceptable salt thereof, wherein the third daily dose is about 20 mg of aficamten; and if the patient has a post-Valsalva LVOT-G <30 mmHg or LVEF <55%, administering to the patient a fourth daily dose of aficamten, or a pharmaceutically acceptable salt thereof, wherein the fourth daily dose is about 15 mg of aficamten (e.g., LVEF ≥50%) or about 10 mg of aficamten (e.g., LVEF <50% but ≥40%), or discontinuing administration for a period of time if LVEF is <40%.

As described herein, in some embodiments, a fourth daily dose is administered for a fourth time period (e.g., about 2 weeks) before a patient is assessed for post-Valsalva LVOT-G and LVEF. In some embodiments, a fourth daily dose is administered for about 2 or more weeks, e.g., about 4 weeks, about 1 month, about 2, 3, 4, 5, 6, 7, 8, 9, 10, 11 or 12 months, or longer. In some embodiments, during a fourth time period, e.g., a fourth time period longer than about 2 weeks, a patient may be assessed one or more times for cardiac structures and/or functions, e.g., post-Valsalva LVOT-G, LVEF, exercise capacity, etc. As described herein, among other things, provided methods can improve exercise capacity.

In some embodiments of the above methods, the method further comprises measuring the one or more components of the third echocardiogram.

In some embodiments of the above methods, the fourth time period is about 2 weeks.

In some embodiments of the above methods, the first predetermined biplane LVEF threshold is 50%, the second predetermined biplane LVEF threshold is 55%, and the predetermined post-Valsalva LVOT-G threshold is 30 mmHg.

In some embodiments, a daily dose is about 15 mg of aficamten. In some embodiments, a third daily dose is about 15 mg of aficamten. In some embodiments, a fourth daily dose is about 15 mg of aficamten. In some embodiments, a daily dose is about 20 mg of aficamten. In some embodiments, a fourth daily dose is about 20 mg of aficamten. In some embodiments, a daily dose is administered as a tablet. In some embodiments, amount of aficamten, which may exist in multiple forms (e.g., free form, pharmaceutically acceptable salt forms, polymorph forms, etc.), in a tablet is about a daily dose as described herein. In some embodiments, the amount of aficamten in a tablet is about 5 mg. In some embodiments, the amount of aficamten in a tablet is about 10 mg. In some embodiments, the amount of aficamten in a tablet is about 15 mg. In some embodiments, the amount of aficamten in a tablet is about 20 mg. In some embodiments, the amount of aficamten in a tablet is about half of a daily dose as described herein. In some embodiments, the amount of aficamten in a tablet is about 2.5 mg. In some embodiments, the amount of aficamten in a tablet is about 5 mg. In some embodiments, the amount of aficamten in a tablet is about 7.5 mg. In some embodiments, the amount of aficamten in a tablet is about 10 mg.

In some embodiments of any of the above methods, the aficamten or pharmaceutically acceptable salt thereof is administered orally. In some embodiments, the aficamten or pharmaceutically acceptable salt thereof is administered as a tablet. In some embodiments, the tablet comprises one or more carriers or excipients selected from the group consisting of mannitol, lactose, starch, magnesium stearate, sodium saccharine, talcum, cellulose, sodium croscarmellose, glucose, gelatin, sucrose, and magnesium carbonate. In some embodiments, the tablet comprises: (i) about 1% by weight to about 50% by weight of the aficamten or pharmaceutically acceptable salt thereof; (ii-1) about 10% by weight to about 60% by weight of mannitol; (ii-2) about 5% by weight to about 45% by weight of microcrystalline cellulose; (iii) about 0.1% by weight to about 10% by weight of hydroxypropyl cellulose; (iv) about 1% by weight to about 10% by weight of croscarmellose sodium; (v) about 0.1% by weight to about 10% by weight of sodium lauryl sulfate; and vi) about 0.1% by weight to about 10% by weight of magnesium stearate, wherein the % by weight excludes the weight of a coating, if present. In some embodiments, the aficamten or pharmaceutically acceptable salt thereof comprises polymorphic one or more of Form I, Form II, Form III, Form IV, Form V, and Form VI of aficamten.

### BRIEF DESCRIPTION OF THE DRAWINGS

FIG. 1 illustrates and exemplary method for treating obstructive hypertrophic cardiomyopathy (oHCM) in a patient that included titrating the daily dose of aficamten, or a pharmaceutically acceptable salt thereof.

FIG. 2 shows a schematic overview of a phase 1 clinical study for aficamten. The study included SAD cohorts, MAD cohorts, a CYP2D6-PM cohort, and a food-effect cohort. The MAD and CYP2D6-PM cohorts began when a tolerated, pharmacologically active dose (reduction in LVEF of approximately 5%) was identified in the SAD cohorts. The food-effect cohort began following completion of the last SAD cohort. Criteria to stop dose escalation were met in the SAD 75-mg dose cohort, and remaining patients in this cohort received 50 mg. Subsequently, the final SAD cohort was completed using 40 mg of aficamten. CYP2D6-PM=cytochrome P450 2D6 poor metabolizer phenotype; d=day; LVEF=left ventricular ejection fraction; MAD=multiple-ascending dose; qd=once daily; SAD=single-ascending dose.

FIG. 3A shows mean (SE) maximum plasma concentration (Cₘₐₓ) of aficamten increased in a dose-proportional manner following single oral doses between 1 mg and 50 mg. FIG. 3B shows exposure (AUC₂₄) (B) of aficamten increased in a dose-proportional manner following single oral doses between 1 mg and 50 mg. AUC₂₄=area under the plasma drug concentration-time curve from 0 to 24 h; Cₘₐₓ=maximum plasma concentration; SE=standard error.

FIG. 4 shows plasma concentration over time with multiple doses of aficamten according to an exemplary clinical trial. Mean (SE) aficamten plasma concentrations are displayed. Data points are offset for clarity. Aficamten plasma concentrations increased between the 5-mg dose and the 2 higher doses; however, by day 2 there was no difference between mean concentrations of the 7.5-mg and 10-mg doses. Clearance was similar for the 5-mg and 10-mg doses, and the accumulation ratio was similar for all 3 doses. For days 7, 8, 10, 11, 12, and 13, only trough measurements are shown. For the 5-mg and 10-mg cohorts, the dosing period was 14 days with a 3-day follow-up. For the 7.5-mg cohort, dosing was extended to 17 days, with a 3-day follow-up, and confirmed that steady state was achieved after 10 to 12 days. SE=standard error.

FIG. 5A shows SAD cohorts and FIG. 5B shows MAD cohorts for an exemplary aficamten clinical trial. Mean (SE) change from baseline in LVEF is displayed. Data points are offset for clarity. In both the SAD and MAD cohorts, reductions in LVEF within the target range (5% to 15% reduction) were observed. In the SAD cohorts, there were generally small decreases in LVEF, with mean maximum reduction of 5.8% in the 50-mg group (at 1.5 h post dose). In the MAD cohort, the greatest mean reduction in LVEF from baseline occurred in the 10-mg group (mean change of 5.0% 1.5 h after dosing on day 14). LVEF=left ventricular ejection fraction; MAD=multiple-ascending dose; qd, once daily; SAD=single-ascending dose; SE=standard error.

FIG. 6A shows analysis of the SAD cohorts according to an exemplary clinical trial, and shows that, as the plasma concentration of aficamten increased, there was a trend toward a decrease in LVEF. FIG. 6B shows analysis of the MAD cohorts from an exemplary clinical trial, and shows minimal suppression of LVEF in most participants at plasma aficamten concentrations of <180 ng/ml. CI=confidence interval; LVEF=left ventricular ejection fraction; MAD=multiple-ascending dose; SAD=single-ascending dose.

FIG. 7 shows resting LVOT-G for treatment and placebo cohorts according to an exemplary clinical trial for aficamten.

FIG. 8 shows post-Valsalva LVOT-G for treatment and placebo cohorts according to an exemplary clinical trial for aficamten.

FIG. 9 shows left arterial volume index (LAVI) changes for treatment and placebo cohorts according to an exemplary clinical trial for aficamten.

FIG. 10 shows changes to lateral E/e′ ratio for treatment and placebo cohorts according to an exemplary clinical trial for aficamten.

FIG. 11 shows changes to mitral valve characteristics, including mitral regurgitation (MR), eccentric MR, and systolic anterior motion (SAM), for treatment and placebo cohorts according to an exemplary clinical trial for aficamten.

FIG. 12 shows changes to resting LVOT-G for treatment and placebo cohorts according to an exemplary clinical trial for aficamten.

FIG. 13 shows changes to resting Valsalva LVOT-G for treatment and placebo cohorts according to an exemplary clinical trial for aficamten.

FIG. 14 shows changes to LVEF for treatment and placebo cohorts according to an exemplary clinical trial for aficamten.

FIG. 15 shows NYHA functional class response for treatment and placebo cohorts according to an exemplary clinical trial for aficamten.

FIG. 16 shows changes in mean NT-proBNP for treatment and placebo cohorts according to an exemplary clinical trial for aficamten.

FIG. 17 shows changes to resting LVOT-G for treatment and placebo cohorts according to an exemplary clinical trial for aficamten.

FIG. 18 shows changes to resting Valsalva LVOT-G for treatment and placebo cohorts according to an exemplary clinical trial for aficamten.

FIG. 19 shows changes to LVEF for treatment and placebo cohorts according to an exemplary clinical trial for aficamten.

FIG. 20 shows hemodynamic response for treatment and placebo cohorts according to an exemplary clinical trial for aficamten.

FIG. 21 shows NYHA functional class response for treatment and placebo cohorts according to an exemplary clinical trial for aficamten.

FIG. 22 shows changes in mean NT-proBNP for treatment and placebo cohorts according to an exemplary clinical trial for aficamten.

FIG. 23 shows hs-troponin for treatment and placebo cohorts according to an exemplary clinical trial for aficamten.

FIG. 24 shows NYHA functional class response for treatment and placebo cohorts according to an exemplary clinical trial for aficamten.

FIG. 25 shows NYHA functional class response for treatment and placebo cohorts according to an exemplary clinical trial for aficamten.

FIG. 26 shows the design of an open label extension of an exemplary clinical trial for aficamten (Example 3a).

FIG. 27 shows the distribution of patients across doses over time in the open label extension of an exemplary clinical trial for aficamten (Example 3a).

FIG. 28 shows resting LVOT-G for patients in the open label extension of an exemplary clinical trial for aficamten (Example 3a).

FIG. 29 shows post-Valsalva LVOT-G for patients in the open label extension of an exemplary clinical trial for aficamten (Example 3a).

FIG. 30 shows changes to LVEF for patients in the open label extension of an exemplary clinical trial for aficamten (Example 3a).

FIG. 31 shows NYHA functional class distribution at various time points for patients in the open label extension of an exemplary clinical trial for aficamten (Example 3a).

FIG. 32 shows NYHA functional class response at various time points for patients in the open label extension of an exemplary clinical trial for aficamten (Example 3a).

FIG. 33 shows changes in mean NT-proBNP for patients in the open label extension of an exemplary clinical trial for aficamten (Example 3a).

FIG. 34 shows changes in mean cardiac troponin I for patients in the open label extension of an exemplary clinical trial for aficamten (Example 3a).

FIG. 35 shows changes in KCCQ scores from baseline for patients in the open label extension of an exemplary clinical trial for aficamten (Example 3a).

FIG. 36 shows proportion of patients with various levels of changes in KCCQ scores from baseline for patients in the open label extension of an exemplary clinical trial for aficamten (Example 3a).

FIG. 37 shows the percent change in hs-troponin I from baseline for treatment and placebo cohorts according to an exemplary clinical trial for aficamten (Example 3a).

FIG. 38 shows the design of an exemplary phase 3 clinical trial for aficamten.

FIG. 39 shows changes in resting LVOT-G from baseline at various time points for patients in the open label extension of exemplary clinical trials for aficamten (Examples 3a and 3b).

FIG. 40 shows changes in post-Valsalva LVOT-G from baseline at various time points for patients in the open label extension of exemplary clinical trials for aficamten (Examples 3a and 3b).

FIG. 41 shows changes in NYHA functional class distribution at various time points for patients in the open label extension of exemplary clinical trials for aficamten (Examples 3a and 3b).

FIG. 42 shows changes in LVEF at various time points for patients in the open label extension of exemplary clinical trials for aficamten (Examples 3a and 3b).

FIG. 43 shows the design of an exemplary Phase 3 clinical trial for aficamten (Example 5).

FIG. 44 shows the baseline doses of beta blockers and calcium channel blockers for 40 patients in the open label extension of exemplary clinical trials for aficamten (Examples 3a and 3b).

FIGS. 45A and 45B show interim changes in NT-proBNP and hs-troponin I, respectively, for patients in the open label extension of exemplary clinical trials for aficamten (Examples 3a and 3b).

FIG. 46 shows resting LVOT-G from baseline to week 12 for an exemplary clinical trial for aficamten (Example 2).

FIG. 47 shows Valsalva LVOT-G from baseline to week 12 for an exemplary clinical trial for aficamten (Example 2).

FIG. 48 shows LVEF from baseline to week 12 for an exemplary clinical trial for aficamten (Example 2).

FIG. 49 shows distribution of patients demonstrating hemodynamic response at week 10 of an exemplary clinical trial for aficamten (Example 2).

FIG. 50 shows distribution of patients demonstrating improvement in NYHA class at week 10 of an exemplary clinical trial for aficamten (Example 2).

FIG. 51 shows mean proportional change from baseline in NT-proBNP and hs-cTnI at week 10 of an exemplary clinical trial for aficamten (Example 2).

FIG. 52 shows doses of aficamten achieved for patients in the open label extension of exemplary clinical trials for aficamten (Examples 3a and 3b).

FIG. 53 shows core-laboratory and site-interpreted mean (SD) changes in resting LVOT-G from baseline at various time points for patients in the open label extension of exemplary clinical trials for aficamten (Example 3a and 3b). Horizontal dashed line represents threshold for designation of severe obstruction.

FIG. 54 shows core-laboratory and site-interpreted mean (SD) changes in Valsalva LVOT-G from baseline at various time points for patients in the open label extension of exemplary clinical trials for aficamten (Example 3a and 3b). Horizontal dashed line represents threshold for designation of severe obstruction.

FIG. 55 shows core-laboratory and site-interpreted mean (SD) changes in LVEF at various time points for patients in the open label extension of exemplary clinical trials for aficamten (Example 3a and 3b). Horizontal dashed line represents low LVEF threshold at 50 mmHg.

FIG. 56 shows proportion of patients demonstrating improvement in NYHA class at various weeks up to week 48 in the open label extension of exemplary clinical trials for aficamten (Example 3a and 3b).

FIG. 57A shows change in NYHA class from baseline to weeks 36-48 for 27 patients in the open label extension of exemplary clinical trials for aficamten (Example 3a and 3b).

FIG. 57B shows change in global longitudinal strain (GLS) from baseline to week 12 and weeks 36-48 for 27 patients undergoing echocardiography at week 12 and week 36-48 in the open label extension of exemplary clinical trials for aficamten (Example 3a and 3b).

FIG. 57C shows change in global longitudinal strain (GLS) from baseline to week 12 and weeks 36-48 for patients undergoing echocardiography at week 12 and week 36-48 and having optimal hemodynamic response (resting LVOT-G) in the open label extension of exemplary clinical trials for aficamten (Example 3a and 3b).

FIG. 57D shows change in global longitudinal strain (GLS) from baseline to week 12 and weeks 36-48 for patients undergoing echocardiography at week 12 and week 36-48 and having optimal hemodynamic response (Valsalva LVOT-G) in the open label extension of exemplary clinical trials for aficamten (Example 3a and 3b).

FIG. 58 shows the design of an exemplary phase 3 clinical trial for aficamten.

FIG. 59A shows an experimental X-ray powder diffraction (XRPD) pattern of polymorphic Form I of aficamten.

FIG. 59B shows differential scanning calorimetry (DSC) and thermogravimetric analysis (TGA) graphs of polymorphic Form I of aficamten.

FIG. 59C shows a Dynamic Vapor Sorption (DVS) graph of polymorphic Form I of aficamten.

FIG. 60A shows an experimental XRPD pattern of polymorphic Form II of aficamten.

FIG. 60B shows DSC and TGA graphs of polymorphic Form II of aficamten.

FIG. 61A shows an experimental XRPD pattern of a mixture of polymorphic Forms I and III of aficamten.

FIG. 61B shows DSC and TGA graphs of a mixture of polymorphic Forms I and III of aficamten.

FIG. 62A shows an experimental XRPD pattern of polymorphic Form IV of aficamten.

FIG. 62B shows DSC and TGA graphs of polymorphic Form IV of aficamten.

FIG. 63 shows an experimental XRPD pattern and two simulated patterns of polymorphic Form V of aficamten (from top to bottom: simulated at 223K; simulated at 273K; experimental).

FIG. 64A shows two experimental XRPD patterns of polymorphic Form VI of aficamten: (a) at top, the XRPD of Form VI taken before drying; and (b) at bottom, after drying (oven, vacuum, 24 hours, at 25° C.).

FIGS. 64B and 64C show TGA graphs of polymorphic Form VI of aficamten. FIG. 64B shows the weight loss of an oven-dried sample (oven, vacuum, overnight, at 25° C.) of Form VI over the range of 25-300° C. FIG. 64C shows the TGA plot over the range of 25-300° C. for a sample of Form VI that was subjected to oven-drying (oven, vacuum, overnight, at 25° C.) and further heating at 150° C. prior to thermogravimetric analysis.

FIGS. 64D and 64E show DSC graphs of polymorphic Form VI of aficamten. FIG. 64D shows the DSC plot of an oven-dried sample (oven, vacuum, overnight, at 25° C.) of Form VI over the range of 25-300° C. FIG. 64E shows the DSC plot over the range of 25-300° C. for a sample of Form VI that was subjected to oven-drying (oven, vacuum, overnight, at 25° C.) and further heating at 150° C. prior to thermogravimetric analysis.

### DETAILED DESCRIPTION

Described herein is a cardiac myosin inhibitor aficamten and various methods, e.g., for treating obstructive hypertrophic cardiomyopathy in a subject in need thereof using aficamten, or a pharmaceutically acceptable salt thereof.

Treatment methods may include adjusting a dose, for example to increase, decrease or maintain a dose, or terminate administration, based on the results of one or more measured biplane left ventricular ejection fraction (LVEF) measurement, and/or post-Valsalva left ventricular outflow tract pressure gradient (LVOT-G) measurements. These measurements may be taken, for example, using an echocardiogram. Methods disclosed herein may be useful as a 1) a first-line therapy for subjects recently diagnosed and/or treatment naïve; or as 2) monotherapy for participants previously receiving standard of care (SOC) medical therapy for symptomatic oHCM. It is to be understood that administering a monotherapy for oHCM indicates that the patient only receives one therapy (e.g., aficamten, or a pharmaceutically acceptable salt thereof) for the treatment of oHCM; however, it is to be understood that the patient may also receive other therapy or therapies for the treatment of other diseases. As described herein, “other therapy or therapies for the treatment of other diseases” excludes SOC therapies for oHCM (e.g., one or more of a beta-blocker, a calcium channel blocker, or disopyramide).

Aficamten is a small molecule cardiac myosin inhibitor having the structure shown below.

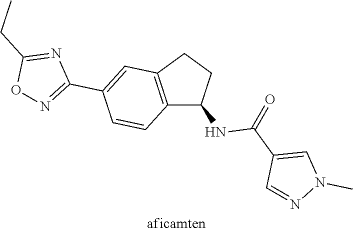 [SMILES: CCc1nc(-c2ccc3c(c2)CC[C@H]3NC(=O)c2cnn(C)c2)no1]

The chemical name of aficamten is (R)—N-(5-(5-ethyl-1,2,4-oxadiazol-3-yl)-2,3-dihydro-1H-inden-1-yl)-1-methyl-1H-pyrazole-4-carboxamide. The small molecule inhibitor may be, for example, orally administered to a patient for the treatment of obstructive hypertrophic cardiomyopathy.

Aficamten has been described in WO 2019/144041, which is incorporated by reference herein. Aficamten, or a pharmaceutically acceptable salt thereof, may be obtained following the methods described therein. Aficamten used in the disclosed methods can be present as a pharmaceutically acceptable salt, solvate, hydrate, polymorph, or combination thereof, and can be formulated into any suitable pharmaceutical formulation. Aficamten can also be present in its free base form. Polymorphs of aficamten have been described in WO 2021/011807, which is incorporated by reference herein. Formulations of aficamten have been described in WO 2021/011808, which is incorporated by reference herein. Aficamten was designed to reduce the hypercontractility that is associated with hypertrophic cardiomyopathy (HCM). Without being bound by theory, in preclinical models, aficamten reduces myocardial contractility by binding directly to cardiac myosin at a distinct and selective allosteric binding site, thereby preventing myosin from entering a force producing state. Aficamten reduces the number of active actin-myosin cross bridges during each cardiac cycle and consequently reduces myocardial contractility. This mechanism of action may be therapeutically effective in conditions characterized by excessive hypercontractility, such as HCM (e.g., obstructive HCM, also referred to as oHCM).

### Definitions

As used in the present specification, the following words and phrases are generally intended to have the meanings as set forth below, except to the extent that the context in which they are used indicates otherwise.

Throughout this application, unless the context indicates otherwise, references to aficamten include amorphous forms thereof or polymorphs thereof, including any one of polymorphic Forms I, II, III, IV, V, or VI as described herein, or a mixture thereof.

Reference to “about” a value or parameter herein includes (and describes) that value or parameter per se, and any value or parameter 5% above or 5% below said parameter. For example, description to “about X” includes description of “X” and “X+/−5%”. For example, a daily dose of “about 5 mg” includes “5 mg+/−5%”, which includes dose of 4.75 mg, 5.25 mg, or any amount therebetween.

“Recently diagnosed” patients refer to patients with a history of oHCM <12 months, with or without the use of standard-of-care therapy for oHCM.

“Treatment naïve” patients refer to patients who have received no previous standard-of-care therapy for oHCM.

“Currently untreated” patients refer to patients with no standard-of-care therapy for oHCM within the past 12 months.

Patients with “chronic oHCM” refers to patients with a history of oHCM >12 months who are a) currently being treated with standard-of-care therapy for oHCM, or b) have received standard-of-care therapy for oHCM within the past 12 months.

“NYHA classification” or “NYHA class” refers to the New York Heart Association functional classification of heart failure symptoms. Descriptions of each of NYHA classes I, II, III, and IV can be found in “Classes of Heart Failure”, American Heart Association, https://www.heartorg/en/health-topics/heart-failure/what-is-heart-failure/classes-of-heart-failure, adapted from: 1) Dolgin M, Association NYH, Fox AC, Gorlin R, Levin RI, New York Heart Association. Criteria Committee. “Nomenclature and criteria for diagnosis of diseases of the heart and great vessels”. 9th ed. Boston, MA: Lippincott Williams and Wilkins; Mar. 1, 1994; and 2) Criteria Committee, New York Heart Association, Inc. Diseases of the Heart and Blood Vessels. Nomenclature and Criteria for diagnosis, 6th edition Boston, Little, Brown and Co. 1964, p 114. Briefly, NYHA class I indicates that the patient has no limitation in ordinary physical activity (e.g., shortness of breath when walking, climbing stairs). NYHA class II indicates that the patient has mild symptoms (e.g., mild shortness of breath and/or angina) and slight limitation during ordinary activity. NYHA class III indicates that the patient has in activity due to symptoms, even during less-than-ordinary activity (e.g., walking short distances [20-100 m]); comfortable only at rest. NYHA class IV indicates that the patient has severe limitations, experiences symptoms even while at rest; mostly bedbound participants.

The term “pharmaceutically acceptable salt” refers to a salt of any of the compounds herein that are known to be non-toxic and are commonly used in the pharmaceutical literature. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J. Pharmaceutical Sciences, January 1977, 66(1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethylamine, diethylamine, triethylamine, tripropylamine, and ethanolamine. In some embodiments, the pharmaceutically acceptable base addition salt is selected from ammonium, potassium, sodium, calcium, and magnesium salts.

If the compound described herein is obtained as an acid addition salt, the free base can be obtained by basifying a solution of the acid salt. Conversely, if the compound is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds (see, e.g., Berge et al., Pharmaceutical Salts, J. Pharmaceutical Sciences, January 1977, 66(1), 1-19). Those skilled in the art will recognize various synthetic methodologies that may be used to prepare pharmaceutically acceptable addition salts.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in pharmaceutical compositions is contemplated. Supplementary active ingredients can also be incorporated into the pharmaceutical compositions.

The terms “patient,” “individual,” and “subject” refer to an animal, such as a mammal. Mammals include, for example, mice, rats, dogs, cats, pigs, sheep, horses, cows and humans. In some embodiments, the patient or subject is a human, for example a human that has been or will be the object of treatment, observation or experiment. The compounds, compositions and methods described herein can be useful in both human therapy and veterinary applications.

The term “therapeutically effective amount” or “effective amount” refers to that amount of a compound disclosed and/or described herein that is sufficient to affect treatment, as defined herein, when administered to a patient in need of such treatment. A therapeutically effective amount of a compound may be an amount sufficient to treat a disease responsive to modulation of the cardiac sarcomere. The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount may be ascertained experimentally, for example by assaying blood concentration of the chemical entity, or theoretically, by calculating bioavailability.

“Treatment” (and related terms, such as “treat”, “treated”, “treating”) includes one or more of: inhibiting a disease or disorder; slowing or arresting the development of clinical symptoms of a disease or disorder; and/or relieving a disease or disorder (i.e., causing relief from or regression of clinical symptoms). The term covers both complete and partial reduction or prevention of the condition or disorder, and complete or partial reduction of clinical symptoms of a disease or disorder. Thus, compounds described and/or disclosed herein may prevent an existing disease or disorder from worsening, assist in the management of the disease or disorder, or reduce or eliminate the disease or disorder.

Reference to any dose amount of a compound or pharmaceutically acceptable salt thereof described herein (e.g., 5 mg, 10 mg, 20 mg, etc. of aficamten) refers to the amount (i.e., equivalent mass) of said compound without any salt.

### Treatment of Obstructive Hypertrophic Cardiomyopathy

As further described herein a therapeutically effective amount of aficamten may be administered to a patient for the treatment of obstructive hypertrophic cardiomyopathy. Aficamten may be administered at a constant dose level. Aficamten may be administered at a titrated dose level. For example, the dose of aficamten may be adjusted depending on the patient's response to the drug. That is, the dose of the aficamten may be periodically increased, decreased, maintained, or terminated depending on the measurement of a drug response, such as one or more of a biplane left ventricular ejection fraction (LVEF) and post-Valsalva LVOT-G measurement.

Results of a recent clinical trial (see Example 1) demonstrated that treatment with aficamten for 10 weeks resulted in substantial and statistically significant reductions from baseline compared to placebo in the average resting left ventricular outflow tract pressure gradient (LVOT-G) (p=0.0003, p=0.0004, Cohort 1 and Cohort 2, respectively) and the average post-Valsalva LVOT-G (p=0.001, p<0.0001, Cohort 1 and Cohort 2, respectively). The majority of patients treated with aficamten (78.6% in Cohort 1 and 92.9% in Cohort 2) achieved the target goal of treatment, defined as resting gradient <30 mmHg and post-Valsalva gradient <50 mmHg at Week 10 compared to placebo (7.7%). Reductions in LVOT-G occurred within two weeks of initiating treatment with aficamten, peaked within two to six weeks of the end of dose titration, and were sustained until the end of treatment at 10 weeks. The observed reductions in LVOT-G were dose dependent, with patients achieving greater reductions of LVOT-G with increasing doses of aficamten.

Treatment with aficamten in the clinical trial was well tolerated. Overall, the incidence of adverse events was similar between treatment arms. No serious adverse events were attributed to aficamten and no treatment interruptions occurred on aficamten. No new cases of atrial fibrillation were reported by the investigators. In this dose-range finding trial, one patient experienced a transient decrease in left ventricular ejection fraction (LVEF) that required dose adjustment but not dose interruption. LVEF returned towards baseline within two weeks after the end of treatment in both cohorts, confirming the reversibility of effect with aficamten as was similarly observed in healthy participants in the Phase 1 study of aficamten.

Aficamten is administered at a therapeutically effective dosage, e.g., a dosage sufficient to provide treatment for the disease state. For a human, the daily dose may be between about 1 mg and about 50 mg. For example, the daily dose may be about 5 mg, about 10 mg, about 15 mg, about 20 mg, or any amount therebetween. A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

In one example, a patient is treated for oHCM by administering to the patient a daily dose of about 5 mg to about 20 mg of aficamten. In one example, a patient is treated for obstructive hypertrophic cardiomyopathy by administering to the patient a daily dose of about 5 mg aficamten. In one example, a patient is treated for obstructive hypertrophic cardiomyopathy by administering to the patient a daily dose of about 10 mg aficamten. In one example, a patient is treated for obstructive hypertrophic cardiomyopathy by administering to the patient a daily dose of about 15 mg aficamten. In one example, a patient is treated for obstructive hypertrophic cardiomyopathy by administering to the patient a daily dose of about 20 mg aficamten.

In some embodiments, provided are methods for the treatment of oHCM comprising administration of aficamten, or a pharmaceutically acceptable salt thereof, or a pharmaceutical composition containing aficamten, or a pharmaceutically acceptable salt thereof, as a monotherapy, wherein the patient has been recently diagnosed with oHCM, the patient is treatment naïve, or the patient has chronic oHCM. In some embodiments, provided are methods for the treatment of oHCM comprising administration of aficamten, or a pharmaceutically acceptable salt thereof, or a pharmaceutical composition containing aficamten, or a pharmaceutically acceptable salt thereof, as a monotherapy, wherein patient has chronic oHCM and has previously received SOC medical therapy for oHCM prior to administering aficamten, or a pharmaceutically acceptable salt thereof. In some embodiments, provided are methods for the treatment of oHCM comprising administration of aficamten, or a pharmaceutically acceptable salt thereof, or a pharmaceutical composition containing aficamten, or a pharmaceutically acceptable salt thereof, as a monotherapy, wherein patient has chronic oHCM and has previously received SOC medical therapy for oHCM prior to administering aficamten, or a pharmaceutically acceptable salt thereof, and has discontinued the SOC medical therapy to oHCM prior to administering aficamten, or a pharmaceutically acceptable salt thereof.

In some embodiments, the patient has LVEF ≥60% and resting LVOT-G ≥30 mm Hg prior to administering aficamten, or a pharmaceutically acceptable salt thereof. In some embodiments, the patient has LVEF ≥60% and post-Valsalva LVOT-G ≥50 mmHg prior to administering aficamten. In some embodiments, the patient has LVEF ≥60%, resting LVOT-G ≥30 mm Hg, and post-Valsalva LVOT-G ≥50 mmHg prior to administering aficamten. In some embodiments, the patient has LVEF ≥60%, resting LVOT-G ≥30 mm Hg, and NYHA Class II or Class III prior to administering aficamten, or a pharmaceutically acceptable salt thereof. In some embodiments, the patient has LVEF ≥60%, post-Valsalva LVOT-G ≥50 mmHg, and NYHA Class II or Class III prior to administering aficamten. In some embodiments, the patient has LVEF ≥60%, resting LVOT-G ≥30 mm Hg, post-Valsalva LVOT-G ≥50 mmHg, and NYHA Class II or Class III prior to administering aficamten.

During the course of treatment for oHCM, the dose of aficamten administered to the patient may be titrated, for example by increasing, decreasing, or maintaining the dose. Titration may occur once during treatment, or may be performed iteratively separated by a period of time. For example, in some implementations, the dose of aficamten is titrated two or more times (e.g., 3, 4, 5 or more) during the course of treatment. In some embodiments, a new daily dose amount is administered to the patient at a constant amount for about 1 week to about 8 weeks (or about 2 weeks to about 6 weeks, or about 4 weeks) before the daily dose amount is titrated. In some embodiments, a new daily dose amount is administered to the patient at a constant amount for about 2 weeks before being titrated. For example, a first daily dose may be administered to the patient for about 2 weeks before a first titration, wherein the daily dose amount is increased, decreased or maintained. The second titration may then occur approximately 2 weeks after the first titration. Titration of the dose allows the dose to be personalized to the patient's response to the drug, thus maximizing the potential treatment effect for patients.

Titration of the dose can be based on one or more of a biplane left ventricular ejection fraction (LVEF) and post-Valsalva LVOT-G measured in the patient. The measurement or measurements may be determined, for example, using an echocardiogram. The echocardiogram is taken following administration of the daily dose, for example about 1 hour to about 3 hours following administration of the dose. In some embodiments, the echocardiogram is taken about 2 hours following administration of the daily dose.

In some embodiments, an initial daily dose of about 5 mg, about 10 mg, about 15 mg, or about 20 mg aficamten, or any amount therebetween, is administered to the patient. After a period of time (e.g., about 2 weeks), biplane LVEF and/or post-Valsalva LVOT-G is measured, for example by echocardiography, following the administration of the dose (for example, about 1-3 hours, or about 2 hours following the administration of the dose). The dose may be decreased or terminated if the biplane LVEF is below a predetermined biplane LVEF threshold (e.g., lower than 50%). For example, the dose may be decreased if the biplane LVEF is below the biplane LVEF threshold and the current dose is not the lowest (e.g., first) dose. The dose may be terminated if the biplane LVEF is below the biplane LVEF threshold and the current dose is the lowest (e.g., first) dose. The dose may be maintained if the biplane LVEF is at or above the predetermined biplane LVEF threshold, and below a second predetermined biplane LVEF threshold (e.g., 50%≤LVEF <55%), or the dose may be maintained if the biplane LVEF is at or above the second predetermined biplane LVEF threshold (e.g., 55% or higher) and the post-Valsalva LVOT-G is below the predetermined LVOT-G threshold (e.g., less than 30%). If the post-Valsalva LVOT-G is at or above a predetermined LVOT-G threshold (e.g., about 30 mmHg or more) and the biplane LVEF is at or above a second predetermined biplane LVEF threshold (e.g., about 55% or more), the daily dose is increased. In some embodiments, the biplane LVEF threshold is about 50%, and the post-Valsalva LVOT-G threshold is about 30 mmHg. If the LVEF is <40% at any time, administration of the daily dose is temporarily interrupted. In some embodiments, titration of the dose of aficamten comprises maintaining the dose at the current dose; increasing the dose by about 1 mg, about 2 mg, about 3 mg, about 4 mg, about 5 mg, or about 10 mg, or any amount therebetween; decreasing the dose by about 1 mg, about 2 mg, about 3 mg, about 4 mg, about 5 mg, or about 10 mg, or any amount therebetween; or terminating administration. In some embodiments, titration of the dose comprises maintaining the dose at the current dose; increasing the dose by about 1 mg, about 2 mg, about 3 mg, about 4 mg, about 5 mg, or about 10 mg, or any amount therebetween; or decreasing the dose by about 1 mg, about 2 mg, about 3 mg, about 4 mg, about 5 mg, or about 10 mg, or any amount therebetween.

After a period of time (e.g., about 2 weeks) of the patient being administered the first titrated dose, the dose may be again titrated (i.e., increased, decreased, or maintained) based on the biplane LVEF and/or post-Valsalva LVOT-G of the patient, for example using the same threshold parameters as discussed above. An exemplary dose titration schedule includes: administration of a first titrated dose for about 2 weeks, about 3 weeks, about 4 weeks, about 5 weeks, about 6 weeks, about 8 weeks, about 10 weeks, or about 12 weeks, or any amount of time therebetween, followed by dose titration based on the biplane LVEF and/or post-Valsalva LVOT-G of the patient, for example using the same threshold parameters as discussed above. Further iterations of administration and dose titration may be conducted accordingly.

Provided herein is a method of reducing post-Valsalva LVOT-G to less than a particular value in a patient with symptomatic obstructive hypertrophic cardiomyopathy (oHCM) which comprises administering to the patient a therapeutically effective amount of aficamten, or a pharmaceutically acceptable salt thereof, wherein the patient has been recently diagnosed with oHCM, the patient is treatment naïve, or the patient has chronic oHCM. The reduction in post-Valsalva LVOT-G to less than the particular value may occur within one week, two weeks, three weeks, four weeks, one month, five weeks, six week, seven weeks, eight weeks, two months, nine weeks, or ten weeks of initiating treatment with aficamten, or a pharmaceutically acceptable salt thereof. The reduction in post-Valsalva LVOT-G may be sustained for at least 10 weeks of treatment. In some embodiments, the reduction in post-Valsalva LVOT-G occurs within two to six weeks of the end of dose titration. In some embodiments, the reduction in post-Valsalva LVOT-G peaks within two to six weeks of the end of dose titration. In some embodiments, the particular post-Valsalva LVOT-G value is: 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mmHg.

Further provided herein is a method of treating obstructive hypertrophic cardiomyopathy (oHCM) in a patient with heart failure symptoms, wherein the patient has been recently diagnosed with oHCM, the patient is treatment naïve, or the patient has chronic oHCM, comprising administering to the patient a therapeutically effective amount of aficamten, or a pharmaceutically acceptable salt thereof, wherein the method results in a reduction of heart failure symptoms as assessed by NYHA classification. In some embodiments of the foregoing, the methods improve heart failure symptoms by at least one NYHA class in the patient, for instance, by one or two NYHA class(es). In some embodiments of the foregoing, the methods convert patients from NYHA class III to class II or class I. In some embodiments of the foregoing, the methods convert patients from NYHA class III to class II. In some embodiments of the foregoing, the methods convert patients from NYHA class III to class I. In some embodiments of the foregoing, the methods convert patients from NYHA class II to class I. In some embodiments of the foregoing, reduction of heart failure symptoms occurs within ten weeks of initiating treatment with aficamten, or a pharmaceutically acceptable salt thereof.

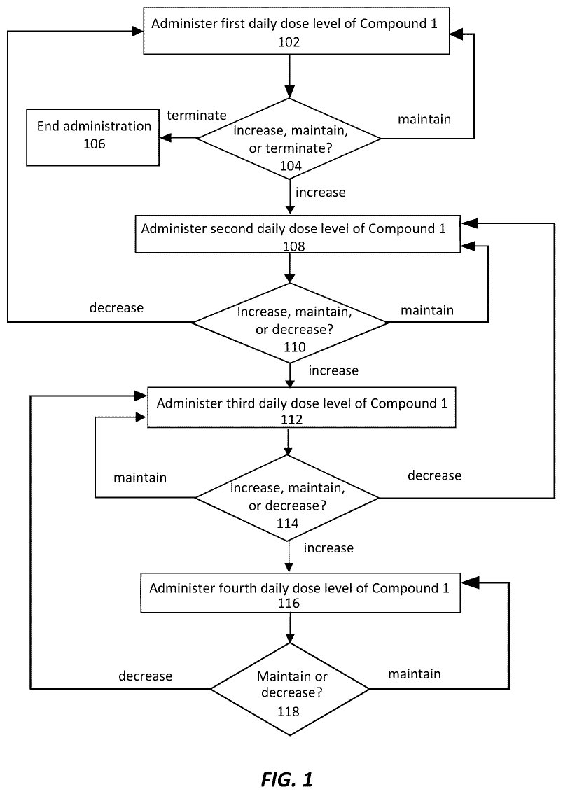

FIG. 1 illustrates and exemplary method for treating obstructive hypertrophic cardiomyopathy (oHCM) in a patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten, that includes titrating the daily dose of aficamten, or a pharmaceutically acceptable salt thereof. The exemplary method shown in FIG. 1 provides four daily dose levels (e.g., about 5 mg, 10 mg, 15 mg, and 20 mg), with a first daily dose level being the lowest daily dose level (e.g., about 5 mg), but may be readily modified to include additional or fewer dose levels. The exemplary method shown in FIG. 1 may be further modified such that the first daily dose level is not the lowest daily dose level. At 102, the first daily dose level (e.g., about 5 mg) of aficamten, or a pharmaceutically acceptable salt thereof, is administered to a patient. After a first time period, the daily dose level is increased or maintained, or the administration is terminated, at 104. The selection may be based on a first echocardiogram acquired for the patient after the first time period. Termination of administration 106 may be selected if the biplane LVEF of the first echocardiogram is below a predetermined biplane LVEF threshold (e.g., 50%), wherein the no further dose of aficamten or pharmaceutically acceptable salt thereof is administered to the patient. Maintenance of the first daily dose level (e.g., about 5 mg) may be selected when either of the following conditions are met on the first echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the first echocardiogram is below a predetermined post-Valsalva LVOT-G threshold (e.g., 30 mmHg). If maintenance is selected, the first daily dose level of aficamten, or a pharmaceutically acceptable salt thereof, is administered to the patient at 102 for a second time period, and, optionally, the daily dose may be re-titrated at 104 after a second time period. The daily dose level may be increased to a second daily dose level (e.g., 10 mg) when either of the following conditions are met on the first echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the first echocardiogram is above a predetermined post-Valsalva LVOT-G threshold (e.g., 30 mmHg). If an increase in the daily dose level is selected, the second daily dose level of aficamten, or a pharmaceutically acceptable salt thereof, is administered to the patient for the second time period at 108.

If the second daily dose level of aficamten, or a pharmaceutically acceptable salt thereof, (e.g., 10 mg) is administered to the patient at 108, the daily dose may be re-titrated (i.e., select an increase, decrease, or maintenance of the daily dose) based on an echocardiogram at 110. The daily dose may be decreased to the first daily dose level (e.g., from about 10 mg to about 5 mg) if the biplane LVEF of the echocardiogram is below a predetermined biplane LVEF threshold (e.g., 50%). If the daily dose is decreased to the first daily dose level, the first daily dose level (e.g., about 5 mg) is administered to the patient at 102. Maintenance of the second daily dose level (e.g., about 10 mg) may be selected when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a predetermined post-Valsalva LVOT-G threshold (e.g., 30 mmHg). If maintenance is selected, the second daily dose level of aficamten, or a pharmaceutically acceptable salt thereof, is administered to the patient at 108 for a further time period, and, optionally, the daily dose may be re-titrated at 110 after said time period. The daily dose level may be increased to a third daily dose level (e.g., 15 mg) when the biplane LVEF is at or above the second predetermined biplane LVEF threshold (e.g., 55%) and the post-Valsalva LVOT-G of the echocardiogram is above a predetermined post-Valsalva LVOT-G threshold (e.g., 30 mmHg). If an increase in the daily dose level is selected, the second daily dose level of aficamten, or a pharmaceutically acceptable salt thereof, is administered to the patient for the time period at 112.

If the third daily dose level of aficamten, or a pharmaceutically acceptable salt thereof, (e.g., 10 mg) is administered to the patient at 112, the daily dose may be re-titrated (i.e., select an increase, decrease, or maintenance of the daily dose) based on an echocardiogram at 114. The daily dose may be decreased to the second daily dose level (e.g., from 15 mg to 10 mg) if the biplane LVEF of the echocardiogram is below a predetermined biplane LVEF threshold (e.g., 50%). If the daily dose is decreased to the second daily dose level, the second daily dose level is administered to the patient at 108. Maintenance of the third daily dose level (e.g., about 15 mg) may be selected when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a predetermined post-Valsalva LVOT-G threshold (e.g., 30 mmHg). If maintenance is selected, the third daily dose level of aficamten, or a pharmaceutically acceptable salt thereof, is administered to the patient at 112 for a further time period, and, optionally, the daily dose may be re-titrated at 114 after said time period. The daily dose level may be increased to a fourth daily dose level (e.g., 20 mg) when the biplane LVEF is at or above the second predetermined biplane LVEF threshold (e.g., 55%) and the post-Valsalva LVOT-G of the echocardiogram is above a second predetermined post-Valsalva LVOT-G threshold (e.g., 30 mmHg). If an increase in the daily dose level is selected, the fourth daily dose level of aficamten, or a pharmaceutically acceptable salt thereof, is administered to the patient for the second time period at 116.

In the exemplary method shown in FIG. 1, the first daily dose level is the minimum dose, and therefore may not be further decreased. Nevertheless, in other embodiments, the first daily dose level may be other than the minimum dose, and therefore may be decreased to a lower dose level (e.g., from 10 mg to 5 mg) if the biplane LVEF of the echocardiogram is below a predetermined biplane LVEF threshold (e.g., 50%).

In the exemplary method shown in FIG. 1, the fourth daily dose level is the maximum dose, and therefore may not be further increased. Nevertheless, in other embodiments, additional dose levels may be available and the daily dose may be further increased at 118. The method shown in FIG. 1, at 118 a selection is made to maintain the fourth daily dose level or decrease the daily dose level based on an echocardiogram. The daily dose may be decreased to the third daily dose level (e.g., from 20 mg to 15 mg) if the biplane LVEF of the echocardiogram is below a predetermined biplane LVEF threshold (e.g., 50%). If the daily dose is decreased to the third daily dose level, the second daily dose level is administered to the patient at 112. Maintenance of the third daily dose level (e.g., about 15 mg) may be selected when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a predetermined post-Valsalva LVOT-G threshold (e.g., 30 mmHg). If maintenance is selected, the third daily dose level of aficamten, or a pharmaceutically acceptable salt thereof, is administered to the patient at 116 for a further time period, and, optionally, the daily dose may be re-titrated at 118 after said time period.

Exemplary daily dose increases include an increase from about 5 mg to about 10 mg aficamten, about 10 mg to about 15 mg aficamten, or about 10 mg to about 20 mg aficamten. Other dose increases may be readily envisioned, for instance, increasing a given initial daily dose by about 1 mg, about 2 mg, about 3 mg, about 4 mg, about 5 mg, or about 10 mg, or any amount therebetween. Exemplary daily dose decreases include a decrease from about 20 mg to about 10 mg, about 15 mg to about 10 mg, or about 10 mg to about 5 mg. Other dose decreases may be readily envisioned, for instance, decreasing a given initial daily dose by about 1 mg, about 2 mg, about 3 mg, about 4 mg, about 5 mg, or about 10 mg, or any amount therebetween.

Exemplary embodiments of the methods described herein comprise administering a first daily dose (e.g., a first daily dose of between about 1 mg and about 20 mg, such as 1 mg, 2 mg, 3 mg, 4 mg, 5 mg, 6 mg, 7 mg, 8 mg, 9 mg, 10 mg, 15 mg, or 20 mg) or any amount therebetween) of aficamten, or a pharmaceutically acceptable salt thereof, for a first period (e.g., about one week, two weeks, three weeks, four weeks, five weeks, six weeks, seven weeks, eight weeks, nine weeks, ten weeks, twelve weeks, or any length of time therebetween), followed by maintaining the daily dose, decreasing the daily dose (e.g., decreasing the daily dose by between about 1 mg and about 10 mg, such as decreasing the daily dose by 1 mg, 2 mg, 3 mg, 4 mg, 5 mg 6 mg, 7 mg, 8 mg, 9 mg, or 10 mg, or any amount therebetween), increasing the daily dose (e.g., increasing the daily dose by between about 1 mg and about 10 mg, such as increasing the daily dose by 1 mg, 2 mg, 3 mg, 4 mg, 5 mg 6 mg, 7 mg, 8 mg, 9 mg, or 10 mg, or any amount therebetween), or terminating administration based on the biplane LVEF and/or post-Valsalva LVOT-G of the patient to arrive at a second daily dose. Another exemplary embodiment of the methods described herein comprises administering a first daily dose of aficamten, or a pharmaceutically acceptable salt thereof, for about two weeks, followed by maintaining the daily dose, decreasing the daily dose by about 5 mg, increasing the daily dose by about 5 mg, or terminating administration based on the biplane LVEF and/or post-Valsalva LVOT-G of the patient to arrive at a second daily dose. Another exemplary embodiment of the methods described herein comprises administering a first daily dose of aficamten, or a pharmaceutically acceptable salt thereof, for about three weeks, followed by maintaining the daily dose, decreasing the daily dose by about 5 mg, increasing the daily dose by about 5 mg, or terminating administration based on the resting LVOT-G, biplane LVEF, and/or post-Valsalva LVOT-G of the patient to arrive at a second daily dose. Another exemplary embodiment of the methods described herein comprises administering a first daily dose of aficamten, or a pharmaceutically acceptable salt thereof, for about two weeks, followed by maintaining the daily dose, decreasing the daily dose by about 10 mg, increasing the daily dose by about 10 mg, or terminating administration based on the biplane LVEF and/or post-Valsalva LVOT-G of the patient to arrive at a second daily dose. Another exemplary embodiment of the methods described herein comprises administering a first daily dose of aficamten, or a pharmaceutically acceptable salt thereof, for about three weeks, followed by maintaining the daily dose, decreasing the daily dose by about 10 mg, increasing the daily dose by about 10 mg, or terminating administration based on the biplane LVEF and/or post-Valsalva LVOT-G of the patient to arrive at a second daily dose. Another exemplary embodiment of the methods described herein comprises administering a first daily dose of aficamten, or a pharmaceutically acceptable salt thereof, for between about 2 and about 12 weeks, followed by maintaining the daily dose, decreasing the daily dose by about 10 mg, increasing the daily dose by about 10 mg, or terminating administration based on the biplane LVEF and/or post-Valsalva LVOT-G of the patient to arrive at a second daily dose.

Treatment for oHCM can result in improved exercise capacity and/or relieve symptoms in patients with hyperdynamic ventricular contraction resulting from obstructive hypertrophic cardiomyopathy. The methods disclosed herein may be useful for treating patients who have been recently diagnosed, are treatment naïve, or have chronic oHCM.

In some embodiments, a therapeutically effective amount of aficamten, or a pharmaceutically acceptable salt thereof, is administered to a patient with obstructive hypertrophic cardiomyopathy, thereby decreasing post-Valsalva LVOT-G in the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In some embodiments, the patient has a baseline post-Valsalva LVOT-G of about 30 mmHg or more, about 40 mmHg or more, 50 mmHg or more, about 60 mmHg or more, or about 70 mmHg or more. In response to administration of the therapeutically effective amount of aficamten, or a pharmaceutically acceptable salt thereof, the post-Valsalva LVOT-G can decrease to less than 50 mmHg, for example to about 45 mmHg or less, about 40 mmHg or less, about 35 mmHg or less, or about 30 mmHg or less. In some embodiments, in response to administration of the therapeutically effective amount of aficamten, or a pharmaceutically acceptable salt thereof, the post-Valsalva LVOT-G decreases by about 10 mmHg or more, about 15 mmHg or more, about 20 mmHg or more, about 25 mmHg or more, about 30 mmHg or more, or about 35 mmHg or more. In some embodiments, in response to administration of the therapeutically effective amount of aficamten, or a pharmaceutically acceptable salt thereof, the post-Valsalva LVOT-G decreases by about 10 mmHg to about 40 mmHg. The decrease in post-Valsalva LVOT-G may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing a left ventricle mass index (LVMI) for the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In response to administration of the therapeutically effective amount of aficamten, or a pharmaceutically acceptable salt thereof, the LVMI can decrease by about 1 g/m² or more, by about 1.5 g/m² or more, by about 2 g/m² or more, by about 2.5 g/m² or more, by about 3 g/m² or more, by about 3.5 g/m² or more, or by about 4 g/m² or more. In some embodiments, in response to administration of the therapeutically effective amount of aficamten, or a pharmaceutically acceptable salt thereof, the LVMI decreases by about 1 g/m² to about 10 g/m², such as by about 1 g/m² to about 6 g/m², or about 2 g/m² to about 5 g/m². The decrease in LVMI may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing a left arterial volume index (LAVI) for the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In response to administration of the therapeutically effective amount of aficamten, or a pharmaceutically acceptable salt thereof, the LAVI can decrease by about 0.5 mL/m² or more, by about 1 mL/m² or more, by about 1.5 mL/m² or more, by about 2 mL/m² or more, or by about 2.5 mL/m² or more. In some embodiments, in response to administration of the therapeutically effective amount of aficamten, or a pharmaceutically acceptable salt thereof, the LAVI decreases by about 0.5 mL/m² to about 5 mL/m² mmHg, such as by about 0.5 mL/m² to about 4 g/m², or about 1 mL/m² to about 3 mL/m². The decrease in LAVI may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing an e′ value for the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In response to administration of the therapeutically effective amount of aficamten, or a pharmaceutically acceptable salt thereof, the e′ value can decrease by about 0.1 cm/s or more, by about 0.15 cm/s or more, by about 0.2 cm/s or more, or by about 0.25 cm/s or more. In some embodiments, in response to administration of the therapeutically effective amount of aficamten, or a pharmaceutically acceptable salt thereof, the e′ value decreases by about 0.05 cm/s to about 0.3 cm/s, such as by about 0.1 cm/s to about 0.25 cm/s, or about 0.15 cm/s to about 0.25 cm/s. The decrease in e′ value may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing a lateral E/e′ ratio for the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In response to administration of the therapeutically effective amount of aficamten, or a pharmaceutically acceptable salt thereof, the lateral E/e′ ratio can decrease by about 0.5 or more, by 1 or more, by about 1.2 or more, by about 1.5 or more, or by about 1.8 or more. In some embodiments, in response to administration of the therapeutically effective amount of aficamten, or a pharmaceutically acceptable salt thereof, the lateral E/e′ ratio decreases by about 0.5 to about 2, such as by about 1 to about 1.8, or about 1.5 to about 1.8. The decrease in lateral E/e′ ratio may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing a level of brain natriuretic peptide or N-terminal prohormone of brain natriuretic peptide (NT-proBNP) in the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. The decrease in a level of brain natriuretic peptide or N-terminal prohormone of brain natriuretic peptide (NT-proBNP) in the patient may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing a level of cardiac troponin I, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. The decrease in a level of cardiac troponin I in the patient may occur after about 1 week, after about 2 weeks, after about 3 weeks, after about 4 weeks, after about 5 weeks, after about 6 weeks, after about 8 weeks, or after about 10 weeks of daily dose administration.

In some embodiments of any of the foregoing, the patient with obstructive hypertrophic cardiomyopathy is classified as NYHA class III when administration with aficamten, or a pharmaceutically acceptable salt thereof, is initiated, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In some embodiments, the patient with obstructive hypertrophic cardiomyopathy is classified as NYHA class II when administration with aficamten, or a pharmaceutically acceptable salt thereof, is initiated.

In some embodiments of any of the foregoing, administration of aficamten, or a pharmaceutically acceptable salt thereof, to a patient with obstructive hypertrophic cardiomyopathy results in an improvement in exercise capacity in the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In some embodiments of any of the foregoing, administration of aficamten, or a pharmaceutically acceptable salt thereof, to a patient with obstructive hypertrophic cardiomyopathy results in an improvement in exercise capacity in the patient, for example as measured by change in peak oxygen uptake (pVO₂) or change in peak oxygen uptake (pVO₂) by cardiopulmonary exercise testing (CPET).

In some embodiments of any of the foregoing, administration of aficamten, or a pharmaceutically acceptable salt thereof, to a patient with obstructive hypertrophic cardiomyopathy results in an improvement in total workload during CPET, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten.

In some embodiments of any of the foregoing, administration of aficamten, or a pharmaceutically acceptable salt thereof, to a patient with obstructive hypertrophic cardiomyopathy results in an improvement in other CPET parameters, including but not limited to one or more of: (1) ventilator efficiency (VE/VCO₂ slope); (2) circulatory power (VO₂×systolic BP); and (3) ventilator anaerobic threshold (VAT), wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten.

In some embodiments of any of the foregoing, administration of aficamten, or a pharmaceutically acceptable salt thereof, to a patient with obstructive hypertrophic cardiomyopathy results in an improvement in patient health status, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-Overall Summary Score (KCCQ-OSS). In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-Clinical Summary Score (KCCQ-CSS). In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-Total Symptom Score (KCCQ-TSS). In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-Physical Limitation Score (KCCQ-PLS). In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-Social Limitation Score (KCCQ-SLS). In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-quality of live (KCCQ-QoL). In some embodiments, administration of aficamten, or a pharmaceutically acceptable salt thereof, to a patient with obstructive hypertrophic cardiomyopathy results in improvement in one or more KCCQ domain scores (such as KCCQ-OSS, KCCQ-CSS, KCCQ-TSS, KCCQ-PLS, KCCQ-SLS, or KCCQ-QoL) by at least about 5 points, at least about 10 points, or at least about 20 points. In some embodiments, administration of aficamten, or a pharmaceutically acceptable salt thereof, to a patient with obstructive hypertrophic cardiomyopathy results in improvement in one or more KCCQ score (such as KCCQ-OSS, KCCQ-CSS, KCCQ-TSS, KCCQ-PLS, KCCQ-SLS, or KCCQ-QoL) by between about 5 points and less than 10 points, between about 10 points and less than 20 points, or by at least 20 points. In some such embodiments, the improvement in the one or more KCCQ domain scores is an improvement in KCCQ-OSS. In some embodiments, the improvement in the one or more KCCQ domain scores is sustained for about 6 months. In some embodiments, administration of aficamten, or a pharmaceutically acceptable salt thereof, to a patient with obstructive hypertrophic cardiomyopathy results in improvement in KCCQ-CSS by 1 point, 2 points, 3 points, 4 points, 5 points, or more than 5 points. In some embodiments of any of the foregoing, administration of aficamten, or a pharmaceutically acceptable salt thereof, to a patient with obstructive hypertrophic cardiomyopathy results in an improvement in patient health status and health-related quality of life as measured by PRO questionnaire, as determined by changes from responses to the EuroQol 5-dimension 5-level instrument (EQ-5D-5L). In some embodiments administration with aficamten, or a pharmaceutically acceptable salt thereof, results in an improvement in one or more HCM symptoms. In some embodiments, improvement in one or more HCM symptoms comprises reduced severity and/or frequency of chest pain, dizziness, shortness of breath, fainting during physical activity, tiredness, low energy, or limitation in physical activity.

Combinations of the foregoing results are also contemplated. For example, in some embodiments of any of the foregoing, administration of aficamten, or a pharmaceutically acceptable salt thereof, to a patient with obstructive hypertrophic cardiomyopathy, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten, results in an improvement in exercise capacity and functional class, for instance, as determined by (1) change from baseline of ≥1.5 mL/kg/min in pVO₂ and >1 class improvement in NYHA Functional Class; or (2) change from baseline of ≥3.0 mL/kg/min in pVO₂ and no worsening of NYHA Functional Class. In some embodiments of any of the foregoing, administration of aficamten, or a pharmaceutically acceptable salt thereof, to a patient with obstructive hypertrophic cardiomyopathy results in resting LVOT-G <30 mmHg, post-Valsalva LVOT-G <50 mmHg, and NYHA Functional Class I in the patient. In some embodiments of any of the foregoing, administration of aficamten, or a pharmaceutically acceptable salt thereof, to a patient with obstructive hypertrophic cardiomyopathy results in resting LVOT-G <30 mmHg, post-Valsalva LVOT-G <50 mmHg, and ≥1 class improvement in NYHA Functional Class in the patient.

In some embodiments, the methods disclosed herein result in one or more effects selected from the group consisting of: reduction in resting LVOT-G to less than 30 mmHg; reduction in post-Valsalva LVOT-G to less than 50 mmHg; improvement in mitral regurgitation; improvement in cardiac relaxation; beneficial cardiac remodeling; reverse cardiac remodeling; beneficial cardiac structural remodeling; beneficial cardiac functional remodeling; reversal of adverse cardiac remodeling; reduction in mean left ventricular mass index (LVMI); improvement in left ventricular (LV) filling pressures; reduction in left atrial volume index (LAVI); reduction in the categorical assessment of systolic anterior motion of the mitral valve leaflet; reduction in systolic anterior motion of the mitral valve leaflet; reduction in the frequency of eccentric mitral regurgitation; reduction in mitral regurgitation; reduction in lateral E/e′; reduction in lateral E/E; reduction in brain natriuretic peptide (BNP) levels; reduction in N-terminal prohormone of brain natriuretic peptide (NT-proBNP) levels; reduction in cardiac troponin I levels; decreased left ventricular wall stress; decreased myocardial injury; and reduction in heart failure symptoms, for example, reduction in NYHA classification.

Methods of Reducing Background Therapy for oHCM

Standard of care (SoC) medications for the treatment of obstructive hypertrophic cardiomyopathy (oHCM) are currently recommended as first-line therapy. Standard of care medications include one or more therapeutics selected from a beta-blocker, a calcium channel blocker, disopyramide and ranolazine. There are however off-target side-effects that often render them unappealing for patients. The off-target side-effects may include hypotension, bradycardia, fatigue, insomnia, and sexual dysfunction. Background therapy reduction and/or withdrawal (BTR/W) can be advantageous to the patient. Withdrawal of beta-blocker treatment in patients with heart failure with preserved ejection fraction (HFpEF) and chronotropic incompetence has been shown to provide a functional benefit of improved pVO₂ and health status benefit measured by Minnesota Living with Heart Failure Questionnaire (MLHFQ) score (Palau et al., Clin. Cardiol. 2020: 45(5):423-429).

In some aspects, provided are methods of treating symptomatic obstructive hypertrophic cardiomyopathy (oHCM) in a patient in need thereof, wherein the patient is receiving one or more background therapies for oHCM. In some embodiments, provided are methods of treating symptomatic obstructive hypertrophic cardiomyopathy (oHCM) in a patient in need thereof, wherein the patient is receiving background therapy for oHCM, comprising (1) administering to the patient a therapeutically effective amount of aficamten, or a pharmaceutically acceptable salt thereof, and (2) reducing and/or stopping at least one background therapy. In some embodiments, the patient is receiving one, two, or three background therapies for oHCM. In some embodiments, the method comprises reducing at least one background therapy. In some embodiments, the method comprises reducing all of the one or more background therapies. In some embodiments, the method comprises stopping at least one background therapy. In some embodiments, the method comprises stopping all of the one or more background therapies. In some embodiments, at least one of the background therapies comprises administering at least one standard-of-care therapeutic selected from the group consisting of beta blockers, non-dihydropyridine calcium channel blockers, and disopyramide. In some embodiments, the patient is administered one or more of a beta-blocker, a non-dihydropyridine calcium channel blocker, and disopyramide, as background therapies. In some embodiments, reducing at least one background therapy comprises administering a lower dose of the at least one background therapy. In some embodiments, the lower dose is 50% or less than the original dose of the background therapy. In some embodiments, (i) initiating administering to the patient a therapeutically effective amount of aficamten, or a pharmaceutically acceptable salt thereof, and (ii) reducing and/or stopping at least one background therapy, are conducted simultaneously. In some embodiments, (i) initiating administering to the patient a therapeutically effective amount of aficamten, or a pharmaceutically acceptable salt thereof, and (ii) reducing and/or stopping at least one background therapy, are conducted sequentially. In some embodiments, reducing and/or stopping at least one background therapy is conducted upon the beginning of the administration of aficamten, or a pharmaceutically acceptable salt thereof. In some embodiments, reducing and/or stopping the at least one background therapy is conducted 2 weeks or more after the beginning of the administration of aficamten, or a pharmaceutically acceptable salt thereof, for example, 2 weeks, 3 weeks, 4 weeks, 5 weeks, 8 weeks, 10 weeks, 12 weeks, 15 weeks, 18 weeks, or 24 weeks after the beginning of the administration of aficamten, or a pharmaceutically acceptable salt thereof, or any amount therebetween. In some embodiments, reducing and/or stopping the at least one background therapy is conducted 12 weeks or more after the beginning of the administration of aficamten, or a pharmaceutically acceptable salt thereof. In some embodiments, reducing and/or stopping the at least one background therapy is conducted when the patient has received a stable dose of aficamten, or a pharmaceutically acceptable salt thereof, for 2 weeks or more, for example, 2 weeks, 3 weeks, 4 weeks, 5 weeks, 8 weeks, 10 weeks, 12 weeks, 15 weeks, 18 weeks, or 24 weeks after the beginning of the administration of aficamten, or a pharmaceutically acceptable salt thereof, or any amount therebetween. In some embodiments, reducing and/or stopping the at least one background therapy is conducted when the patient has received a stable dose of aficamten, or a pharmaceutically acceptable salt thereof, for 4 weeks or more, for example, 4 weeks, 5 weeks, 8 weeks, 10 weeks, 12 weeks, 15 weeks, 18 weeks, or 24 weeks after the beginning of the administration of aficamten, or a pharmaceutically acceptable salt thereof, or any amount therebetween.

In another aspect, provided are methods of treating symptomatic obstructive hypertrophic cardiomyopathy (oHCM) in a patient in need thereof, comprising: (1) administering to the patient a therapeutically effective amount of aficamten, or a pharmaceutically acceptable salt thereof, in combination with a first dose of a standard-of-care therapeutic for oHCM, for a first time period; and (2) administering to the patient a therapeutically effective amount of aficamten, or a pharmaceutically acceptable salt thereof, in combination with a second dose of the standard-of-care therapeutic for oHCM, for a second time period. In some embodiments, the second dose of the standard-of-care therapeutic for oHCM is lower than the first dose of the standard-of-care therapeutic. In some embodiments, the second dose of the standard-of-care therapeutic for oHCM is 50% of the first dose of the standard-of-care therapeutic, or lower. In some embodiments, the first time period is at least 12 weeks. In some embodiments, aficamten, or a pharmaceutically acceptable salt thereof, is administered at a constant amount for at least about four weeks before the start of the second time period.

In another aspect, provided are methods of treating symptomatic obstructive hypertrophic cardiomyopathy (oHCM) in a patient in need thereof, comprising: (1) administering to the patient a therapeutically effective amount of aficamten, or a pharmaceutically acceptable salt thereof, in combination with a standard-of-care therapeutic for oHCM, for a first time period; and (2) administering to the patient a therapeutically effective amount of aficamten, or a pharmaceutically acceptable salt thereof, as a monotherapy, for a second time period. In some embodiments, the second dose of the standard-of-care therapeutic for oHCM is 50% of the first dose of the standard-of-care therapeutic, or lower. In some embodiments, the first time period is at least 12 weeks. In some embodiments, aficamten, or a pharmaceutically acceptable salt thereof, is administered at a constant amount for at least about four weeks before the start of the second time period.

In another aspect, provided are methods of treating symptomatic obstructive hypertrophic cardiomyopathy (oHCM) in a patient in need thereof, comprising: (1) administering to the patient a background therapy comprising at least one standard-of-care therapeutic selected from the group consisting of beta blockers, non-dihydropyridine calcium channel blockers, and disopyramide; (2) administering to the patient a therapeutically effective amount of aficamten, or a pharmaceutically acceptable salt thereof, in combination with the background therapy, for a first time period; and (3) administering to the patient a therapeutically effective amount of aficamten, or a pharmaceutically acceptable salt thereof, in combination with a second dose of the standard-of-care therapeutic for oHCM, for a second time period, wherein the second dose of the standard-of-care therapeutic for oHCM is lower than the first dose of the standard-of-care therapeutic. In some embodiments, the second dose of the standard-of-care therapeutic for oHCM is 50% of the first dose of the standard-of-care therapeutic, or lower. In some embodiments, the first time period is at least 12 weeks. In some embodiments, aficamten, or a pharmaceutically acceptable salt thereof, is administered at a constant amount for at least about four weeks before the start of the second time period.

In another aspect, provided are methods of treating symptomatic obstructive hypertrophic cardiomyopathy (oHCM) in a patient in need thereof, comprising: (1) administering to the patient a background therapy comprising at least one standard-of-care therapeutic selected from the group consisting of beta blockers, non-dihydropyridine calcium channel blockers, and disopyramide; (2) administering to the patient a therapeutically effective amount of aficamten, or a pharmaceutically acceptable salt thereof, in combination with the background therapy, for a first time period; and (3) administering to the patient a therapeutically effective amount of aficamten, or a pharmaceutically acceptable salt thereof, as a monotherapy, for a second time period. In some embodiments, the first time period is at least 12 weeks. In some embodiments, aficamten, or a pharmaceutically acceptable salt thereof, is administered at a constant amount for at least about four weeks before the start of the second time period.

In another aspect, provided are methods of improving myocardial mechanics in a patient in need thereof, comprising administering to the patient a therapeutically effective amount of aficamten, or a pharmaceutically acceptable salt thereof. In some aspects, provided are methods of improving myocardial mechanics in a patient having obstructive hypertrophic cardiomyopathy, comprising administering to the patient a therapeutically effective amount of aficamten, or a pharmaceutically acceptable salt thereof. In some embodiments, the method comprises long-term administration of aficamten or a pharmaceutically acceptable salt thereof. For example, in some embodiments, the method comprises administering aficamten for at least about 12 weeks, at least about 24 weeks, at least about 36 weeks, at least about 48 weeks, at least about 1 year, at least about 2 years, at least about 3 years, at least about 4 years, at least about 5 years, or at least about 10 years. In some embodiments, the method comprises administering aficamten for at least about 12 weeks. In some embodiments, the method comprises administering aficamten for at least about 36 weeks. In some embodiments, the method comprises administering aficamten for at least about 48 weeks. In some embodiments, the method further results in an improvement in resting LVOT-G. In some embodiments, the method results in a resting LVOT-G of less than 30 mm Hg. In some embodiments, the method further results in an improvement in Valsalva LVOT-G. In some embodiments, the method results in a Valsalva LVOT-G of less than 50 mm Hg. In some embodiments, the method comprises administering aficamten, or a pharmaceutically acceptable salt thereof, to a patient having an optimal hemodynamic response to treatment with aficamten, or a pharmaceutically acceptable salt thereof. For example, in some embodiments, the method comprises administering aficamten to a patient having a resting LVOT-G of less than 30 mmHg. In some embodiments, the method comprises administering aficamten to a patient having a Valsalva LVOT-G of less than 50 mmHg. In some embodiments, the patient has a resting LVOT-G of less than 30 mmHg as a result of the treatment with aficamten, or a pharmaceutically acceptable salt thereof. In some embodiments, the patient has a Valsalva LVOT-G of less than 50 mmHg as a result of the treatment with aficamten, or a pharmaceutically acceptable salt thereof. In some embodiments, the patient has an optimal hemodynamic response to treatment with aficamten, or a pharmaceutically acceptable salt thereof. In some embodiments, the improvement in myocardial mechanics occurs after the patient has an optimal hemodynamic response to treatment with aficamten, or a pharmaceutically acceptable salt thereof. In some embodiments, the improvement in myocardial mechanics occurs about 4 weeks, about 6 weeks, about 8 weeks, about 12 weeks, or about 36 weeks (or any amount of weeks therebetween) after the patient achieves a resting LVOT-G of less than 30 mmHg and/or a Valsalva LVOT-G of less than 50 mmHg in response to treatment with aficamten or a pharmaceutically acceptable salt thereof.

In another aspect, provided are methods of improving global longitudinal strain (GLS) in a patient in need thereof, comprising administering to the patient a therapeutically effective amount of aficamten, or a pharmaceutically acceptable salt thereof. In some aspects, provided are methods of improving global longitudinal strain (GLS) in a patient having obstructive hypertrophic cardiomyopathy, comprising administering to the patient a therapeutically effective amount of aficamten, or a pharmaceutically acceptable salt thereof. In some embodiments, the method comprises long-term administration of aficamten or a pharmaceutically acceptable salt thereof. For example, in some embodiments, the method comprises administering aficamten for at least about 12 weeks, at least about 24 weeks, at least about 36 weeks, at least about 48 weeks, at least about 1 year, at least about 2 years, at least about 3 years, at least about 4 years, at least about 5 years, or at least about 10 years. In some embodiments, the method comprises administering aficamten for at least about 12 weeks. In some embodiments, the method comprises administering aficamten for at least about 36 weeks. In some embodiments, the method comprises administering aficamten for at least about 48 weeks. In some embodiments, the method further results in an improvement in resting LVOT-G. In some embodiments, the method results in a resting LVOT-G of less than 30 mm Hg. In some embodiments, the method further results in an improvement in Valsalva LVOT-G. In some embodiments, the method results in a Valsalva LVOT-G of less than 50 mm Hg. In some embodiments, the method comprises administering aficamten, or a pharmaceutically acceptable salt thereof, to a patient having an optimal hemodynamic response to treatment with aficamten, or a pharmaceutically acceptable salt thereof. For example, in some embodiments, the method comprises administering aficamten to a patient having a resting LVOT-G of less than 30 mmHg. In some embodiments, the method comprises administering aficamten to a patient having a Valsalva LVOT-G of less than 50 mmHg. In some embodiments, the patient has a resting LVOT-G of less than 30 mmHg as a result of the treatment with aficamten, or a pharmaceutically acceptable salt thereof. In some embodiments, the patient has a Valsalva LVOT-G of less than 50 mmHg as a result of the treatment with aficamten, or a pharmaceutically acceptable salt thereof. In some embodiments, the patient has an optimal hemodynamic response to treatment with aficamten, or a pharmaceutically acceptable salt thereof. In some embodiments, the improvement in GLS occurs after the patient has an optimal hemodynamic response to treatment with aficamten, or a pharmaceutically acceptable salt thereof. In some embodiments, the improvement in GLS occurs about 4 weeks, about 6 weeks, about 8 weeks, about 12 weeks, or about 36 weeks (or any amount of weeks therebetween) after the patient achieves a resting LVOT-G of less than 30 mmHg and/or a Valsalva LVOT-G of less than 50 mmHg in response to treatment with aficamten or a pharmaceutically acceptable salt thereof.

In some embodiments of any of the foregoing, the therapeutically effective amount of aficamten, or a pharmaceutically acceptable salt thereof, is selected by titrating a daily dose of aficamten, or a pharmaceutically acceptable salt thereof, administered to the patient, as described herein with respect to the methods of treating oHCM.

In some embodiments, the patient has a comorbidity. In some embodiments, the patient has one or more of hypertension, diabetes, permanent atrial fibrillation, and paroxysmal atrial fibrillation.

Administration of the compounds and compositions disclosed and/or described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compound or composition is administered orally or intravenously. In some embodiments, the compound or composition disclosed and/or described herein is administered orally. In some embodiments, the compound or composition disclosed and/or described herein is administered by injection. In some embodiments, the compound or composition disclosed and/or described herein is administered intranasally. In some embodiments, the compound or composition disclosed and/or described herein is administered transdermally.

Pharmaceutically acceptable compositions include solid, semi-solid, liquid and aerosol dosage forms, such as tablet, capsule, powder, liquid, suspension, suppository, and aerosol forms. The compounds disclosed and/or described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

Aficamten can be administered either alone or in combination with one or more conventional pharmaceutical carriers or excipients (e.g., mannitol, lactose, starch, magnesium stearate, sodium saccharine, talcum, cellulose, sodium croscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, see Remington's Pharmaceutical Sciences, Mack Publishing Company, Easton, Pennsylvania. Suitable formulations of aficamten are disclosed in WO 2021/011808, which is incorporated by reference herein in its entirety.

In some embodiments, aficamten is provided in a formulation comprising: (i) aficamten or a pharmaceutically acceptable salt thereof; (ii) a filler; (iii) a binder; (iv) a disintegrant; (v) a surfactant; and (vi) a lubricant. In some embodiments, aficamten is provided in a formulation comprising: (i) aficamten; (ii) a filler; (iii) a binder; (iv) a disintegrant; (v) a surfactant; and (vi) a lubricant. In some embodiments, the filler is selected from the group consisting of powdered cellulose, microcrystalline cellulose, silicified microcrystalline cellulose, kaolin, corn starch, maize starch, starch derivatives, pregelatinized starch, calcium phosphate, calcium hydrogen phosphate, dicalcium phosphate, tricalcium phosphate, compressible sugar, sugar alcohol, mannitol, sorbitol, maltitol, xylitol, lactitol, lactose, dextrose, maltose, sucrose, glucose, fructose, saccharose, raffinose, dextrates, trehalose, maltodextrines, and mixtures of any of the foregoing.

In some embodiments, the binder is selected from the group consisting of arabic gum, acacia gum, alginate, alginic acid, corn starch, copolyvidone, polyvinylpyrrolidone, gelatin, glyceryl behenate, hydroxyethylcellulose, hydroxypropyl cellulose, carboxymethylcellulose, carboxymethylcellulose calcium, carboxymethylcellulose sodium, methylcellulose, hypromellose, lactose, polyvinyl alcohol, povidone, polyethylene oxide, polyacrylates, potato starch, pregelatinized starch, sodium alginate, sodium starch, sodium carboxy methyl cellulose, starch, and mixtures of any of the foregoing.

In some embodiments, the disintegrant is selected from the group consisting of alginic acid, croscarmellose sodium, cellulose, carboxymethylcellulose calcium, carboxymethylcellulose sodium, microcrystalline cellulose, crospovidone, sodium starch glycolate, low-substituted hydroxypropyl cellulose, polacrillin potassium, pregelatinized starch, partially hydrolyzed starch, sodium carboxymethyl starch, starch, sodium alginate, sodium carboxy methyl cellulose, and mixtures of any of the foregoing.

In some embodiments, the surfactant is selected from the group consisting of cetylpyridine chloride, heptadecaethylene oxycetanol, lecithins, polyoxyethylene stearate, nonoxynol 9, nonoxynol 10, octoxynol 9, sorbitan fatty acid esters, Span 20, Span 40, Span 60, Span 80, Span 85, polysorbates, polysorbate 20, polysorbate 21, polysorbate 40, polysorbate 60, polysorbate 61, polysorbate 65, polysorbate 80, sodium salts of fatty alcohol sulfates, sodium lauryl sulfate, sodium salts of sulfosuccinates, sodium dioctylsulfosuccinate, partial esters of fatty acids with alcohols, glycerine monostearate, glyceryl monooleate, ethers of fatty alcohols with polyoxyethylene, esters of fatty acids with polyoxyethylene, copolymers of ethylenoxide and propylenoxide (Pluronic®), benzalkonium chloride, ethoxylated triglycerides, and mixtures of any of the foregoing.

In some embodiments, the lubricant is selected from the group consisting of hydrogenated castor oil, magnesium stearate, glyceryl monostearate, calcium stearate, glyceryl behenate, glycerol distearate, glyceryl dipalmitostearate, behenoyl polyoxyl-8 glycerides, sodium stearyl fumarate, stearic acid, talc, zinc stearate, mineral oil, polyethylene glycol, polaxamer, sodium lauryl sulfate, and mixtures of any of the foregoing.

In some embodiments, the formulation comprises: (i) about 1% by weight to about 80% by weight of the aficamten or a pharmaceutically acceptable salt thereof; (ii) about 15% by weight to about 90% by weight of the filler; (iii) about 0.1% by weight to about 10% by weight of the binder; (iv) about 1% by weight to about 10% by weight of the disintegrant; (v) about 0.1% by weight to about 10% by weight of the surfactant; and (vi) about 0.1% by weight to about 10% by weight of the lubricant.

In some embodiments, the formulation comprises: (i) about 1% by weight to about 50% by weight of the aficamten or pharmaceutically acceptable salt thereof; (ii) about 40% by weight to about 80% by weight of the filler; (iii) about 0.5% by weight to about 5% by weight of the binder; (iv) about 2% by weight to about 8% by weight of the disintegrant; (v) about 0.5% by weight to about 5% by weight of the surfactant; and (vi) about 0.5% by weight to about 5% by weight of the lubricant.

In some embodiments, the formulation comprises: (i) about 10% by weight to about 30% by weight of the aficamten, or pharmaceutically acceptable salt thereof; (ii) about 60% by weight to about 80% by weight of the filler; (iii) about 1% by weight to about 3% by weight of the binder; (iv) about 4% by weight to about 6% by weight of the disintegrant; (v) about 1% by weight to about 3% by weight of the surfactant; and (vi) about 0.5% by weight to about 1.5% by weight of the lubricant.

In some embodiments, the formulation comprises: (i) about 1% by weight to about 10% by weight of the aficamten, or pharmaceutically acceptable salt thereof; (ii) about 70% by weight to about 90% by weight of the filler; (iii) about 1% by weight to about 3% by weight of the binder; (iv) about 4% by weight to about 6% by weight of the disintegrant; (v) about 1% by weight to about 3% by weight of the surfactant; and (vi) about 0.5% by weight to about 1.5% by weight of the lubricant.

In some embodiments, the formulation comprises: (i) about 5% by weight of the aficamten, or pharmaceutically acceptable salt thereof; (ii) about 85% by weight of the filler; (iii) about 2% by weight of the binder; (iv) about 5% by weight of the disintegrant; (v) about 2% by weight of the surfactant; and (vi) about 1% by weight of the lubricant.

In some embodiments, the formulation comprises: (i) about 10% by weight of the aficamten, or pharmaceutically acceptable salt thereof; (ii) about 80% by weight of the filler; (iii) about 2% by weight of the binder; (iv) about 5% by weight of the disintegrant; (v) about 2% by weight of the surfactant; and (vi) about 1% by weight of the lubricant.

In some embodiments, the formulation comprises: (i) about 1% by weight to about 50% by weight of aficamten, or pharmaceutically acceptable salt thereof; (ii-1) about 10% by weight to about 60% by weight of mannitol; (ii-2) about 5% by weight to about 45% by weight of microcrystalline cellulose; (iii) about 0.1% by weight to about 10% by weight of hydroxypropyl cellulose; (iv) about 1% by weight to about 10% by weight of croscarmellose sodium; (v) about 0.1% by weight to about 10% by weight of sodium lauryl sulfate; and (vi) about 0.1% by weight to about 10% by weight of magnesium stearate.

In some embodiments, the formulation comprises: (i) about 10% by weight to about 30% by weight of the aficamten, or pharmaceutically acceptable salt thereof; (ii-1) about 40% by weight to about 50% by weight of mannitol; (ii-2) about 20% by weight to about 30% by weight of microcrystalline cellulose; (iii) about 1% by weight to about 3% by weight of hydroxypropyl cellulose; (iv) about 4% by weight to about 6% by weight of croscarmellose sodium; (v) about 1% by weight to about 3% by weight of sodium lauryl sulfate; and (vi) about 0.5% by weight to about 1.5% by weight of magnesium stearate.

In some embodiments, the formulation comprises: (i) about 1% by weight to about 10% by weight of aficamten, or pharmaceutically acceptable salt thereof; (ii-1) about 50% by weight to about 60% by weight of mannitol; (ii-2) about 25% by weight to about 35% by weight of microcrystalline cellulose; (iii) about 1% by weight to about 3% by weight of hydroxypropyl cellulose; (iv) about 4% by weight to about 6% by weight of croscarmellose sodium; (v) about 1% by weight to about 3% by weight of sodium lauryl sulfate; and (vi) about 0.5% by weight to about 1.5% by weight of the magnesium stearate.

In some embodiments, the formulation comprises: (i) about 20% by weight of the aficamten, or pharmaceutically acceptable salt thereof; (ii-1) about 44% by weight of mannitol; (ii-2) about 26% by weight of microcrystalline cellulose; (iii) about 2% by weight of hydroxypropyl cellulose; (iv) about 5% by weight of croscarmellose sodium; (v) about 2% by weight of sodium lauryl sulfate; and (vi) about 1% by weight of magnesium stearate.

In some embodiments, the formulation comprises: (i) about 10% by weight of the aficamten, or pharmaceutically acceptable salt thereof; (ii-1) about 50% by weight of mannitol; (ii-2) about 30% by weight of microcrystalline cellulose; (iii) about 2% by weight of hydroxypropyl cellulose; (iv) about 5% by weight of croscarmellose sodium; (v) about 2% by weight of sodium lauryl sulfate; and (vi) about 1% by weight of magnesium stearate.

In some embodiments, the formulation comprises: (i) about 5% by weight of the aficamten, or pharmaceutically acceptable salt thereof; (ii-1) about 54% by weight of mannitol; (ii-2) about 31% by weight of microcrystalline cellulose; (iii) about 2% by weight of hydroxypropyl cellulose; (iv) about 5% by weight of croscarmellose sodium; (v) about 2% by weight of sodium lauryl sulfate; and (vi) about 1% by weight of the magnesium stearate.

In some embodiments, the aforementioned formulations are in the form of a tablet. In some embodiments, the tablet further comprises a coating (e.g., a film coating as described elsewhere herein). In such embodiments, the weight percentages herein are provided with respect to the core tablet, not including the weight of the coating.

In some embodiments, aficamten or pharmaceutical composition containing aficamten will take the form of a pill or tablet and thus the composition may contain, along with a compounds disclosed and/or described herein, one or more of a diluent (e.g., lactose, sucrose, dicalcium phosphate), a lubricant (e.g., magnesium stearate), and/or a binder (e.g., starch, gum acacia, polyvinylpyrrolidine, gelatin, cellulose, cellulose derivatives). Other solid dosage forms include a powder, marume, solution or suspension (e.g., in propylene carbonate, vegetable oils or triglycerides) encapsulated in a gelatin capsule.

In some embodiments, aficamten is provided in a tablet comprising: (i) a core having a total core weight comprising: (a) an intra-granular component comprising: (a-i) aficamten, or pharmaceutically acceptable salt thereof; (a-ii) an intra-granular filler; (a-iii) an intra-granular binder; (a-iv) an intra-granular disintegrant; and (a-v) an intra-granular surfactant; and (b) an extra-granular component comprising: (b-i) an extra-granular filler; (b-ii) an extra-granular disintegrant; and (b-iii) an extra-granular lubricant; and optionally (ii) a coating layer comprising a coating agent.

In some embodiments, the intra-granular filler is selected from the group consisting of powdered cellulose, microcrystalline cellulose, silicified microcrystalline cellulose, kaolin, corn starch, maize starch, starch derivatives, pregelatinized starch, calcium phosphate, calcium hydrogen phosphate, dicalcium phosphate, tricalcium phosphate, compressible sugar, sugar alcohol, mannitol, sorbitol, maltitol, xylitol, lactitol, lactose, dextrose, maltose, sucrose, glucose, fructose, saccharose, raffinose, dextrates, trehalose, maltodextrines, and mixtures of any of the foregoing.

In some embodiments, the intra-granular binder is selected from the group consisting of arabic gum, acacia gum, alginate, alginic acid, corn starch, copolyvidone, polyvinylpyrrolidone, gelatin, glyceryl behenate, hydroxyethylcellulose, hydroxypropyl cellulose, carboxymethylcellulose, carboxymethylcellulose calcium, carboxymethylcellulose sodium, methylcellulose, hypromellose, lactose, polyvinyl alcohol, povidone, polyethylene oxide, polyacrylates, potato starch, pregelatinized starch, sodium alginate, sodium starch, sodium carboxy methyl cellulose, starch, and mixtures of any of the foregoing.

In some embodiments, the intra-granular disintegrant is selected from the group consisting of alginic acid, croscarmellose sodium, cellulose, carboxymethylcellulose calcium, carboxymethylcellulose sodium, microcrystalline cellulose, crospovidone, sodium starch glycolate, low-substituted hydroxypropyl cellulose, polacrillin potassium, pregelatinized starch, partially hydrolyzed starch, sodium carboxymethyl starch, starch, sodium alginate, sodium carboxy methyl cellulose, and mixtures of any of the foregoing.

In some embodiments, the intra-granular surfactant is selected from the group consisting of cetylpyridine chloride, heptadecaethylene oxycetanol, lecithins, polyoxyethylene stearate, nonoxynol 9, nonoxynol 10, octoxynol 9, sorbitan fatty acid esters, Span 20, Span 40, Span 60, Span 80, Span 85, polysorbates, polysorbate 20, polysorbate 21, polysorbate 40, polysorbate 60, polysorbate 61, polysorbate 65, polysorbate 80, sodium salts of fatty alcohol sulfates, sodium lauryl sulfate, sodium salts of sulfosuccinates, sodium dioctylsulfosuccinate, partial esters of fatty acids with alcohols, glycerine monostearate, glyceryl monooleate, ethers of fatty alcohols with polyoxyethylene, esters of fatty acids with polyoxyethylene, copolymers of ethylenoxide and propylenoxide (Pluronic®), benzalkonium chloride, ethoxylated triglycerides, and mixtures of any of the foregoing.

In some embodiments, the extra-granular filler is selected from the group consisting of powdered cellulose, microcrystalline cellulose, silicified microcrystalline cellulose, kaolin, corn starch, maize starch, starch derivatives, pregelatinized starch, calcium phosphate, calcium hydrogen phosphate, dicalcium phosphate, tricalcium phosphate, compressible sugar, sugar alcohol, mannitol, sorbitol, maltitol, xylitol, lactitol, lactose, dextrose, maltose, sucrose, glucose, fructose, saccharose, raffinose, dextrates, trehalose, maltodextrines, and mixtures of any of the foregoing.

In some embodiments, the extra-granular disintegrant is selected from the group consisting of alginic acid, croscarmellose sodium, cellulose, carboxymethylcellulose calcium, carboxymethylcellulose sodium, microcrystalline cellulose, crospovidone, sodium starch glycolate, low-substituted hydroxypropyl cellulose, polacrillin potassium, pregelatinized starch, partially hydrolyzed starch, sodium carboxymethyl starch, starch, sodium alginate, sodium carboxy methyl cellulose, and mixtures of any of the foregoing.

In some embodiments, the extra-granular lubricant is selected from the group consisting of hydrogenated castor oil, magnesium stearate, glyceryl monostearate, calcium stearate, glyceryl behenate, glycerol distearate, glyceryl dipalmitostearate, behenoyl polyoxyl-8 glycerides, sodium stearyl fumarate, stearic acid, talc, zinc stearate, mineral oil, polyethylene glycol, polaxamer, sodium lauryl sulfate, and mixtures of any of the foregoing.

In some embodiments, the core comprises: (a) an intra-granular component comprising: (a-i) about 1% to about 80% of the total core weight of the aficamten, or pharmaceutically acceptable salt thereof; (a-ii) about 10% to about 80% of the total core weight of the intra-granular filler; (a-iii) about 0.1% to about 10% of the total core weight of the intra-granular binder; (a-iv) about 0.1% to about 5% of the total core weight of the intra-granular disintegrant; and (a-v) about 0.1% to about 5% of the total core weight of the intra-granular surfactant; and (b) an extra-granular component comprising: (b-i) about 5% to about 15% of the total core weight of the extra-granular filler; (b-ii) about 0.1% to about 5% of the total core weight of the extra-granular disintegrant; and (b-iii) about 0.1% to about 5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 80% of the total core weight of the aficamten, or pharmaceutically acceptable salt thereof; (a-ii) about 10% to about 80% of the total core weight of the intra-granular filler; (a-iii) about 0.1% to about 10% of the total core weight of the intra-granular binder; (a-iv) about 0.1% to about 5% of the total core weight of the intra-granular disintegrant; and (a-v) about 0.1% to about 5% of the total core weight of the intra-granular surfactant; and (b) an extra-granular component comprising: (b-i) about 5% to about 15% of the total core weight of the extra-granular filler; (b-ii) about 0.1% to about 5% of the total core weight of the extra-granular disintegrant; and (b-iii) about 0.1% to about 5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 50% of the total core weight of the aficamten, or pharmaceutically acceptable salt thereof; (a-ii) about 40% to about 80% of the total core weight of the intra-granular filler; (a-iii) about 1% to about 5% of the total core weight of the intra-granular binder; (a-iv) about 1% to about 5% of the total core weight of the intra-granular disintegrant; and (a-v) about 1% to about 5% of the total core weight of the intra-granular surfactant; and (b) an extra-granular component comprising: (b-i) about 5% to about 15% of the total core weight of the extra-granular filler; (b-ii) about 1% to about 5% of the total core weight of the extra-granular disintegrant; and (b-iii) about 0.1% to about 2% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% to about 30% of the total core weight of the aficamten, or pharmaceutically acceptable salt thereof; (a-ii) about 50% to about 70% of the total core weight of the intra-granular filler; (a-iii) about 1% to about 3% of the total core weight of the intra-granular binder; (a-iv) about 2% to about 4% of the total core weight of the intra-granular disintegrant; and (a-v) about 1% to about 3% of the total core weight of the intra-granular surfactant; and (b) an extra-granular component comprising: (b-i) about 5% to about 15% of the total core weight of the an extra-granular filler; (b-ii) about 1% to about 3% of the total core weight of the extra-granular disintegrant; and (b-iii) about 0.1% to about 1.5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 10% of the total core weight of the aficamten, or pharmaceutically acceptable salt thereof; (a-ii) about 60% to about 80% of the total core weight of the intra-granular filler; (a-iii) about 1% to about 3% of the total core weight of the intra-granular binder; (a-iv) about 2% to about 4% of the total core weight of the intra-granular disintegrant; and (a-v) about 1% to about 3% of the total core weight of the intra-granular surfactant; and (b) an extra-granular component comprising: (b-i) about 5% to about 15% of the total core weight of the an extra-granular filler; (b-ii) about 1% to about 3% of the total core weight of the extra-granular disintegrant; and (b-iii) about 0.1% to about 1.5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 5% of the total core weight of the aficamten, or pharmaceutically acceptable salt thereof; (a-ii) about 74% of the total core weight of the intra-granular filler; (a-iii) about 2% of the total core weight of the intra-granular binder; (a-iv) about 3% of the total core weight of the intra-granular disintegrant; and (a-v) about 2% of the total core weight of the intra-granular surfactant; and (b) an extra-granular component comprising: (b-i) about 11% of the total core weight of the an extra-granular filler; (b-ii) about 2% of the total core weight of the extra-granular disintegrant; and (b-iii) about 1% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% of the total core weight of the aficamten, or pharmaceutically acceptable salt thereof; (a-ii) about 69% of the total core weight of the intra-granular filler; (a-iii) about 2% of the total core weight of the intra-granular binder; (a-iv) about 3% of the total core weight of the intra-granular disintegrant; and (a-v) about 2% of the total core weight of the intra-granular surfactant; and (b) an extra-granular component comprising: (b-i) about 11% of the total core weight of the an extra-granular filler; (b-ii) about 2% of the total core weight of the extra-granular disintegrant; and (b-iii) about 1% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 20% of the total core weight of the aficamten, or pharmaceutically acceptable salt thereof; (a-ii) about 59% of the total core weight of the intra-granular filler; (a-iii) about 2% of the total core weight of the intra-granular binder; (a-iv) about 3% of the total core weight of the intra-granular disintegrant; and (a-v) about 2% of the total core weight of the intra-granular surfactant; and (b) an extra-granular component comprising: (b-i) about 11% of the total core weight of the an extra-granular filler; (b-ii) about 2% of the total core weight of the extra-granular disintegrant; and (b-iii) about 1% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 50% of the total core weight of the aficamten, or pharmaceutically acceptable salt thereof; (a-ii-1) about 40% to about 60% of the total core weight of mannitol; (a-ii-2) about 10% to about 30% of the total core weight of microcrystalline cellulose; (a-iii) about 1% to about 5% of the total core weight of hydroxypropyl cellulose; (a-iv) about 1% to about 5% of the total core weight of croscarmellose sodium; and (a-v) about 1% to about 5% of the total core weight of sodium lauryl sulfate; and (b) an extra-granular component comprising: (b-i) about 5% to about 15% of the total core weight of microcrystalline cellulose; (b-ii) about 1% to about 5% of the total core weight of croscarmellose sodium; and (b-iii) about 0.1% to about 2% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% to about 30% of the total core weight of the aficamten, or pharmaceutically acceptable salt thereof; (a-ii-1) about 40% to about 50% of the total core weight of mannitol; (a-ii-2) about 10% to about 20% of the total core weight of microcrystalline cellulose; (a-iii) about 1% to about 3% of the total core weight of hydroxypropyl cellulose; (a-iv) about 2% to about 4% of the total core weight of croscarmellose sodium; and (a-v) about 1% to about 3% of the total core weight of sodium lauryl sulfate; and (b) an extra-granular component comprising: (b-i) about 5% to about 15% of the total core weight of microcrystalline cellulose; (b-ii) about 1% to about 3% of the total core weight of croscarmellose sodium; and (b-iii) about 0.1% to about 1.5% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% by weight to about 10% of the total core weight of the aficamten, or pharmaceutically acceptable salt thereof; (a-ii-1) about 50% to about 60% of the total core weight of mannitol; (a-ii-2) about 15% to about 25% of the total core weight of microcrystalline cellulose; (a-iii) about 1% to about 3% of the total core weight of hydroxypropyl cellulose; (a-iv) about 2% to about 4% of the total core weight of croscarmellose sodium; and (a-v) about 1% to about 3% of the total core weight of sodium lauryl sulfate; and (b) an extra-granular component comprising: (b-i) about 5% to about 15% of the total core weight of microcrystalline cellulose; (b-ii) about 1% to about 3% of the total core weight of croscarmellose sodium; and (b-iii) about 0.1% to about 1.5% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 20% of the total core weight of the aficamten, or pharmaceutically acceptable salt thereof; (a-ii-1) about 44% of the total core weight of mannitol; (a-ii-2) about 15% of the total core weight of microcrystalline cellulose; (a-iii) about 2% of the total core weight of hydroxypropyl cellulose; (a-iv) about 3% of the total core weight of croscarmellose sodium; and (a-v) about 2% of the total core weight of sodium lauryl sulfate; and (b) an extra-granular component comprising: (b-i) about 11% of the total core weight of microcrystalline cellulose; (b-ii) about 2% of the total core weight of croscarmellose sodium; and (b-iii) about 1% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% of the total core weight of the aficamten, or pharmaceutically acceptable salt thereof; (a-ii-1) about 50% of the total core weight of mannitol; (a-ii-2) about 19% of the total core weight of microcrystalline cellulose; (a-iii) about 2% of the total core weight of hydroxypropyl cellulose; (a-iv) about 3% of the total core weight of croscarmellose sodium; and (a-v) about 2% of the total core weight of sodium lauryl sulfate; and (b) an extra-granular component comprising: (b-i) about 11% of the total core weight of microcrystalline cellulose; (b-ii) about 2% of the total core weight of croscarmellose sodium; and (b-iii) about 1% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 5% by weight of the aficamten, or pharmaceutically acceptable salt thereof; (a-ii-1) about 54% by weight of mannitol; (a-ii-2) about 20% by weight of microcrystalline cellulose; (a-iii) about 2% by weight of hydroxypropyl cellulose; (a-iv) about 3% by weight of croscarmellose sodium; and (a-v) about 2% by weight of sodium lauryl sulfate; and (b) an extra-granular component comprising: (b-i) about 11% by weight of microcrystalline cellulose; (b-ii) about 2% by weight of croscarmellose sodium; and (b-iii) about 1% by weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the tablet comprises a coating layer comprising a coating agent. In some embodiments, the coating layer surrounds the total core of the tablet. In some embodiments, the coating agent is selected from the group consisting of Opadry QX White 21A180025, Opadry I, and Opadry II. In some embodiments, the coating agent is Opadry QX White 21A180025. In some embodiments, the tablet comprises about 0.5% to about 10% of the total core weight of coating agent. In some embodiments, the tablet comprises about 1% to about 5% of the total core weight of coating agent. In some embodiments, the tablet comprises about 2% to about 4% of the total core weight of coating agent. In some embodiments, the tablet comprises about 3% of the total core weight of coating agent. In some embodiments, the tablet comprises about 0.5% to about 10% of the total core weight of Opadry QX White 21A180025. In some embodiments, the tablet comprises about 1% to about 5% of the total core weight of Opadry QX White 21A180025. In some embodiments, the tablet comprises about 2% to about 4% of the total core weight of Opadry QX White 21A180025. In some embodiments, the tablet comprises about 3% of the total core weight of Opadry QX White 21A180025.

In some embodiments, the amount of aficamten (which may exist in various forms) in a formulation, e.g., a tablet, is about a daily dose as described herein, e.g., of about 5 mg, about 10 mg, about 15 mg, or about 20 mg of aficamten. In some embodiments, the amount of aficamten (which may exist in various forms) in a formulation (e.g., a tablet) is about half of a daily dose as described herein. In some embodiments, the amount of aficamten in a formulation (e.g., a tablet) is about 2.5 mg, about 5 mg, about 7.5 mg, or about 10 mg of aficamten.

In some embodiments, aficamten exists in its free base form in various formulations, e.g., tablets. In some embodiments, a method, e.g., a method described in an Example, uses such tablets.

Liquid pharmaceutically administrable compositions can, for example, be prepared by dissolving, dispersing or suspending etc. a compound disclosed and/or described herein and optional pharmaceutical additives in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of the compound contained in such parenteral compositions depends, for example, on the physical nature of the compound, the activity of the compound and the needs of the subject. However, percentages of active ingredient of 0.01% to 10% in solution are employable, and may be higher if the composition is a solid which will be subsequently diluted to another concentration. In some embodiments, the composition will comprise from about 0.2 to 2% of a compound disclosed and/or described herein in solution.

Pharmaceutical compositions of the compounds disclosed and/or described herein may also be administered to the respiratory tract as an aerosol or solution for a nebulizer, or as a microfine powder for insufflation, alone or in combination with an inert carrier such as lactose. In such a case, the particles of the pharmaceutical composition may have diameters of less than 50 microns, or in some embodiments, less than 10 microns. Pharmaceutical composition of the compounds disclosed and/or described herein may also be administered transdermally, e.g., in the form of a patch, a cream, an ointment or other formulation suitable for administration to the skin.

In addition, pharmaceutical compositions can include a compound disclosed and/or described herein and one or more additional medicinal agents, pharmaceutical agents, adjuvants, and the like. Suitable medicinal and pharmaceutical agents include those described herein.

### Polymorphs

In some embodiments, aficamten is in a polymorph form of (R)—N-(5-(5-ethyl-1,2,4-oxadiazol-3-yl)-2,3-dihydro-1H-inden-1-yl)-1-methyl-1H-pyrazole-4-carboxamide. Suitable polymorphs of aficamten include those disclosed in and may be prepared according to WO 2021/011807, which is incorporated by reference herein in its entirety. The polymorphs may have properties such as bioavailability and stability under certain conditions that are suitable for medical or pharmaceutical uses. Variations in the crystal structure of a pharmaceutical drug substance may affect the dissolution rate (which may affect bioavailability, etc.), manufacturability (e.g., ease of handling, ease of purification, ability to consistently prepare doses of known strength, etc.) and stability (e.g., thermal stability, shelf life (including resistance to degradation), etc.) of a pharmaceutical drug product. Such variations may affect the methods of preparation or formulation of pharmaceutical compositions in different dosage or delivery forms, such as solid oral dosage forms including tablets and capsules. Compared to other forms such as non-crystalline or amorphous forms, polymorphs may provide desired or suitable hygroscopicity, particle size control, dissolution rate, solubility, purity, physical and chemical stability, manufacturability, yield, reproducibility, and/or process control. Thus, polymorphs of (R)—N-(5-(5-ethyl-1,2,4-oxadiazol-3-yl)-2,3-dihydro-1H-inden-1-yl)-1-methyl-1H-pyrazole-4-carboxamide may provide advantages of improving the manufacturing process of an active agent or the stability or storability of a drug product form of the active agent, or having suitable bioavailability and/or stability as an active agent. In some embodiments, the aficamten in a formulation, e.g., a tablet, is one of polymorphic Form I, Form II, Form III, Form IV, Form V, or Form VI, or a combination thereof. In some embodiments, the aficamten is one of polymorphic Form I, Form II, Form III, Form IV, Form V, or Form VI. In some embodiments, the aficamten is polymorphic Form I or Form IV. In some embodiments, the aficamten is polymorphic Form I. In some embodiments, the aficamten is polymorphic Form IV.

### Form I

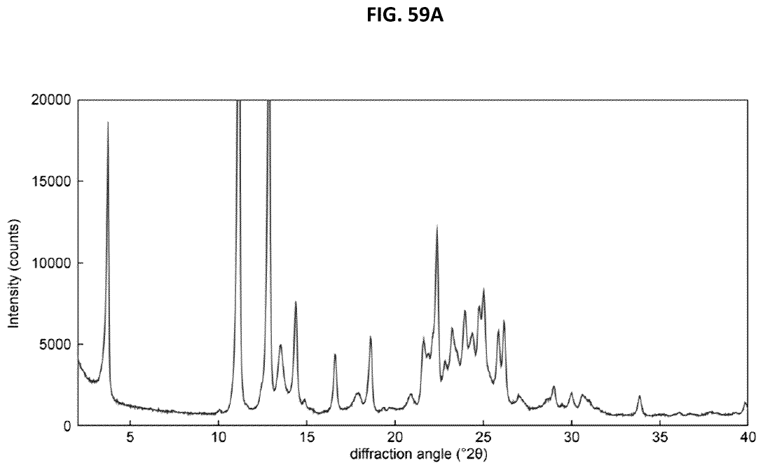

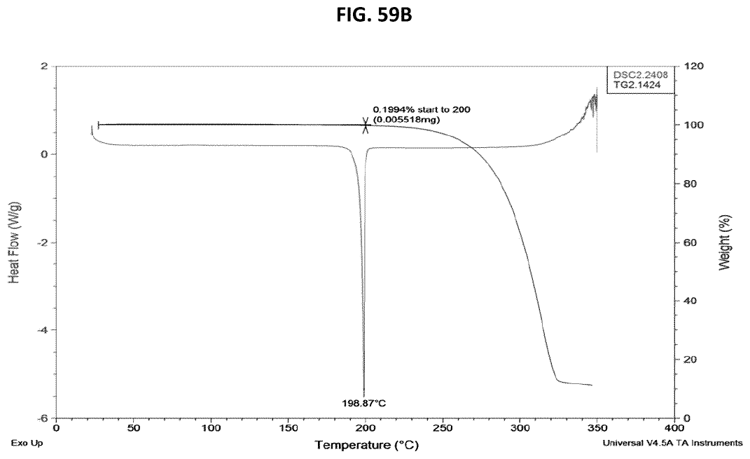

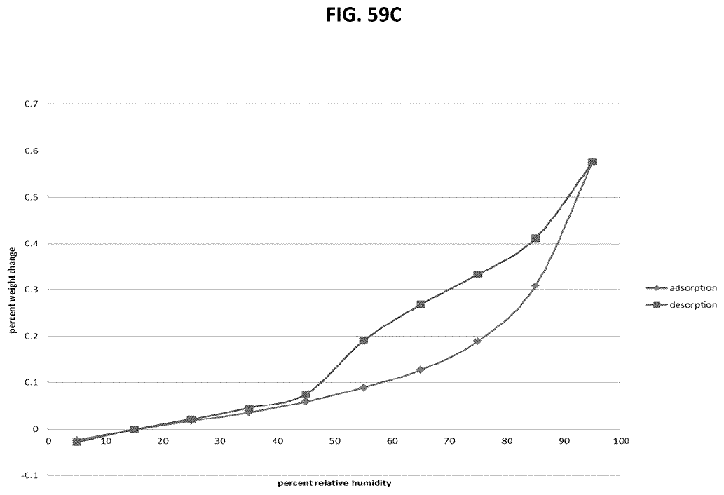

In some embodiments, the aficamten is polymorphic Form I of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form I using XRPD are shown in Table P-1. In some embodiments, Form I has an XRPD pattern substantially as shown in FIG. 59A.

TABLE P-1

| Angle (°2θ) | d value (Å) | Intensity (counts) |
|---|---|---|
| 3.7 | 23.6 | 55228.97 |
| 11.2 | 7.9 | 126123.6 |
| 12.9 | 6.9 | 94231.09 |
| 13.5 | 6.6 | 12945.13 |
| 14.4 | 6.2 | 22543.29 |
| 14.9 | 6.0 | 1982.81 |
| 16.6 | 5.3 | 12426.88 |
| 17.8 | 5.0 | 3641.86 |
| 18.6 | 4.8 | 15100.54 |
| 21.6 | 4.1 | 10927.77 |
| 22.2 | 4.0 | 10386.49 |
| 22.4 | 4.0 | 29345.32 |
| 22.8 | 3.9 | 4094.45 |
| 23.2 | 3.8 | 9276.26 |
| 23.9 | 3.7 | 12944.35 |
| 24.4 | 3.7 | 8288.4 |
| 24.7 | 3.6 | 14182.35 |
| 25.0 | 3.6 | 16601.84 |
| 25.8 | 3.4 | 11350.3 |
| 26.1 | 3.4 | 14448.7 |
| 28.6 | 3.1 | 3088.71 |
| 29.0 | 3.1 | 4033.18 |
| 29.4 | 3.0 | 2451.42 |
| 29.9 | 3.0 | 3631.24 |
| 30.6 | 2.9 | 4172.12 |
| 33.8 | 2.6 | 3752.58 |
| 36.1 | 2.5 | 543.33 |
| 36.8 | 2.4 | 405.69 |
| 37.8 | 2.4 | 482.5 |
| 39.8 | 2.3 | 2685.52 |

In some embodiments, polymorphic Form I has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 59A or as provided in Table P-1. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form I, can vary by about ±0.6 degrees, ±0.4 degrees, ±0.2 degrees, or ±0.1 degrees 2-theta.

In some embodiments, polymorphic Form I has an XRPD pattern comprising peaks at angles 2-theta of 3.7±0.2, 11.2±0.2, 12.9±0.2, 13.5±0.2, 14.4±0.2, 14.9±0.2, 16.6±0.2, 17.8±0.2, 18.6±0.2, 21.6±0.2, 22.2±0.2, 22.4±0.2, 22.8±0.2, 23.2±0.2, 23.9±0.2, 24.4±0.2, 24.7±0.2, 25.0±0.2, 25.8±0.2, 26.1±0.2, 28.6±0.2, 29.0±0.2, 29.4±0.2, 29.9±0.2, 30.6±0.2, 33.8±0.2, 36.1±0.2, 36.8±0.2, 37.8±0.2, and 39.8±0.2 degrees. In some embodiments, polymorphic Form I has an XRPD pattern comprising peaks at angles 2-theta of 3.7±0.2, 11.2±0.2, 12.9±0.2, 13.5±0.2, 14.4±0.2, 18.6±0.2, 22.4±0.2, 24.7±0.2, 25.0±0.2, and 26.1±0.2 degrees. In some embodiments, polymorphic Form I has an XRPD pattern comprising peaks at angles 2-theta of 3.7±0.2, 11.2±0.2, 12.9±0.2, 14.4±0.2, and 22.4±0.2 degrees. It is to be understood that additional peaks in the XRPD pattern other than those shown in FIG. 59A or as provided in Table P-1 may be observed, for instance, due to the presence of impurities, solvent, or other polymorphs or amorphic forms present in the test sample.

In some embodiments, Form I has a differential DSC graph substantially as shown in FIG. 59B. In some embodiments, Form I is characterized as having an endotherm onset at about 199° C. as determined by DSC. In some embodiments, Form I is characterized as having an endotherm onset at 199±2° C. (e.g., 199±1.9° C., 199±1.8° C., 199±1.7° C., 199±1.6° C., 199±1.5° C., 199±1.4° C., 199±1.3° C., 192±1.2° C., 199±1, 199±0.9° C., 199±0.8° C., 199±0.7° C., 199±0.6° C., 199±0.5° C., 199±0.4° C., 199±0.3° C., 199±0.2° C., or 199±0.1° C.) as determined by DSC.

In some embodiments, Form I has a TGA graph substantially as shown in FIG. 59B.

In some embodiments, Form I has a DVS graph substantially as shown in FIG. 59C.

In some embodiments of Form I, at least one, at least two, at least three, at least four, at least five, or all of the following (a)-(f) apply:
(a) Form I has an XRPD pattern comprising peaks at angles 2-theta of 3.7±0.2, 11.2±0.2, 12.9±0.2, 14.4±0.2, and 22.4±0.2 degrees; an XRPD pattern comprising peaks at angles 2-theta of 3.7±0.2, 11.2±0.2, 12.9±0.2, 13.5±0.2, 14.4±0.2, 18.6±0.2, 22.4±0.2, 24.7±0.2, 25.0±0.2, and 26.1±0.2 degrees; or an XRPD pattern comprising peaks at angles 2-theta of 3.7±0.2, 11.2±0.2, 12.9±0.2, 13.5±0.2, 14.4±0.2, 14.9±0.2, 16.6±0.2, 17.8±0.2, 18.6±0.2, 21.6±0.2, 22.2±0.2, 22.4±0.2, 22.8±0.2, 23.2±0.2, 23.9±0.2, 24.4±0.2, 24.7±0.2, 25.0±0.2, 25.8±0.2, 26.1±0.2, 28.6±0.2, 29.0±0.2, 29.4±0.2, 29.9±0.2, 30.6±0.2, 33.8±0.2, 36.1±0.2, 36.8±0.2, 37.8±0.2, and 39.8±0.2 degrees;
(b) Form I has an XRPD pattern substantially as shown in FIG. 59A;
(c) Form I has a DSC graph substantially as shown in FIG. 59B;
(d) Form I is characterized as having an endotherm onset at about 199° C. as determined by DSC;
(e) Form I has a TGA graph substantially as shown in FIG. 59B; and
(f) Form I has a DVS graph substantially as shown in FIG. 59C.

### Form II

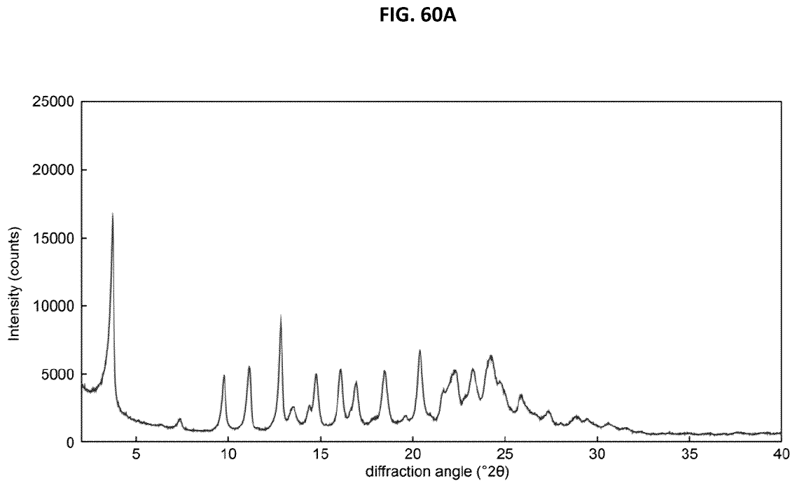

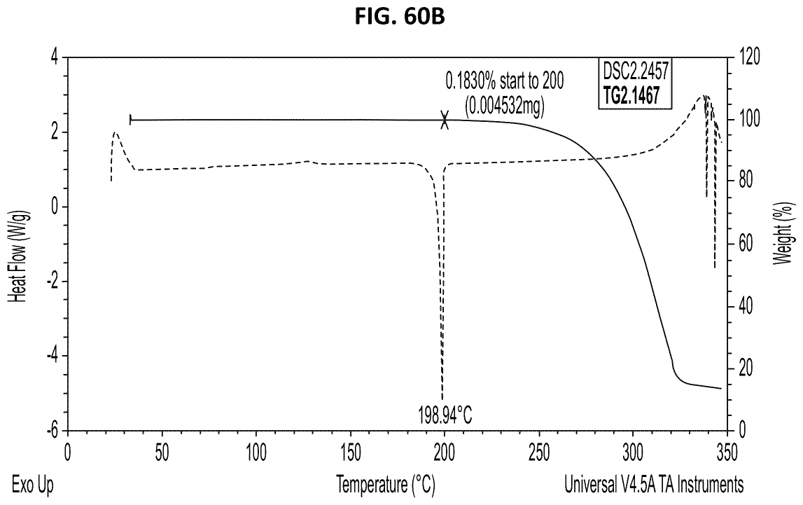

In some embodiments, the aficamten is polymorphic Form II of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form II using XRPD are shown in Table P-2. In some embodiments, Form II has an XRPD pattern substantially as shown in FIG. 60A.

TABLE P-2

| Angle (°2θ) | d value (Å) | Intensity (counts) |
|---|---|---|
| 3.7 | 23.6 | 46289.09 |
| 7.4 | 12.0 | 2626.2 |
| 9.8 | 9.0 | 13093.89 |
| 11.1 | 7.9 | 15067.72 |
| 12.8 | 6.9 | 24560.2 |
| 13.5 | 6.6 | 4597.35 |
| 14.4 | 6.2 | 4359.38 |
| 14.7 | 6.0 | 12272.37 |
| 16.1 | 5.5 | 12841.63 |
| 17.0 | 5.2 | 8725.7 |
| 18.5 | 4.8 | 11802.76 |
| 20.4 | 4.4 | 16037.66 |
| 21.6 | 4.1 | 4907.93 |
| 22.3 | 4.0 | 10307.6 |
| 23.3 | 3.8 | 8940.83 |
| 24.0 | 3.7 | 6680.21 |
| 24.3 | 3.7 | 7959.39 |
| 24.8 | 3.6 | 6336.24 |
| 25.8 | 3.4 | 4019.98 |
| 27.4 | 3.3 | 1872.66 |
| 28.8 | 3.1 | 1783 |
| 29.5 | 3.0 | 1203.02 |
| 30.5 | 2.9 | 1080.7 |

In some embodiments, polymorphic Form II has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 60A or as provided in Table P-2. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form II, can vary by about ±0.6 degrees, ±0.4 degrees, ±0.2 degrees, or ±0.1 degrees 2-theta.

In some embodiments, polymorphic Form II has an XRPD pattern comprising peaks at angles 2-theta of 3.7±0.2, 7.4±0.2, 9.8±0.2, 11.1±0.2, 12.8±0.2, 13.5±0.2, 14.4±0.2, 14.7±0.2, 16.1±0.2, 17.0±0.2, 18.5±0.2, 20.4±0.2, 21.6±0.2, 22.3±0.2, 23.3±0.2, 24.0±0.2, 24.3±0.2, 24.8±0.2, 25.8±0.2, 27.4±0.2, 28.8±0.2, 29.5±0.2, and 30.5±0.2 degrees. In some embodiments, polymorphic Form II has an XRPD pattern comprising peaks at angles 2-theta of 3.7±0.2, 9.8±0.2, 11.1±0.2, 12.8±0.2, 14.7±0.2, 16.1±0.2, 18.5±0.2, 20.4±0.2, 22.3±0.2, and 23.3±0.2 degrees. In some embodiments, polymorphic Form II has an XRPD pattern comprising peaks at angles 2-theta of 3.7±0.2, 9.8±0.2, 11.1±0.2, 12.8±0.2, and 20.4±0.2 degrees. It is to be understood that additional peaks in the XRPD pattern other than those shown in FIG. 60A or as provided in Table P-2 may be observed, for instance, due to the presence of impurities, solvent, or other polymorphs or amorphic forms present in the test sample.

In some embodiments, Form II has a DSC graph substantially as shown in FIG. 60B. In some embodiments, Form II is characterized as having an endotherm onset at about 199° C. as determined by DSC. In some embodiments, Form II is characterized as having an endotherm onset at about 199±2° C. (e.g., 199±1.9° C., 199±1.8° C., 199±1.7° C., 199±1.6° C., 199±1.5° C., 199±1.4° C., 199±1.3° C., 199±1.2° C., 199±1, 199±0.9° C., 199±0.8° C., 199±0.7° C., 199±0.6° C., 199±0.5° C., 199±0.4° C., 199±0.3° C., 199±0.2° C., or 199±0.1° C.) as determined by DSC.

In some embodiments, Form II has a TGA graph substantially as shown in FIG. 60B.

In some embodiments of Form II, at least one, at least two, at least three, at least four, or all of the following (a)-(e) apply:
(a) Form II has an XRPD pattern comprising peaks at angles 2-theta of 3.7±0.2, 9.8±0.2, 11.1±0.2, 12.8±0.2, and 20.4±0.2 degrees; an XRPD pattern comprising peaks at angles 2-theta of 3.7±0.2, 9.8±0.2, 11.1±0.2, 12.8±0.2, 14.7±0.2, 16.1±0.2, 18.5±0.2, 20.4±0.2, 22.3±0.2, and 23.3±0.2 degrees; or an XRPD pattern comprising peaks at angles 2-theta of 3.7±0.2, 7.4±0.2, 9.8±0.2, 11.1±0.2, 12.8±0.2, 13.5±0.2, 14.4±0.2, 14.7±0.2, 16.1±0.2, 17.0±0.2, 18.5±0.2, 20.4±0.2, 21.6±0.2, 22.3±0.2, 23.3±0.2, 24.0±0.2, 24.3±0.2, 24.8±0.2, 25.8±0.2, 27.4±0.2, 28.8±0.2, 29.5±0.2, and 30.5±0.2 degrees;
(b) Form II has an XRPD pattern substantially as shown in FIG. 60A;
(c) Form II has a DSC graph substantially as shown in FIG. 60B;
(d) Form II is characterized as having a melting endotherm onset at about 199° C. as determined by DSC; and
(e) Form II has a TGA graph substantially as shown in FIG. 60B.

### Form III

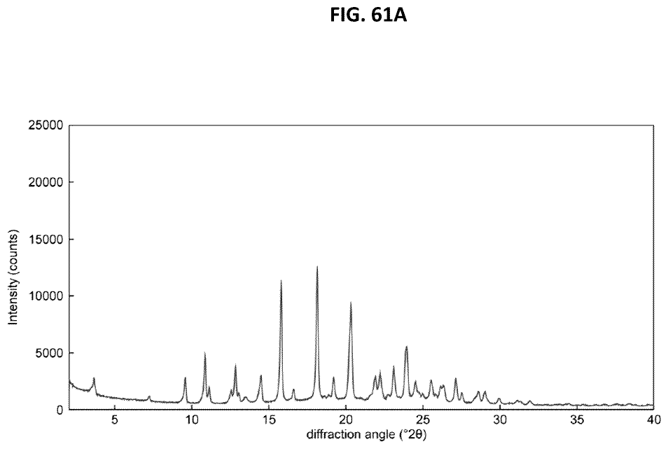

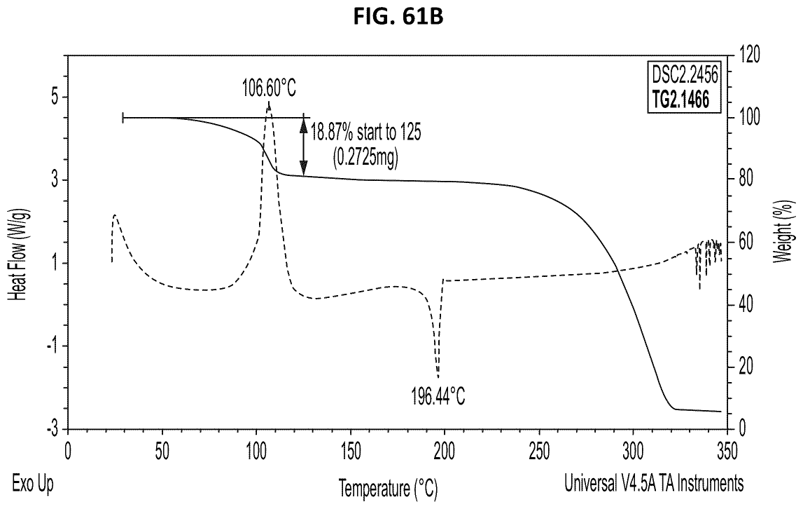

In some embodiments, the aficamten is polymorphic Form III of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for a mixture of Forms I and III using XRPD are shown in Table P-3. In some embodiments, a mixture of Forms I and III has an XRPD pattern substantially as shown in FIG. 61A.

TABLE P-3

| Angle (°2θ) | d value (Å) | Intensity (counts) |
|---|---|---|
| 3.6 | 24.3 | 4294.42 |
| 7.2 | 12.2 | 1591.53 |
| 9.6 | 9.2 | 7874.85 |
| 10.9 | 8.1 | 14071.97 |
| 11.1 | 7.9 | 4718.33 |
| 12.6 | 7.0 | 3488.46 |
| 12.8 | 6.9 | 11281.44 |
| 13.1 | 6.8 | 2684.87 |
| 13.5 | 6.5 | 1648.15 |
| 14.5 | 6.1 | 7367.71 |
| 15.8 | 5.6 | 35234.75 |
| 16.6 | 5.3 | 3504.83 |
| 18.1 | 4.9 | 41037.79 |
| 18.9 | 4.7 | 1062.55 |
| 19.2 | 4.6 | 6379.73 |
| 20.2 | 4.4 | 16176.19 |
| 20.3 | 4.4 | 17371.66 |
| 21.9 | 4.1 | 5995.19 |
| 22.2 | 4.0 | 6813.28 |
| 22.7 | 3.9 | 994.33 |
| 23.1 | 3.8 | 8769.11 |
| 23.9 | 3.7 | 16157.73 |
| 24.5 | 3.6 | 4099.43 |
| 24.9 | 3.6 | 927.84 |
| 25.5 | 3.5 | 5405.35 |
| 25.8 | 3.4 | 1470.89 |
| 26.1 | 3.4 | 2594.53 |
| 26.3 | 3.4 | 4109.63 |
| 27.1 | 3.3 | 6802.97 |
| 27.5 | 3.2 | 2786.18 |
| 28.6 | 3.1 | 2999.86 |
| 29.0 | 3.1 | 3257.84 |
| 29.9 | 3.0 | 1863.20 |
| 31.2 | 2.9 | 1010.52 |
| 31.9 | 2.8 | 1185.03 |
| 34.4 | 2.6 | 417.85 |
| 36.7 | 2.4 | 364.18 |
| 37.6 | 2.4 | 414.02 |
| 38.4 | 2.3 | 560.53 |

In some embodiments, polymorphic Form III has an XRPD pattern comprising peaks at angles 2-theta of 9.6±0.2, 10.9±0.2, 15.8±0.2, and 18.1±0.2 degrees. In some embodiments, polymorphic Form III has an XRPD pattern comprising peaks at angles 2-theta of 9.6±0.2, 10.9±0.2, 14.5±0.2, 15.8±0.2, and 18.1±0.2 degrees. In some embodiments, polymorphic Form III has an XRPD pattern comprising peaks at angles 2-theta of 9.6±0.2, 10.9±0.2, 14.5±0.2, 15.8±0.2, 18.1±0.2, and 20.2±0.2 degrees. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form III, can vary by about ±0.6 degrees, ±0.4 degrees, ±0.2 degrees, or ±0.1 degrees 2-theta.

In some embodiments, a mixture of polymorphic Forms I and III has a DSC graph substantially as shown in FIG. 61B.

In some embodiments, a mixture of polymorphic Forms I and III has a TGA graph substantially as shown in FIG. 61B.

### Form IV

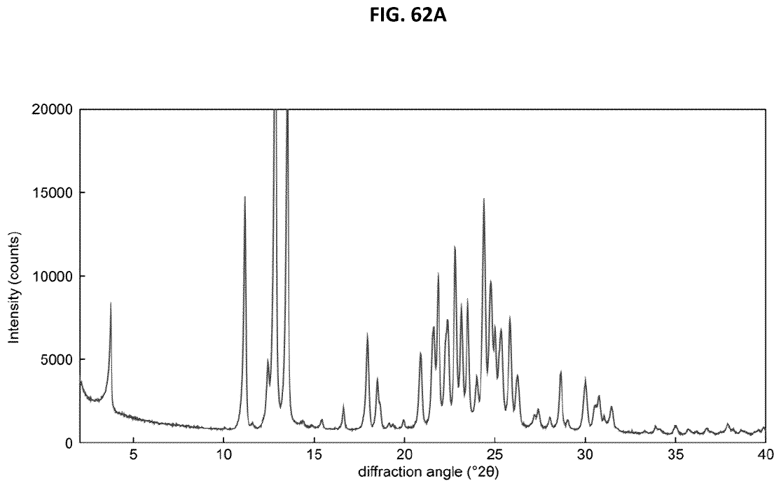

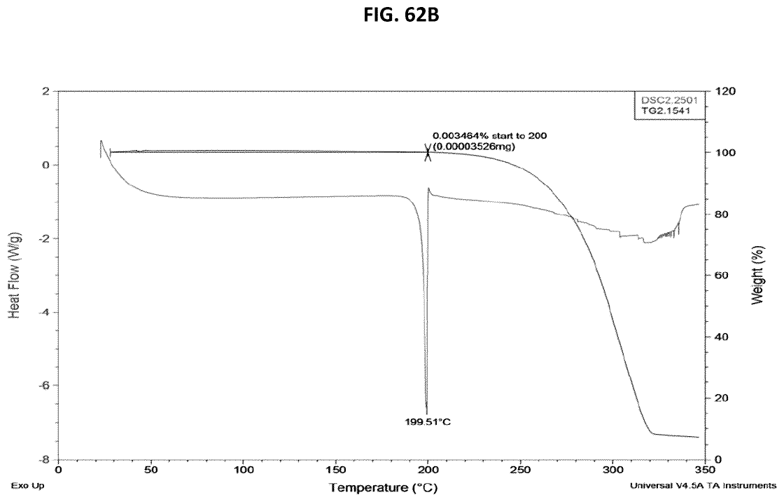

In some embodiments, the aficamten is polymorphic Form IV of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form IV using XRPD are shown in Table P-4. In some embodiments, Form IV has an XRPD pattern substantially as shown in FIG. 62A.

TABLE P-4

| Angle (°2θ) | d value (Å) | Intensity (counts) |
|---|---|---|
| 3.7 | 23.6 | 20546.07 |
| 7.7 | 11.5 | 316.33 |
| 11.1 | 7.9 | 50282.89 |
| 12.4 | 7.1 | 9463.20 |
| 12.8 | 6.9 | 128952.37 |
| 13.5 | 6.6 | 73004.53 |
| 14.3 | 6.2 | 1107.15 |
| 15.5 | 5.7 | 1601.21 |
| 16.6 | 5.3 | 4393.72 |
| 17.9 | 4.9 | 18738.68 |
| 18.5 | 4.8 | 10243.00 |
| 18.6 | 4.8 | 5141.69 |
| 19.1 | 4.6 | 807.92 |
| 19.9 | 4.4 | 1686.97 |
| 20.9 | 4.2 | 14781.60 |
| 21.5 | 4.1 | 11233.06 |
| 21.6 | 4.1 | 8729.09 |
| 21.9 | 4.1 | 29321.27 |
| 22.3 | 4.0 | 16474.92 |
| 22.4 | 4.0 | 12987.42 |
| 22.8 | 3.9 | 35633.79 |
| 23.1 | 3.8 | 21970.49 |
| 23.5 | 3.8 | 24153.01 |
| 23.9 | 3.7 | 6592.59 |
| 24.4 | 3.6 | 42430.12 |
| 24.8 | 3.6 | 27600.62 |
| 25.0 | 3.6 | 18197.26 |
| 25.3 | 3.5 | 17487.34 |
| 25.8 | 3.4 | 20166.11 |
| 26.2 | 3.4 | 9283.12 |
| 27.1 | 3.3 | 1860.56 |
| 27.4 | 3.3 | 3534.99 |
| 28.0 | 3.2 | 2537.61 |
| 28.6 | 3.1 | 13746.55 |
| 29.0 | 3.1 | 1991.34 |
| 30.0 | 3.0 | 11236.64 |
| 30.5 | 2.9 | 5185.28 |
| 30.8 | 2.9 | 7657.41 |
| 31.0 | 2.9 | 2753.63 |
| 31.4 | 2.8 | 5359.89 |
| 33.8 | 2.6 | 1085.12 |
| 35.0 | 2.6 | 1833.47 |
| 35.7 | 2.5 | 1218.65 |
| 36.1 | 2.5 | 496.93 |
| 36.7 | 2.4 | 1373.07 |
| 37.9 | 2.4 | 2127.34 |
| 38.1 | 2.4 | 883.05 |
| 39.8 | 2.3 | 1200.46 |

In some embodiments, polymorphic Form IV has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 62A or as provided in Table P-4. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form IV, can vary by about ±0.6 degrees, ±0.4 degrees, ±0.2 degrees, or ±0.1 degrees 2-theta.

In some embodiments, polymorphic Form IV has an XRPD pattern comprising peaks at angles 2-theta of 3.7±0.2, 7.7±0.2, 11.1±0.2, 12.4±0.2, 12.8±0.2, 13.5±0.2, 14.3±0.2, 15.5±0.2, 16.6±0.2, 17.9±0.2, 18.5±0.2, 18.6±0.2, 19.1±0.2, 19.9±0.2, 20.9±0.2, 21.5±0.2, 21.6±0.2, 21.9±0.2, 22.3±0.2, 22.4±0.2, 22.8±0.2, 23.1±0.2, 23.5±0.2, 23.9±0.2, 24.4±0.2, 24.8±0.2, 25.0±0.2, 25.3±0.2, 25.8±0.2, 26.2±0.2, 27.1±0.2, 27.4±0.2, 28.0±0.2, 28.6±0.2, 29.0±0.2, 30.0±0.2, 30.5±0.2, 30.8±0.2, 31.0±0.2, 31.4±0.2, 33.8±0.2, 35.0±0.2, 35.7±0.2, 36.1±0.2, 36.7±0.2, 37.9±0.2, 38.1±0.2, 39.8±0.2 degrees. In some embodiments, polymorphic Form IV has an XRPD pattern comprising peaks at angles 2-theta of 3.7±0.2, 11.1±0.2, 12.8±0.2, 13.5±0.2, 21.9±0.2, 22.8±0.2, 23.1±0.2, 23.5±0.2, 24.4±0.2, and 24.8±0.2 degrees. In some embodiments, polymorphic Form IV has an XRPD pattern comprising peaks at angles 2-theta of 11.1±0.2, 12.8±0.2, 13.5±0.2, 22.8±0.2, and 24.4±0.2 degrees. It is to be understood that additional peaks in the XRPD pattern other than those shown in FIG. 62A or as provided in Table P-4 may be observed, for instance, due to the presence of impurities, solvent, or other polymorphs or amorphic forms present in the test sample.

In some embodiments, Form IV has a DSC graph substantially as shown in FIG. 62B. In some embodiments, Form IV is characterized as having an endotherm onset at about 200° C. as determined by DSC. In some embodiments, Form IV is characterized as having a melting endotherm onset at about 200±2° C. (e.g., 200±1.9° C., 200±1.8° C., 200±1.7° C., 200±1.6° C., 200±1.5° C., 200±1.4° C., 200±1.3° C., 200±1.2° C., 200±1, 200±0.9° C., 200±0.8° C., 200±0.7° C., 200±0.6° C., 200±0.5° C., 200±0.4° C., 200±0.3° C., 200±0.2° C., or 200±0.1° C.) as determined by DSC.

In some embodiments, Form IV has a TGA graph substantially as shown in FIG. 62B.

In some embodiments of Form IV, at least one, at least two, at least three, at least four, or all of the following (a)-(e) apply:
(a) Form IV has an XRPD pattern comprising peaks at angles 2-theta of 11.1±0.2, 12.8±0.2, 13.5±0.2, 22.8±0.2, and 24.4±0.2 degrees; an XRPD pattern comprising peaks at angles 2-theta of 3.7±0.2, 11.1±0.2, 12.8±0.2, 13.5±0.2, 21.9±0.2, 22.8±0.2, 23.1±0.2, 23.5±0.2, 24.4±0.2, and 24.8±0.2 degrees; or an XRPD pattern comprising peaks at angles 2-theta of 3.7±0.2, 7.7±0.2, 11.1±0.2, 12.4±0.2, 12.8±0.2, 13.5±0.2, 14.3±0.2, 15.5±0.2, 16.6±0.2, 17.9±0.2, 18.5±0.2, 18.6±0.2, 19.1±0.2, 19.9±0.2, 20.9±0.2, 21.5±0.2, 21.6±0.2, 21.9±0.2, 22.3±0.2, 22.4±0.2, 22.8±0.2, 23.1±0.2, 23.5±0.2, 23.9±0.2, 24.4±0.2, 24.8±0.2, 25.0±0.2, 25.3±0.2, 25.8±0.2, 26.2±0.2, 27.1±0.2, 27.4±0.2, 28.0±0.2, 28.6±0.2, 29.0±0.2, 30.0±0.2, 30.5±0.2, 30.8±0.2, 31.0±0.2, 31.4±0.2, 33.8±0.2, 35.0±0.2, 35.7±0.2, 36.1±0.2, 36.7±0.2, 37.9±0.2, 38.1±0.2, 39.8±0.2 degrees;
(b) Form IV has an XRPD pattern substantially as shown in FIG. 62A;
(c) Form IV has a DSC graph substantially as shown in FIG. 62B;
(d) Form IV is characterized as having a melting endotherm onset at about 200° C. as determined by DSC; and
(e) Form IV has a TGA graph substantially as shown in FIG. 62B.

### Form V

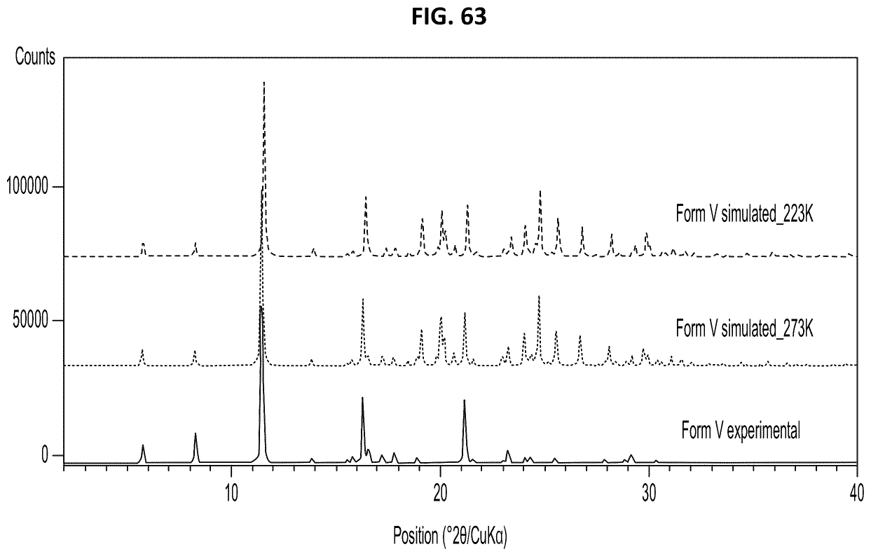

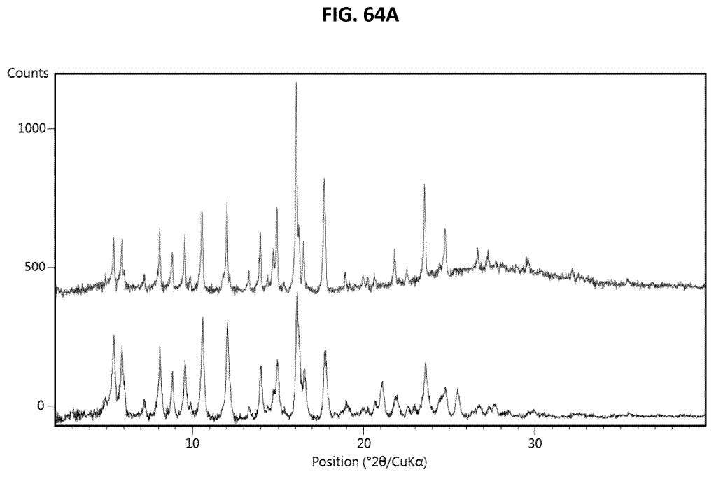

In some embodiments, the aficamten is polymorphic Form V of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form V using XRPD are shown in Table P-5. In some embodiments, Form V has an XRPD pattern substantially as shown in FIG. 63.

TABLE P-5

| Angle (°2θ) | d value (Å) | Intensity (counts) |
|---|---|---|
| 5.7 | 15.4 | 920.22 |
| 8.3 | 10.7 | 902.83 |
| 11.5 | 7.7 | 9978.6 |
| 13.8 | 6.4 | 370.79 |
| 15.5 | 5.7 | 100.26 |
| 15.8 | 5.6 | 290.17 |
| 16.3 | 5.4 | 3772.89 |
| 16.6 | 5.4 | 397.64 |
| 17.2 | 5.1 | 500.92 |
| 17.8 | 5.0 | 473.82 |
| 18.5 | 4.8 | 237.44 |
| 18.9 | 4.7 | 388.87 |
| 19.1 | 4.6 | 2061.82 |
| 19.8 | 4.5 | 391.52 |
| 20.0 | 4.4 | 2661.48 |
| 20.2 | 4.4 | 1338.2 |
| 20.7 | 4.3 | 650.6 |
| 21.2 | 4.2 | 3071.64 |
| 21.6 | 4.1 | 295.42 |
| 23.0 | 3.9 | 446.39 |
| 23.1 | 3.9 | 165.78 |
| 23.3 | 3.8 | 1093.75 |
| 24.0 | 3.7 | 1807.48 |
| 24.2 | 3.7 | 176.77 |
| 24.3 | 3.7 | 472.09 |
| 24.6 | 3.6 | 216.92 |
| 24.7 | 3.6 | 3905.17 |
| 25.2 | 3.5 | 138.38 |
| 25.6 | 3.5 | 2004.45 |
| 26.7 | 3.3 | 1751.94 |
| 27.1 | 3.3 | 53.39 |
| 27.3 | 3.3 | 55.93 |
| 27.5 | 3.2 | 25.62 |
| 27.9 | 3.2 | 217.7 |
| 28.1 | 3.2 | 1093.17 |
| 28.4 | 3.1 | 215.32 |
| 28.9 | 3.1 | 222.86 |
| 29.2 | 3.1 | 615.11 |
| 29.7 | 3.0 | 856.96 |
| 29.8 | 3.0 | 361.62 |
| 29.9 | 3.0 | 544.6 |
| 30.4 | 2.9 | 248.45 |
| 30.6 | 2.9 | 181.85 |
| 31.1 | 2.9 | 481.57 |
| 31.3 | 2.9 | 57.68 |
| 31.5 | 2.8 | 312.95 |
| 32.0 | 2.8 | 174.91 |
| 32.9 | 2.7 | 101.41 |
| 33.0 | 2.7 | 61.9 |
| 33.2 | 2.7 | 71.42 |
| 33.5 | 2.7 | 99.57 |
| 34.4 | 2.6 | 162.66 |
| 34.6 | 2.6 | 42.98 |
| 34.9 | 2.6 | 15.79 |
| 35.3 | 2.5 | 33.86 |
| 35.7 | 2.5 | 241.09 |
| 36.0 | 2.5 | 67.72 |
| 36.2 | 2.5 | 17.68 |
| 36.5 | 2.5 | 12.09 |
| 36.6 | 2.5 | 100.18 |
| 37.0 | 2.4 | 39.38 |
| 37.1 | 2.4 | 63.03 |
| 37.5 | 2.4 | 49.61 |
| 37.8 | 2.4 | 59.59 |
| 37.9 | 2.4 | 68.66 |
| 38.3 | 2.4 | 18.63 |
| 38.4 | 2.3 | 28.07 |
| 38.7 | 2.3 | 21.55 |
| 38.8 | 2.3 | 52.54 |
| 39.3 | 2.3 | 31.1 |
| 39.4 | 2.3 | 123.61 |
| 39.6 | 2.3 | 51.44 |
| 39.9 | 2.3 | 74.51 |

In some embodiments, polymorphic Form V has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 63 or as provided in Table P-5. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form V, can vary by about ±0.6 degrees, ±0.4 degrees, ±0.2 degrees, or ±0.1 degrees 2-theta.

In some embodiments, polymorphic Form V has an XRPD pattern comprising peaks at angles 2-theta of 5.7±0.2, 8.3±0.2, 11.5±0.2, 13.8±0.2, 15.5±0.2, 15.8±0.2, 16.3±0.2, 16.6±0.2, 17.2±0.2, 17.8±0.2, 18.5±0.2, 18.9±0.2, 19.1±0.2, 19.8±0.2, 20.0±0.2, 20.2±0.2, 20.7±0.2, 21.2±0.2, 21.6±0.2, 23.0±0.2, 23.1±0.2, 23.3±0.2, 24.0±0.2, 24.2±0.2, 24.3±0.2, 24.6±0.2, 24.7±0.2, 25.2±0.2, 25.6±0.2, 26.7±0.2, 27.1±0.2, 27.3±0.2, 27.5±0.2, 27.9±0.2, 28.1±0.2, 28.4±0.2, 28.9±0.2, 29.2±0.2, 29.7±0.2, 29.8±0.2, 29.9±0.2, 30.4±0.2, 30.6±0.2, 31.1±0.2, 31.3±0.2, 31.5±0.2, 32.0±0.2, 32.9±0.2, 33.0±0.2, 33.2±0.2, 33.5±0.2, 34.4±0.2, 34.6±0.2, 34.9±0.2, 35.3±0.2, 35.7±0.2, 36.0±0.2, 36.2±0.2, 36.5±0.2, 36.6±0.2, 37.0±0.2, 37.1±0.2, 37.5±0.2, 37.8±0.2, 37.9±0.2, 38.3±0.2, 38.4±0.2, 38.7±0.2, 38.8±0.2, 39.3±0.2, 39.4±0.2, 39.6±0.2, and 39.9±0.2 degrees. In some embodiments, polymorphic Form V has an XRPD pattern comprising peaks at angles 2-theta of 5.7±0.2, 8.3±0.2, 11.5±0.2, 16.3±0.2, 17.2±0.2, 19.1±0.2, 20.0±0.2, 20.2±0.2, 20.7±0.2, 21.2±0.2, 23.3±0.2, 24.0±0.2, 24.7±0.2, 25.6±0.2, 26.7±0.2, 28.1±0.2, 29.2±0.2, 29.7±0.2, 29.9±0.2, and 31.1±0.2 degrees. In some embodiments, polymorphic Form V has an XRPD pattern comprising peaks at angles 2-theta of 11.5±0.2, 16.3±0.2, 19.1±0.2, 20.0±0.2, 20.2±0.2, 21.2±0.2, 24.0±0.2, 24.7±0.2, 25.6±0.2, and 26.7±0.2 degrees. In some embodiments, polymorphic Form V has an XRPD pattern comprising peaks at angles 2-theta of 11.5±0.2, 16.3±0.2, 20.0±0.2, 21.2±0.2, and 24.7±0.2 degrees. It is to be understood that additional peaks in the XRPD pattern other than those shown in FIG. 63 or as provided in Table P-5 may be observed, for instance, due to the presence of impurities, solvent, or other polymorphs or amorphic forms present in the test sample.

In some embodiments of Form V, at least one or both of the following (a)-(b) apply: (a) Form V has an XRPD pattern comprising peaks at angles 2-theta of 11.5±0.2, 16.3±0.2, 20.0±0.2, 21.2±0.2, and 24.7±0.2 degrees; an XRPD pattern comprising peaks at angles 2-theta of 11.5±0.2, 16.3±0.2, 19.1±0.2, 20.0±0.2, 20.2±0.2, 21.2±0.2, 24.0±0.2, 24.7±0.2, 25.6±0.2, and 26.7±0.2 degrees; or an XRPD pattern comprising peaks at angles 2-theta of 5.7±0.2, 8.3±0.2, 11.5±0.2, 16.3±0.2, 17.2±0.2, 19.1±0.2, 20.0±0.2, 20.2±0.2, 20.7±0.2, 21.2±0.2, 23.3±0.2, 24.0±0.2, 24.7±0.2, 25.6±0.2, 26.7±0.2, 28.1±0.2, 29.2±0.2, 29.7±0.2, 29.9±0.2, and 31.1±0.2 degrees; and
(b) Form V has an XRPD pattern substantially as shown in FIG. 63.

### Form VI

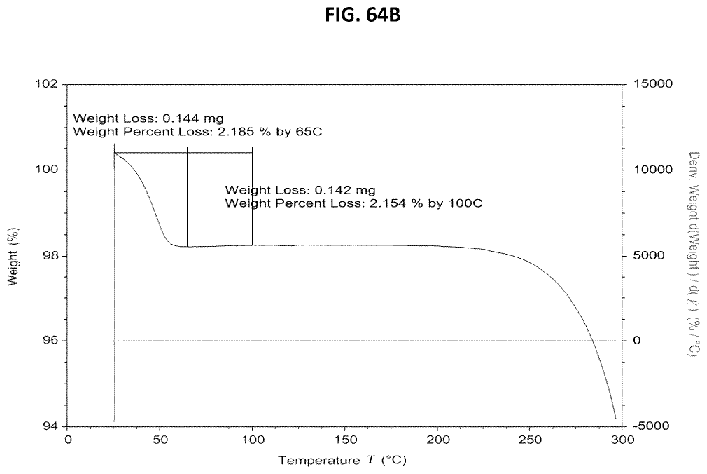

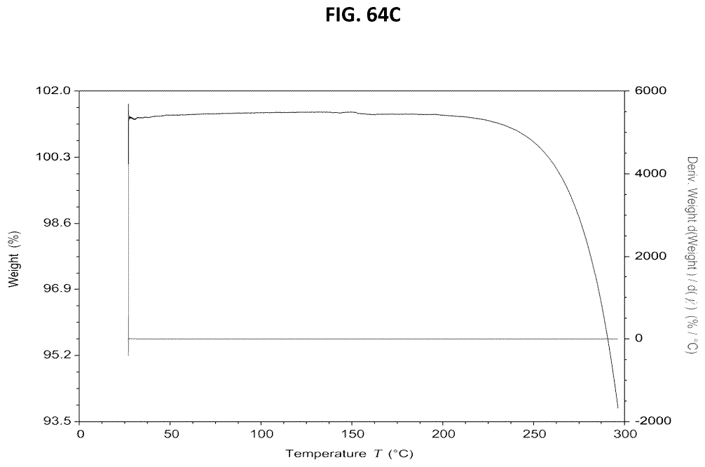

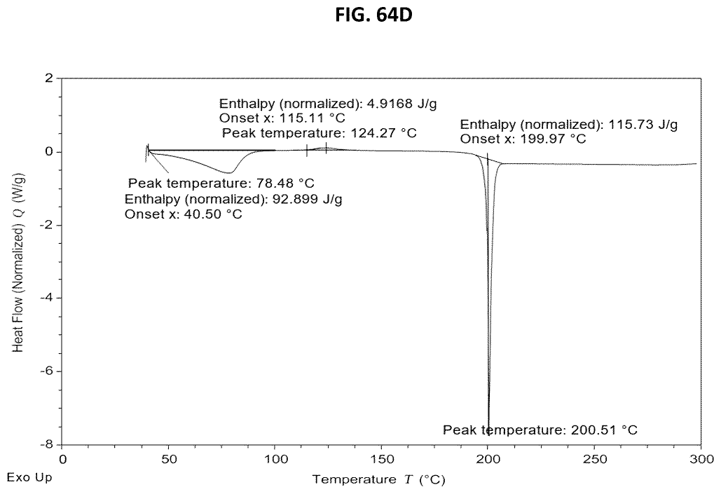

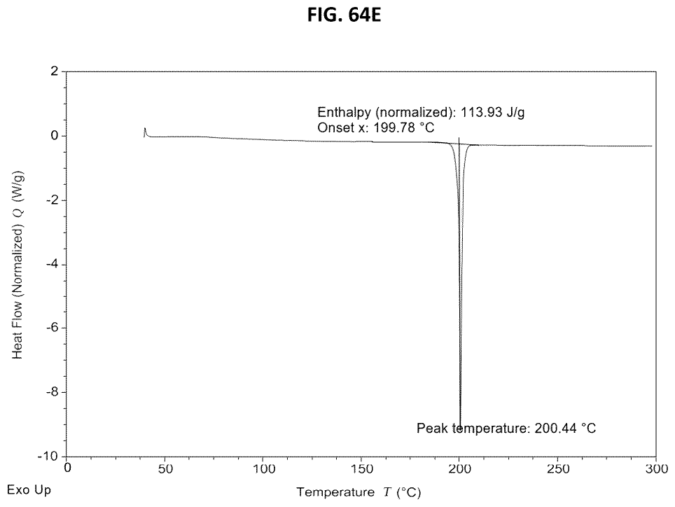

In some embodiments, the aficamten is polymorphic Form VI of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form VI using XRPD are shown in Table P-6. In some embodiments, Form VI has an XRPD pattern substantially as shown in FIG. 64A.

TABLE P-6

| Angle (°2θ) | d value (Å) | Intensity (counts) |
|---|---|---|
| 3.0 | 29.4 | 54.91 |
| 5.0 | 17.7 | 127.50 |
| 5.4 | 16.3 | 453.97 |
| 5.9 | 14.9 | 587.10 |
| 7.2 | 12.3 | 125.69 |
| 8.1 | 10.9 | 567.03 |
| 8.9 | 10.0 | 345.88 |
| 9.6 | 9.2 | 432.70 |
| 9.9 | 8.9 | 79.69 |
| 10.6 | 8.3 | 788.69 |
| 12.1 | 7.3 | 797.99 |
| 13.3 | 6.6 | 159.40 |
| 14.0 | 6.3 | 476.49 |
| 14.4 | 6.1 | 77.78 |
| 14.7 | 6.0 | 220.91 |
| 15.0 | 5.9 | 593.87 |
| 15.4 | 5.8 | 46.97 |
| 16.1 | 5.5 | 1350.47 |
| 16.5 | 5.4 | 388.67 |
| 17.8 | 5.0 | 805.27 |
| 18.9 | 4.7 | 55.59 |
| 19.0 | 4.7 | 100.12 |
| 19.2 | 4.6 | 53.05 |
| 19.6 | 4.5 | 24.26 |
| 20.0 | 4.4 | 98.96 |
| 20.3 | 4.4 | 55.64 |
| 20.7 | 4.3 | 145.05 |
| 21.1 | 4.2 | 226.01 |
| 21.9 | 4.1 | 246.53 |
| 22.6 | 3.9 | 115.35 |
| 22.9 | 3.9 | 78.78 |
| 23.6 | 3.8 | 399.26 |
| 23.6 | 3.8 | 373.80 |
| 23.8 | 3.7 | 58.05 |
| 24.4 | 3.6 | 146.88 |
| 24.8 | 3.6 | 348.39 |
| 25.5 | 3.5 | 226.63 |
| 26.4 | 3.4 | 42.15 |
| 26.7 | 3.3 | 137.53 |
| 27.3 | 3.3 | 112.85 |
| 27.6 | 3.2 | 120.57 |
| 28.2 | 3.2 | 55.34 |
| 28.5 | 3.1 | 60.37 |
| 29.0 | 3.1 | 26.44 |
| 29.6 | 3.0 | 70.01 |
| 29.9 | 3.0 | 60.95 |
| 30.4 | 2.9 | 49.00 |
| 30.9 | 2.9 | 14.91 |
| 31.6 | 2.8 | 12.97 |
| 32.2 | 2.8 | 38.90 |
| 32.6 | 2.7 | 40.95 |
| 33.1 | 2.7 | 16.26 |
| 33.3 | 2.7 | 16.56 |
| 34.5 | 2.6 | 10.35 |
| 35.0 | 2.6 | 16.35 |
| 35.5 | 2.5 | 36.10 |
| 38.5 | 2.3 | 20.00 |

In some embodiments, polymorphic Form VI has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 64A or as provided in Table P-6. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form VI, can vary by about ±0.6 degrees, ±0.4 degrees, ±0.2 degrees, or ±0.1 degrees 2-theta.

In some embodiments, polymorphic Form VI has an XRPD pattern comprising peaks at angles 2-theta of 3.0±0.2, 5.0±0.2, 5.4±0.2, 5.9±0.2, 7.2±0.2, 8.1±0.2, 8.9±0.2, 9.6±0.2, 9.9±0.2, 10.6±0.2, 12.1±0.2, 13.3±0.2, 14.0±0.2, 14.4±0.2, 14.7±0.2, 15.0±0.2, 15.4±0.2, 16.1±0.2, 16.5±0.2, 17.8±0.2, 18.9±0.2, 19.0±0.2, 19.2±0.2, 19.6±0.2, 20.0±0.2, 20.3±0.2, 20.7±0.2, 21.1±0.2, 21.9±0.2, 22.6±0.2, 22.9±0.2, 23.6±0.2, 23.8±0.2, 24.4±0.2, 24.8±0.2, 25.5±0.2, 26.4±0.2, 26.7±0.2, 27.3±0.2, 27.6±0.2, 28.2±0.2, 28.5±0.2, 29.0±0.2, 29.6±0.2, 29.9±0.2, 30.4±0.2, 30.9±0.2, 31.6±0.2, 32.2±0.2, 32.6±0.2, 33.1±0.2, 33.3±0.2, 34.5±0.2, 35.0±0.2, 35.5±0.2, and 38.5±0.2 degrees. In some embodiments, polymorphic Form VI has an XRPD pattern comprising peaks at angles 2-theta of 5.4±0.2, 5.9±0.2, 8.1±0.2, 9.6±0.2, 10.6±0.2, 12.1±0.2, 14.0±0.2, 15.0±0.2, 16.1±0.2, and 17.8±0.2 degrees. In some embodiments, polymorphic Form VI has an XRPD pattern comprising peaks at angles 2-theta of 10.6±0.2, 12.1±0.2, 15.0±0.2, 16.1±0.2, and 17.8±0.2 degrees. It is to be understood that additional peaks in the XRPD pattern other than those shown in FIG. 64A or as provided in Table P-6 may be observed, for instance, due to the presence of impurities, solvent, or other polymorphs or amorphic forms present in the test sample.

In some embodiments, Form VI has a TGA graph substantially as shown in FIG. 64B or a TGA graph substantially as shown in FIG. 64C. In some embodiments, Form VI exhibits a weight loss of about 2%±0.5% between 25° C. and 200° C. as determined by TGA.

In some embodiments, Form VI has a DSC graph substantially as shown in FIG. 64D or a DSC graph substantially as shown in FIG. 64E. In some embodiments, Form VI is characterized as having a melting endotherm onset at about 200±2° C. (e.g., 200±1.9° C., 200±1.8° C., 200±1.7° C., 200±1.6° C., 200±1.5° C., 200±1.4° C., 200±1.3° C., 200±1.2° C., 200±1, 200±0.9° C., 200±0.8° C., 200±0.7° C., 200±0.6° C., 200±0.5° C., 200±0.4° C., 200±0.3° C., 200±0.2° C., or 200±0.1° C.) as determined by DSC. In some embodiments, Form VI is characterized as having an endotherm onset at about 200±2° C. (e.g., 200±1.9° C., 200±1.8° C., 200±1.7° C., 200±1.6° C., 200±1.5° C., 200±1.4° C., 200±1.3° C., 200±1.2° C., 200±1, 200±0.9° C., 200±0.8° C., 200±0.7° C., 200±0.6° C., 200±0.5° C., 200±0.4° C., 200±0.3° C., 200±0.2° C., or 200±0.1° C.), an exotherm onset at about 115±2° C. (e.g., 115±1.9° C., 115±1.8° C., 115±1.7° C., 115±1.6° C., 115±1.5° C., 115±1.4° C., 115±1.3° C., 115±1.2° C., 115±1, 115±0.9° C., 115±0.8° C., 115±0.7° C., 115±0.6° C., 115±0.5° C., 115±0.4° C., 115±0.3° C., 115±0.2° C., or 115±0.1° C.), or an endotherm onset at about 41±2° C. (e.g., 41±1.9° C., 41±1.8° C., 41±1.7° C., 41±1.6° C., 41±1.5° C., 41±1.4° C., 41±1.3° C., 41±1.2° C., 41±1, 41±0.9° C., 41±0.8° C., 41±0.7° C., 41±0.6° C., 41±0.5° C., 41±0.4° C., 41±0.3° C., 41±0.2° C., or 41±0.1° C.), or any combinations thereof.

In some embodiments of Form VI, at least one, at least two, at least three, at least four, at least five, at least six or all of the following (a)-(g) apply:
(a) Form VI has an XRPD pattern comprising peaks at angles 2-theta of 10.6±0.2, 12.1±0.2, 15.0±0.2, 16.1±0.2, and 17.8±0.2 degrees; an XRPD pattern comprising peaks at angles 2-theta of 5.4±0.2, 5.9±0.2, 8.1±0.2, 9.6±0.2, 10.6±0.2, 12.1±0.2, 14.0±0.2, 15.0±0.2, 16.1±0.2, and 17.8±0.2 degrees; or an XRPD pattern comprising peaks at angles 2-theta of 3.0±0.2, 5.0±0.2, 5.4±0.2, 5.9±0.2, 7.2±0.2, 8.1±0.2, 8.9±0.2, 9.6±0.2, 9.9±0.2, 10.6±0.2, 12.1±0.2, 13.3±0.2, 14.0±0.2, 14.4±0.2, 14.7±0.2, 15.0±0.2, 15.4±0.2, 16.1±0.2, 16.5±0.2, 17.8±0.2, 18.9±0.2, 19.0±0.2, 19.2±0.2, 19.6±0.2, 20.0±0.2, 20.3±0.2, 20.7±0.2, 21.1±0.2, 21.9±0.2, 22.6±0.2, 22.9±0.2, 23.6±0.2, 23.8±0.2, 24.4±0.2, 24.8±0.2, 25.5±0.2, 26.4±0.2, 26.7±0.2, 27.3±0.2, 27.6±0.2, 28.2±0.2, 28.5±0.2, 29.0±0.2, 29.6±0.2, 29.9±0.2, 30.4±0.2, 30.9±0.2, 31.6±0.2, 32.2±0.2, 32.6±0.2, 33.1±0.2, 33.3±0.2, 34.5±0.2, 35.0±0.2, 35.5±0.2, and 38.5±0.2 degrees;
(b) Form VI has an XRPD pattern substantially as shown in FIG. 64A;
(c) Form VI has a TGA graph substantially as shown in FIG. 64B or FIG. 64C.
(d) Form VI has a weight loss of about 2%±0.5% between 25° C. and 200° C. as determined by TGA;
(e) Form VI has a DSC graph substantially as shown in FIG. 64D or FIG. 64E; and
(f) Form IV is characterized as having a melting endotherm onset at about 200° C. as determined by DSC; and
(g) Form VI is characterized as having an endotherm onset at about 200° C., an exotherm onset at about 115° C., or an endotherm onset at about 41° C., or any combination thereof, as determined by DSC.

### Kits

Also provided are articles of manufacture and kits containing any of the compounds or pharmaceutical compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing a compound or composition described herein and instructions for use. The kits may contain instructions for use in the treatment of a heart disease in an individual or subject in need thereof. A kit may additionally contain any materials or equipment that may be used in the administration of the compound or composition, such as vials, syringes, or IV bags. A kit may also contain sterile packaging.

In some embodiments, the present disclosure provides methods for manufacturing a medicament comprising aficamten, comprising:
manufacturing a first collection of tablets the amounts of aficamten (which may exist in various forms) in which are about, or are about half of, a first daily dose;
manufacturing a second collection of tablets the amounts of aficamten (which may exist in various forms) in which are about, or are about half of, a second daily dose;
optionally manufacturing a third collection of tablets the amounts of aficamten (which may exist in various forms) in which are about, or are about half of, a third daily dose; and
optionally manufacturing a fourth collection of tablets the amounts of aficamten (which may exist in various forms) in which are about, or are about half of, a fourth daily dose.
   In some embodiments, a method comprises manufacturing a third collection of tablets the amounts of aficamten (which may exist in various forms) in which are about, or are about half of, a third daily dose. In some embodiments, a method comprises manufacturing a fourth collection of tablets the amounts of aficamten (which may exist in various forms) in which are about, or are about half of, a fourth daily dose.

In some embodiments, the present disclosure provides methods for manufacturing medicament comprising aficamten, comprising:
manufacturing a first collection of tablets the amounts of aficamten (which may exist in various forms) in which are about a first daily dose;
manufacturing a second collection of tablets the amounts of aficamten (which may exist in various forms) in which are about a second daily dose;
optionally manufacturing a third collection of tablets the amounts of aficamten (which may exist in various forms) in which are about a third daily dose; and
optionally manufacturing a fourth collection of tablets the amounts of aficamten (which may exist in various forms) in which are about a fourth daily dose.

In some embodiments, a method comprises manufacturing a third collection of tablets the amounts of aficamten (which may exist in various forms) in which are about a third daily dose. In some embodiments, a method comprises manufacturing a fourth collection of tablets the amounts of aficamten (which may exist in various forms) in which are about a fourth daily dose.

Various first, second, third and fourth daily doses are described herein. In some embodiments, a daily dose is about 5 mg of aficamten. In some embodiments, a first daily dose is about 5 mg of aficamten. In some embodiments, a daily dose is about 10 mg of aficamten. In some embodiments, a daily dose is about 15 mg of aficamten. In some embodiments, a daily dose is about 20 mg of aficamten. In some embodiments, tablets with at least two different daily doses are manufactured. In some embodiments, tablets the amounts of aficamten (which may exist in various forms) in which are about 15 mg are manufactured. In some embodiments, tablets the amounts of aficamten (which may exist in various forms) in which are about 2.5 mg are manufactured. In some embodiments, tablets the amounts of aficamten (which may exist in various forms) in which are about 7.5 mg are manufactured. Those skilled in the art will appreciate that in addition to aficamten, tablets may comprise various other components as described herein, e.g., filler, binder, disintegrant, surfactant, lubricant, etc. In some embodiments, tablets are film-coated.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1

This first-in-human study of aficamten (also referred to as CK-274) was undertaken to evaluate its safety, pharmacokinetic, and pharmacodynamic profile, including effects of food or a CYP2D6 poor metabolizer (CYP2D6-PM) phenotype. Aficamten, a selective cardiac myosin inhibitor, reduced measures of left ventricular contractility preclinically in vitro and in vivo, and therefore, may have therapeutic potential for the management of obstructive hypertrophic cardiomyopathy. This phase 1, double-blind, randomized, placebo-controlled study enrolled healthy adults, aged 18 to 55 years, to receive single-ascending doses or multiple-ascending doses (14 or 17 days) of aficamten or placebo. In addition to standard safety and pharmacokinetic assessments, pharmacodynamic effects were assessed by echocardiography. The study enrolled 102 participants (57 in single-dose, 24 in multiple-dose, 9 in CYP2D6-PM, and 12 in food-effect cohorts). At single doses of aficamten ≤50 mg and multiple doses ≤10 mg, adverse events were generally mild and no more frequent than with placebo. In single-ascending dose cohorts, plasma concentrations of aficamten increased in a dose-proportional manner; the half-life of aficamten was 75 to 85 h. Neither food nor CYP2D6-PM phenotype had a clinically meaningful impact on pharmacokinetics. With a single 50-mg dose, mean left ventricular ejection fraction (LVEF) decreased from baseline by 5.5% (p=0.0001). With multiple doses, a mean reduction in LVEF of 5.0% was observed after 14 days of 10 mg of aficamten once daily. Aficamten appears safe and well tolerated at doses evaluated. A pharmacodynamic effect on LVEF was demonstrated, providing support for further clinical investigations of aficamten.

### Methods

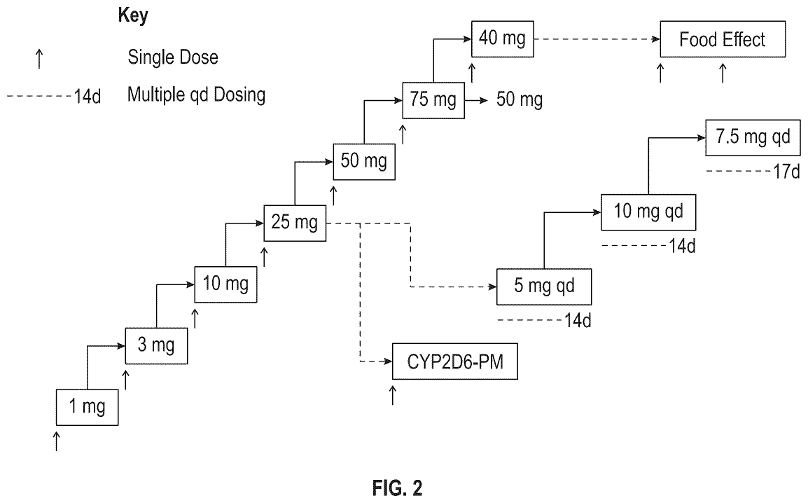

Study Overview and Ethics. The study used a randomized, placebo-controlled, single-ascending dose (SAD) and multiple-ascending dose (MAD) design (FIG. 2). The study was not designed to identify a maximum tolerated dose, but rather to identify a pharmacologically active dose range, defined as giving an absolute reduction in left ventricular ejection fraction (LVEF) from baseline in the range of 5% to 15% (such as, a baseline LVEF value of 70% reduced to between 55% to 65%). Dose escalation was to be stopped when this range was achieved, or when a non-tolerated dose was identified, if earlier.

Participants and Treatments. To be eligible for this study, participants were to be healthy adults aged 18 to 55 years, with body mass index of 18.0 to 32.0 kg/m², normal electrocardiogram (ECG) and clinical laboratory values, or only minor abnormalities that were deemed not clinically significant. Participants also had to have normal cardiac structure and function, with LVEF ≥60% for the first 4 SAD cohorts, ≥65% for subsequent SAD cohorts, all MAD cohorts, and the food-effect cohort, and ≥55% for the CYP2D6-PM cohort. Before the study, participants were not allowed to use any prescription medication within 14 days, over-the-counter medication within 7 days (except acetaminophen), or tobacco or nicotine within 3 months; in addition, they were not permitted to consume alcohol, caffeine, or grapefruit within 48 h before study check-in.

A randomization schedule was centrally generated for each cohort and treatment period. In all cohorts, aficamten or matching placebo was administered in granule form in a capsule with ˜240 ml of water. Study drug was administered following an overnight fast, except during the fed period in the food-effect cohort.

Single-ascending dose (SAD) cohorts. The SAD portion of the study used a randomized, double-blind, placebo-controlled, sequential, escalating-dose design, in which participants received single-ascending oral doses of the study drug. Seven cohorts were dosed in sequence (FIG. 2). Of the 8 participants within each cohort, the first 2 were randomly assigned (1:1) to aficamten or placebo and followed for a minimum of 2 days before the remainder of the group was dosed. The remaining 6 participants were then randomly assigned (5:1) to receive either oral single doses of aficamten (1, 3, 10, 25, 40, 50, or 75 mg) or placebo.

The initial dose of aficamten was selected using criteria from the United States Food and Drug Administration guidance based on prior animal studies and employing a safety margin of ≥10-fold. Dose escalation would stop when results identified a pharmacologically active dose range that reduced LVEF by 5% to 15% or a non-tolerated dose, whichever occurred first.

Recommendations regarding dose escalation in the SAD cohorts—and in the MAD cohorts described below—were made by the treating investigator (who was blinded to treatment group) and endorsed or not by the Dose Level Review Committee (DLRC), who were unblinded. Decisions were made when ≥6 participants had been treated and followed for ≥3 days, including collection of clinical, laboratory, ECG, and telemetry data, and echocardiograms suitable for assessing LV function around the time of maximum plasma drug concentration (Cₘₐₓ). Criteria for escalation included that no more than 2 participants in a dose group developed an LVEF <50% and no individual developed an LVEF <45%. Dose escalation criteria were as follows: (1) no individual had sustained a cardiac serious adverse event related to the study drug; (2) no 2 individuals had experienced similar, non-cardiac serious adverse events in the same organ system that seemed to be related to the study drug; (3) no 2 individuals treated with aficamten experienced a decrease in left ventricular ejection fraction (LVEF) ≥15% in comparison with the last pre-dose value (determined by the Dose Level Review Committee [DLRC]); (4) no individual developed an LVEF <45% (unless determined not to be related to the study drug by the DLRC and the treating investigator); and (5) both the treating investigator and DLRC approved the escalation and next level dose based on their clinical judgment.

Multiple-ascending dose (MAD) cohorts. The MAD cohorts also used a randomized, double-blind, placebo-controlled, sequential design. Enrollment in the MAD cohorts began when the SAD cohorts identified a single oral dose that was well tolerated and associated with an observed PD effect. Each of the 3 MAD cohorts included 8 participants, randomized (6:2) to aficamten or placebo. Participants received oral doses of the study drug once daily for 14 days (in the cohorts comparing 5 or 10 mg of aficamten vs. placebo) or 17 days (for the cohort comparing 7.5 mg of aficamten vs. placebo).

CYP2D6 poor metabolizer cohort. A separate cohort was enrolled to evaluate the potential impact of CYP2D6 genetic variants on the PK properties of aficamten. The CYP2D6 gene encodes the cytochrome P450 2D6 enzyme, described as the most extensively characterized polymorphic drug-metabolizing enzyme, and prior in vitro studies had implicated CYP2D6 as a potential metabolizing enzyme of aficamten.

CYP2D6 genotypes were determined at screening for all study participants; those identified as CYP2D6-PMs were excluded from the SAD and MAD cohorts but were invited to participate in the CYP2D6-PM cohort. The first individual in the CYP2D6-PM cohort was dosed after the SAD 25-mg cohort (FIG. 2). Each participant received a single dose of aficamten (10 mg) or placebo. Nine participants were randomized (7:2) with a sentinel dosing group consisting of the first 2 participants treated.

Food-effect cohort. To assess the effect of food on the PK of aficamten, a separate cohort was enrolled after completion of the last SAD cohort, with enrollment of 8 to 12 participants planned. In an open-label, 2-way crossover design, participants were to receive 2 single doses of 10 mg of aficamten, separated by ≥14 days. Participants were randomized in a 1:1 ratio to 1 of 2 sequences: fasted/fed or fed/fasted. In the fasted period, aficamten was administered after an overnight fast; in the fed period, aficamten was administered 30 minutes after the start of a high-fat breakfast.

### Assessment

Safety and tolerability. Safety was assessed by the incidence of adverse events (AEs) and by incidence of reduced LVEF. Treatment-emergent AEs (TEAE) were defined as AEs that began or increased after study drug administration. All AEs were coded using the Medical Dictionary for Regulatory Activities version 21.1 and graded using the National Cancer Institute Common Terminology Criteria for AEs (version 4.03) 5-point severity scale. Each AE was judged as either related or unrelated to the study drug by the treating investigator. Clinical laboratory tests were obtained at regular intervals in all cohorts.

For safety monitoring, participants in all cohorts had periodic echocardiograms, which were assessed by a cardiologist. In the SAD and MAD cohorts, echocardiograms were also reviewed by an echocardiography core laboratory for PD assessments, as described below. In addition, participants in all cohorts were monitored with continuous 12-lead ECG recording using Holter monitors. For safety monitoring, a single 12-lead ECG was extracted at screening, pre-dose, and periodically throughout follow-up, and interpreted by the investigator. In the SAD, MAD, and CYP2D6-PM cohorts, cardiodynamic ECGs (triplicate 10-second, 12-lead ECG recordings) were obtained prior to the corresponding PK blood and ECG intervals quantitated by qualified readers.

Pharmacokinetic analyses. For all study groups, blood samples for PK assessment were obtained pre-dose, up to 12 times daily on day 1, and then at regular intervals throughout the study. Blood samples were collected according to the following schedule: SAD Cohorts: Day 1: pre-dose and 0.25, 0.5, 1, 1.5, 2, 2.5, 3, 4, 6, 8, 12, 16, 24, 36, 48, 72, 96, and 216 h post dose. MAD Cohorts (14-day Dosing): Day 1: pre-dose and 0.25, 0.5, 1, 1.5, 2, 2.5, 3, 4, 6, 8, and 12 h post dose; Days 2, 4, 5, 6, and 9: pre-dose (corresponding to trough samples following dosing on days 1, 3, 4, 5, and 8) and 1.5 h post dose; Days 3, 7, 8, 10, 11, 12, and 13: pre-dose (corresponding to trough samples following dosing on days 2, 6, 7, 9, 10, 11, and 12); Day 14: pre-dose and 0.25, 0.5, 1.5, 2, 2.5, 3, 4, 6, 8, 12, 16, 24, 36, 48, 72, and 168 h post dose. MAD Cohort (17-Day Dosing): Day 1: pre-dose and 0.25, 0.5, 1, 1.5, 2, 2.5, 3, 4, 6, 8, and 12 h post dose; Days 2, 4, 5, 6, and 9: pre-dose (corresponding to trough samples following dosing on days 1, 3, 4, 5, and 8) and 1.5 h post dose; Days 3, 7, 8, 10, 11, 12, 13, 14, 15, and 16: pre-dose (corresponding to trough samples following dosing on days 2, 6, 7, 9, 10, 11, and 12); Day 17: pre-dose and 0.25, 0.5, 1.5, 2, 2.5, 3, 5, 7, 9, 12, 24, 36, 48, 72, and 168 h post dose. CYPD6-PM Cohort: Day 1: pre-dose and 0.25, 0.5, 1, 1.5, 2, 2.5, 3, 4, 6, 8, 12, 16, 24, 36, 48, 72, 96, 216, 312, and 552 h post dose. Food-Effect Cohort: Day 1: pre-dose and 0.25, 0.5, 1, 1.5, 2, 2.5, 3, 4, 6, 8, 12, 16, 24, 36, 48, 72, 96, 144, and 216 h post dose. Standard non-compartmental methods were used to calculate PK parameters using Phoenix® WinNonlin® Version 7.0; actual sample collection times were utilized.

Plasma concentrations of aficamten were measured utilizing high performance liquid chromatography-tandem mass spectrometry methods validated for accuracy, precision, linearity, sensitivity, and specificity at Celerion (Lincoln, Nebraska). The analytical range (the lower to upper limits of quantitation) for aficamten was 1.00 to 500 ng/ml.

Echocardiography. For PD assessments of LVEF, echocardiograms for the SAD and MAD cohorts were interpreted by the echocardiography core laboratory and used for all data analysis and dose level review decisions while immediate local interpretation of the echocardiograms was performed for safety monitoring. In the SAD cohorts receiving 1, 3, or 10 mg of aficamten, echocardiograms were obtained on day −1, pre-dose on day 1, and at 1.5, 4, and 24 h post dose. For SAD cohorts receiving 25, 40, 50, or 75 mg of aficamten, echocardiograms were obtained on day −1, pre-dose on day 1, and at 1.5, 6, and 24 h post dose. Echocardiograms on day 3 (48 h after dosing) were obtained only if the 24 h LVEF had not returned to near or above baseline, as determined by the investigator. In the MAD cohorts, echocardiograms were obtained on day −1, pre-dose on day 1, and at 1.5 h post dose on days 2, 4, and 9, and at 1.5, 24, and 72 h post dose on day 14 (for the 5-mg and 10-mg cohorts) or on day 17 (for the 7.5-mg cohort). Echocardiograms were obtained 3 days after the last dose (on day 17 or 20) only if the participant's prior LVEF was not near or above baseline, as determined by the investigator.

Statistical Analysis. The sample size chosen for this study was based upon precedent set by other first-in-human PK studies of similar nature and was not based on power calculations. All participants who received ≥1 dose of the study drug (aficamten or placebo) were included in safety analyses. All participants who received ≥1 dose of the study drug and had ≥1 evaluable PK plasma profile were included in the PK analysis set.

The PK analyses were intended to assess single-dose kinetics, multiple-dose (steady state) kinetics, the influence of the CYP2D6 phenotype on absorption and elimination of aficamten, and the influence of food on the absorption and elimination of aficamten. For the SAD cohort, dose proportionality of aficamten was evaluated using a power model on day 1. For the MAD cohort, dose proportionality was evaluated using a power model on day 1 and day 14 or 17. Several considerations were taken into account when assessing dose proportionality of the drug, such as results derived from the power model statistical analysis (e.g., the slope estimate and width of the 2-sided 95% confidence intervals [CIs]), qualitative assessment specific to the PK of the drug, and clinical relevance. For the SAD cohort, the parameters used to assess dose proportionality were area under the plasma drug concentration-time curve (AUC) from time 0 to the time of the last measurable concentration (AUCₗₐₛₜ), AUC from time 0 extrapolated to infinity (AUC_{inf}), AUC from time 0 to 24 h (AUC₂₄), and maximum plasma concentration (Cₘₐₓ). For the MAD cohort, the parameters were AUC₂₄ and Cₘₐₓ on day 1, plus AUC to the end of the dosing period (AUCₜₐᵤ) and Cₘₐₓ on day 14 or 17. The statistical linear relationship between the ln-transformed PK parameters and the ln-transformed dose was verified by including the quadratic (ln Dose)² and cubic (ln Dose)³ effects. The statistical linear relationship was established if the quadratic and cubic effects were not statistically significant, using a 5% level of significance, or if the effects were statistically significant, but of such small magnitude that they were not clinically relevant. The dose-proportionality analysis was performed using SAS® PROC MIXED. If a statistical linear relationship was shown and if the 2-sided 95% CIs around the slope estimate parameters included the value of 1 for dose-dependent parameters, then dose proportionality was established.

In the MAD cohort, a steady-state analysis for aficamten was performed on the ln-transformed plasma trough concentration (C trough) values using Helmert contrasts. An analysis of variance (ANOVA) model was conducted separately for each dose level; day was included as the fixed effect. Helmert contrasts were developed such that each time-point was compared with the mean of the subsequent time-points. Steady state was established at the time-point where no statistical difference (alpha=5%, 2-sided) was observed with the subsequent time-points.

All participants who received ≥1 dose of study drug and had ≥1 pre-dose and ≥1 post-dose echocardiographic measurement were included in the PD analysis set. Descriptive analyses included absolute reduction in LVEF relative to baseline and categorical LVEF responses (proportions of participants with reduction in LVEF from baseline of ≥5%, ≥10%, and ≥15% and proportions of participants with LVEF <50% and <45%). Descriptive statistics of the echocardiographic parameters were generated using SAS® Version 9.3 or higher.

Dose-response analysis was performed using analysis of covariance (ANCOVA) to identify the least-squares mean difference (aficamten minus placebo). To analyze the impact of drug dose on echocardiographic parameters in the SAD and MAD cohorts, an inferential analysis was conducted on the PD analysis set using linear mixed models for repeated-measures analysis of covariance (ANCOVA). The ANCOVA used baseline value as a covariate, included treatment, time-point, and time-point-by-treatment interaction as fixed effects, and change from baseline as the dependent variable. The unstructured variance-covariance structure was used, and the model accounted for the time-point repeated measures. The ANCOVA analysis was conducted separately for each study part and for each PD parameter. The least-squares means, the difference in least squares means (active minus placebo), and the associated 2-sided 95% CIs were presented for each comparison.

Analyses of concentration ‘bin’ and exposure-response were also performed using ANCOVA. SAS® PROC MIXED was used for all comparative analyses. An additional inferential analysis was performed in the SAD and MAD cohorts, to evaluate the relationship between concentrations of aficamten and LVEF for participants in the PK/PD analysis set. A concentration bin ANCOVA was conducted using linear mixed models for repeated-measures analyses with concentration bin group as a fixed effect, baseline PD parameters as a covariate, change from baseline as the dependent variable, and a random intercept to adjust for the repeated measures. The unstructured variance-covariance structure was used. Plasma concentrations of aficamten were paired with coincident PD parameters. The ANCOVA compared the change in PD parameters between each bin versus the pooled placebo group. The least-squares means, the difference in least squares means (bin group minus placebo), and the associated 2-sided 95% CIs were presented for each comparison. The ANCOVA analysis was conducted separately for each study part. For all time-points at which both PK data and PD measures were available, the time-points were pooled for the analysis. In each part of the study, aficamten concentrations with time-matched PD data were pooled and sorted in increasing order. From least to greatest, the data were then divided into 5 groups of observations (‘bins’), each consisting of 20% of the data points. Each bin was treated as a separate group. Concentration bins consisted of a placebo group and 5 bin groups based on the pool of concentrations from all time-points on aficamten treatment.

The above analysis was then repeated using concentration as a continuous variable to estimate the exposure-response trend. Both a random intercept effect and a random concentration effect were added to the ANCOVA. The estimate of the concentration slope, with corresponding 2-sided 95% CIs, and the ANCOVA analyses were presented for each study part.

For all time-points at which both PK data and PD measures were available, the time points were pooled for the analysis. A nominal significance level of 5% was used for statistical comparisons, without adjustment for multiplicity.

### Results

Study Population. A total of 102 participants were enrolled: 57 in the SAD cohorts, 24 in the MAD cohorts, 9 in the CYP2D6-PM cohort, and 12 in the food-effect cohort. All participants completed the study. Mean age ranged between 32 and 40 years across cohorts, and the majority of participants were male (Table 1).

TABLE 1
Baseline Characteristics

| | SAD | | MAD | | CYP2D6-PM | | Food-Effect | |
|---|---|---|---|---|---|---|---|---|
| | (n = 57) | | (n = 24) | | (n = 9) | | (n = 12) | |
| Age, years | 39.6 | (18-55) | 40.4 | (28-54) | 32.0 | (20-47) | 39.6 | (25-51) |
| Male | 41 | (72) | 18 | (75) | 9 | (100) | 9 | (75) |
| White race | 52 | (91) | 17 | (71) | 9 | (100) | 11 | (92) |
| Hispanic or Latino ethnicity | 42 | (74) | 13 | (54) | 4 | (44) | 8 | (67) |

| | | | | |
|---|---|---|---|---|
| Weight, kg | 79.4 ± 10.3 | 77.0 ± 9.1 | 84.8 ± 12.0 | 76.4 ± 11.1 |
| Height, cm | 169.0 ± 9.4 | 168.9 ± 10.0 | 177.8 ± 7.8 | 168.8 ± 9.2 |
| BMI, kg/m² | 27.8 ± 2.7 | 27.0 ± 2.3 | 26.8 ± 3.3 | 26.8 ± 3.0 |
| LVEF, % | 65.8 ± 2.4 | 67.5 ± 1.2 | 61.0 ± 2.6 | 67.2 ± 1.2 |

Values are mean (range), n (%), or mean ± SD.
BMI = body mass index; LVEF = left ventricular ejection fraction; MAD = multiple-ascending dose; CYP2D6-PM = cytochrome P450 2D6 poor metabolizer; SAD = single-ascending dose; SD = standard deviation.

For the SAD cohorts, there were no safety concerns that prohibited dose escalation between 1 mg and 25 mg. With the next planned dose (50 mg), 1 participant had a post-dose LVEF <50% (46.2%); however, this did not meet the dose escalation stopping rules and the 75-mg cohort was initiated. The sentinel participant in the 75-mg cohort had LVEF <45% post dose; consequently, no further participants were dosed at 75 mg. As a result, the 50-mg group was expanded, and an additional 5 participants were dosed in this cohort. Following the expansion, 1 participant in the 50-mg dose group experienced LVEF <45%, which was also a decrease of ≥15%. Therefore, no further participants were dosed at ≥50 mg. The DLRC determined the appropriate dose for the final single-dose cohort was 40 mg.

Following results from the 1-mg to 25-mg SAD cohorts, the first MAD cohort was initiated at 5 mg of aficamten once daily for 14 days. There were no safety concerns, and the next cohort was initiated at 10 mg once daily for 14 days. In this cohort, 2 participants met the stopping criteria based on echocardiography results. The DLRC decided the next treatment level should be 7.5 mg, to better characterize the PK at steady state; thus, the dosing period was extended from 14 to 17 days to ensure PK had reached steady state by the last day of dosing.

Safety and Tolerability. There were no serious AEs, and no participants discontinued the study due to AEs. The TEAEs that were observed were generally mild (grade 1) and no more frequent with aficamten than with placebo for both single-dose and multiple-dose administration (Table 2 and Table 3). Overall, the most common TEAE was headache in both the SAD and MAD cohorts (Table 2 and Table 3).

TABLE 2
Treatment-Emergent Adverse Events in the SAD Cohorts

| | Pooled | | Aficamten SAD Cohort, mg | | | | | | | | | | |
|---|---|---|---|---|---|---|---|---|---|---|---|---|---|
| | Placebo | | 1 | 3 | 10 | 25 | 40 | 50 | | 75 | | Total | |
| | (n = 15) | | (n = 6) | (n = 6) | (n = 6) | (n = 6) | (n = 6) | (n = 11) | | (n = 1) | | (N = 57) | |
| Participants with any TEAE | 4 | (27) | 1 (17) | — | 1 (17) | 5 (83) | 1 (17) | 4 | (36) | 1 | (100) | 17 | (30) |
| Most common TEAEs | | | | | | | | | | | | | |
| Headache | 1 | (7) | — | — | 1 (17) | 2 (33) | — | 1 | (9) | — | | 5 | (9) |
| Ejection fraction decreased | | | — | — | — | — | 1 (17) | 1 | (9) | 1 | (100) | 3 | (5) |
| Abdominal tenderness | | | — | — | — | 1 (17) | — | 1 | (9) | — | | 2 | (4) |
| Chest pain | | | — | — | — | — | — | 1 | (9)* | 1 | (100)^{†} | 2 | (4) |

| | | | | | | | | | | |
|---|---|---|---|---|---|---|---|---|---|---|
| Dyspepsia | — | — | — | 1 (17) | — | 1 | — | — | 2 | (4) |
| Flatulence | — | — | — | 1 (17) | — | 1 | — | — | 2 | (4) |

| | | | | | | | | | | | |
|---|---|---|---|---|---|---|---|---|---|---|---|
| Nausea | | | — | — | — | 2 (33) | — | — | — | 2 | (4) |
| Upper abdominal pain | | | — | — | — | 2 (33) | — | — | — | 2 | (4) |
| Upper respiratory tract infection | 2 | (13) | — | — | — | — | — | — | — | 2 | (4) |

Values are n (%). Most common TEAEs based on preferred terms, reported in ≥2 participants in the total cohort.
*‘Bubbling sensation to the left chest’ consistent with a gastrointestinal association rather than a cardiac association.
^{†}Onset approximately 60 h post dose.
SAD = single-ascending dose; TEAE = treatment-emergent adverse event.

TABLE 3
Treatment-Emergent Adverse Events in the MAD Cohorts

| | | Aficamten MAD Cohorts | | | |
|---|---|---|---|---|---|
| | Pooled | 5 mg qd × | 7.5 mg qd × | 10 mg qd × | |
| | Placebo | 14 Days | 17 Days | 14 Days | Total |
| | (n = 6) | (n = 6) | (n = 6) | (n = 6) | (N = 24) |
| Participants with any TEAE | 1 (17) | 1 (17) | — | 2 (33) | 4 (17) |
| TEAEs | | | | | |
| Headache | 1 (17) | — | — | 1 (17) | 2 (8) |
| Chapped lips | — | 1 (17) | — | — | 1 (4) |
| Cough | — | 1 (17) | — | — | 1 (4) |
| Feeling hot | 1 (17) | — | — | — | 1 (4) |
| Nausea | 1 (17) | — | — | — | 1 (4) |
| Salivary hypersecretion | — | 1 (17) | — | — | 1 (4) |
| Upper respiratory tract infection | — | — | — | 1 (17) | 1 (4) |
| Vomiting | 1 (17) | — | — | — | 1 (4) |

Values are n (%).
TEAEs based on preferred terms, reported in ≥1 participant in the total cohort.
MAD = multiple-ascending dose; qd = once daily; TEAE = treatment-emergent adverse event.

Echocardiogram-related AEs of decreased ejection fraction <45% based on the study echocardiogram expert assessment were reported in 3 participants: 1 each in the SAD 40-mg, 50-mg, and 75-mg cohorts (Table 4). All were grade 1, and all resolved at the next echocardiogram assessment (within 2.5 to 4.6 h). The single participant who received 75 mg of aficamten had LVEF of 34.6% at 1.5 h post dose, which was a reduction in LVEF of 31.5% and led to concluding escalation of doses in the SAD portion of the study as discussed above. At the following assessment 2.5 h later, LVEF had returned to 51.9%. No AEs of decreased ejection fraction <45% were reported in the MAD, CYP2D6-PM, or food-effect cohorts.

In all cohorts, mean safety ECG parameters at the assessed time-points were within normal limits. No clinically notable changes from baseline were observed among any of the parameters. The QT interval corrected for heart rate using Fridericia's formula (QTcF) did not exceed 450 ms either at baseline or at any assessment during the dosing interval, with the exception of 2 individuals whose baseline QTcF was ≥440 ms and whose QTcF values increased by 3 and 13 ms, respectively. In all cohorts, there were no increases in QTcF interval >30 ms, with the exception of 1 participant in the SAD placebo group whose QTcF interval increased by 33 ms on day 5 (427 ms vs. a baseline value of 394 ms). In the cardiodynamic assessments, categorical analysis of ECG parameters revealed no cardiac safety concerns, and there was no evidence of a positive QT effect following single or multiple doses of aficamten.

All vital signs were within normal limits at the post-dose time-points. No clinically significant serum chemistry, hematology, or urinalysis findings were observed during the study.

TABLE 4
Echocardiogram-Related AEs of Decreased Ejection Fraction <45%

| | | | | LVEF | |
|---|---|---|---|---|---|
| Cohort, | Dose | Grade | Onset* | Deemed AE^{†} | Outcome^{‡} |
| SAD, | 40 mg | 1 (mild) | 1.4 h | 42.2% | Resolved 4.6 h later |
| 42-year-old | | | | | (6 h after drug dose), |
| white female | | | | | with LVEF recorded |
| | | | | | at 58.4% |
| SAD, | 50 mg | 1 (mild) | 1.5 h | 42.1% | Resolved 2.5 h later |
| 46-year-old | | | | | (4 h after drug dose), |
| white male | | | | | with LVEF recorded |
| | | | | | at 50.2% |
| SAD, | 75 mg | 1 (mild) | 1.5 h | 34.6% | Resolved 2.5 h later |
| 36-year-old | | | | | (4 h after drug dose), |
| white/Hispanic | | | | | with LVEF recorded |
| male | | | | | at 51.9% |

*Time after drug dose.
^{†}LVEF values for safety assessments were determined by the study cardiologist.
^{‡}In the SAD cohorts, post-dose echocardiograms were performed at 1.5, 4, 6, 24, and 48 h post dose.
AE = adverse event; LVEF = left ventricular ejection fraction; SAD = single-ascending dose.

### Pharmacokinetics

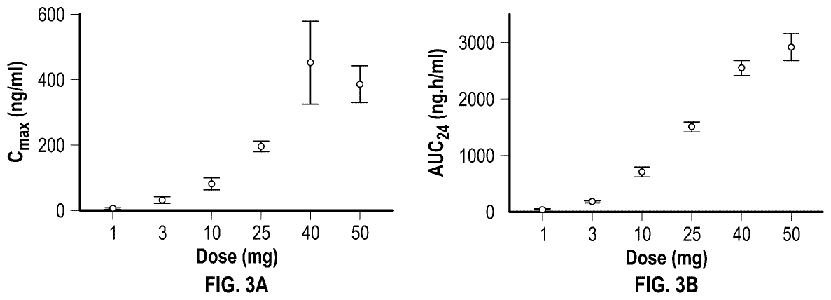

Single-dose kinetics. The plasma aficamten profiles were generally well characterized for all dose levels with the exception of the lowest dose of 1 mg (due to concentrations close to the lower limit of quantitation) and the highest dose of 75 mg, which was administered to only 1 participant, as discussed above. Over the dose range of 1 mg to 50 mg, mean maximal plasma concentrations and exposure increased in a dose-proportional manner, as demonstrated by the rise in Cₘₐₓ and area under the plasma concentration-time curve from time 0 to 24 h (AUC₂₄) with increasing doses (FIG. 3A-3B and Table 5). Mean clearance and volume of distribution were similar across the doses. Median time to maximum observed concentration occurred between 0.5 and 2.8 h, with a maximum time of 4.0 h across all participants. Mean half-life ranged from 75 to 85 h.

TABLE 5
Summary of Plasma Aficamten Pharmacokinetics Following Single Oral Dose Administration

| | Aficamten SAD Cohort, mg | | | | | | |
|---|---|---|---|---|---|---|---|
| | 1 | 3 | 10 | 25 | 40 | 50 | 75 |
| Cₘₐₓ | 5.8 (38.4) | 26.1 (82.4) | 70.5 (69.0) | 192.6 (23.3) | 383.2 (67.9) | 359.4 (38.9) | 1,220 (—) |
| (ng/ml) | n = 6 | n = 6 | n = 6 | n = 6 | n = 6 | n = 11 | n = 1 |
| Tₘₐₓ (h) | 1.0 (0.5, 4.0) | 1.3 (0.5, 2.6) | 2.8 (0.5, 3.0) | 1.8 (0.5, 4.0) | 1.0 (0.5, 1.5) | 1.0 (0.5, 2.5) | 0.5 (0.5, 0.5) |
| | n = 6 | n = 6 | n = 6 | n = 6 | n = 6 | n = 11 | n = 1 |
| AUC₂₄ | 50 (20) | 174 (27) | 679 (35) | 1,493 (15) | 2,532 (13) | 2,833 (26) | 4,556 (—) |
| (ng · h/ml) | n = 6 | n = 6 | n = 6 | n = 6 | n = 6 | n = 11 | n = 1 |
| AUC_{inf} | — | 823 (61) | 3,434 (24) | 7,113 (15) | 11,860 (20) | 13,740 (35) | |
| (ng · h/ml) | — | n = 5 | n = 4 | n = 6 | n = 5 | n = 9 | |
| Vz/F (L) | — | 392.7 ± 122.2 | 361.7 ± 108.0 | 405.3 ± 56.6 | 381.7 ± 66.9 | 431.3 ± 122.9 | |
| | — | n = 5 | n = 4 | n = 6 | n = 5 | n = 9 | |
| t_{1/2} (h) | — | 75.1 ± 21.8 | 84.7 ± 17.7 | 79.8 ± 10.0 | 79.2 ± 18.5 | 80.4 ± 14.9 | |
| | — | n = 5 | n = 4 | n = 6 | n = 4 | n = 9 | |
| CL/F (L/h) | — | 4.2 ± 3.1 | 3.0 ± 0.7 | 3.6 ± 0.5 | 3.4 ± 0.6 | 3.8 ± 1.1 | |
| | — | n = 5 | n = 4 | n = 6 | n = 5 | n = 9 | |

Cₘₐₓ and AUC values are presented as geometric mean and geometric CV %, Tₘₐₓ as median (range), and all other parameters as arithmetic mean ± standard deviation. AUC₂₄ = area under the plasma drug concentration-time curve from time 0 to 24 h; AUC_{inf} = area under the plasma drug concentration-time curve from time 0 extrapolated to infinity; CL/F = apparent total body clearance; Cₘₐₓ = maximum plasma concentration; CV % = percent coefficient of variation; t_{1/2} = half-life; Tₘₐₓ = time to maximum plasma concentration; Vz/F = apparent volume of distribution.

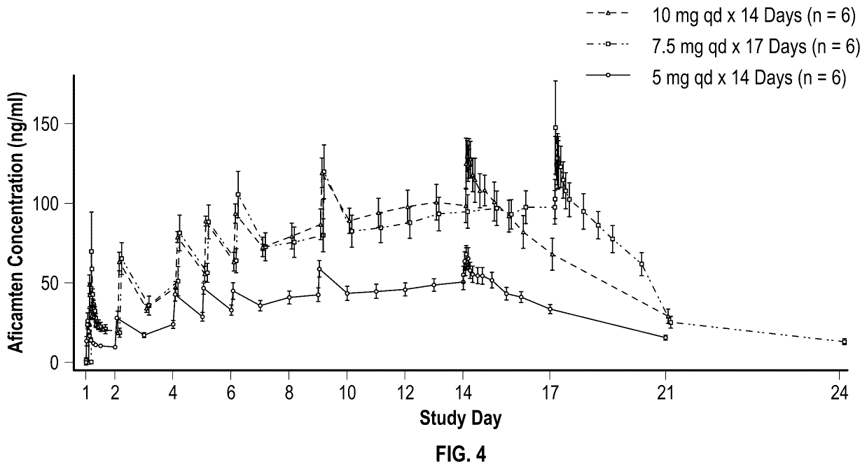

Multiple-dose kinetics. With once-daily dosing, mean plasma concentrations increased between the 5-mg dose and the 2 higher doses (7.5 mg and 10 mg); however, by day 2 there was little difference between mean concentrations of the 7.5-mg and 10-mg doses (FIG. 4). Plasma PK parameters are displayed in Table 6. By the end of the treatment period (day 14 or 17), the mean plasma concentration was between 2 and 2.5 times that of day 1. Terminal elimination half-life estimates were consistent across doses, ranging between 77 and 86 h. Clearance was similar for the 5-mg and 10-mg doses, and the accumulation ratio was similar for the 3 doses. Consistent with the observed terminal elimination half-life estimates, steady state was achieved after 10 to 12 days (FIG. 4).

TABLE 6
Summary of Plasma Aficamten Pharmacokinetics
Following Multiple Oral Dose Administration

| Multiple-Ascending Dose (MAD) Cohorts | | |
|---|---|---|
| 5 mg qd × | 7.5 mg qd × | 10 mg qd × |
| 14 Days | 17 Days | 14 Days |
| (n = 6) | (n = 6) | (n = 6) |

Day 1

| | | | | | | |
|---|---|---|---|---|---|---|
| Cₘₐₓ (ng/ml) | 28.0 | (30.1) | 69.3 | (66.5) | 54.4 | (25.1) |
| Tₘₐₓ (h) | 1.3 | (0.5, 2.5) | 0.8 | (0.5, 1.5) | 1.0 | (1.0, 2.5) |
| AUC₂₄ | 278 | (21) | 551 | (33) | 547 | (11) |
| (ng · h/ml) | | | | | | |

Day 14/17

| | | | | | | |
|---|---|---|---|---|---|---|
| C_{max, ss} (ng/ml) | 69.0 | (23) | 147.6 | (39.5) | 141.2 | (19.7) |
| T_{max, ss} (h) | 2.8 | (1.5, 4.0) | 1.0 | (0.5, 5.0) | 2.5 | (0.5, 3.0) |
| AUCₜₐᵤ | 1320 | (23) | 2518 | (26) | 2632 | (23) |
| (ng · h/ml) | | | | | | |

| | | | |
|---|---|---|---|
| t_{1/2} (h) | 86.4 ± 11.9 | 76.9 ± 14.5 | 79.8 ± 14.1 |
| CLₛₛ/F (h) | 3.9 ± 0.9 | 3.1 ± 0.7 | 3.9 ± 0.8 |
| RA,_{AUC} | 4.8 ± 0.2 | 4.6 ± 0.7 | 4.9 ± 0.9 |

AUC₂₄, Cₘₐₓ, AUCₜₐᵤ, and C_{max, ss} values are presented as geometric mean and geometric CV %, Tₘₐₓ and T_{max, ss} values as median (range), and all other parameters as arithmetic mean ± standard deviation. AUC₂₄ = area under the plasma drug concentration-time curve from time 0 to 24 h; AUCₜₐᵤ = AUC to the end of the dosing period; CLₛₛ/F = apparent total body clearance after oral administration (at steady state); Cₘₐₓ = maximum plasma concentration; C_{max, ss} = maximum plasma concentration at steady state; CV % = percent coefficient of variation; RA,_{AUC} = accumulation ratio calculated from AUCₜₐᵤ at steady and following a single dose; t_{1/2} = half-life; Tₘₐₓ = time to maximum plasma concentration; T_{max, ss} = time to reach maximum plasma concentration following drug administration at steady state.

CYP2D6 poor metabolizer cohort. In CYP2D6-PMs, mean half-life was prolonged to 110 h, compared with 85 h in extensive metabolizers (i.e., the 10-mg SAD cohort); however, no increase in AUC was observed in this group, with geometric mean AUC₂₄ of 495 ng·h/ml (geometric percent coefficient of variation [CV %] 19) (Table 7), compared with 679 ng·h/ml (geometric CV % 35) in extensive metabolizers (Table 5). The CYP2D6-PMs did not appear to have reduced clearance that resulted in a clinically meaningful difference in exposures.

TABLE 7
Summary of Plasma Aficamten Pharmacokinetics
in the CYP2D6 Poor Metabolizer Cohort

| | CYP2D6 Poor Metabolizers | |
|---|---|---|
| PK Parameter | (n = 7) | |
| Cₘₐₓ (ng/ml) | 57.3 | (55.0) |
| Tₘₐₓ (h) | 1.0 | (0.5, 4.0) |
| AUC₂₄ (ng · h/ml) | 495 | (19) |
| AUC_{inf} (ng · h/ml) | 2966 | (46) |

| | |
|---|---|
| t_{1/2} (h) | 110.2 ± 47.3 |
| CL/F (L/h) | 3.6 ± 1.4 |
| Vz/F (L) | 506.0 ± 84.4 |

Aficamten dosage was 10 mg. AUC and Cₘₐₓ values are presented as geometric mean and geometric CV %, Tₘₐₓ values as median (range), and all other parameters as arithmetic mean ± standard deviation. AUC₂₄ = area under the plasma drug concentration-time curve from time 0 to 24 h; AUC_{inf} = AUC from time 0 extrapolated to infinity; CL/F = apparent total body clearance; Cₘₐₓ = maximum plasma concentration; CV % = percent coefficient of variation; PK = pharmacokinetic; t_{1/2} = half-life; Tₘₐₓ = time to maximum plasma concentration; Vz/F = apparent volume of distribution.

Effect of food. The PK parameters of aficamten in the food-effect cohort are displayed in Table 8. When taken with food, the Cₘₐₓ of aficamten increased by approximately 30% and the time to maximum observed concentration was reduced (1.5 vs. 2.3 h). However, food had little effect on AUC, with geometric mean AUC₂₄ (geometric CV %) in the fasted state of 601 (33) ng·h/ml versus 631 (25) in the fed state.

TABLE 8
Summary of Plasma Aficamten Pharmacokinetics
in the Food-Effect Cohort

| | | |
|---|---|---|
| PK Parameter | Fasted (n = 10)* | Fed (n = 12)^{†} |

| | | | | |
|---|---|---|---|---|
| Cₘₐₓ (ng/ml) | 50.6 | (47.2) | 65.0 | (69.5) |
| Tₘₐₓ (h) | 2.3 | (0.6, 6.0) | 1.5 | (0.5, 6.0) |
| AUC₂₄ (ng · h/ml) | 601 | (33) | 631 | (25) |
| AUC_{inf} (ng · h/ml) | 3674 | (24) | 3413 | (32) |

| | | |
|---|---|---|
| t_{1/2} (h) | 84.4 ± 16.4 | 78.0 ± 8.1 |
| CL/F (L/h) | 2.8 ± 0.6 | 3.1 ± 0.9 |
| Vz/F (L) | 345.7 ± 133.7 | 339.7 ± 100.4 |

Aficamten dosage was 10 mg in each cohort.
*n = 4 for AUC_{inf}, t_{1/2}, CL/F, and Vz/F.
^{†}n = 7 for AUC_{inf}, t_{1/2}, CL/F, and Vz/F.
AUC and Cₘₐₓ values are presented as geometric mean and geometric CV %, Tₘₐₓ as median (range), and all other parameters as arithmetic mean ± standard deviation. AUC₂₄ = area under the plasma drug concentration-time curve from 0 to 24 h; AUC_{inf} = AUC from time 0 extrapolated to infinity; CL/F = apparent total body clearance; Cₘₐₓ = maximum plasma concentration; CV % = percent coefficient of variation; t_{1/2} = half-life; Tₘₐₓ = time to maximum plasma concentration; Vz/F = apparent volume of distribution.

### Pharmacodynamics

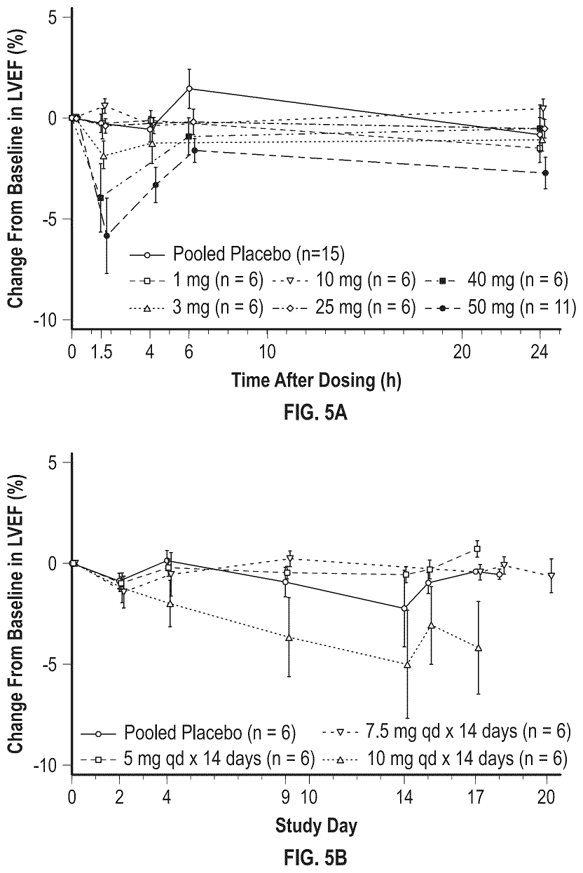

Left ventricular ejection fraction. At baseline, mean LVEF ranged from 61.0% to 67.5% across cohorts (Table 1). In the SAD cohorts, mean decreases in LVEF were observed in the groups receiving the highest doses of aficamten (FIG. 5A). Maximum mean reduction from baseline was seen in the 50-mg cohort at 1.5 h post dose (least-squares mean difference 5.5%, p=0.0001). LVESV and LVEDV were statistically significantly increased by 8.1 and 6.6 mL, respectively (Table 9). Other echocardiographic parameters such as stroke volume, cardiac output, cardiac time intervals, and measures reflective of diastolic function did not significantly change (Table 9). The single participant who received 75 mg of aficamten exhibited reduction in LVEF of 31.5% at 1.5 h post dose, which resolved 2.5 h after onset but led to concluding escalation of doses in the SAD portion of the study, as discussed above. In the MAD cohorts, a clear decrease in LVEF emerged as dosing continued in the 10-mg cohort (FIG. 5B). The largest mean maximum percent reduction from baseline, of 5.0%, was seen in the 10-mg cohort at 1.5 h post dose on day 14 (FIG. 5B). The placebo-corrected reduction of 3.2% (least-squares mean difference) did not reach statistical significance (p=0.21), likely due to the lack of statistical power in this small group comparison.

TABLE 9
Echocardiographic Parameters

| | Placebo (n = 15) | | | | | | 50 mg (n = 11) | | | | | | |
|---|---|---|---|---|---|---|---|---|---|---|---|---|---|
| | Baseline | | 1.5 h | | CFB | | Baseline | | 1.5 h | | CFB | | p value |
| | Mean | SD | Mean | SD | Mean | SD | Mean | SD | Mean | SD | Mean | SD | CFB v CFB |
| Heart rate (bpm) | 61.6 | 5.6 | 61.4 | 7.6 | −0.2 | 5.6 | 61.7 | 11.5 | 62.3 | 10.6 | 0.5 | 5.8 | NS |
| LVEF (%) | 66 | 2 | 65.8 | 2 | −0.3 | 1.8 | 66.3 | 3.8 | 60.5 | 7.3 | −5.8 | 6.1 | 0.003 |
| LV ESV (mL) | 32 | 5.5 | 31.1 | 5.5 | −0.9 | 3.6 | 31.6 | 6.8 | 39.7 | 10.4 | 8.1 | 8.4 | 0.001 |
| LV EDV (mL) | 93.6 | 15.2 | 90.6 | 14.4 | −3 | 8.1 | 93.2 | 13.2 | 99.8 | 13.4 | 6.6 | 10.6 | 0.016 |
| LV SV (mL) | 56.7 | 6.3 | 56.1 | 9.7 | −0.6 | 6.8 | 58.9 | 8 | 52.6 | 10.5 | −6.2 | 7.6 | NS |
| LV CO (mL) | 3470 | 299 | 3450 | 655 | −21.4 | 538 | 3580 | 597 | 3240 | 681 | −346 | 498 | NS |
| IVCT (msec) | 46.9 | 2.4 | 46.9 | 6.4 | 0.4 | 5.9 | 42.3 | 1.6 | 42.6 | 1.8 | 0.4 | 2.9 | NS |
| IVRT (msec) | 72.1 | 10.6 | 73.9 | 7 | 1.9 | 11.3 | 68.7 | 3 | 69.3 | 3.3 | 0.5 | 3.8 | NS |
| LV ET (msec) | 332 | 32.2 | 324 | 26.3 | −6.9 | 12.5 | 344 | 35.1 | 328 | 34.1 | 1.9 | 12.8 | NS |
| LAV (mL) | 36 | 8.4 | 35.3 | 9.3 | −1 | 7.6 | 35.7 | 9.9 | 35.7 | 7.2 | 0 | 4.7 | NS |
| E (cm/sec) | 73.6 | 6.7 | 68.2 | 8.2 | −5.3 | 5.7 | 66.7 | 14.7 | 64 | 14.7 | −2.7 | 4.5 | NS |
| A (cm/sec) | 49.4 | 11.4 | 47.7 | 10.3 | −1.7 | 5.6 | 52 | 12.8 | 54.5 | 17.5 | 2.5 | 6.6 | NS |
| e′ lateral (cm/sec) | 12.7 | 2.5 | 12.3 | 2.7 | −0.7 | 1.5 | 11.5 | 3.1 | 10.2 | 2.4 | −1.3 | 1.6 | NS |
| E/A ratio | 1.6 | 0.4 | 1.5 | 0.4 | −0.1 | 0.2 | 1.3 | 0.1 | 1.2 | 0.2 | −0.1 | 0.1 | NS |
| E/e′ ratio | 6 | 1.2 | 5.8 | 1.3 | −0.1 | 0.7 | 6.3 | 2.8 | 6.8 | 3.3 | 0.5 | 1.5 | NS |

The table compares the placebo data of echocardiographic parameters at baseline and 1.5 h (the time of the echocardiogram closest to peak of plasma concentrations of aficamten) to those at 50 mg of aficamten (the highest well tolerated single dose).
A = peak A wave velocity,
Bpm = beat per minute,
CFB = change from baseline,
CO = cardiac output,
E = peak E wave velocity,
EDV = end-systolic volume,
ESV = end-systolic volume,
ET = ejection time,
e′ lateral = tissue doppler velocity of the lateral wall,
IVCT = isovolumic contraction time,
IVRT = isovolumic relaxation time
LAV = left atrial volume,
LVEF = left ventricular ejection fraction,
NS = not significant (p > 0.05),
SD = Standard deviation,
SV = stroke volume

Categorical LVEF responses. In the SAD cohorts, absolute reductions in LVEF of ≥5% from baseline were observed in 1 of 15 participants in the placebo cohort (7%), 1 of 6 (17%) in the 3-mg cohort, 2 of 6 (33%) in the 40-mg cohort, 7 of 11 (64%) in the 50-mg cohort, and 1 of 1 (100%) in the 75-mg cohort, while no participants in the 1-, 10-, or 25-mg cohorts had a reduction ≥5%. Absolute reductions in LVEF of ≥10% occurred in 1 of 6 participants (17%) in the 40-mg cohort, 2 of 11 (18%) in the 50-mg cohort, and 1 (100%) in the 75-mg cohort. Reduction in LVEF to <50% was observed for 2 of 11 participants (18%) in the 50-mg cohort (48.2% and 45.5% per core laboratory assessment), and 1 of 1 (100%) in the 75-mg cohort. Only the participant in the 75-mg cohort experienced LVEF <45%.

In the MAD cohort, absolute reduction in LVEF of ≥5% from baseline was observed in 4 participants: 1 of 6 participants (17%) receiving placebo, 1 of 6 (17%) receiving 7.5 mg of aficamten once daily, and 2 of 6 (33%) receiving 10 mg of aficamten once daily. Of these, reductions were ≥10% in the 2 participants in the 10-mg cohort. Reduction in LVEF to <50% was not observed in any of the MAD cohorts per core laboratory assessment.

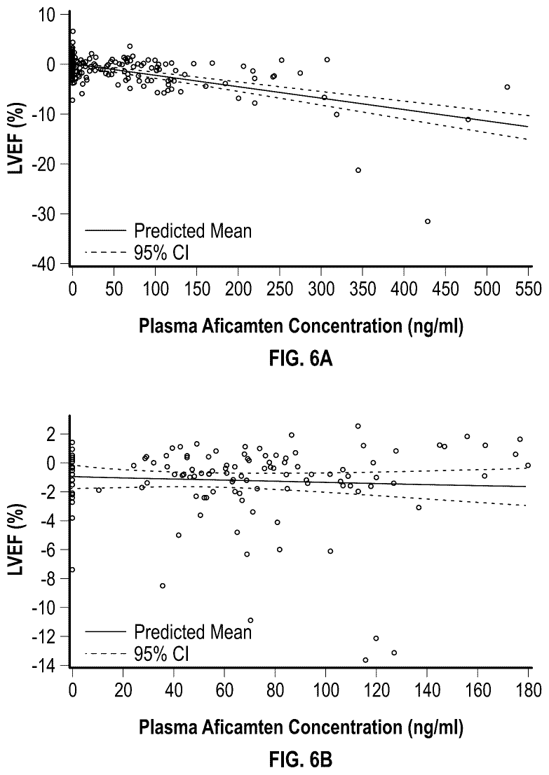

Relationship of Plasma Concentration to Change in LVEF. The PK/PD relationship for aficamten is illustrated by plotting the plasma concentration of aficamten versus change in LVEF for the SAD and MAD cohorts (FIG. 6A and FIG. 6B). In the SAD cohort, as plasma concentration of aficamten increased, there was a trend toward a decrease in LVEF. The relationship of LVEF to the plasma concentration of aficamten was statistically significant, both in the bin concentration analysis for the highest plasma concentration bin (122 to 524 ng/ml, p<0.0001) and in the concentration-slope analysis (p=0.0027). In the MAD cohorts, the relationship of LVEF to plasma aficamten did not reach statistical significance in the bin concentration analysis or the linear regression analysis, likely due to the more limited range of plasma concentrations explored and the small group sizes.

### Discussion

This phase 1, first-in-human study has established the doses (up to 50 mg as a single oral dose or up to 10 mg following multiple doses) at which aficamten was both physiologically effective at reducing LVEF and was well tolerated in healthy participants, identifying pharmacologically active doses that will serve as starting doses for a study in patients with HCM. In addition, single oral doses of 10 mg were well tolerated among individuals with the CYP2D6-PM phenotype and there was no significant effect of food on the PK of aficamten. Collectively, these observations support the continued development of aficamten for patients with HCM and provide a roadmap for phase 2 studies.

Safety of aficamten. No serious AEs were observed in the study and all participants completed intended dosing as planned. Generally, AEs were mild and similar in frequency between participants treated with aficamten and placebo. Importantly, there were no associated symptoms or adverse changes in vital signs for participants whose LVEF fell below 50% and the LVEF in these cases returned to baseline within 24 h. This study was not intended to find a maximum tolerated dose and hence dose escalation stopped once a clear PD effect was observed in the SAD and MAD portions of the study; thus, a dose that was not tolerated due to AEs was not identified.

Effect on LVEF. In the SAD cohorts, a dose of 50 mg produced a mean reduction in LVEF of 5.8% while in the MAD cohort, 10 mg once daily for 14 days produced a mean absolute reduction in LVEF of approximately 5%. The proportion of participants with absolute reductions in LVEF of ≥5% from baseline increased as the dose increased; up to 64% of participants in the 50-mg SAD cohort and 33% in the 10-mg MAD cohort had absolute reductions in LVEF of ≥10% from baseline. In the SAD cohorts, where the broadest range of exposures of aficamten was explored, there was a statistically significant decrease in LVEF as plasma concentrations of aficamten increased. Thus, the study achieved its secondary objective of identifying a pharmacologically active dose and describing its PK/PD relationship.

Three participants had decreases in LVEF to <50% that were rapidly reversible upon study drug discontinuation. Following a single dose of 50 mg, 2 (18%) participants experienced LVEF <50% (48.2% and 45.5%). After a single dose of 75 mg, 1 participant experienced reduction of LVEF to 34.1%. In all cases, the event was noted approximately 1.5 h after dosing, and LVEF recovered to >50% by 4 to 6 h after dosing. The SAD results informed dose selection for the other portions of the study, and there were no echocardiographic AEs in the MAD, CYP2D6-PM, or food-effect cohorts.

Implications of PK results. Aficamten demonstrated linear kinetics over the dose range of 1 mg to 50 mg; half-life was independent of concentration, and clearance was independent of dose. Steady state was achieved by the end of day 10 with the 10-mg dose and by the end of day 12 with the 5-mg and 7.5-mg doses. There was no effect of food suggestive of a need to alter dosing. These findings support once-daily dosing in either the fasted or fed state.

The relationship between plasma concentration and LVEF suggests a broad therapeutic index, which will facilitate optimization of individual doses in patients with HCM, who are expected to be titrated through an escalating range of doses until the desired PD effect is achieved. In addition, the half-life of aficamten (75 to 85 h following a single dose; 77 to 86 h following multiple doses) and observed reversibility of effect offers a potential advantage in that steady state is achieved within 2 weeks and excessive effects on LVEF are readily reversed.

Conclusions. Aficamten demonstrated a favorable safety profile in healthy participants, without serious AEs or meaningful changes in laboratory tests, ECGs, or health assessments. Any decreases in LVEF to values <50% were reversible within 6 h following single doses. Pharmacologically active doses of aficamten that may serve as starting doses for a study in patients with HCM were identified.

### Example 2

A multi-center, randomized, placebo-controlled, double-blind, dose finding phase 2 clinical trial of aficamten in patients with symptomatic obstructive HCM (oHCM) was conducted. The primary objective of the trial was to determine the safety and tolerability of aficamten. The secondary objectives were to describe the concentration-response relationship of aficamten on the resting and post-Valsalva left ventricular outflow tract gradient as measured by echocardiography during 10 weeks of treatment, to describe the dose response relationship of aficamten, and to evaluate the plasma concentrations of aficamten in patients with oHCM. Seventeen investigative sites in North America and Europe screened for patients to enroll in Cohorts 1 and 2. A third cohort (Cohort 3) was also studied to evaluate the safety and efficacy of aficamten in combination with disopyramide, a class IA antiarrhythmic drug.

The first two cohorts (Cohort 1 and Cohort 2) excluded patients receiving disopyramide. Cohort 3 included patients receiving disopyramide. Within each of the first two cohorts, patients were randomized 2:1 to active or placebo treatment and received up to three escalating doses of aficamten or placebo based on echocardiographic guidance. In the third cohort, all patients received up to three escalating doses of aficamten based on echocardiographic guidance. Overall, the treatment duration was 10 weeks with a 4-week follow-up period after the last dose.

Since patient characteristics vary substantially in this disease, individualized dose titration to a pharmacodynamics (PD) response (reduction of the LVOT-G to <30 mmHg with preservation of LVEF >50%) was employed to maximize efficacy and safety.

Patients were eligible to be included in the study only if all the following criteria apply: 1. Able to comprehend and willing to sign an informed consent form (ICF) and willing to comply with all study procedures and restrictions for the duration specified in the Schedule of Activities; 2. Males and females between 18 and 85 years of age at screening; 3. Body weight was ≥45 kg at screening; 4. Diagnosed with oHCM per the following criteria: (a) as LV hypertrophy and non-dilated LV chamber in the absence of other cardiac disease; and (b) had minimal wall thickness ≥15 mm (minimal wall thickness ≥13 mm was acceptable with a positive family history of HCM or with a known disease-causing gene mutation); 5. Adequate acoustic windows for echocardiography; 6. Had LVOT-G during screening as follows for Cohorts 1 and 2: (a) resting gradient ≥50 mmHg; or (b) resting gradient ≥30 mmHg and <50 mmHg with post-Valsalva LVOT-G ≥50 mmHg; or as follows for Cohort 3: persistent resting LVOT obstruction (≥30 mmHg) and provoked LVOT obstruction (≥50 mmHg); 7. Left ventricular ejection fraction (LVEF) ≥60% at screening; 8. New York Heart Association (NYHA) Class II or III at screening; 9. Patients on beta-blockers, verapamil, diltiazem, or ranolazine should have been on stable doses for ≥4 weeks prior to randomization and anticipate remaining on the same medication regimen during the study; 10. Male patients were eligible to participate if they agreed to the following during the study and for at least 10 weeks after the last dose: (a) refrain from donating sperm; plus either (b)(i) be abstinent from heterosexual intercourse as their preferred and usual lifestyle (abstinent on a long term and persistent basis) and agree to remain abstinent; or (b)(i) must agree to use a male condom and, when his female partner is a woman of childbearing potential, have his female partner use a highly effective method of contraception; 11. A female patient was eligible to participate if she is not pregnant or breastfeeding, and at least one of the following conditions applies: (a)(i) is not a woman of childbearing potential, or (a)(ii) is a woman of childbearing potential and using a highly effective method of contraceptive during the study and for at least 4 weeks after the last dose; and (b) a woman of childbearing potential must have a negative pregnancy test (urine or serum as required by local regulations) within 3 days before the first dose of study intervention; 12. Able to complete all screening procedures; and 13. Taking stable doses of disopyramide for >4 weeks prior to screening (Cohort 3 only).

Patients were excluded from the study if any of the following criteria apply: 1. Aortic stenosis or fixed subaortic obstruction; 2. Known infiltrative or storage disorder causing cardiac hypertrophy that mimics oHCM (e.g., Noonan syndrome, Fabry disease, amyloidosis); 3. History of left ventricular (LV) systolic dysfunction (LVEF <45%) at any time during their clinical course; 4. Documented history of current obstructive coronary artery disease (>70% stenosis in one or more epicardial coronary arteries) or documented history of myocardial infarction; 5. Has been treated with septal reduction therapy (surgical myectomy or percutaneous alcohol septal ablation) or has plans for either treatment during the study period; 6. Prior treatment with cardiotoxic agents such as doxorubicin or similar; 7. For Cohorts 1 and 2: Has been treated with disopyramide or antiarrhythmic drugs that have negative inotropic activity within 4 weeks prior to screening, and for Cohort 3: Has been treated with an antiarrhythmic drug other than disopyramide that has negative inotropic activity within 4 weeks prior to screening; 8. Has any ECG abnormality considered by the investigator to pose a risk to patient safety (e.g., second degree atrioventricular block type II); 9. Paroxysmal atrial fibrillation or flutter documented during the screening period; 10. Paroxysmal or permanent atrial fibrillation requiring rhythm restoring treatment (e.g., direct-current cardioversion, ablation procedure, or antiarrhythmic therapy) <6 months prior to screening, except that this exclusion does not apply if atrial fibrillation has been treated with anticoagulation and adequately rate-controlled for >6 months; 11. History of syncope or sustained ventricular tachyarrhythmia with exercise within 6 months prior to screening; 12. Implantable cardioverter defibrillator (ICD) placement within 3 months prior to screening or planned ICD placement during the study; 13. History of appropriate ICD shock for life-threatening ventricular arrhythmia within six months prior to screening; 14. Recipient of a major organ transplant (e.g., heart, lung, liver, bone marrow, renal) or anticipated transplantation within 12 months from randomization); 15. Hepatic impairment defined by a total bilirubin (TBL) ≥1.5×the upper limit of normal (ULN), or alanine aminotransferase (ALT) or aspartate aminotransferase (AST) ≥3×ULN at screening, except that patients with documented Gilbert syndrome and TBL ≥1.5×ULN due to unconjugated hyperbilirubinemia, without other hepatic disease, were permitted; 16. History or evidence of any other clinically significant disorder, malignancy, active infection, other condition, or disease that, in the opinion of the investigator or the Medical Monitor, would pose a risk to patient safety or interfere with the study evaluation, procedures, or completion; 17. Hemoglobin <10.0 g/dL at screening; 18. Estimated glomerular filtration rate (eGFR) <30 mL/min/1.73 m² (by the modified Modification of Diet in Renal Disease equation) at screening; 19. Currently participating in another investigational device or drug study or received an investigational device or drug <1 month (or 5 half-lives for drugs, whichever is longer) prior to screening; 20. Has received prior treatment with aficamten or is currently receiving mavacamten; 21. Has a known hypersensitivity to any excipients in aficamten Tablets, Film-Coated (e.g., mannitol, microcrystalline cellulose, croscarmellose, hydroxypropyl cellulose, sodium lauryl sulfate, magnesium stearate, Opadry QX White 21A180025).

In each cohort, a patient received up to three escalating doses of aficamten as shown in Table 11. Each patient received Dose 1 once daily for 2 weeks. At Week 2, the patient had an echocardiogram 2 hours following administration of their dose. Patients up-titrated to Dose 2 if either of the following conditions were met on echocardiography: (1) resting LVOT-G ≥30 mmHg and the biplane LVEF ≥50%; or (2) resting LVOT-G <30 mmHg, post-Valsalva LVOT-G ≥50 mmHg, and the biplane LVEF ≥50%. Otherwise, the patient remains on Dose 1. If LVEF is <50% at Week 2, the patient was down-titrated to placebo. The dose-adjustment algorithm is shown below in Table 10.

After 2 more weeks on the assigned dose (i.e., Week 4), each patient had an echocardiogram 2 hours following administration of their dose. Patients were escalated to the next higher dose if either of the following conditions were met on echocardiography: (1) resting LVOT-G ≥30 mmHg and the biplane LVEF ≥50%; or (2) resting LVOT-G <30 mmHg, post-Valsalva LVOT-G ≥50 mmHg, and the biplane LVEF ≥50%. Otherwise, the patient remained on the same dose. If LVEF was <50% at Week 4, the patient was returned to a prior dose level or to placebo if the patient was on Dose 1.

After 2 more weeks on the assigned dose (i.e., Week 6), each patient had an echocardiogram 2 hours following administration of their dose. If LVEF was <50% at Week 6, the patient was down-titrated to a prior dose level or to placebo if the patient was on Dose 1.

TABLE 10
Dose Adjustment Algorithm

| Biplane | | | |
|---|---|---|---|
| LVEF | | LVOT Gradient | Action |
| <40% | | | Permanent discontinuation |
| <50% | | | Dose reduce to next lower dose |
| | | | or placebo (if on lowest dose) |
| ≥50% | and | Resting LVOT gradient <30 mmHg | Dose unchanged |
| | | AND | |
| | | Valsalva LVOT gradient <50 mmHg | |
| ≥50% | and | Rest LVOT gradient ≥30 mmHg | Dose escalate to next dose level |
| | | AND/OR | |
| | | Valsalva LVOT gradient ≥50 mmHg | |

If at any time, a patient's dose was down-titrated to placebo, then they remained on placebo for the duration of the study.

TABLE 11
Dosing Scheme

| Cohort | Dose 1 | Dose 2 | Dose 3 |
|---|---|---|---|
| Cohort 1 | 5 mg | 10 mg | 15 mg |
| Cohort 2 | 10 mg | 20 mg | 30 mg |
| Cohort 3 | 5 mg | 10 mg | 15 mg |

Baseline characteristics of patients in Cohort 1, Cohort 2, and Cohort 3 are shown in Table 12 and Table 13.

TABLE 12
Baseline Characteristics of Patients in Phase 2 Clinical Trial

| | Cohort 1 | | Cohort 2 | | Cohort 3 | |
|---|---|---|---|---|---|---|
| Characteristic | (N = 21) | | (N = 20) | | (N = 13) | |
| Age (Years), Mean (SD) [Range] | 56.7 (12.1) | | 57.0 (13.0) | | 59.4 (14.4) | |
| | [33, 69] | | [35, 78] | | [23, 82] | |
| Female, n (%) | 10 | (47.6) | 13 | (65.0) | 7 | (53.8) |
| BMI (kg/m2), Mean (SD) [Range] | 29.1 (5.3) | | 30.3 (6.6) | | 30.1 (6.2) | |
| | [20.2, 39.9] | | [22.3, 48.4] | | [22.7, 41.2] | |
| NYHA Class, n (%) | | | | | | |
| Class II | 16 | (76.2) | 12 | (60.0) | 5 | (38.5) |
| Class III | 5 | (23.8) | 8 | (40.0) | 8 | (61.5) |
| Beta Blocker Use, n (%) | 16 | (76.2) | 16 | (80.0) | 11 | (84.5) |
| LVEF at Screening (%), Mean (SD) | 72.9 | (7.9) | 71.8 | (7.0) | 69.9 | (7.1) |
| LVOT-G, Rest at Screening | 61.0 | (28.4) | 67.1 | (30.4) | 52.3 | (21.7) |
| (mmHg), Mean (SD) | | | | | | |
| LVOT-G, Valsalva at Screening | 88.2 | (24.6) | 93.0 | (35.1) | 97.1 | (36.4) |
| (mmHg), Mean (SD) | | | | | | |
| NT-proBNP (pg/mL), | 368.5 | (255.0) | 573.8 | (145.5) | 1050.6 | (147.3) |
| Geometric Mean (CV %) | | | | | | |

TABLE 13
Baseline Demographic and Clinical Characteristics of
Patients in Phase 2 Clinical Trial, Cohorts 1 and 2

| | Pooled | | | | | | Pooled | |
|---|---|---|---|---|---|---|---|---|
| | Placebo | | Cohort 1 | | Cohort 2 | | Aficamten | |
| Characteristics | (N = 13) | | (N = 14) | | (N = 14) | | (N = 28) | |
| Age, y | 57 ± 10 | | 54 ± 14 | | 59 ± 14 | | 57 ± 14 | |
| Female, n (%) | 8 | (62) | 4 | (29) | 11 | (79) | 15 | (54) |
| Race, white, n (%) | 12 | (92) | 14 | (100) | 14 | (100) | 28 | (100) |
| BMI, kg/m² | 29 ± 6 | | 30 ± 5 | | 30 ± 6 | | 30 ± 6 | |
| NYHA FC, n (%) | | | | | | | | |
| Class II | 11 | (85) | 10 | (71) | 7 | (50) | 17 | (61) |
| Class III | 2 | (15) | 4 | (29) | 7 | (50) | 11 | (39) |
| NT-proBNP, pg/mL, | 395 | (227) | 344 | (254) | 695 | (124) | 520 | (189) |
| Geometric Mean (% CV) | | | | | | | | |
| High sensitivity cardiac | 15 | (416) | 8 | (496) | 10 | (115) | 9 | (306) |
| troponin I, ng/ml (% CV) | | | | | | | | |
| Basal interventricular septal | 15 | (3) | 18 | (3) | 16 | (3) | 17 | (3) |
| wall thickness, mm (SD) | | | | | | | | |
| Posterior wall thickness, | 12 | (2) | 13 | (2) | 12 | (2) | 12 | (2) |
| mm (SD) | | | | | | | | |
| LAVI, mL/m² (SD) | 31.4 | (7.5) | 31.9 | (9.2) | 33.0 | (7.2) | 32.5 | (8.1) |
| LVED, mm (SD) | 39 | (3) | 39 | (5) | 39 | (4) | 39 | (4) |
| LVEF, % (SD) * | 74 | (6) | 71 | (8) | 72 | (8) | 72 | (8) |
| LVOT gradient at rest, | 70 | (28) | 52 | (23) | 70 | (34) | 61 | (30) |
| mmHg (SD) * | | | | | | | | |
| LVOT gradient with | 93 | (27) | 83 | (19) | 96 | (40) | 89 | (31) |
| Valsalva, mmHg (SD) * | | | | | | | | |
| Lateral E/e′ ratio, Mean | 17.4 | (10.0) | | | | | 13.8 | (6.3) |
| (SD) | | | | | | | | |
| Lateral e′ (cm/s), Mean | 5.8 | (2.1) | | | | | 6.7 | (2.3) |
| (SD) | | | | | | | | |
| LAVI (mL/m²), Mean (SD) | 31.4 | (7.5) | | | | | 32.5 | (8.1) |
| LVMI (g/m²), Mean (SD) | 103.3 | (25.1) | | | | | 109.8 | (28.6) |
| Presence of MR, n (%) | 11 | (84.6%) | | | | | 26 | (92.9%) |
| Presence of Eccentric MR, | 3 | (25.0%) | | | | | 12 | (42.9%) |
| n (%) | | | | | | | | |
| Presence of SAM, n (%) | 12 | (92.3%) | | | | | 24 | (85.7%) |
| Medications | | | | | | | | |
| Beta-blocker, n (%) | 11 | (85) | 10 | (71) | 11 | (79) | 21 | (75) |
| Calcium channel-blocker, n | 2 | (15) | 5 | (36) | 2 | (14) | 7 | (25) |
| (%) | | | | | | | | |

* = derived from individual investigator site echocardiographic assessments. BMI = body mass index; CV = coefficient of variation; LVEF = left ventricular ejection fraction; LA = left atrium; LVED = left ventricular end diastolic dimension; LVOT = left ventricular outflow tract; NT-proBNP = N-terminal pro B-type natriuretic peptide; NYHA FC = New York Heart Association functional class.

Echocardiograms obtained every 2 weeks and 2 weeks after the last dose were analyzed for several key structural and physiologic metrics and N-terminal prohormone of brain natriuretic peptide (NT-proBNP).

Initial results from the clinical study included data from two sequentially conducted cohorts, Cohort 1 (n=21) and Cohort 2 (n=20) which randomized treatment of patients 2:1 to aficamten or placebo. Patients received up to three escalating doses of aficamten once daily (5, 10, 15 mg in Cohort 1 and 10, 20, 30 mg in Cohort 2) or placebo. Patients had an echocardiogram after two weeks of treatment at each dose to determine potential up-titration to the next higher dose. Overall, treatment duration for each patient in the study was 10 weeks with an echocardiogram conducted 2 weeks after the last dose.

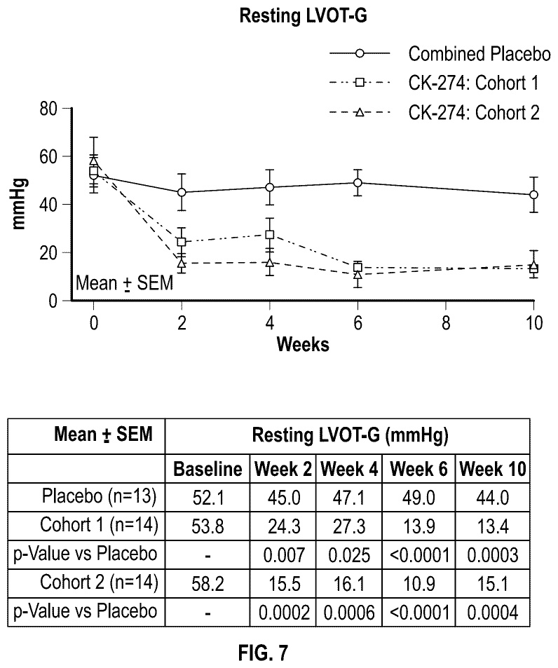

For patients on aficamten in Cohort 1 (n=14), the average resting LVOT-G changed from 53.8 mmHg at baseline to 13.4 mmHg at 10 weeks; for patients on aficamten in Cohort 2 (n=14) the average resting LVOT-G changed from 58.2 mmHg at baseline to 15.1 mmHg at 10 weeks; and for patients in the combined placebo group (n=13) the average resting LVOT-G changed from 52.1 at baseline to 44.0 mmHg at 10 weeks (FIG. 7, p=0.0003 for Cohort 1, p=0.0004 for Cohort 2 in comparison to placebo at 10 weeks).

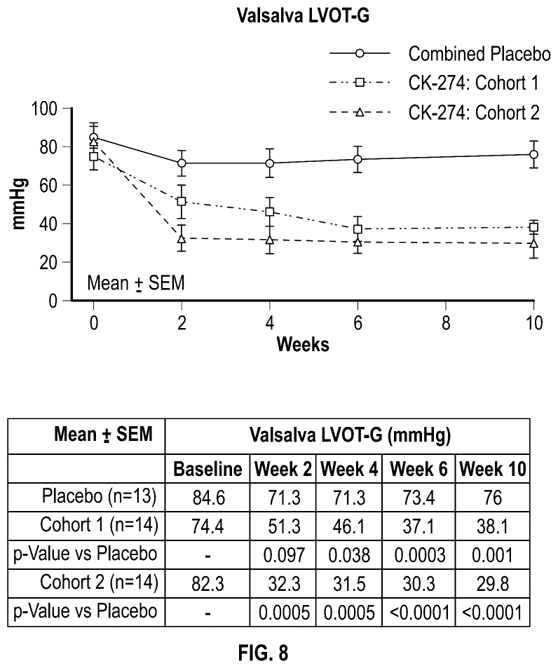

For patients on aficamten in Cohort 1 (n=14) the average Valsalva LVOT-G changed from 77.4 mmHg at baseline to 38.1 mmHg at 10 weeks; for patients on aficamten in Cohort 2 (n=14) the average Valsalva LVOT-G changed from 82.3 mmHg at baseline to 29.8 mmHg at 10 weeks; and for patients in the combined placebo group (n=13) the average Valsalva LVOT-G changed from 84.6 at baseline to 76.0 mmHg at 10 weeks (FIG. 8; p=0.001 for Cohort 1, p<0.0001 for Cohort 2 in comparison to placebo at 10 weeks).

The average ejection fraction for patients on aficamten in Cohort 1 (n=14) changed from 72.8% at baseline to 67.3% at 10 weeks; for patients on aficamten in Cohort 2 (n=14) the average ejection fraction changed from 75.4% at baseline to 64.1% at 10 weeks, and for patients in the combined placebo group (n=13) the average ejection fraction changed from 74.5% at baseline to 74.9% at 10 weeks (p=0.01 for Cohort 1, p=<0.0001 for Cohort 2 in comparison to placebo at 10 weeks).

Overall, the incidence of adverse events was similar between treatment arms. Treatment with aficamten in the study was well tolerated with adverse events reported as mild or moderate in severity. There were no treatment related serious adverse events reported by investigators.

No patients who received aficamten in Cohort 1 had an LVEF <50%. In Cohort 2, one patient with an LVEF at baseline of 58% was up titrated to 20 mg of aficamten and experienced transient LVEF reduction to <50% (remaining above 40%) requiring down titration. No interruptions or discontinuations of treatment with aficamten occurred in any patients across both cohorts.

The distribution of patients across doses of aficamten in the study (Cohorts 1 and 2) is shown in Table 14. The distribution of patients across doses of aficamten in Cohort 3 of the study is shown in Table 15.

TABLE 14

| | | Final Dose Achieved (N) | | | | | |
|---|---|---|---|---|---|---|---|
| | | Cohort 1 | | | Cohort 2 | | |
| | Placebo | 5 mg | 10 mg | 15 mg | 10 mg | 20 mg | 30 mg |
| Cohort 1 + 2 | 13 | 4 | 5 | 5 | 9 | 4 | 1 |
| (N = 41) | | | | | | | |

TABLE 15

| Final Dose Achieved (N) | | | | |
|---|---|---|---|---|
| 5 mg | 10 mg | 15 mg | 20 mg | 30 mg |

| | | | | |
|---|---|---|---|---|
| N = 13 | Cohort 3 | 1 | 6 | 6 |

Secondary results from the clinical study included data from Cohort 3 (n=13). All patients received up to three escalating doses of aficamten once daily (5, 10, 15 mg). Patients had an echocardiogram after two weeks of treatment at each dose to determine potential up-titration to the next higher dose. Overall, treatment duration for each patient in the study was 10 weeks with an echocardiogram conducted 2 weeks after the last dose. Efficacy endpoints included resting and provoked LVOT gradients, NYHA class, and NT-proBNP.

In Cohort 3, 13 patients were enrolled (59±14 years of age; 54% female) with NYHA class II (n=5) and III (n=8). Compared with Cohorts 1 and 2, patients in Cohort 3 had similar demographics, LVEF and severity of obstruction, but were more symptomatic and had higher baseline NT-proBNP. Cohort 3 patients had symptomatic obstructive HCM and a resting or post-Valsalva left ventricular outflow tract gradient (LVOT-G) of ≥50 mmHg, and had previously been treated with disopyramide and, in the majority, a beta-adrenergic blocker. All patients received up to three escalating doses of aficamten once daily (5, 10, 15 mg), titrated based on echocardiographic guidance, as discussed above. Overall treatment duration was 10 weeks with a 4-week follow up period after the last dose. In total, thirteen patients were enrolled and all patients completed the study on treatment.

Results from Cohort 3 showed that substantial reductions in the average resting LVOT-G as well as the post-Valsalva LVOT-G (defined as resting gradient <30 mmHg and post-Valsalva gradient <50 mmHg) were achieved. These clinically relevant decreases in pressure gradients were achieved with only modest decreases in average left ventricular ejection fraction (LVEF); there were no patients whose LVEF fell below the prespecified safety threshold of 50%. New York Heart Association functional class was improved in the majority of patients participating in Cohort 3 of the trial. Pharmacokinetic data were similar to those observed in Cohorts 1 and 2. In addition, the safety and tolerability of aficamten were consistent with no treatment interruptions and no serious adverse events attributed to treatment reported by the investigators.

Aficamten in combination with disopyramide may present a treatment option for the most severe and treatment refractory oHCM patients.

Results after 10 Weeks of Therapy

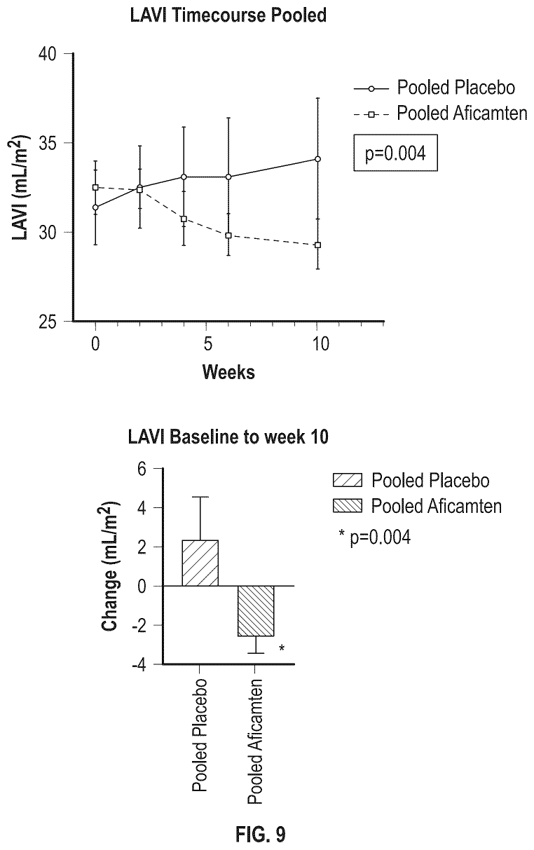

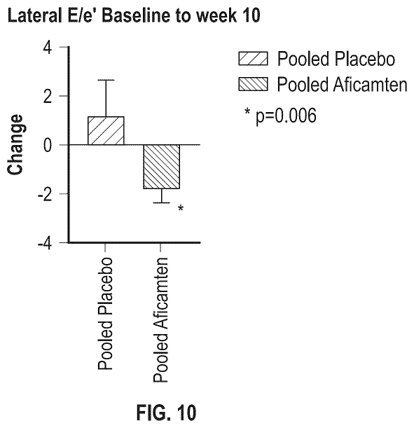

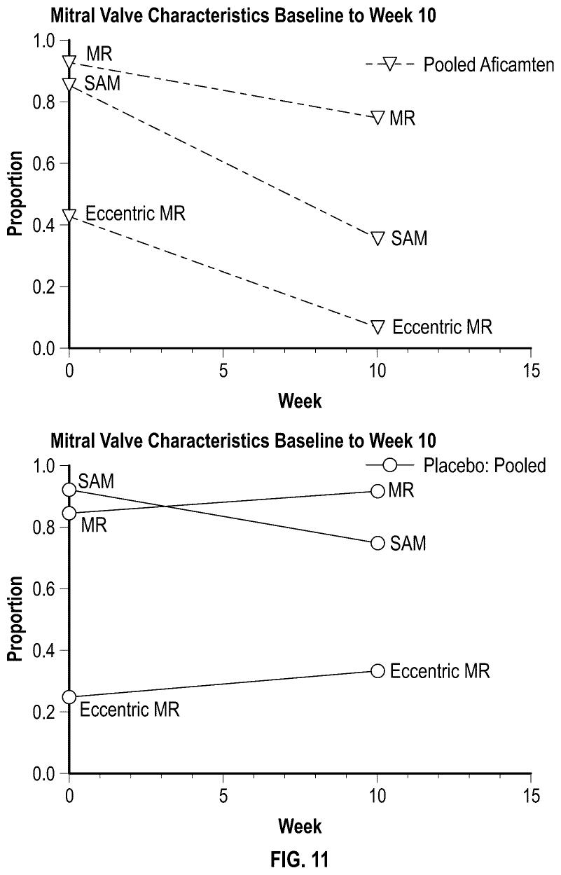

There was no significant difference at baseline in the key echocardiographic metrics and NT-proBNP values between aficamten and placebo. Compared to placebo, patients on aficamten had a trend toward reduction in mean left ventricular mass index (LVMI) (−4.8 g/m² (±2.4) vs 3.3 g/m² (±3.6); mean difference: 8.1 g/m², p=0.063 value). Indices of left ventricular (LV) filling pressures improved in the aficamten treated group including left atrial volume index (LAVI) (−2.9 mL/m² (±1.5) vs 2.2 mL/m² (±1.5), P=0.004) (FIG. 9); e′ (0.5 cm/s (±0.4) vs −0.5 cm/s (±0.3), p=0.03), and lateral E/e′ (−2.0 (±1.1) vs 1.8 (±0.8), p=0.006) (FIG. 10) from baseline to week 10. Similarly, a reduction relative to baseline in the categorical assessment of systolic anterior motion of the mitral valve leaflet (SAM; −50% vs −17.3%) (FIG. 11) and frequency of eccentric mitral regurgitation (MR; −35.8% vs +13.3%) (FIG. 11) was observed in the aficamten versus placebo, respectively, at week 10. There was a significantly greater reduction in NT-proBNP with aficamten versus placebo (geometric least squares mean ratio 0.38 (0.25-0.56), p=0.0002) at week 10.

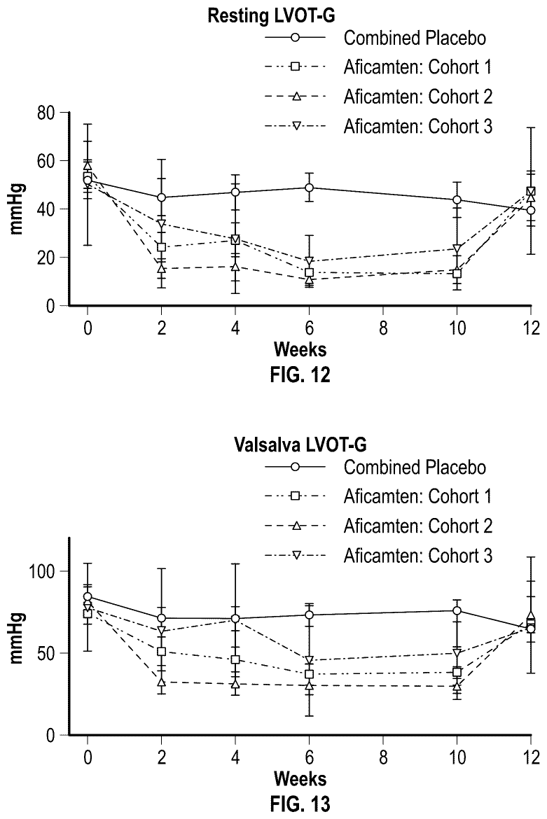

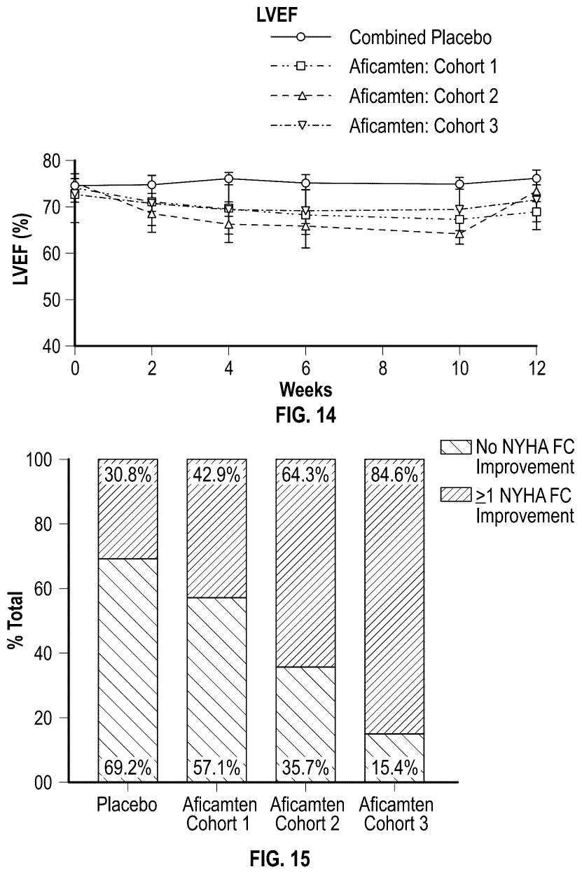

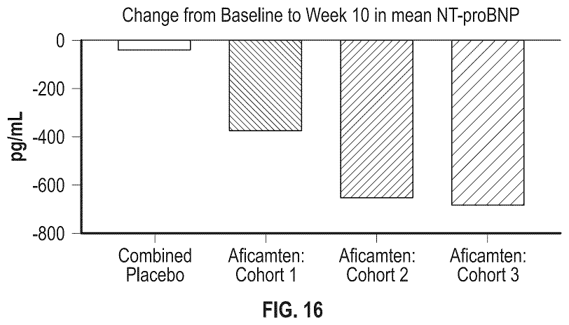

FIG. 12 shows changes to resting LVOT-G for treatment and placebo cohorts. FIG. 13 shows changes to resting Valsalva LVOT-G for treatment and placebo cohorts. Effect on LVOT-G was mostly attenuated with aficamten treatment in patients treated with disopyramide (Cohort 3) when compared with Cohort 1 patients (same aficamten dose). FIG. 14 shows changes to LVEF for treatment and placebo cohorts. FIG. 15 shows NYHA functional class response for treatment and placebo cohorts. FIG. 16 shows changes in mean NT-proBNP for treatment and placebo cohorts.

The safety profile for all three treatment cohorts is presented in Table 16, In Cohort 3, six moderate adverse events were recorded, including pneumonia, pertussis, lung mass, back pain, shortness of breath, and orthopnea. One adverse event of asymptomatic atrial fibrillation in patient with known prior history was recorded. The remaining adverse events included gastrointestinal symptoms (a known side effect of disopyramide) and other adverse events seen in Cohorts 1 and 2 (headaches, dizziness). Overall safety profile in Cohort 3 supports the combined use of aficamten and disopyramide.

TABLE 16
Safety Profile for Patients Treated with aficamten in a Phase 2 Clinical Trial

| | Cohort 1 | | Cohort 2 | | Cohort 3 | | | |
|---|---|---|---|---|---|---|---|---|
| | N (%) | | N (%) | | N (%) | | Total | |
| Patients Randomized | 21 | (100) | 20 | (100) | 13 | (100) | 54 | (100) |
| Patients with at least one TEAE | 17 | (81) | 15 | (75) | 9 | (69) | 41 | (75.9) |
| Patients with TESAEs | 2 | (9.5) | 0 | | 0 | | 2 | (3.7) |
| Patients with fatal TEAEs | 0 | | 0 | | 0 | | 0 | |
| Patients with TEAEs leading to | 0 | | 0 | | 0 | | 0 | |
| drug interruption or termination | | | | | | | | |
| Patients with AEs related to study | 3 | (14.3) | 1 | (5.0) | 2 | (15.4) | 6 | (11.1) |
| drug (per investigator) | | | | | | | | |

| | | | | | | |
|---|---|---|---|---|---|---|
| TEAE of LVEF <50% (Core Lab) | 0 | 2 | (10) | 0 | 2 | (3.7) |

These findings indicate that aficamten treatment resulted in salutary early cardiac remodeling associated with reductions in LVMI, LAVI, lateral E/e′, SAM, eccentric mitral regurgitation, and brain natriuretic peptide with increment in e′ velocity, and further indicate that aficamten favorably impacts cardiac remodeling in oHCM.

Results for Cohort 1 and Cohort 2

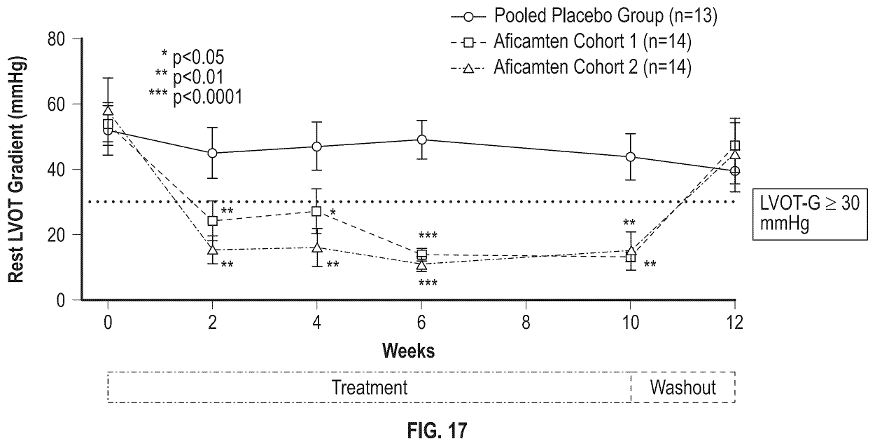

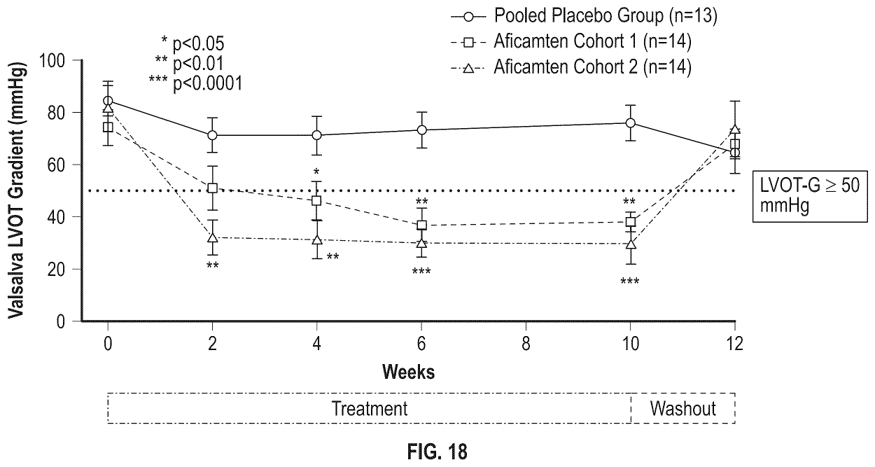

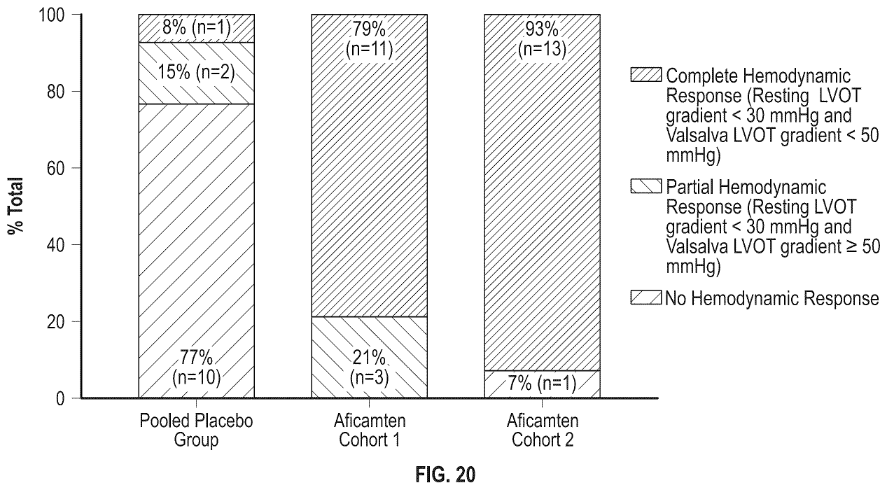

With reference to Cohorts 1 and 2, a complete hemodynamic response (resting LVOT gradient <30 mmHg and Valsalva gradient <50 mmHg at Week 10) occurred in 11 out of 14 patients (79%) in aficamten Cohort 1 and 13 out of 14 patients (93%) in aficamten Cohort 2, compared with only 1 out 12 (8%) in the pooled placebo group (FIG. 17, FIG. 18, FIG. 20).

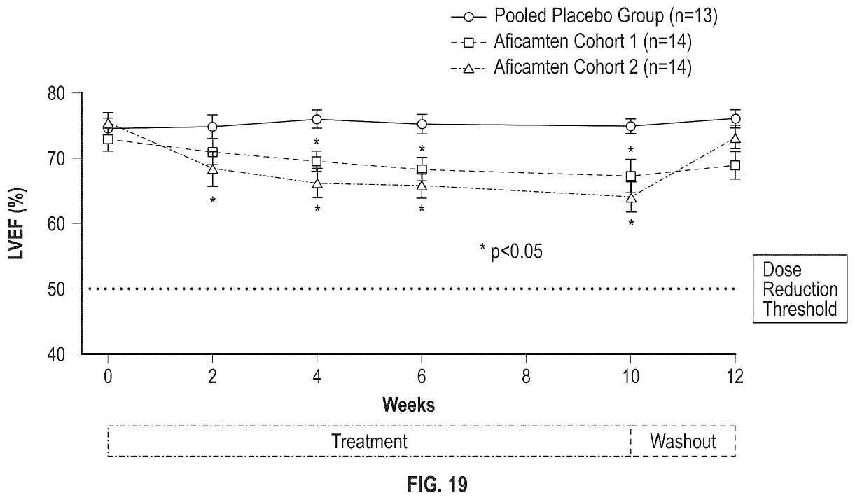

Over the treatment period, EF decreased in aficamten Cohort 1 from 73±6% to 67±9% (LSMean difference vs. placebo p=0.007) and in aficamten Cohort 2 from 75±6% to 64±8% (LSMean difference vs. placebo p<0.001), with no change in the placebo group (75±6% to 75±4%; p=0.5). (FIG. 19) Analysis of the relationship between aficamten dose and EF over time revealed a dose-dependent decrease with a mean reduction in EF of −0.6% (SE 0.084) per mg of aficamten.

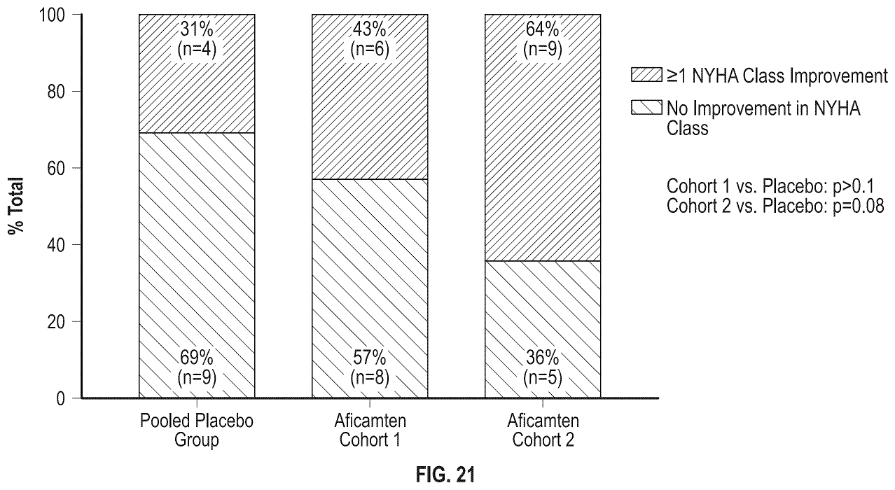

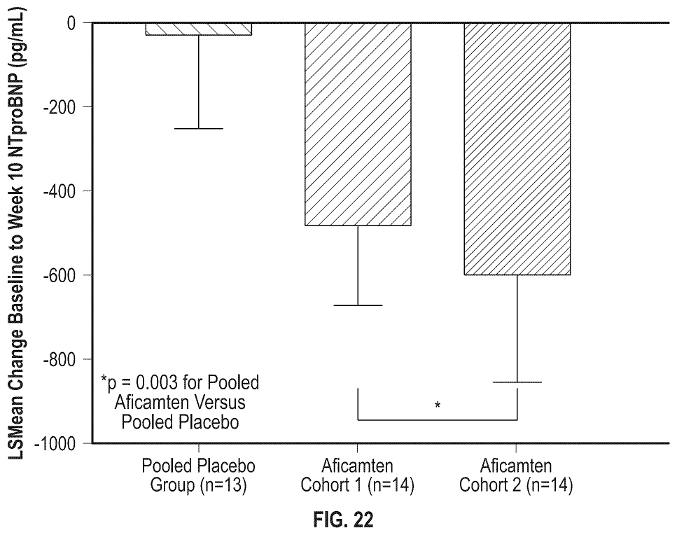

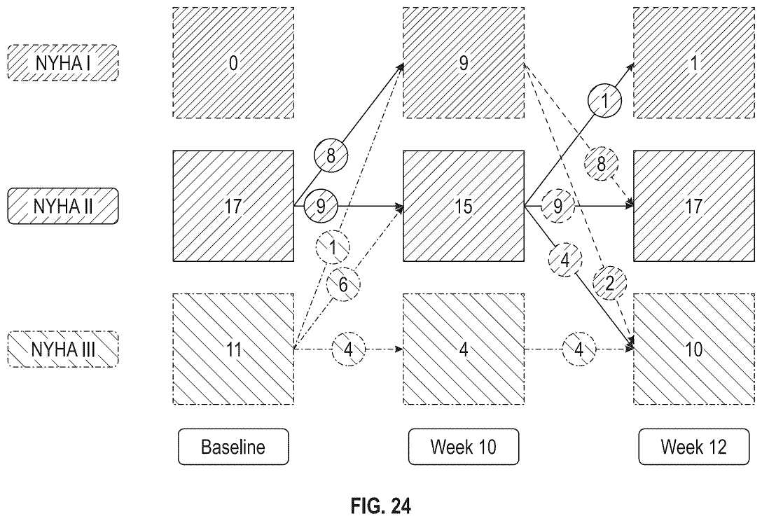

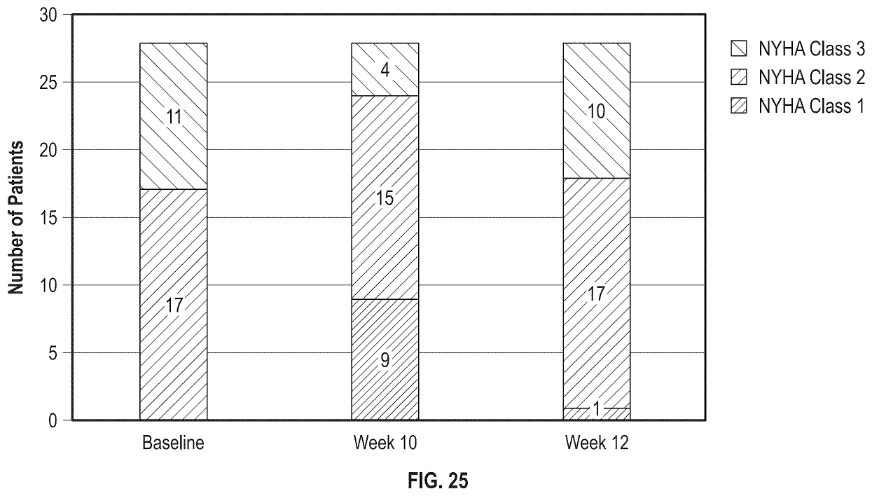

In the pooled aficamten treatment group (Cohorts 1 and 2), 15 of 28 (53%) patients experienced a change in NYHA class of one or more classes (FIG. 21), including: 6 patients who improved from class III to II, 8 from class II to I and one from III to I. (FIG. 24).

Aficamten treatment was associated with a 62% proportional reduction in NT-proBNP levels at Week 10 compared to placebo (p<0.001). Importantly, 25 out of the 27 patients on aficamten (93%) experienced at least some reduction in NT-proBNP levels compared to only 6 of 12 placebo treated patients (50%).

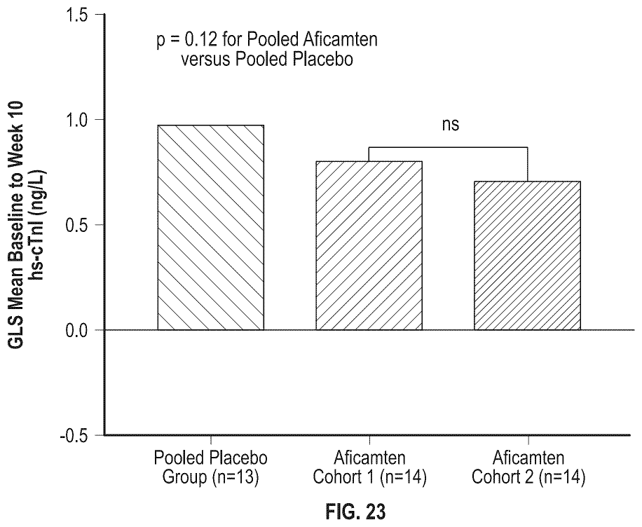

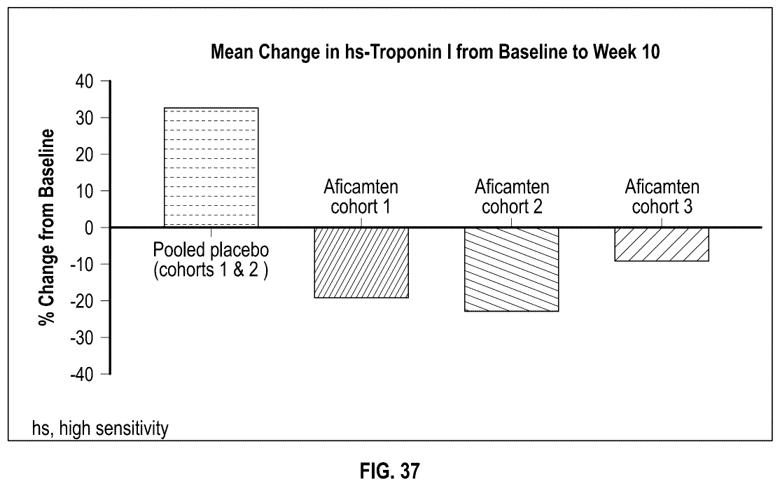

Baseline levels of hs-Trop were 17 ng/L (% CV 290) for the pooled aficamten group and 17 ng/L (% CV 290) for pooled placebo. (FIG. 23) At Week 10, patients receiving aficamten in Cohort 1 experienced a 18% relative reduction (p=0.29) compared with pooled placebo, and patients in Cohort 2, a 26% relative reduction (p=0.097) compared with pooled placebo. Change in levels of hs-Troponin I in the pooled placebo groups, as well as patients receiving aficamten in cohort 1, cohort 2, and cohort 3, are shown in FIG. 37.

The early and sustained hemodynamic effect of aficamten was accompanied by marked clinical benefit in heart failure symptoms in most patients. Symptom improvement in NYHA class by one or more classes occurred in more than half of patients treated with aficamten, including 64% in Cohort 2 with most of that improvement resulting in patients transitioning from class II to becoming entirely asymptomatic (class I). It is notable that aficamten converted 7 patients from advanced heart failure symptoms (class III) to less symptomatic status (class II or I).

This robust hemodynamic response is particularly notable since aficamten converted the majority of obstructive HCM patients to gradient levels that are below the current threshold for consideration of septal reduction therapies, such as myectomy or alcohol septal ablation. This is a particularly relevant point since one of the strengths of septal reduction therapy is the opportunity to convert patients with advanced limiting symptoms (class III) to asymptomatic or mildly symptomatic status.

Aficamten was also associated with marked reductions in NT-proBNP and hs-troponin, underscoring that the compound may result in other potential downstream pathophysiologic benefits including decreases in LV wall stress and reduction in myocardial injury.

### Results for Cohort 3

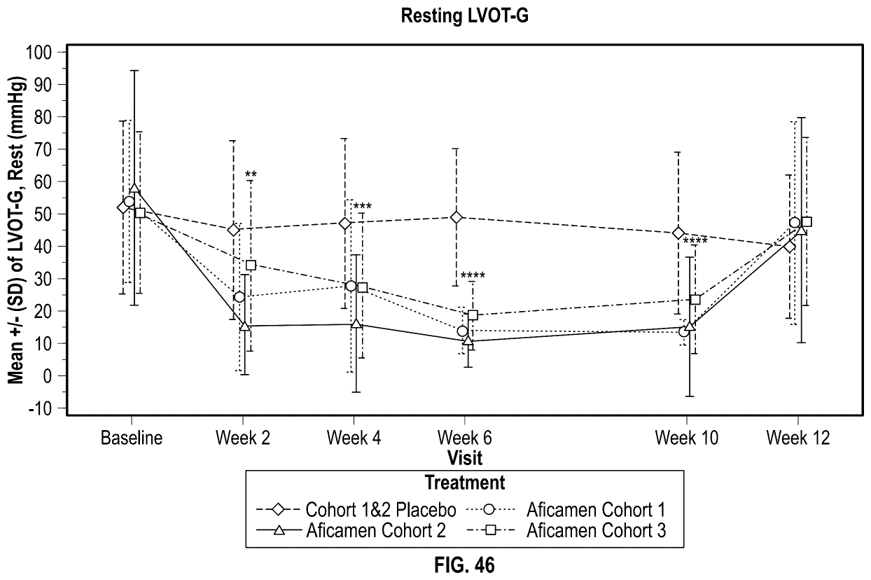

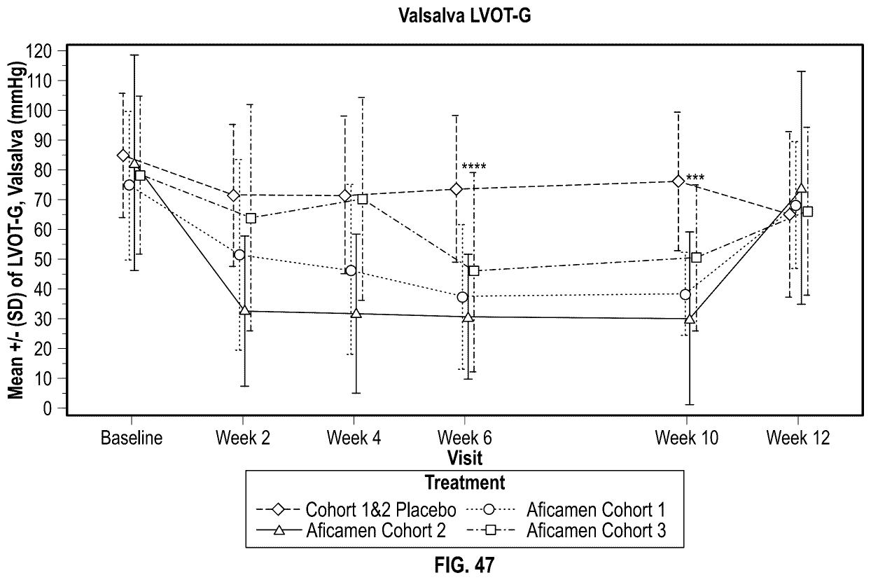

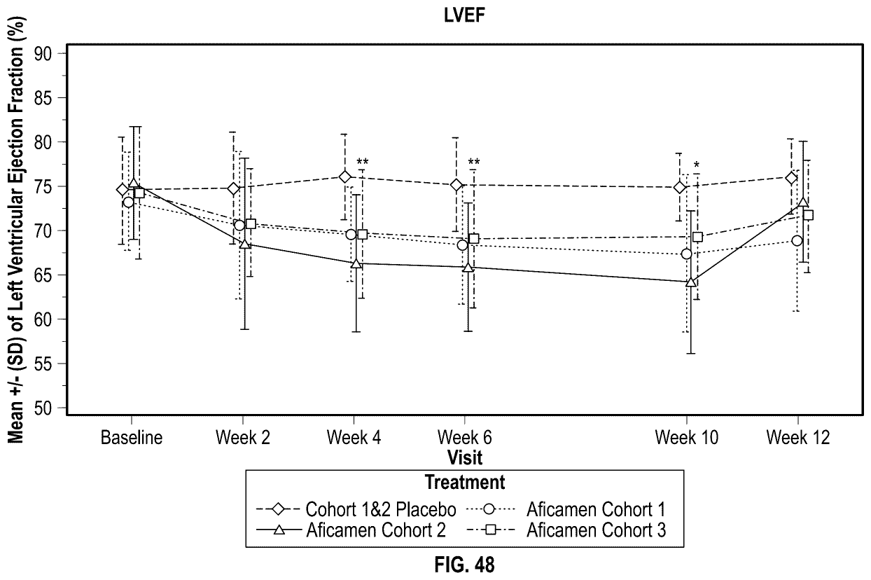

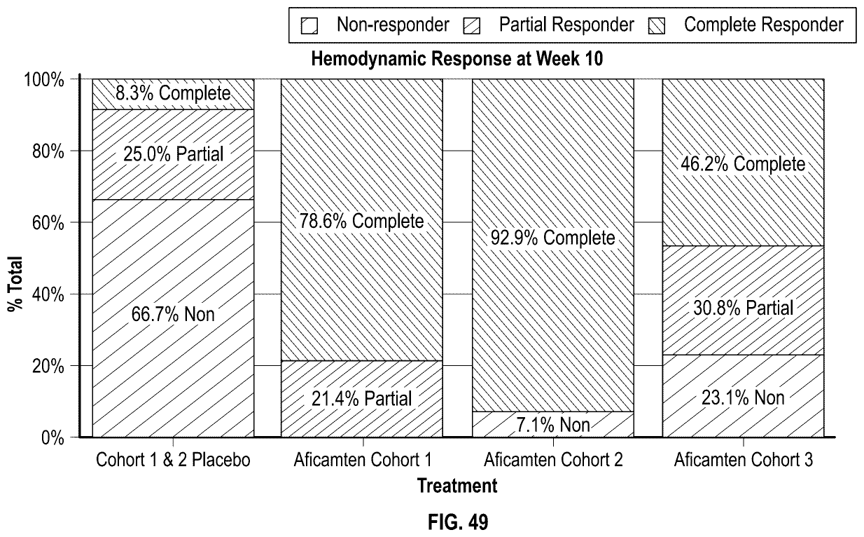

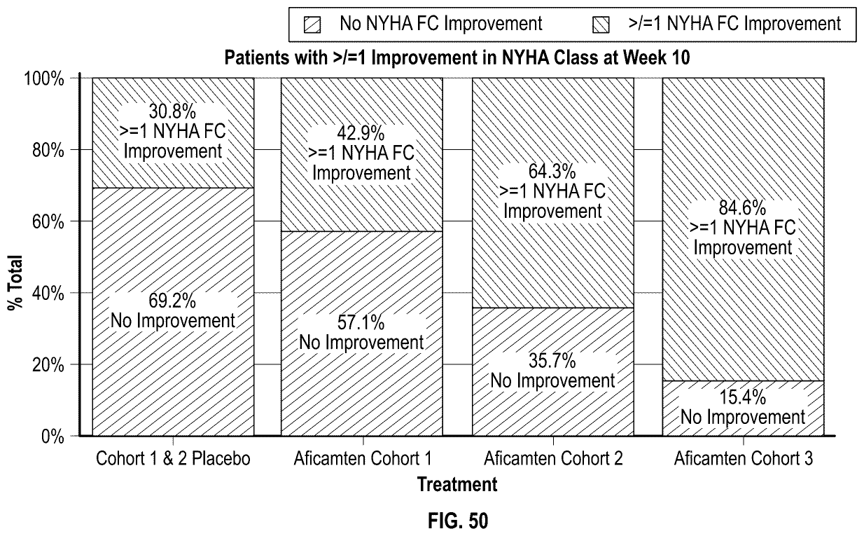

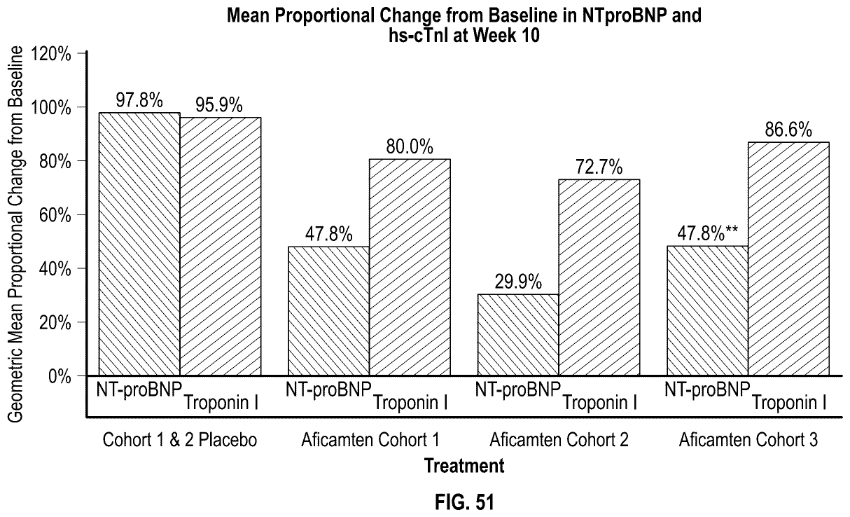

Symptomatic (New York Heart Association [NYHA] class II/III) oHCM patients (resting or Valsalva LVOT gradient ≥50 mmHg and LVEF ≥60%) on standard of care medical therapy that included a BB and/or CBB (calcium channel blockers) plus disopyramide were enrolled in an open-label fashion. Thirteen patients (mean age 59.4 (SD 14.4), 53.8% female) were enrolled. Ten (77%) were on BB; 2 (15.4%), on CCB, and 1 (7.7%) was on both. The median (min, max) disopyramide dose was 300 mg/day (100, 600 mg/day). At baseline, mean (SD) LVEF was 74 (7.5)%; resting and Valsalva LVOT-Gs were 50 (25) mmHg and 78 (27) mmHg, respectively. Five (38.5%) were NYHA Class II, and 8 (62%) were NYHA Class III with baseline serum NT-proBNP (Geometric mean 1050 pg/mL, % CV 110). The final dose achieved was 5 mg in 2 patients, 10 mg in 5 patients, and 15 mg in 6 patients. Resting and Valsalva LVOT gradients (FIG. 46, 47) were substantially reduced over the 10-week treatment period with mean (SD) Week 10 resting LVOT-G reduced by 27 mmHg (22) [p<0.0001], and mean Valsalva LVOT-G reduced by 28 mmHg (32) [p=0.0002]. There was a modest reduction in mean LVEF from 74±7.5% to 69±7.2% [p=0.018] (FIG. 48), which returned to baseline after the 2-week washout. Ten of 13 patients (77%) displayed complete or partial response in LVOT gradients at end of treatment (FIG. 49). Three patients (23%) achieved temporary complete or partial response during the treatment period with overall improvement in gradient and symptoms at end of treatment. Eleven patients (85%), including all 8 with baseline NYHA Class III, demonstrated ≥1 NYHA class improvement (FIG. 50). The 2 patients who did not report an improvement in NYHA class experienced a partial hemodynamic response. Changes in hemodynamics and NYHA class were mirrored by reductions in cardiac biomarkers, with similar improvements to those observed in Cohorts 1 and 2 (patients receiving beta-blockers and/or calcium channel blockers but excluding disopyramide) (FIG. 51). There were no dose interruptions, treatment discontinuations, or serious adverse events. No patients experienced LVEF <50%, and there were no significant changes in QTc interval, or vital signs.

In this open label study of mostly NYHA class III individuals, the addition of aficamten significantly reduced LVOT gradients in 77% of patients and resulted in improved symptoms in 85% of patients, and all baseline NYHA class III patients improved by ≥1 class. Importantly, aficamten was well tolerated and demonstrated reversibility in this severely obstructed cohort of patients. Reduction in LVEF was similar to that observed in cohort 2 patients who were not on disopyramide. Aficamten showed efficacy as an add-on therapy in medically refractory oHCM patients and may offer an alternative to SRT.

### Example 3a

An open-label extension clinical trial of aficamten in patients with symptomatic oHCM was initiated. The primary objective of the trial was to determine the safety and tolerability of aficamten over a 5 year period.

Patients who completed the study as described in Example 2 and had not developed atrial fibrillation were eligible to enroll in the study. Echo-guided dose titration based on site reads was managed by the investigator and could occur at any time during the trial, as described below.

### Study Design

Each patient received Dose 1 of aficamten once daily for 2 weeks. At Week 2, each patient had a truncated echocardiogram 2 hours following administration of their dose. Patients were up-titrated to Dose 2 if either of the following conditions were met on echocardiography: (1) resting LVOT-G ≥30 mmHg and the biplane LVEF ≥50%; or (2) resting LVOT-G <30 mmHg, post-Valsalva LVOT-G ≥50 mmHg, and the biplane LVEF ≥50%. Otherwise, the patient remained on 5 mg of aficamten. If LVEF was <50%, treatment was discontinued. If LVEF was <40%, treatment is interrupted.

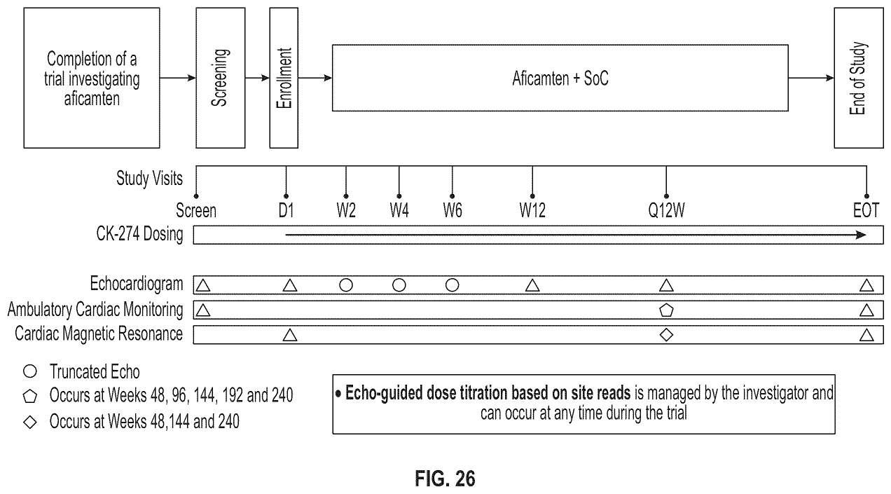

At Weeks 4, 6, 12, and every 12 weeks thereafter, each patient had an echocardiogram or truncated echocardiogram 2 hours following administration of their dose (a truncated echocardiogram at weeks 4 and 6; and an echocardiogram at week 12 and every 12 weeks thereafter) to determine whether additional dose titration is needed (see Table 17 and Table 18). Ambulatory cardiac monitoring is performed on weeks 48, 96, 144, 192 and 240. Cardiac magnetic resonance is monitored at weeks 48, 144 and 240. (See FIG. 26). Baseline characteristics for the patients enrolled in the open-label extension study are shown in Table 19.

TABLE 17
Dose Adjustment Algorithm

| Biplane | | | |
|---|---|---|---|
| LVEF | | LVOT Gradient | Action |
| <40% | | | Permanent discontinuation |
| <50% | | | Dose reduce to next lower dose, |
| | | | or discontinuation if on lowest |
| | | | dose |
| ≥50% | and | Resting LVOT gradient <30 mmHg | Dose unchanged |
| | | AND | |
| | | Valsalva LVOT gradient <50 mmHg | |
| ≥50% | and | Rest LVOT gradient ≥30 mmHg | Dose escalate to next dose level |
| | | AND/OR | |
| | | Valsalva LVOT gradient ≥50 mmHg | |

TABLE 18
Dosing Scheme

| Dose 1 | Dose 2 | Dose 3 | Dose 4 |
|---|---|---|---|
| 5 mg | 10 mg | 15 mg | 20 mg |

TABLE 19
Baseline characteristics for open-label extension study

| Baseline Characteristics | N = 38 | |
|---|---|---|
| Age (Years), Mean (SD) [Range] | 59.9 | (13.0) [23-82] |
| Female, n (%) | 22 | (57.9%) |
| BMI (kg/m2), Mean (SD) [Range] | 30.1 | (6.5) [22-51] |
| NYHA Class, n (%) | | |
| Class II | 18 | (47.4%) |
| Class III | 20 | (52.6%) |
| Positive family history of HCM, n (%) | 9 | (23.7%) |
| Background HCM Therapy, n (%) | | |
| Beta Blocker | 30 | (78.9%) |
| Calcium Channel Blocker | 11 | (28.9%) |
| Disopyramide | 10 | (26.3%) |
| LVEF* at Screening (%), Mean (SD) [Range] | 69.7 | (4.1) [60-78] |
| LVOT-G*, Rest at Screening (mmHg), Mean (SD) | 47.0 | (26.6) [10-95] |
| [Range] | | |
| LVOT-G*, Valsalva at Screening (mmHg), Mean (SD) | 81.1 | (29.1) [23-150] |
| [Range] | | |
| NT-proBNP (pg/mL), Geometric Mean (% CV) [Range] | 628.1 | (163.7) [70-8333] |
| Cardiac Troponin I (ng/L), Geometric Mean (% CV) | 13.8 | (287.5) [3.4-2017] |
| [Range] | | |
| Duration on Treatment in Weeks, Mean (SD) [Range] | 25.7 | (11.7) [5-47] |

### Preliminary Results

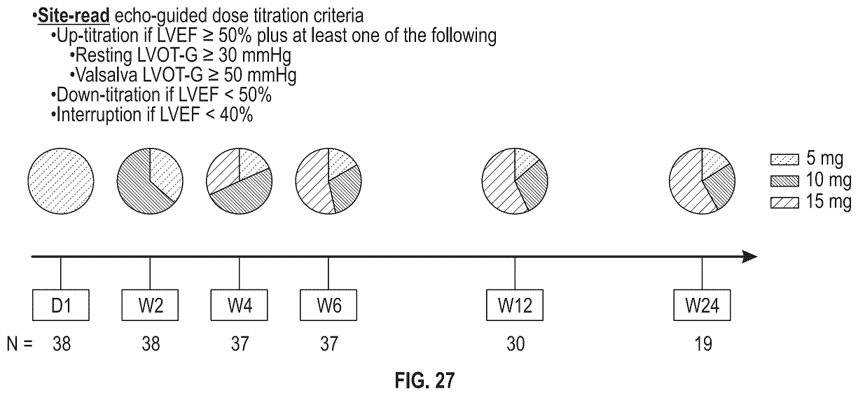

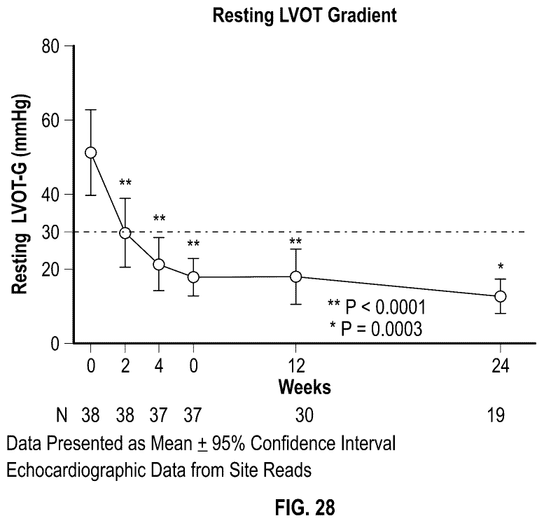

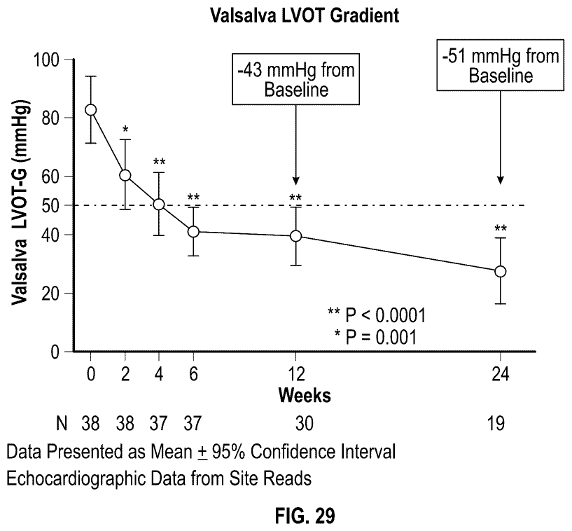

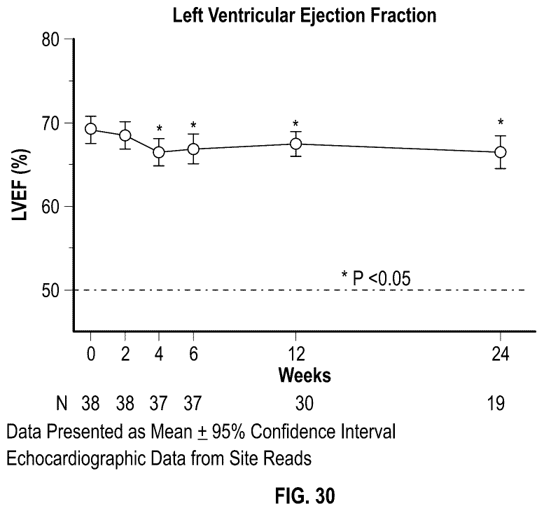

Preliminary results are shown in FIGS. 27-34. FIG. 27 shows the distribution of patients across doses over time. At the time of data collection, 38 patients had enrolled in the trial, and all 38 patients had reached at least week 2 of dosing; 37 of the 38 patients had reached at least week 6 of dosing; 30 of the 38 patients had reached at least week 12 of dosing; and 19 of the 38 patients had reached at least week 24 of dosing. The percentage of patients receiving each dose level (5, 10, or 15 mg) at each time point is shown in FIG. 27. Significant reduction in resting LVOT-G and Valsalva LVOT-G over time based on echocardiographic data from site reads of these patients is shown in FIG. 28 and FIG. 29; significant and sustained reduction in LVOT gradients was identified at Weeks 2-24. In addition, minimal and stable reductions in LVEF were noted through Week 24, as shown in FIG. 30.

At baseline, 53% of patients were NYHA class III, and 47% of patients were NYHA class II.

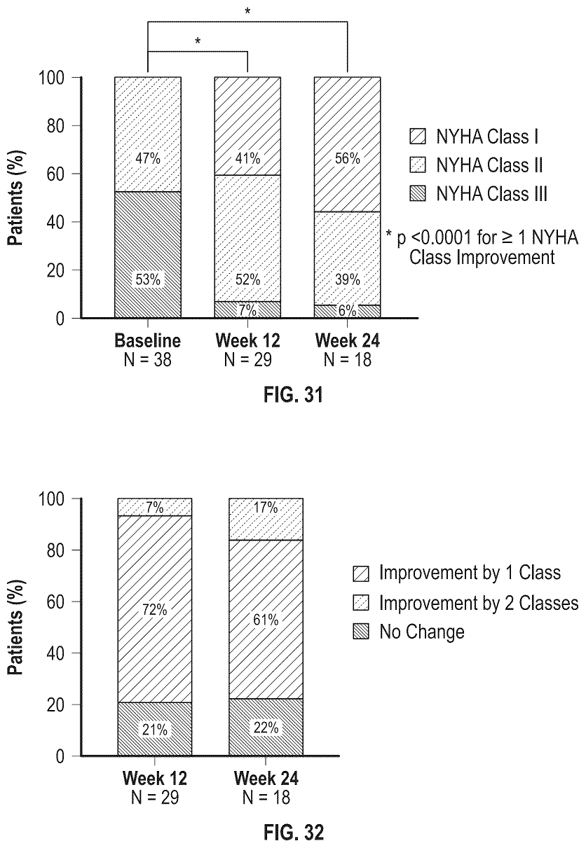

Of the patients that had reached at least week 12 of dosing: at week 12, only 7% of patients were NYHA class III; 52% of patients were NYHA class II; and 41% of patients were NYHA class I (FIG. 31). 72% of patients had experienced an improvement by one NYHA class, and 7% of patients improved by two NYHA classes relative to baseline (FIG. 32).

Of the patients that had reached at least week 24 of dosing: at week 24, only 6% of patients were NYHA class III; 39% of patients were NYHA class II; and 56% of patients were NYHA class I (FIG. 31). 61% of patients had experienced an improvement by one NYHA class, and 17% of patients had experienced an improvement by two NYHA classes relative to baseline (FIG. 32).

No patients in the study showed worsening in NYHA class from baseline.

### Preliminary Safety

TABLE 20

| | N = 38 | |
|---|---|---|
| Patients with at Least One TEAE | 28 | (74%) |
| Patients with at Least One Related TEAE | 8 | (21%) |
| Patients with at Least One TESAE | 2 | (5%) |
| Patients with at Least One Severe TEAE | 1 | (3%) |
| Patients with TEAE Leading to Drug Interruption | 1 | (3%) |
| Patients with TEAE Leading to Dose Reduction | 2 | (5%) |

TEAE: Treatment Emergent Adverse Event
TESAE: Treatment Emergent Serious Adverse Event
Cardiac AEs: Atrial fibrillation (2); Angina pectoris (1); Bradycardia (1); Decreased ejection fraction (1); Mitral valve stenosis (1); QTc prolongation (1)

One patient with LVEF <50% and TESAE had a history of alcohol induced atrial fibrillation prior to study with reduced LVEF <50%. On 15 mg of aficamten, recurrent episode of alcohol induced atrial fibrillation with similar reduction of LVEF to 47%; aficamten was down-titrated. The patient subsequently developed worsening atrial fibrillation and had a failed cardioversion; aficamten was interrupted. This patient is back in sinus rhythm on amiodarone, abstinent from alcohol, with LVEF 60% with evidence of obstruction and has restarted aficamten at dose 1 (5 mg).

One patient experienced temporary down-titration due to Investigator concern about QTc prolongation in a subject with abnormal baseline EKG. Temporary aficamten down-titration was performed pending core-lab QTc interpretation. It was confirmed that the QTc was normal, and aficamten was subsequently increased.

One subject with Severe TESAE showed altered mental status prior to planned cardioversion for worsening atrial fibrillation on DOAC (direct-acting oral anticoagulants), leading to hospitalization. MRI showed presumed embolic stroke. Patient was subsequently diagnosed with congenital cardiac abnormality (secundum atrial septal defect). No aficamten down-titration or interruption was needed.

### Kansas City Cardiomyopathy Questionnaire (KCCQ)

Participants' health status was assessed with the KCCQ prior to starting aficamten in the OLE and at 12 and 24 weeks of treatment. Change in KCCQ scores from baseline, including OSS=overall summary score, CSS=clinical summary score, TSS=total symptom score, PLS=physical limitation score, SLS=social limitation score, QoL=quality of life, were determined. Patients were categorized with worsened (≤−5 points), unchanged (−5 to <5 points), small improvement (5 to <10 points), moderate to large improvement (10 to <20 points), and large to very large improvement (≥20 points) relative to baseline.

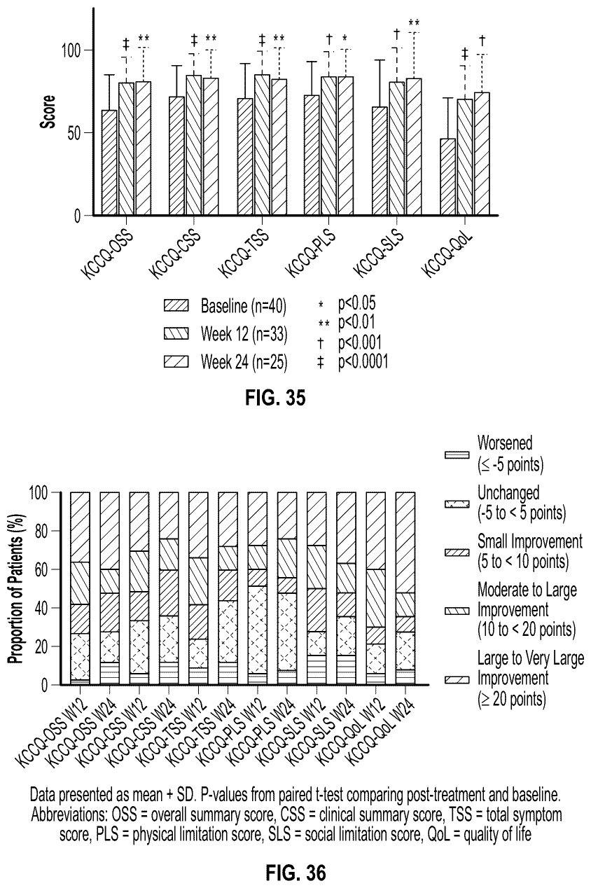

The results display the marked improvements in KCCQ scores among OLE participants at 12 weeks that were sustained to 24 weeks. The proportion of participants with clinically-important improvement (5 points on the Overall Summary Score) was 72.7% at Week 12 and 72.0% at Week 24. Very large clinical improvements WO points) were seen in 36.4% at Week 12 and 40.0% at Week 24 (FIG. 35, FIG. 36). Treatment with aficamten resulted in a marked and sustained improvement in all KCCQ domain scores for up to 6 months.

### Conclusions

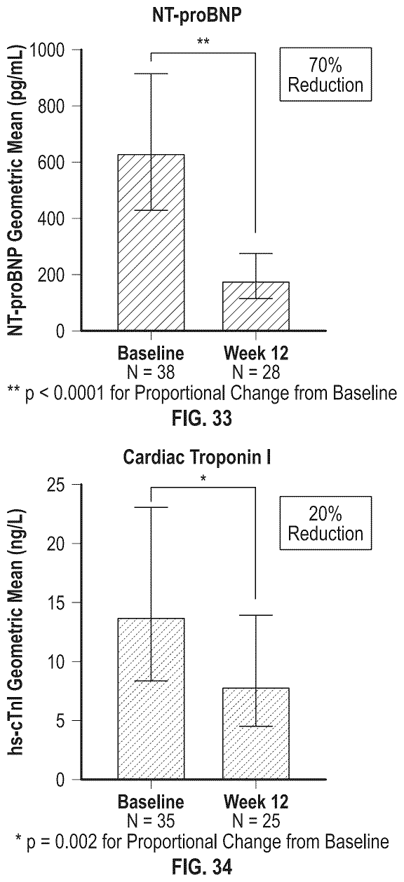

In this open label extension study of patients with obstructive HCM treated with background medical therapy, including disopyramide in some cases: aficamten was associated with significant and sustained reductions in LVOT gradients (FIG. 28 and FIG. 29) and substantial improvement in heart failure symptoms (about 80% of patients had NYHA class improvement of one or more classes) (FIG. 31 and FIG. 32), as well as significant reduction in cardiac biomarkers (NT-proBNP and hs-cTnI) (FIG. 33 and FIG. 34). Aficamten was well tolerated, with no events of LVEF <50% attributed to aficamten. These data demonstrate that the treatment effect of aficamten is durable for up to 6 months.

### Example 3b

An open-label extension clinical trial of aficamten in patients with symptomatic oHCM was initiated. The treatment duration is anticipated to be multiple years. The primary objective of the trial is to determine the safety and tolerability of aficamten over a 5-year period.

Up to 275 patients may be enrolled in this study. After (up to) 56-days of screening, eligible patients are administered a daily dose of aficamten. The highest maximum tolerated dose is informed by the ongoing conduct of other studies of aficamten. Each patient starts at the lowest prespecified dose and undergo echocardiography-guided dose titration to their maximum tolerated dose (not to exceed the highest prespecified dose). Dose adjustment may be made no more frequently than every 2 weeks. Approximately 95 investigative sites in the US, Europe, and Middle East participate in this study.

Inclusion Criteria. (1) Completion of a study investigating aficamten (i.e., in accordance with Example 2 or Example 5); (2) able to comprehend and willing to sign an ICF and willing to comply with all study procedures and restrictions for the duration specified in the Schedule of Activities; (3) Left ventricular ejection fraction ≥55%.

Exclusion Criteria. (1) Has received treatment with mavacamten. (2) Has participated in another investigational device or drug study or received an investigational device or drug <1 month (or 5 half-lives for drugs, whichever is longer) prior to screening. Other investigational procedures while participating in this study are not permitted. (3) Has any acute or serious comorbid condition (e.g., major infection or hematologic, oncologic, cardiac, renal, metabolic, gastrointestinal, or endocrine dysfunction) that, in the opinion of the site Principal Investigator/designee or Medical Monitor, would pose a risk to patient safety or interfere with the study evaluation, procedures, or completion. (4) Has developed new-onset paroxysmal or permanent atrial fibrillation requiring rhythm restoring treatment (e.g., direct-current cardioversion, ablation procedure, or antiarrhythmic therapy) within 30 days prior to screening. Patient may re-screen after 30 days if rate (HR <100 bpm) and/or rhythm is stable >30 days. (This exclusion does not apply if atrial fibrillation has been treated with anticoagulation and adequately rate-controlled for ≥14 days.) (5) Has undergone septal reduction therapy (surgical myectomy or transcatheter alcohol ablation) since the completion of a prior study of aficamten. (6) Has current obstructive coronary artery disease (>70% stenosis documented in one or more arteries). (7) Has moderate or severe aortic valve stenosis. (8) Had a confirmed LVEF <40% with an associated dose interruption during the previous a previous study investigating aficamten (i.e., in accordance with Example 2 or Example 5). If data from the participant's cohort has been unblinded, the patient may be considered for entry. (9) History of syncope or sustained ventricular tachyarrhythmia with exercise within 30 days prior to screening. (10) History of ICD placement within 30 days prior to screening. (11) Hypersensitivity to excipients in the aficamten tablets (e.g., mannitol, microcrystalline cellulose, croscarmellose, hydroxypropyl cellulose, sodium lauryl sulfate, magnesium stearate, Opadry QX White 21A180025).

### Study Design

On Day 1, each patient has an echocardiogram; each patient receives Dose 1 of aficamten once daily for 2 weeks. At Week 2, each patient has a truncated echocardiogram following administration of their dose. Patients are up-titrated to Dose 2 if post-Valsalva LVOT-G ≥30 mmHg, and the biplane LVEF ≥55%. Otherwise, the patient remains on 5 mg of aficamten. If LVEF is <50%, treatment is discontinued.

At Weeks 4, 6, 12, and every 12 weeks thereafter, each patient has an echocardiogram or truncated echocardiogram following administration of their dose (a truncated echocardiogram at weeks 4 and 6; and an echocardiogram at week 12 and every 12 weeks thereafter) to determine whether additional dose titration is needed (see Table 21 and Table 22). Patient reported outcomes, laboratory assessments, measurements of NT-proBNP, hs-cTnI, and other biomarkers are performed at week 12 and every 12 weeks thereafter. Ambulatory cardiac monitoring is performed on weeks 48, 96, 144, 192 and 240 Cardiac magnetic resonance is monitored at weeks 48, 144 and 240. If a patient develops an LVEF <40% after the Week 2 visit, a repeat echocardiogram may be performed to confirm the initial finding (preferably within 24 hours). If the echocardiography findings are confirmed, the patient will undergo a ≥2-day drug holiday. The patient may continue aficamten at a reduced, prior tolerated dose once a local echocardiogram documents an LVEF ≥55%.

TABLE 21
Dose Adjustment Algorithm

| | | Post-Valsalva | |
|---|---|---|---|
| Biplane LVEF | | LVOT-G | Action |
| <40% | | | Discontinuation |
| <50% | | | Dose reduce to next |
| | | | lower dose, or |
| | | | discontinuation |
| | | | if on lowest dose |
| ≥50% and <55% | | | Dose unchanged |
| ≥55% | and | <30 mmHg | Dose unchanged |
| ≥55% | and | ≥30 mmHg | Dose escalate to |
| | | | next dose level |

TABLE 22
Dosing Scheme

| Dose 1 | Dose 2 | Dose 3 | Dose 4 |
|---|---|---|---|
| 5 mg | 10 mg | 15 mg | 20 mg |

### Echocardiography

Echocardiographic parameters to be measured at least include the left ventricular parameters (resting left ventricular outflow tract pressure gradient (LVOT-G), post-Valsalva LVOT-G, LVEF, LVFS, left ventricular strain, left ventricular end diastolic volume (LVEDV), left ventricular end systolic volume (LVESV), LV Stroke Volume), septal and free wall thickness, E/e′, E/A, LA volume.

### Cardiac Magnetic Resonance

A cardiac magnetic resonance (CMR) imaging sub-study assesses the effects of long-term administration of aficamten dosing on cardiac morphology, function, and fibrosis in those patients who are eligible and elect to participate. CMR is performed at baseline any time during the screening period and may be performed within 8 weeks prior to the first dose of aficamten given on Day 1. Patients that screen fail and rescreen do not need a repeat baseline CMR. Baseline CMR does not need to be repeated if subject has enrolled in the Example 5 CMR sub-study. Patients with eGFR <30 mL/min/1.73 m² or an allergy to gadolinium may only be given non-contrast CMR. Patients may also choose only non-contrast CMR evaluations for any other reason. Subsequent CMR studies are performed ±30 days of the Week 48 and Week 144 visits, or within 60 days prior to the Week 240 or end of treatment (EOT) visit.

NYHA Functional Classification is recorded. When available, patients will complete patient-reported outcome (PRO) questionnaires: Kansas City Cardiomyopathy Questionnaire (KCCQ), Seattle Angina Questionnaire-7 (SAQ-7), EuroQol 5-Dimension 5-Level Instrument (EQ-5D-5L), Patient's Global Impression of Change (PGI-C) Scale, Clinical Global Impressions (CGI) Scale, SF-36 Physical Functioning Subscale (SF-36 PFS) as specified in Table 23.

TABLE 23
Schedule of health status and health-related quality of life

| | Day 1 | W12 | Q12W | W48 | W96 | W144 | W192 | W240 | EOT |
|---|---|---|---|---|---|---|---|---|---|
| KCCQ | X | X | X | X | X | X | X | X | X |
| SAQ-7 | X | X | X | X | X | X | X | X | X |
| EQ-5D-5L | X | X | X | X | X | X | X | X | X |
| PGI-C | | X | X | X | X | X | X | X | X |
| CGI | | X | X | X | X | X | X | X | X |
| SF-36 PFS | X | | | X | X | X | X | X | X |

### Primary Endpoints

The primary objective of the trial is to determine the safety and tolerability of aficamten over a 5 year period, including assessments of cardiac structure and function, during chronic dosing with aficamten. Safety and tolerability are evaluated by (i) patient incidence of adverse events, (ii) patient incidence of serious adverse events, (iii) patient incidence of LVEF <50%.

### Secondary Endpoints

The secondary objectives are to assess long-term effects of aficamten on LVOT-G in patients with oHCM, as evaluated by peak LVOT-G at rest and with Valsalva provocation; proportion of patients with: a) resting LVOT-G <50 mmHg, b) resting LVOT-G <30 mmHg; c) post-Valsalva LVOT-G <50 mmHg, d) post-Valsalva LVOT-G <30 mmHg, or e) LVEF ≥50%, resting LVOT-G <30 mmHg, and post-Valsalva LVOT-G <50 mmHg; time to first resting LVOT-G <50 mmHg; time to first resting LVOT-G <30 mmHg; time to first post-Valsalva LVOT-G <50 mmHg; time to first post-Valsalva LVOT-G <30 mmHg; and time to first LVEF ≥50%, resting LVOT-G <30 mmHg, and post-Valsalva LVOT-G <50 mmHg

### Exploratory Endpoints

Exploratory objectives of the study include (i) assessing steady-state pharmacokinetics during long-term administration of aficamten, as monitored by C_{trough} at 1 year intervals through end of participation; (ii) assessing long-term effects of aficamten on cardiac biomarkers, as evaluated by change from baseline values in NT-proBNP, hs-cTnI, Galectin-3, PINP, TIMP-1, CITP, Soluble ST2, and other biomarkers at 12-week intervals through end of participation; (iii) assessing effect on functional outcomes such as shift from baseline in NYHA functional class at 12-week intervals through end of participation; (iv) assessing effect on symptoms of oHCM, as evaluated by change from baseline to end of participation, in 12-week intervals, for EQ-5D-5L, PGI-C, CGI, Kansas City Cardiomyopathy Questionnaire (KCCQ), SAQ-7, and SF-36 Physical Functioning Subscale (SF-36 PFS); (v) assessing the pharmacodynamic effect of aficamten on cardiac function and structure, as evaluated by change from baseline to end of participation at 12-week intervals in the following echocardiographic cardiac function and structure measurements: LVEF; Left ventricular fractional shortening (LVFS); Left ventricular stroke volume (LVSV); Left ventricular end-systolic and end-diastolic volumes (LVESV and LVEDV, respectively); Septal, free wall, and maximal wall thickness; Left atrial volume; Left ventricular strain (longitudinal, circumferential, radial); Diastolic indices: E/e′, E/A; (vi) assessing the effect of aficamten on electrocardiographic indices of abnormal myocardial repolarization, as evaluated by change from baseline in the proportion of patients with an LV Strain pattern on ECG to the end of participation at 12-week intervals; (vii) assessing the effect of aficamten on cardiac structure, as evaluated by change from baseline to 1, 3 and 5 years and end of participation in cardiac morphology and structural measurements as assessed by cardiac magnetic resonance (CMR) imaging RV and LV mass; Septal, free wall, and maximal wall thicknesses; Left atrial volume; End-diastolic volume (EDV); End-systolic volume (ESV); (viii) assessing the effect of aficamten on cardiac function, as evaluated by change from baseline to 1, 3 and 5 years and end of participation in biventricular function as assessed by CMR imaging: Stroke volume (SV); Ejection fraction (EF); Cardiac output (CO); (ix) assessing the effect of aficamten on cardiac fibrosis as evaluated by change from baseline to 1, 3 and 5 years and end of participation in the following CMR parameters with Late Gadolinium Enhancement (LGE): LGE mass (g), LGE mass % (as % of LV mass).

### Interim Results for Examples 3a and 3b

Patients were dosed following the protocol of Example 3a or Example 3b. Patients were classified as receiving SoC therapy (both first line and second line) if treated with at least one beta-blocker (BB), non-dihydropyridine calcium channel blocker (CCB), or disopyramide. Patients were eligible for background therapy reduction/withdrawal (BTR/W) at the discretion of the site investigator and once receiving a stable dose of aficamten for at ≥4-weeks and after Week 12 of the study. Successful BTR/W was defined as at least a dose-reduction of one medication to ≤50% of baseline. Serial metrics were collected per protocol but side effects specifically related to SoC therapy were not.

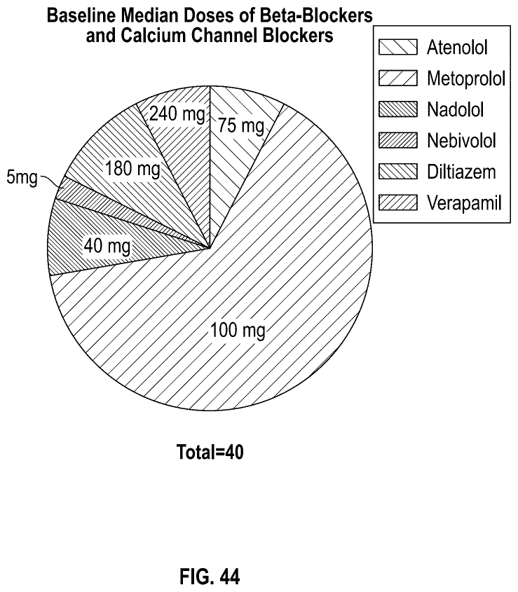

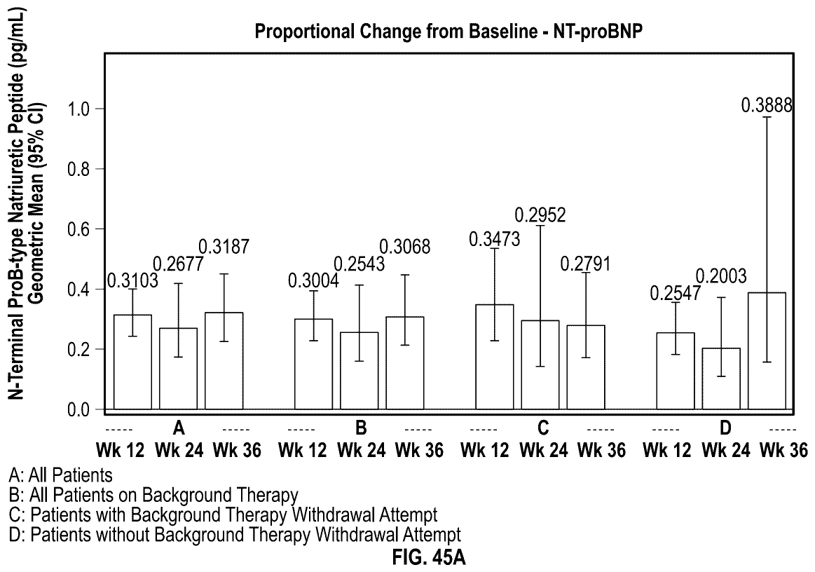

Of first 42 patients enrolled, 39 (93%) were taking ≥1 SoC medication, and of those 27 (69%) were receiving BB only, 4 (10%) CCB only, 7 (18%) CCB or BB and disopyramide, and 1 patient on triple therapy (3%). Among the patients who had experienced ≥12 weeks of treatment with aficamten (N=35), BTR/W was attempted in 20 patients (57%), and 17 (85%) achieved any successful BTR/W. 10 patients completely discontinued at least one medication, and 5 withdrew from all SoC (aficamten monotherapy). 3 patients underwent attempted BTR/W unsuccessfully, with reinstitution of BB as a result recurrence of symptoms or elevated left ventricular outflow tract gradients (LVOT-G). Baseline characteristics of all enrolled patients, and the subgroups in this analysis are shown in Table 24A. Baseline median doses of beta-blockers and calcium channel blockers are shown in FIG. 44.

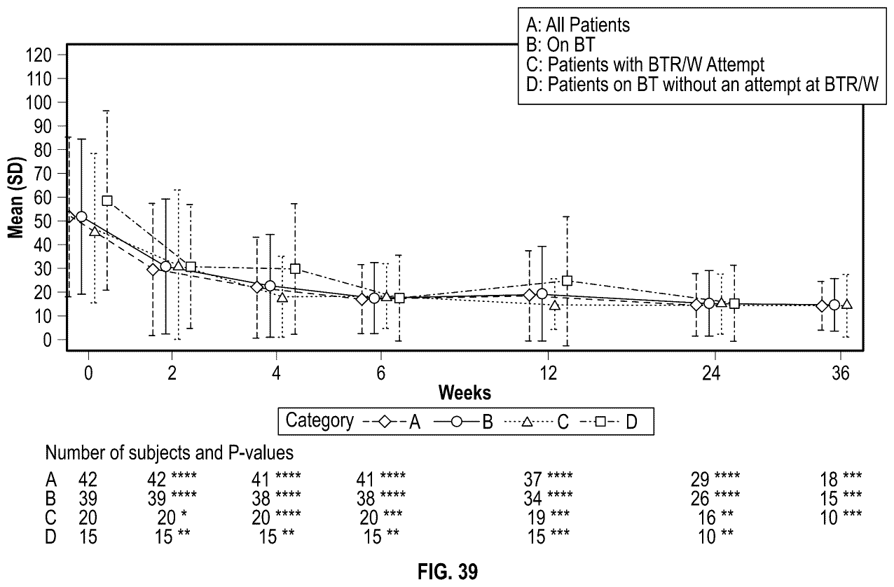

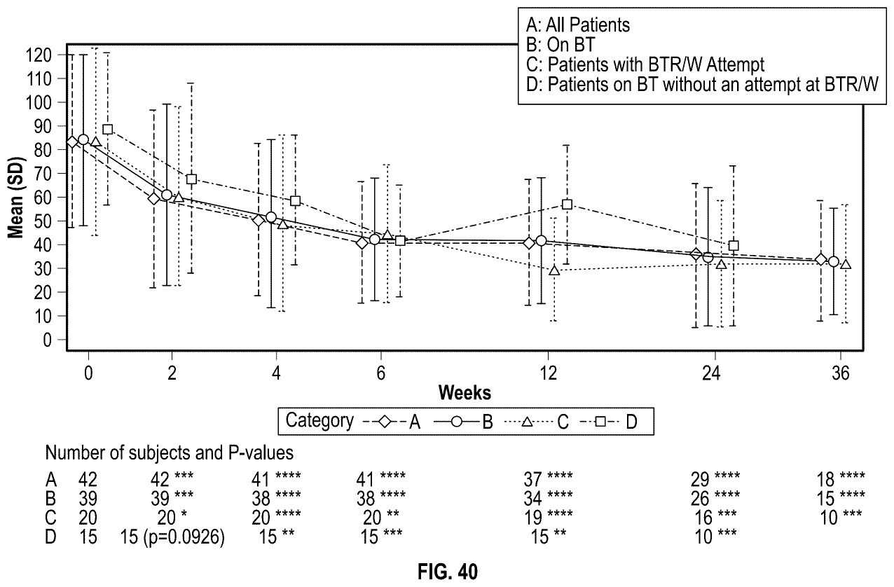

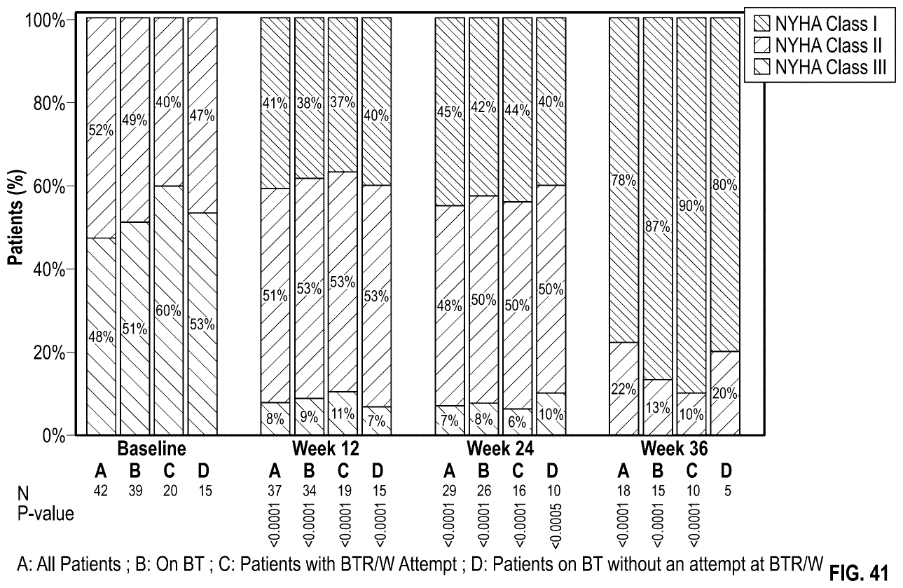

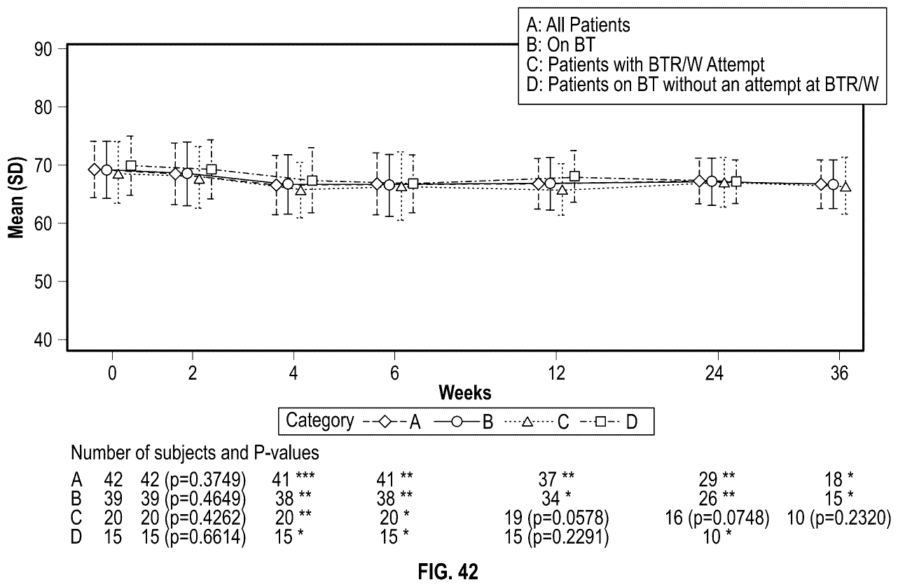

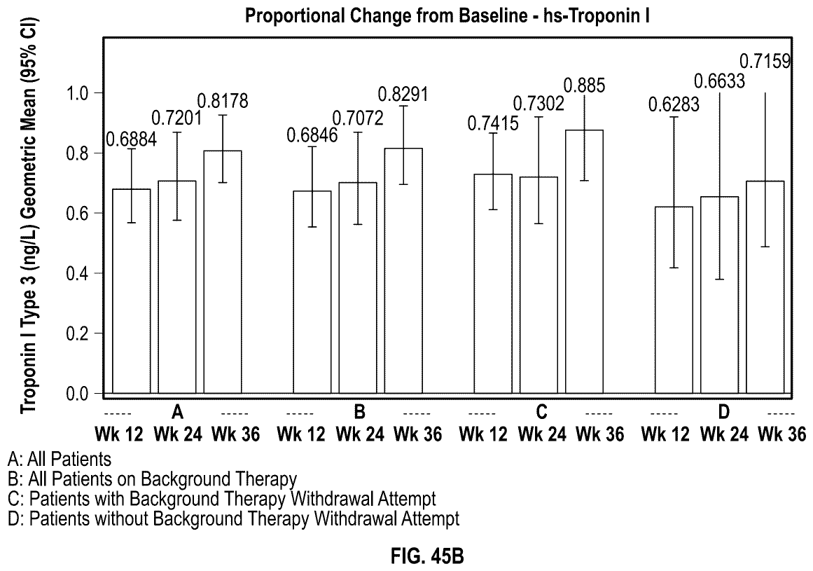

All subgroups demonstrated rapid and sustained relief of obstruction, improvement in cardiac biomarkers, and symptoms which were paralleled by modest decreases in LVEF (FIGS. 39-42). In patients where there was an available assessment both pre- and post-BTR/W (N=14), BTR/W resulted in an increased resting HR of 12 bpm (mean HR=74±10 bpm, p=0.0001) and Valsalva LVOT-G of 15 mmHg (mean Valsalva LVOT-G=42±26 mmHg, p=0.02). All other metrics, including NT-pro B-type Natriuretic Peptide (pre-BTR/W 321±424 pg/dL and post-BTR/W 532±934 pg/mL, p=0.43) and high-sensitivity troponin I levels (pre-BTR/W 4.8±1.8 ng/mL and post-BTR/W 5.1±1.6 ng/mL, p=0.56), were similar. There were no safety events deemed related to aficamten therapy by investigators. NT-proBNP and hs-Troponin I levels are shown in FIGS. 45A and 45B.

TABLE 24A
Baseline Characteristics

| | | | | | No Attempt | | Attempt at | |
|---|---|---|---|---|---|---|---|---|
| | All Patients | | On BT | | at BTR/W | | BTR/W | |
| Metric, (SD) | (N = 42) | | (N = 39) | | (N = 15) | | (N = 20) | |
| Age, years | 59.2 | (12.7) | 59.0 | (13.1) | 57.7 | (13.1) | 61.3 | (13.6) |
| Female (%) | 25 | (60) | 23 | (59) | 7 | (47) | 13 | (65.0) |
| Body mass index | 29.7 | (6.4) | 30.1 | (6.5) | 32.2 | (7.9) | 29.2 | (5.1) |
| HR, BPM | 65 | (11) | 64 | (11) | 64 | (9) | 64 | (12) |
| SBP, mmHg | 122 | (19) | 123 | (19) | 116 | (19) | 129 | (18) |
| DBP, mmHg | 69 | (12) | 68 | (12) | 66 | (13) | 70 | (12) |

Medical History

| | | | | | | | | |
|---|---|---|---|---|---|---|---|---|
| HCM History | | | | | | | | |
| Positive family | 10 | (24) | 10 | (26) | 6 | (40) | 3 | (15) |
| history (%) | | | | | | | | |
| Know pathogenic | 9 | (21) | 9 | (23) | 4 | (33) | 1 | (5%) |
| gene (%) | | | | | | | | |
| Median Time | 2.6 | (1, 24) | 2.9 | (1, 24) | 3.1 | (1, 19) | 2.3 | (1, 13) |
| since diagnosis, | | | | | | | | |
| years (Min, Max) | | | | | | | | |
| HCM Medication History | | | | | | | | |
| Beta-Blocker Use | 33 | (79) | 33 | (85) | 12 | (80) | 18 | (90) |
| (%) | | | | | | | | |
| Calcium Channel | 7 | (17) | 7 | (18) | 3 | (20) | 3 | (15) |
| Blocker (%) | | | | | | | | |
| Disopyramide (%) | 8 | (19) | 8 | (21) | 2 | (13) | 6 | (30) |
| Other Cardiovascular | | | | | | | | |
| History | | | | | | | | |
| Hypertension (%) | 14 | (33) | 14 | (36) | 5 | (33) | 8 | (40) |
| Coronary artery | 7 | (17) | 7 | (18) | 1 | (7) | 5 | (25) |
| disease | | | | | | | | |
| Atrial fibrillation | 7 | (17) | 6 | (15) | 3 | (20) | 3 | (15) |

Hemodynamics

| | | | | | | | | |
|---|---|---|---|---|---|---|---|---|
| LVEF, % | 69 | (5) | 69 | (5) | 70 | (5) | 69 | (5) |
| Resting LVOT-G, mmHg | 52 | (34) | 52 | (33) | 58 | (38) | 47 | (31) |
| Valsalva LVOT-G, | 82 | (35) | 82 | (35) | 87 | (31) | 82 | (38) |
| mmHg | | | | | | | | |

Symptoms

| | | | | | | | | |
|---|---|---|---|---|---|---|---|---|
| NYHA Class (%) | | | | | | | | |
| I | 0 | | 0 | | 0 | | 0 | |
| II | 22 | (52) | 19 | (49) | 7 | (47) | 8 | (40) |
| III | 20 | (48) | 20 | (51) | 8 | (53) | 12 | (60) |
| KCCQ-OSS | 64.9 | (21) | 64.1 | (22) | 65.5 | (19) | 58.8 | (23) |

Cardiac Biomarkers

| | | | | | | | | |
|---|---|---|---|---|---|---|---|---|
| NT-proBNP, pg/mL, | 568 | (284, 1874) | 809 | (306, 1895) | 453 | (306, 1921) | 800 | (308, 1885) |
| median (IQR) | | | | | | | | |
| hs-cTnI, ng/L, median | 10.6 | (4.8, 22.9) | 10.8 | (5, 33.5) | 17.7 | (10.1, 75.5) | 8.4 | (4.5, 12.0) |
| (IQR) | | | | | | | | |

Data presented as mean (SD) except where indicated, BT = Background Therapy, BTR/W = Background Therapy Reduction/Withdrawal, HR = Heart Rate, BPM = Beats per minute, SBP = Systolic blood pressure, DBP = Diastolic blood pressure, LVEF = left ventricular ejection fraction, LVOT-G = Left ventricular outflow tract gradient, NYHA = New York Heart Association, KCCQ-OSS = Kansas City Cardiomyopathy Questionnaire
Overall symptom score, NT-proBNP = N-terminal pro B-type natriuretic peptide, hs-cTnI = high-sensitivity cardiac troponin-I
Second Interim Results at up to 48 weeks

A depiction of the baseline characteristics of the first 45 patients enrolled is shown in Table 24B.

TABLE 24B
Baseline Characteristics

| Baseline Characteristics | Overall (N = 45) | |
|---|---|---|
| Age, years, mean (SD) [range] | 59.0 | (12.8) [23-82] |
| Female, n (%) | 26 | (58) |

Race, n (%)

| | | |
|---|---|---|
| White | 42 | (93.3) |
| Black | 2 | (4.4) |
| Asian | 1 | (2.2) |
| BMI (kg/m2), mean (SD) [range] | 29.9 | (6.3) [22-51] |

NYHA class, n (%)

| | | |
|---|---|---|
| Class II | 24 | (53.3) |
| Class III | 21 | (46.7) |
| Positive family history of HCM, n (%) | 10 | (22.2) |
| Time since initial HCM diagnosis, | 5.3 | (5.4) [1-24] |
| years, mean (SD) | | |
| [range] | | |
| Beta-blocker use, n (%) | 35 | (78) |
| Calcium channel blocker use, n (%) | 8 | (18) |
| Disopyramide use, n (%) | 10 | (22) |
| LVEF* at screening (%), mean (SD) | 68.9 | (4.73) |
| LVOT-G*, Rest at | 47 | (26) |
| screening (mmHg), mean (SD) | | |
| LVOT-G*, Valsalva a | 80 | (30) |
| t screening(mmHg), mean (SD) | | |
| Eligible for septal | 19 | (42) |
| reduction therapy (SRT), n (%) | | |

*Site read of screening echocardiogram

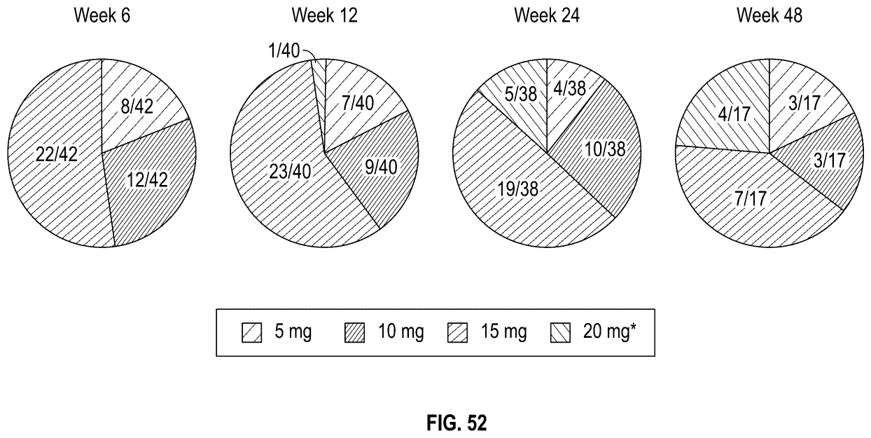

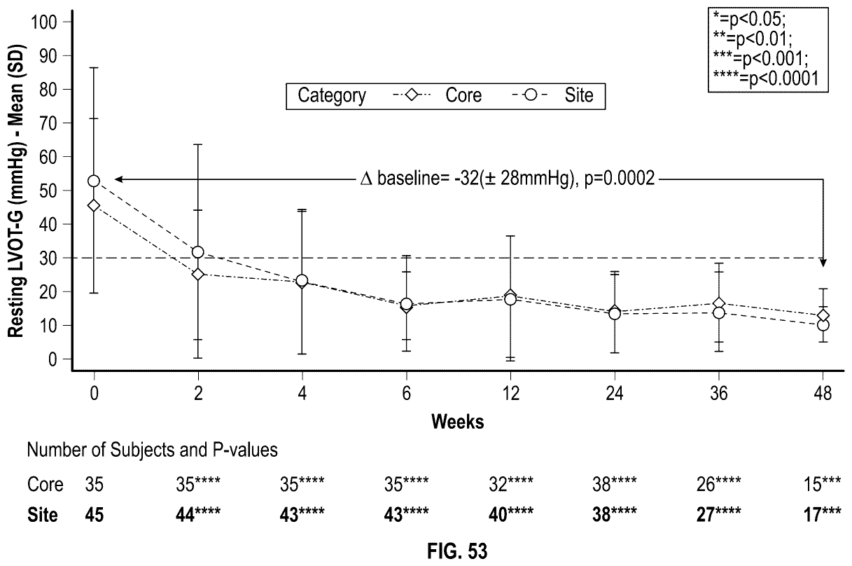

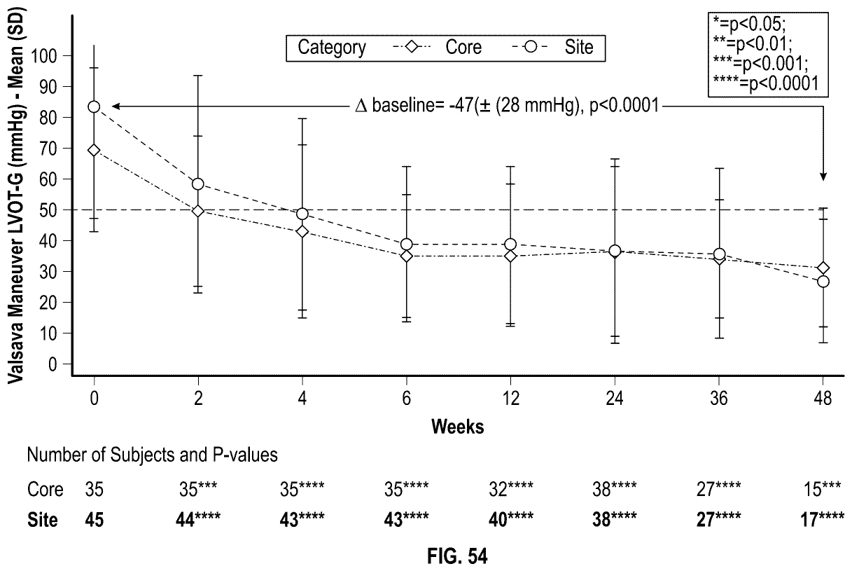

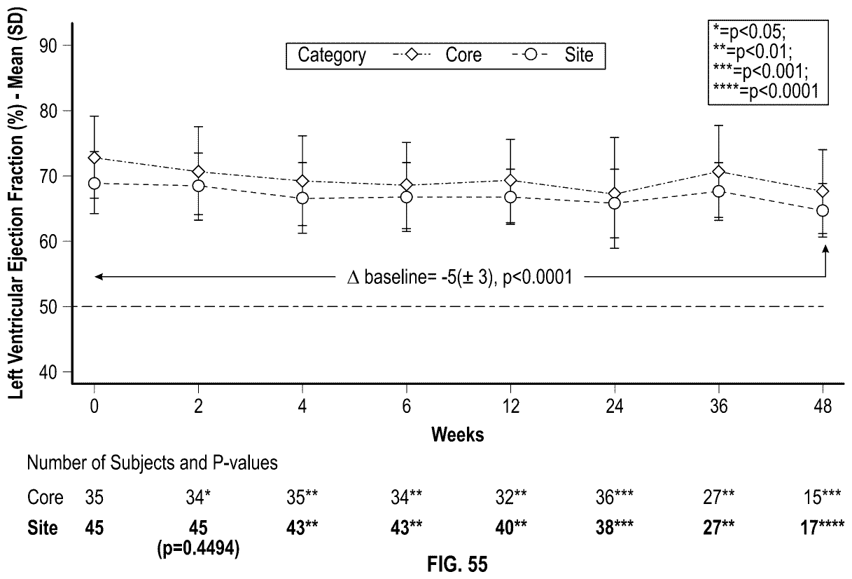

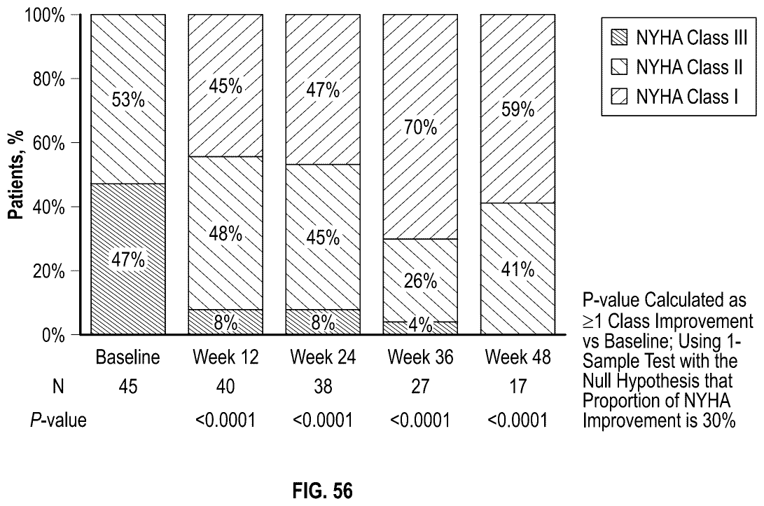

The number of patients and the dosing achieved at each timepoint is shown in FIG. 52. There was a substantial reduction in peak resting and Valsalva LVOT-G from baseline to week 48 [ΔResting mean: −32 mmHg (±28); Δ Valsalva mean: −47 mmHg (±28)] (see FIG. 53 and FIG. 54, respectively). There was a modest reduction in LVEF from baseline to Week 48 (Δ −5±3%) and no patients experienced aficamten related reduction in LVEF <50% (see FIG. 55). There was substantial improvement in NYHA class: by week 48, 88% of patients experienced ≥1 NYHA FC improvement while none had NYHA FC worsening (see FIG. 56). At baseline, 19 patients (42%) were eligible for SRT per guideline criteria of symptoms and hemodynamics, despite receiving beta-blocker, (78%) calcium channel blocker (18%), and disopyramide (22%). (SRT eligibility: NYHA Class III or Class IV and LVOT gradient ≥50 mmHg at rest or with exertion from Valsalva or exercise, or NYHA Class II with exertional symptoms of syncope or near syncope and elevated gradients.) By the Week 48 visit, none of these patients met these criteria. NT pro-BNP decreased from baseline to Week 48 (from a geometric mean [CV %] of 651.0 [160.4] to 111.1 [93.4] pg/mL), representing a 70% reduction from baseline (p<0.0001).

### Interim Global Longitudinal Strain (GLS) Results

Patients (n=27; baseline characteristics in Table 24C) enrolled in the aficamten open label extension trial (FOREST HCM) underwent echocardiography (GE E-95 ultrasound machines) at baseline, week 12 and week 36-48. GLS analysis was performed using a vendor-neutral analysis package (TOMTEC® Imaging Systems).

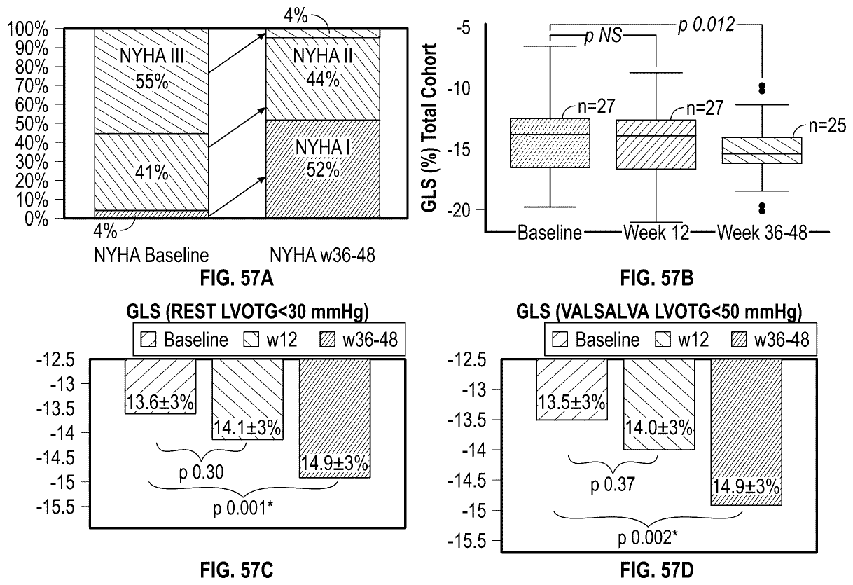

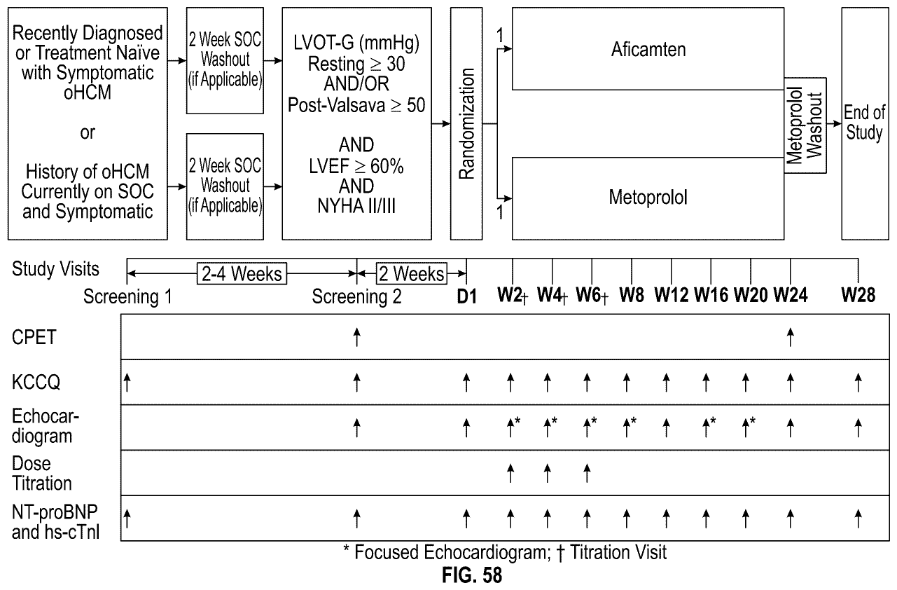

Change in NYHA functional class is shown in FIG. 57A. Compared with baseline, GLS was similar at week 12 (−13.9%±3.2 vs. −14.3±3.0, p 0.32) followed by a significant improvement in GLS at week 36-48 (−13.9%±3.2 vs. −14.9%±2.7, p 0.012) (see FIG. 57B). These improvements were more pronounced in those with optimal hemodynamic response (rest LVOTg <30 and Valsalva LVOTg <50 mmHg; n=21) (see FIG. 57C and FIG. 57D).

TABLE 24C
Baseline characteristics
Clinical data (N = 27)

| Age, years (mean ± SD) | 60.3 ± 12.9 | |
|---|---|---|
| Female, n (%) | 13 | (48%) |

Medical history, n (%)

| | | |
|---|---|---|
| Family history of HCM | 4 | (15%) |
| Atrial fibrillation | 6 | (22%) |
| Hypertension | 16 | (59%) |
| Hyperlipidemia | 17 | (63%) |
| Coronary artery disease | 4 | (15%) |
| Type 2 Diabetes | 3 | (11%) |

Background HCM therapy, n (%)

| | | |
|---|---|---|
| β blocker | 21 | (78%) |
| Calcium channel blocker | 7 | (26%) |
| Disopyramide | 3 | (11%) |
| ICD present, n (%) | 6 | (22%) |
| NYHA Functional Class II, n (%) | 11 | (41%) |
| NYHA Functional Class III, n (%) | 15 | (56%) |

Baseline Echo Parameters (N = 27) (mean ± SD)

| | |
|---|---|
| LVEF, % | 71.3 ± 7.4 |
| Maximum left ventricular wall | 1.9 ± 0.4 |
| thickness, cm | |
| LVOT gradient, rest, mmHg | 54.0 ± 29.7 |
| LVOT gradient, Valsalva, mmHg | 97.3 ± 35.0 |
| LVOT gradient, post-exercise, mmHg | 114.0 ± 52.4 |
| § | |
| Left atrial volume index, mL/m² | 44.9 ± 15.0 |
| E/A | 1.2 ± 0.6 |
| Average E/E′ | 15.2 ± 7.4 |

Severity of MR, n (%)

| | | |
|---|---|---|
| Mild | 19 | (73%) |
| Moderate | 4 | (15%) |
| Severe | 1 | (4%) |

| | |
|---|---|
| Peak pulmonary artery pressure, | 31.3 ± 12.3 |
| mmHg | |

§ No data for 5 patients

### Conclusions

According to the interim results, withdrawal or decrease in SoC medical therapies after treatment with aficamten was generally effective, safe, and associated with continued substantial improvement in clinically relevant measures of efficacy.

Treatment with aficamten was associated with rapid and sustained improvements in echocardiographic hemodynamics paralleled by significant improvements in NYHA class. Aficamten eliminated SRT eligibility in all patients who were guideline eligible at baseline. There were no instances of systolic dysfunction (LVEF <50%) attributed to aficamten.

Aficamten therapy favorably impacted myocardial mechanics (e.g., GLS) after long term treatment and these changes were most pronounced in those with optimal hemodynamic response (rest LVOTg <30 and Valsalva LVOTg <50 mmHg). Without being bound by theory, potential underling mechanisms may include long-term afterload reduction secondary to decrease of the LVOTg, resulting in other pathobiologic changes, since the strain improvements occurred several weeks after gradient reduction.

### Example 4

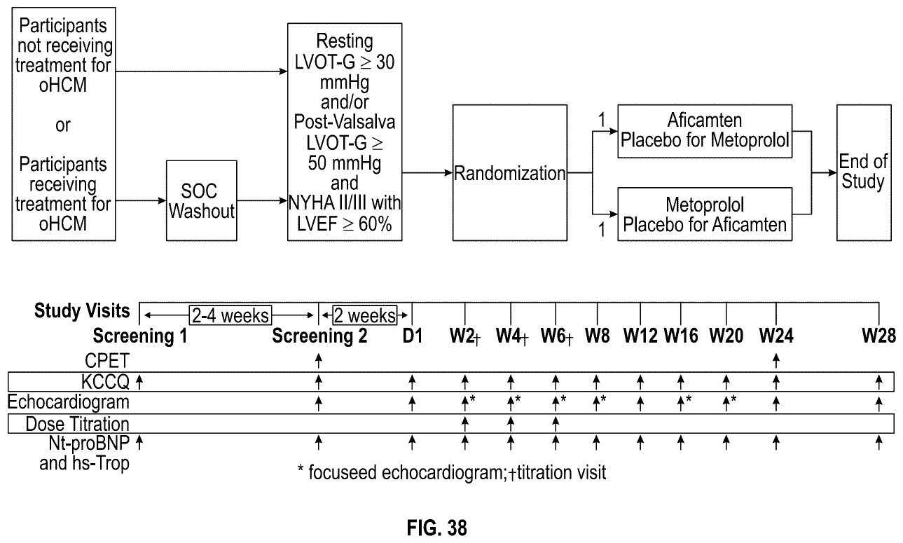

This is a Phase 3, multi-center, randomized, double-blind, active-comparator trial in participants with symptomatic oHCM and elevated LVOT-G (see FIG. 38 and FIG. 58). Approximately 170 eligible participants are randomized in a 1:1 ratio to aficamten or metoprolol. Randomization is stratified by CPET exercise modality (treadmill/bicycle), and recently diagnosed vs chronic oHCM. Randomization is stratified as follows: 1) Participants who are treatment naïve or currently untreated (no SOC medical therapy within the past 12 months), or recently diagnosed participants (history of oHCM ≤12 months with or without use of SOC therapy); and 2) Participants with chronic oHCM (>12 months) currently treated or having received SOC therapy within the past 12 months. The number of participants using the bicycle CPET exercise modality is capped at approximately 50%.

The overall objective of this active comparator trial is to evaluate the safety and efficacy of aficamten as: 1) first-line therapy for participants who are recently diagnosed and/or treatment naïve; or as 2) monotherapy for participants previously receiving standard of care (SOC) medical therapy for symptomatic oHCM.

The primary objective of this trial is to evaluate the effect of aficamten on exercise capacity in patients with symptomatic oHCM. The indicated endpoint is a change in peak oxygen uptake (pVO₂) by cardiopulmonary exercise testing (CPET) from baseline to Week 24.

A secondary objective of this trail is to evaluate the effect of aficamten on New York Heart Association (NYHA) Functional Classification, as determined by the proportion of patients with ≥1 class improvement in NYHA Functional Class from baseline to Week 12 and Week 24.

A further secondary objective of this trial is to evaluate the effect of aficamten on patient health status, as determined by changes in the Kansas City Cardiomyopathy Questionnaire-Clinical Summary Score (KCCQ-CSS) from baseline to Week 12 and Week 24.

A further secondary objective of this trial is to evaluate the effect of aficamten on structural remodeling, as determined by changes in left ventricular mass index (LVMI) or left atrial volume index (LAVI) from baseline to Week 24.

A further secondary objective of this trial is to evaluate the effect of aficamten on N-terminal prohormone brain natriuretic peptide (NT-proBNP) levels from baseline to Week 24.

A further secondary objective of this trial is to evaluate the effect of aficamten on post-Valsalva left ventricular outflow tract gradients (LVOT-G) from baseline to Week 24.

To evaluate the safety and tolerability profile of aficamten in patients with symptomatic oHCM, the following are recorded: (1) incidence of reported major adverse cardiac events (cardiovascular [CV] death, cardiac arrest, non-fatal stroke, non-fatal myocardial infarction, CV hospitalization); (2) incidence of adverse events (AE); and (3) incidence of left ventricular ejection fraction (LVEF) <50%.

An exploratory objective of this trial is to evaluate the effect of aficamten on exercise capacity and functional class, as determined by comparing with baseline, the number of patients at Week 24 achieving either (1) change from baseline of ≥1.5 mL/kg/min in pVO₂ AND ≥1 class improvement in NYHA Functional Class; or (2) Change from baseline of ≥3.0 mL/kg/min in pVO₂ AND no worsening of NYHA Functional Class.

A further exploratory objective of this trial is to evaluate the effect of aficamten on patient response over time, as determined by (1) proportion of patients with improvement in KCCQ-CSS at Weeks 12 and 24; (2) proportion of patients with resting LVOT-G <30 mmHg, post-Valsalva LVOT-G <50 mmHg, and NYHA Functional Class I at Weeks 12 and 24; and (3) proportion of patients with resting LVOT-G <30 mmHg, post-Valsalva LVOT-G <50 mmHg, and ≥1 class improvement in NYHA Functional Class at Weeks 12 and 24.

A further exploratory objective of this trial is to evaluate the effect of aficamten on cardiac troponin levels, as determined by change in high sensitivity cardiac troponin I (hs-cTnI) from baseline to Week 24.

A further exploratory objective of this trial is to evaluate the effect of aficamten on a measure of diastolic function, as determined by change in ratio between early mitral inflow velocity and mitral annular early diastolic velocity (E/e′ [lateral wall]) from baseline to Week 24.

A further exploratory objective of this trial is to evaluate the effect of aficamten on interventricular septal thickness (IVST) remodeling, as determined by change in IVST from baseline to Week 24.

A further exploratory objective of this trial is to evaluate the effect of aficamten on other CPET parameters, as determined by changes from baseline to Week 24 in: (1) ventilator efficiency/Carbon dioxide production (VE/VCO₂ slope); (2) circulatory power (VO₂×systolic blood pressure [SBP]); (3) ventilator anaerobic threshold (VAT); total workload (watts); and heart rate response.

A further exploratory objective of this trial is to evaluate the effect of aficamten on health status and health-related quality of life as measured by PRO questionnaire, as determine by changes from baseline to Week 24 in individual responses to the EuroQol 5-dimension 5-level instrument (EQ-5D-5L), Clinical Global Impression (CGI), Patient Global Impression of Change (PGI-C), and Seattle Angina Questionnaire-7 (SAQ-7).

A further exploratory objective of this trial is to assess the pharmacokinetics of aficamten and its metabolites, as determined by pharmacokinetic parameters through Week 24.

### Inclusion Criteria

Patients are eligible to be included in the trial only if all the following criteria apply: (1) Able to comprehend and willing to sign an ICF and willing to comply with all trial procedures and restrictions for the duration specified in the Schedule of Activities. (2) Males and females between 18 and 85 years of age, inclusive, at screening. (3) Body mass index <35 kg/m². (4) Diagnosed with oHCM per the following criteria by cardiac magnetic resonance imaging (CMR) or echocardiography: (a) has LV hypertrophy with non-dilated LV chamber in the absence of other cardiac disease, and (b) has minimal wall thickness ≥15 mm (minimal wall thickness ≥13 mm is acceptable with a known-disease-causing genetic variant or a positive family history of HCM). (5) Has resting LVOT-G ≥30 mmHg and/or post-Valsalva LVOT-G ≥50 mmHg during screening as determined by the echocardiography core laboratory. (6) Has LVEF ≥60% at screening as determined by the echocardiography core laboratory. (7) New York Heart Association (NYHA) Functional Class II or III at screening visit 2. (8) Hemoglobin ≥10 g/dL at screening. (9) Respiratory exchange ratio (RER) ≥1.05 and pVO₂ <100% predicted on the screening CPET per the core laboratory at Screening Visit 2. (10) Has adequate acoustic windows for echocardiography. (11) Male patients are eligible to participate if they agree to the following during the trial and for at least 4 weeks after the last dose of aficamten: (a) Refrain from donating sperm, plus (b) either (i) be abstinent from heterosexual intercourse as their preferred and usual lifestyle (abstinent on a long term and persistent basis) and agree to remain abstinent, or (ii) must agree to use a male condom and, when his female partner is a woman of childbearing potential, have his female partner use a highly effective method of contraception. (12) A female patient is eligible to participate if she is not pregnant, breastfeeding or planning to donate eggs, and at least one of the following conditions applies: (a) is not a woman of childbearing potential (WOCBP), or is a WOCBP and using a highly effective method of contraception and male partner agrees to use a condom, during the trial and for at least 4 weeks after the last dose of aficamten, and (b) a WOCBP must have a negative pregnancy test (urine or serum as required by local regulations) at Day 1, prior to the first dose of study aficamten. (13) Able to complete all screening procedures. (14) Has KCCQ-CSS score of ≥35 and ≤90 at screening. (15) Patients previously exposed to mavacamten are allowed to participate but must be off mavacamten for at least 8 weeks prior to signing of informed consent and must be approved by the Medical Monitor prior to enrollment. Approximately 10% of oHCM patients who were previously treated with mavacamten can participate in the study with Medical Monitor approval.

### Exclusion Criteria

Patients are excluded from the trial if any of the following criteria apply: (1) Medical indication for either beta blocker or calcium-channel blockers prohibiting drug discontinuation other than oHCM. (2) History of intolerance or medical contraindication to beta blocker therapy. (3) Resting SBP of >160 mmHg at the time of screening. (4) Resting heart rate of >100 beats per minute (bpm) at the time of screening. (5) Significant valvular heart disease (per investigator judgment), including moderate-severe valvular aortic stenosis or fixed subaortic obstruction, and/or mitral regurgitation not due to systolic anterior motion of the mitral valve. (6) Known or suspected infiltrative, genetic or storage disorder causing cardiac hypertrophy that mimics oHCM (e.g., Noonan syndrome, Fabry disease, amyloidosis). (7) History of LV systolic dysfunction (LVEF <45%) or stress cardiomyopathy at any time during their clinical course. (8) Inability to exercise on a treadmill or bicycle (e.g., orthopedic limitations). (9) Has been treated with septal reduction therapy (surgical myectomy or percutaneous alcohol septal ablation) within 6 months of screening (note patients who have had septal reduction therapy >6 months before screening are allowed, up to approximately 10% total), or has plans for either treatment during the trial period that cannot be deferred. (10) History of paroxysmal or persistent atrial fibrillation or atrial flutter (atrial flutter treated with radio frequency ablation without recurrence within the last 6 months prior to screening is allowed). (11) Current or recent (<4 weeks) therapy with disopyramide. (12) History of syncope, symptomatic ventricular arrhythmia, or sustained ventricular tachyarrhythmia with exercise within 6 months prior to screening. (13) ICD placement within 3 months prior to screening or planned ICD placement during the trial. (14) Estimated glomerular filtration rate (eGFR) <30 mL/min/1.73 m² (by the modified Modification of Diet in Renal Disease equation) at screening. (15) Has received prior treatment with aficamten or previously intolerant (reduced LVEF requiring permanent drug discontinuation) to mavacamten.

### Overall Design.

The screening period begins at the time of informed consent and will include both the pre- (Screening Visit 1) and post-washout (Screening Visit 2) period clinical assessments. Those not currently on medical therapy for oHCM do not require a washout period and will only participate in Screening Visit 2. After meeting eligibility criteria, participants are randomized to receive either aficamten and placebo for metoprolol, or metoprolol and placebo for aficamten.

Participants enrolled in this trial are required to have an LVEF ≥60% prior to randomization. Randomization is stratified by CPET exercise modality (treadmill/bicycle), and recently diagnosed vs chronic oHCM as follows: (1) Participants who are treatment naïve or currently untreated (no SOC medical therapy within the past 12 months), or recently diagnosed participants (history of oHCM ≤12 months with or without use of SOC therapy); and (2) Participants with chronic oHCM (>12 months) currently treated or having received SOC therapy within the past 12 months. All randomized participants may receive up to 4 escalating doses of IP over the initial 6 weeks of the trial as Table 27 below.

Participants will have IP dispensed in a double-blinded fashion at each trial visit. During the initial 6 weeks of the treatment period, IP doses are individually titrated at Weeks 2, 4, and 6 according to echocardiographic and vital sign criteria (see table 26). At these visits, an unblinded echocardiologist will review the echocardiographic (LVEF and LVOT-G) and vital sign (SBP and resting heart rate) data, and either the unblinded echocardiologist or unblinded designee will enter the data into the interactive web response system (IWRS), and dose adjustment is carried out according to the prespecified algorithm in a blinded fashion.

Participants receiving aficamten will start at a dose of 5 mg once daily (Dose 1) and may escalate to doses of 10, 15, and 20 mg once daily if they continue to meet the 2 echocardiographic escalation criteria or will remain at their current dose when escalation criteria are not met. If the LVEF is <50% at any time, the dose of aficamten is down-titrated, and if the LVEF is <40% at any time, aficamten is temporarily interrupted.

Participants receiving metoprolol will start at a dose of 50 mg once daily (Dose 1) and may escalate to doses of 100, 150, and 200 mg once daily if they continue to meet the 2 echocardiographic and 2 vital sign escalation criteria or will remain at their current dose when escalation criteria are not met.

If at any time during the trial a participant experiences an intolerable adverse event (AE), which in the investigator's judgment is drug-related and compels the participant to request IP discontinuation, the dose of IP may be reduced to the previous dose level. For participants receiving Dose 1, IP is discontinued.

The treatment duration is 24 weeks with a 4-week follow-up period after the last dose (Weeks 24 through 28). The primary endpoint of pVO₂ is measured by CPET at randomization and at Week 24.

TABLE 25
Vital Sign and Echocardiography Criteria for IP Dose Adjustment

| | | |
|---|---|---|
| Dose | | |
| Adjustment | | |
| Metric | Aficamten | Metoprolol |

All criteria must be met for dose escalation. For down-titration
or IP discontinuation, only one criterion must
be met. If down titration occurs, no further up titration is
allowed. IP cannot be up-titrated after Week 6, but
may be down-titrated to the next lowest dose.

| | | |
|---|---|---|
| SBP | NA | ≥90 mmHg - can dose increase |
| | | <90 mmHg - dose reduce (any visit) |
| Heart Rate | NA | ≥55 bpm - can dose increase |
| | | 50-54 bpm - no dose change |
| | | <50 bpm - dose reduce (any visit) |
| LVEF | ≥55% - can dose increase | ≥55% - can dose increase |
| | <50% - dose reduce (any visit) | 50-54% - no dose change |
| | <40% - temporary discontinuation (any | <50% - dose reduce (any visit) |
| | visit) | <40% - temporary discontinuation (any |
| | | visit) |
| Post-Valsalva | ≥30 mmHg - can dose increase | ≥30 mmHg - can dose increase |
| LVOT-G | <30 mmHg - no change if LVEF | <30 mmHg - no change if LVEF |
| | ≥50% | ≥50% |

bpm = beats per minute; IP = investigational product; LVEF = left ventricular ejection fraction; LVOT-G = left ventricular outflow tract gradients; NA = not applicable; SBP = systolic blood pressure

TABLE 26
Dose Titration Criteria

| | | Post-Valsalva | |
|---|---|---|---|
| Biplane LVEF | | LVOT-G | Action |
| <40% | | Any | Temporary |
| | | | discontinuation |
| <50% | | Any | Reduce Dose^{a} |
| ≥50%-55% | | Any | No Dose Change |
| ≥55% | and | <30 mmHg | No Dose Change |
| ≥55% | and | ≥30 mmHg | Increase Dose |

^{a}Once a patient's aficamten dose is down titrated, no further escalation is permitted.

TABLE 27
Dosing Scheme

| IP | Dose 1 | Dose 2 | Dose 3 | Dose 4 |
|---|---|---|---|---|
| Aficamten | 5 mg | 10 mg | 15 mg | 20 mg |
| Metoprolol | 50 mg | 100 mg | 150 mg | 200 mg |

After randomization, each patient receives Dose 1 (5 mg aficamten, 50 mg metoprolol) once daily for two weeks. At the Week 2 visit, the patient has an echocardiogram 2 hours following administration of their dose. Patients will up-titrate to Dose 2 (10 mg aficamten, 100 mg metoprolol) if the following conditions are met on echocardiography: Post-Valsalva LVOT-G ≥30 mmHg, and the biplane LVEF ≥55%. Otherwise, the patient will remain on Dose 1, except that if LVEF is <50% at Week 2, the IWRS will assign the patient to placebo.

After two more weeks on the assigned dose, at the Week 4 visit each patient has an echocardiogram 2 hours following administration of their dose. Patients will up-titrate to the next higher dose if the following conditions are met on echocardiography: Post-Valsalva LVOT-G ≥30 mmHg, and the biplane LVEF ≥55%. Otherwise, the patient will remain on the same dose, except that if LVEF is <50% at Week 4, the IWRS will assign the patient to the prior dose level or to placebo if the patient was on Dose 1.

After 2 more weeks on the assigned dose, at the Week 6 visit each patient has an echocardiogram 2 hours following administration of their dose. Patients will up-titrate to the next higher dose if the following conditions are met on echocardiography: post-Valsalva LVOT-G ≥30 mmHg, and the biplane LVEF ≥55%. Otherwise, the patient will remain on the same dose, except that if LVEF is <50% at Week 6, the IWRS will assign the patient to the prior dose level or to placebo if the patient was on Dose 1.

At the Week 8, 12, 16, 20, and 24 visits each patient has an echocardiogram 2 hours following administration of their dose of aficamten to ensure the LVEF is ≥50%. If the LVEF is <50% at Week 8, the IWRS will assign the patient to the next lower dose or to placebo if the patient was on Dose 1.

All criteria must be met for dose escalation, but only one criterion must be met for down-titration or IP discontinuation. If down titration occurs, no further up titration is allowed. IP cannot be up-titrated after Week 6 but may be down-titrated to the next lowest dose. Dose escalation for each participant will occur based on the criteria described in Tables 25-27). Importantly, the lower limit of LVEF for dose escalations is increased from 50% to 55% to provide a safety margin from the threshold of LVEF (<50%) that will trigger dose reduction. If the LVEF is <50% at any time, the dose of aficamten is down-titrated, and if the LVEF is <40% at any time, aficamten is temporarily interrupted.

### LVEF Safety Threshold

If the unblinded echocardiologist observes that the LVEF has crossed the defined safety threshold of <40% or deems that the participant requires urgent medical attention, the unblinded echocardiologist or unblinded designee will enter the LVEF value in the IWRS and discuss the results with the blinded investigator or qualified designee. The Medical Monitor is informed in these cases.

If a participant's LVEF is <40% at any time, the following steps should occur after consultation with the Medical Monitor: IP should be stopped and held for at least 7 days. Repeat echocardiograms should be performed per investigator judgment until a normal LVEF (≥55%) has been documented at which point the participant can be re started on IP after being down-titrated. Document dose interruption in the eCRF and include the reason for interruption, the date of the last dose, and the restart date.

If a temporary IP interruption occurred because a safety threshold was met, blinded treatment is resumed at least 7 days later, either at a lower dose or with a permanent switch to placebo if the participant was at 5 mg aficamten or 50 mg metoprolol, as determined by the IWRS.

### Scheduled Down-Titration of Metoprolol at Week 24

The last dose of aficamten and placebo for aficamten will occur on-site at Week 24 (or at a delayed Week 24 visit). The dosing of aficamten and placebo for aficamten will be stopped. To maintain the blind, all participants will down-titrate metoprolol or placebo for metoprolol the next day at home after the Week 24 visit.

Cardiopulmonary Exercise Testing (CPET). All patients undergo CPET with gas-exchange analysis and the methodology is standardized across all participating sites, as described in the CPET manual. Testing includes continuous ECG monitoring by trained personnel and be performed in an area that is equipped for cardiopulmonary resuscitation. Treadmill is the preferred modality for exercise testing. For CPET laboratories that do not perform treadmill testing, cycle ergometry is an acceptable alternative. Exercise protocols for both modalities are provided in the CPET manual. Patients must use the same testing modality for all exercise tests during the trial. Whenever possible, CPET is administered by the same trial personnel using the same equipment and performed after the other trial procedures on that visit day (including echocardiogram, KCCQ, EQ-5D-5L, CGI, PGI-C, NYHA class, SAQ-7, vital signs, ECG, blood sampling, IP administration). Patients naïve to exercise protocols are familiarized with the technique during screening.

All CPET testing is symptom-limited and patients are strongly encouraged to achieve maximal exertion and an RER ≥1.05. The reason(s) for termination of sub-maximal exercise tests are documented. A test is identified as being maximal effort if the RER is ≥1.05.

The Week 24 CPET should be performed at approximately the same time of day (eg, morning, mid-day, afternoon) as the baseline CPET at screening, at a consistent time after the last dose of beta-blocker and IP. Whenever possible, patients should perform exercise testing between three and ten hours after taking beta blocking agents.

If a life-threatening arrhythmia, early ischemia, severe hypotension or other serious finding is identified by the investigator during CPET, the patient is asked to stop the exercise test, and his/her physicians is notified of the results. If the patient is performing the screening test, s/he will not be randomized to the trial. Enrolled patients who have a non-life-threatening event or finding that stops the test can resume testing when it is safe to do so and after appropriate treatment, per the investigator.

Echocardiography. Echocardiography is done during screening and prior to dosing on Day 1. Full or focused echocardiography is performed 2 hours (±60 min) after dosing in the clinic on Weeks 2, 4, 6, 8, 12, 16, 20, and 24. Full echocardiography will also be performed at Week 28.

Certified sonographers will perform echocardiography using standard high-quality, high-fidelity machines. Whenever possible, the same sonographer will perform all studies for a single patient. Echocardiograms are performed after the patient has been resting in a supine position for at least 10 minutes and in accordance with the echocardiography manual. Instructions for the performance of the Valsalva maneuver and imaging the LVOT-G will also be included in the echocardiography manual.

When echocardiograms are scheduled at the same time as blood draws, vital signs, and/or ECGs, the order of evaluation is vital signs, ECGs, blood draw and echocardiogram. The blood draw should be obtained at the scheduled time point and the echocardiograms will follow.

Echocardiographic parameters to be measured at least include the left ventricular parameters (resting left ventricular outflow tract pressure gradient (LVOT-G), post-Valsalva LVOT-G, LVEF, LVFS, global longitudinal strain (GLS), left ventricular end diastolic diameter (LVEDD), left ventricular end diastolic volume (LVEDV), left ventricular end systolic diameter (LVESD), left ventricular end systolic volume (LVESV), left ventricular cardiac output (LVCO), LV Stroke Volume, LVOT velocity time integral (VTI), interventricular septum thickness (IVST), isovolumic contraction time (IVCT), IVRT, E/E, ratio (septal and lateral), and left atrial volume (LAV)) in addition to right heart function metrics detailed in the echocardiography protocol.

Unscheduled echocardiograms may be obtained when clinically indicated, for example to assess an AE or follow-up a clinically significant change in a prior echocardiogram, as determined by the investigator. Results are interpreted by the unblinded Echo Cardiologist at the investigational site.

All echocardiograms (including unscheduled) are sent to the core laboratory for interpretation. On-site interpretation of LVEF and LVOT-G are used for dose escalation and reduction decisions via IWRS. The core laboratory quantification of the echocardiograms are used for all statistical analyses.

Optional HCM Participant Experience Sub-Study. Participants who have consented to the HCM Participant Experience Optional Sub-study will have two semi-structured, qualitative (entry and exit) interviews (see Table 28) conducted remotely by trained personnel from an independent vendor. The entry interview may occur any time after signing informed consent and before Day 1. The exit interview may occur at any time after the Week 20 visit but before the last dose of IP at Week 24. The interviews will be performed remotely. The entry interview will collect information in the participants' words about their own perceptions of baseline functionality, symptom burden, activities of daily living and expectations going into the study. The exit interview will collect information in the participant's words about their experience with any change in functionality and impact of oHCM, symptom burden, activities of daily living and overall treatment experience. Additionally, all participants will complete surveys remotely on Day 1, Weeks 2, 4, 6, 8, 12, 16, 20 and 24. These surveys will track self-selected personal goals to capture aspects of oHCM that are most meaningful or troublesome to them throughout the course of the trial. These goals will use the Specific, Measurable, Achievable, Relevant, Time-bound (SMART) framework.

TABLE 28
HCM Patient Experience Questionnaire

| | | |
|---|---|---|
| 1 | Over the past 7 days, how severe were your HCM symptoms? | All timepoints |
| | No symptoms; Mild; Moderate; Severe; Very severe | |
| 2 | Over the past 7 days, how much did HCM symptoms make it more difficult | All timepoints |
| | to do activities you wanted to do? | |
| | Not at all; A little bit; A moderate amount; A great deal; An extreme amount | |
| 3 | Over the past 7 days, how much did HCM symptoms make it more difficult | All timepoints |
| | to do activities you needed to do? | |
| | Not at all; A little bit; A moderate amount; A great deal; An extreme amount | |
| 4 | Over the past 7 days, how often did HCM symptoms stop you from doing | All timepoints |
| | activities you wanted to do? | |
| | Not at all; A little bit of the time; Some of the time; Most of the time; | |
| 5 | Over the past 7 days, how often did HCM symptoms stop you from doing | All timepoints |
| | activities you needed to do? | |
| | Not at all; A little bit of the time; Some of the time; Most of the time; | |
| 6 | Over the past 7 days, has your mood been negatively affected by HCM | All timepoints |
| | symptoms? | |
| | Not at all; A little bit; A moderate amount; A great deal; An extreme amount | |
| 7 | Over the past 7 days, has your energy been limited by HCM symptoms? | All timepoints |
| | Not at all; A little bit; A moderate amount; A great deal; An extreme amount | |
| 8 | Over the past 7 days, has your physical activity been limited by HCM | All timepoints |
| | symptoms? | |
| | Not at all; A little; A moderate amount; A great deal; An extreme amount | |
| 9 | Over the past 7 days, has your ability to participate in social activities been | All timepoints |
| | limited by HCM symptoms? | |
| | Not at all; A little; A moderate amount; A great deal; An extreme amount | |
| 10 | Over the past 7 days, has your productivity (at home, work and/or school) | All timepoints |
| | been limited by your HCM symptoms? | |
| | Not at all; A little bit; A moderate amount; A great deal; An extreme amount | |
| 11 | Overall, how much has HCM impacted your life? | Baseline only |
| | Not at all; A little bit; A moderate amount; A great deal; An extreme amount | |
| 12 | Since starting the study medication, how much has HCM impacted your | End of |
| | life? | treatment only |
| | Not at all; A little bit; A moderate amount; A great deal; An extreme amount | |
| 13 | How satisfied are you with the study medication? | End of |
| | Very satisfied, Satisfied, Neither satisfied or dissatisfied; Dissatisfied; Very | treatment only |
| | dissatisfied | |

### Example 5

The following example describes a phase 3, multi-center, randomized, double-blind, placebo-controlled trial to evaluate the efficacy and safety of aficamten in adults with symptomatic hypertrophic cardiomyopathy and left ventricular outflow tract obstruction. This trial evaluates the effects of treatment with aficamten over a 24-week period on cardiopulmonary exercise capacity and health status in patients with symptomatic oHCM. This trial is intended to establish the efficacy and safety of aficamten with respect to improvements in exercise capacity and patient symptoms, as well as reduction in left ventricular outflow tract gradient (LVOT-G) in patients with oHCM.

This is a Phase 3 randomized, placebo-controlled, double-blind, multi-center trial in patients with symptomatic oHCM. Approximately 270 eligible patients are randomized in a 1:1 ratio to receive aficamten or placebo. Doses of 5, 10, 15, or 20 mg or matching placebo are administered in an escalating manner using echocardiography to guide dose titration. Randomization is stratified by use of beta-blockers and CPET exercise modality.

The trial comprises three periods. The screening period is up to 6 weeks in duration. The double-blind placebo-controlled treatment period lasts 24 weeks. Following the final dose of aficamten, there is a 4-week safety follow-up period. aficamten is administered orally once daily. During the initial six weeks of the treatment period, aficamten doses are individually titrated at Weeks 2, 4, and 6 using echocardiography. Dose escalation at the Weeks 2, 4, and 6 visits occur only if a patient has a post-Valsalva LVOT-G ≥30 mmHg and a biplane LVEF ≥55%. An echocardiogram is performed at each subsequent visit during the trial and the dose down-titrated if necessary. The primary endpoint of pVO₂ is measured by CPET at screening and at end of treatment (Week 24). If applicable, patients continue taking background HCM medications consistent with regional clinical practice guidelines during the trial.

A CMR imaging sub-study is open to approximately 40 patients who consent to participate.

Genetics sub-study: Patients who consent have DNA analyzed using whole-genome sequencing, whole-exome sequencing, next-generation sequencing, and/or other methods to identify genetic variants.

The main mitigation strategy is facilitated by an individualized dose titration scheme based on each patient's PD response to aficamten with application of pre-specified echocardiographic criteria, including LVEF thresholds for dose escalation, down-titration, and drug discontinuation.

Patients enrolled in this trial are required to have an LVEF ≥60% prior to randomization, as confirmed by the central echocardiography laboratory. A low starting dose of 5 mg and a maximum dose of 20 mg were chosen as these were found to be well-tolerated in the Phase 2 study (CY 6021) of patients with oHCM and effective at reducing the LVOT-G without adversely impacting overall LVEF. Dose escalation is performed on an individualized basis only if the following criteria are met: both post-Valsalva LVOT-G ≥30 mmHg and biplane LVEF ≥55%. Importantly, in contrast to CY 6021, the lower limit of LVEF for dose escalations is increased from 50% to 55% to provide a safety margin from the threshold of LVEF (<50%) that will trigger dose reduction. If the LVEF is <50% at any time, the dose of aficamten will be down-titrated, and if the LVEF is <40% at any time, aficamten will be temporarily interrupted.

The primary objective of this trial is to evaluate the effect of aficamten on exercise capacity in patients with symptomatic oHCM. The indicated endpoint is a change in peak oxygen uptake (pVO₂) by cardiopulmonary exercise testing (CPET) from baseline to Week 24.

A secondary objective of this trial is to evaluate the effect of aficamten on patient health status, as determined by changes in the Kansas City Cardiomyopathy Questionnaire-Clinical Summary Score (KCCQ-CSS) from baseline to Week 12 and Week 24.

A further secondary objective of this trail is to evaluate the effect of aficamten on New York Heart Association (NYHA) Functional Classification, as determined by the proportion of patients with ≥1 class improvement in NYHA Functional Class from baseline to Week 12 and Week 24.

A further secondary objective is to evaluate the effect of aficamten on post-Valsalva left ventricular outflow tract gradients (LVOT-G), as determined by change in post-Valsalva LVOT-G from baseline to Week 12 and Week 24, and the proportion of patients with post-Valsalva LVOT-G <30 mmHg at Week 12 and Week 24.

A further secondary objective is to evaluate the effect of aficamten on exercise capacity, as determined by change in total workload during CPET from baseline to Week 24.

To evaluate the safety and tolerability profile of aficamten in patients with symptomatic oHCM, the following are recorded: (1) incidence of reported major adverse cardiac events (cardiovascular [CV] death, cardiac arrest, non-fatal stroke, non-fatal myocardial infarction, CV hospitalization); (2) incidence of new onset persistent atrial fibrillation; (3) incidence of appropriate implantable cardiac defibrillator (ICD) discharges and aborted sudden cardiac death; (4) incidence of left ventricular ejection fraction (LVEF) <50%; and (5) incidence of treatment emergent adverse events.

An exploratory objective of this trial is to evaluate the effect of aficamten on exercise capacity and functional class, as determined by comparing with baseline, the number of patients at Week 24 achieving either (1) change from baseline of ≥1.5 mL/kg/min in pVO₂ AND ≥1 class improvement in NYHA Functional Class; or (2) Change from baseline of ≥3.0 mL/kg/min in pVO₂ AND no worsening of NYHA Functional Class.

A further exploratory objective of this trial is to evaluate the effect of aficamten on patient response over time, as determined by (1) proportion of patients with improvement in KCCQ-CSS >5 points at Weeks 12 and 24; (2) proportion of patients with resting LVOT-G <30 mmHg, post-Valsalva LVOT-G <50 mmHg, and NYHA Functional Class I at Weeks 12 and 24; and (3) proportion of patients with resting LVOT-G <30 mmHg, post-Valsalva LVOT-G <50 mmHg, and ≥1 class improvement in NYHA Functional Class at Weeks 12 and 24.

A further exploratory objective of this trial is to evaluate the effect of aficamten on other CPET parameters, as determined by changes from baseline to Week 24 in: (1) ventilator efficiency (VE/VCO₂ slope); (2) circulatory power (VO₂×systolic BP); and (3) ventilator anaerobic threshold (VAT).

A further exploratory objective of this trial is to evaluate the effect of aficamten on health status and health-related quality of life as measured by PRO questionnaire, as determine by changes from baseline to Week 24 in individual responses to the EuroQol 5-dimension 5-level instrument (EQ-5D-5L).

A further exploratory objective of this trial is to evaluate the effect of aficamten on cardiac function and structure, as determined by change from baseline to Week 24 in echocardiographic measurements of cardiac structure and of systolic function including: LVEF, left ventricular end-systolic and end-diastolic volumes (LVESV and LVEDV, respectively), and left atrial volume.

A further exploratory objective of this trial is to evaluate the effect of aficamten on biomarker levels, as determined by changes from baseline values in NT-pro-BNP, hs-cardiac-TnI and other biomarkers through Week 24

A further exploratory objective of this trial is to evaluate the effect of aficamten on left ventricular mass, function, and structure by cardiac magnetic resonance (CMR) imaging, as determined by changes from baseline to Week 24 in CMR measurements of left ventricular (LV) mass index, LVEF, septal and free wall thickness, left arterial volume index, LVESV, and LVEDV.

A further exploratory objective of this trial is to assess the pharmacokinetics of aficamten and its metabolites, as determined by pharmacokinetic parameters through Week 24.

Overall Design. This is a Phase 3, randomized, placebo-controlled, double-blind, multi-center trial in patients with symptomatic oHCM. Approximately 270 eligible patients are randomized in a 1:1 ratio to receive aficamten or placebo. Randomization will be stratified by use of beta-blockers (yes or no) and CPET exercise modality (treadmill or bicycle) and implemented in the Interactive Web Response System (IWRS). A cap on the number of patients taking beta-blockers and will not exceed approximately 70% of total enrollment. The number of patients with persistent atrial fibrillation at screening is also capped at approximately 15%, and the number of patients using the bicycle CPET exercise modality will be capped at approximately 50% as well.

Aficamten is administered orally once daily with or without food. During the initial six weeks of the treatment period, aficamten doses are individually titrated at Weeks 2, 4, and 6 using echocardiography. Dose escalation at Weeks 2, 4, and 6 occur only if a patient has a post-Valsalva LVOT-G ≥30 mmHg and a biplane LVEF ≥55%. Echocardiograms are performed at each subsequent visit during the trial and the dose down titrated if necessary. The primary endpoint of pVO₂ is measured by CPET at screening and at end of treatment (Week 24). If applicable, patients continue taking background HCM medications consistent with regional clinical practice guidelines during the trial.

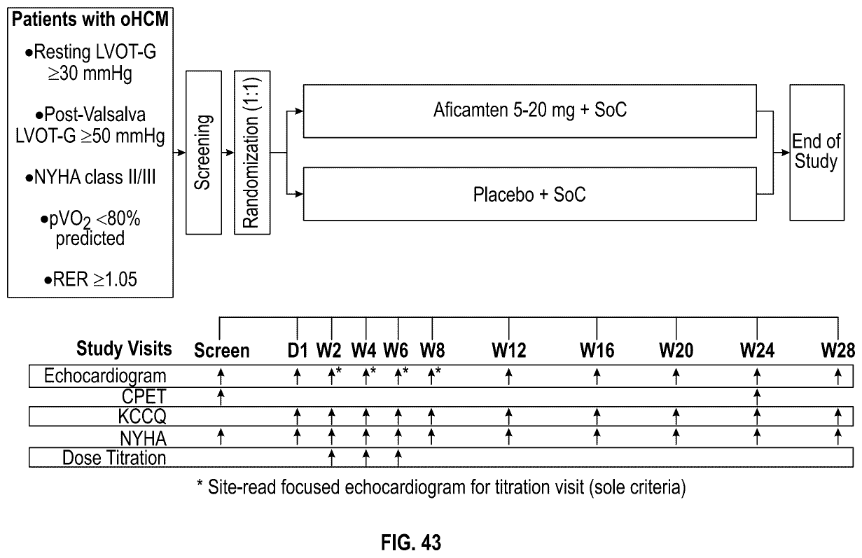

All patients are followed according to the Schedule of Activities (SoA) (see trial design, FIG. 43) from randomization through the date of their final visit irrespective of whether the patient is continuing to receive aficamten, unless the patient has discontinued prematurely from the trial or withdrawn consent. An early discontinuation visit is performed for patients that discontinue prematurely from the trial.

This trial is designed to provide data supporting the clinical efficacy and safety of aficamten in patients with symptomatic oHCM and an LVOT-G ≥50 mmHg post-Valsalva. Reduction of the LVOT-G is expected to correlate with improvement in the patients' symptoms, health status and exercise capacity. Since patient characteristics vary substantially in this disease, individualized dose titration to a PD response (reduction of the post-Valsalva LVOT-G to <30 mmHg with preservation of LVEF ≥55%) is being employed to maximize efficacy and safety. The eligibility criteria are designed to enable enrollment of a patient population representative of the general population of patients with oHCM while ensuring the safety of the patients in this trial. A placebo control and double-blinded approach are being employed in this trial to avoid bias in data collection, including the safety assessments and PD measures that comprise the primary and secondary endpoints.

Patients are eligible to be included in the trial only if all the following criteria apply: (1) Able to comprehend and willing to sign an ICF and willing to comply with all trial procedures and restrictions for the duration specified in the Schedule of Activities. (2) Males and females between 18 and 85 years of age, inclusive, at screening. (3) Body mass index <35 kg/m². (4) Diagnosed with HCM per the following criteria: (a) has LV hypertrophy and non-dilated LV chamber in the absence of other cardiac disease, and (b) has an end-diastolic LV wall thickness as measured by the echocardiography core laboratory of: ≥15 mm in one or more myocardial segments OR ≥13 mm in one or more wall segments and a known-disease-causing gene mutation or positive family history of HCM. (5) Has resting LVOT-G ≥30 mmHg and post-Valsalva LVOT-G ≥50 mmHg during screening as determined by the echocardiography core laboratory. (6) LVEF ≥60% at screening as determined by the echocardiography core laboratory. (7) New York Heart Association (NYHA) Functional Class II or III at screening. (8) Hemoglobin ≥10 g/dL at screening. (9) Respiratory exchange ratio (RER) ≥1.05 and pVO2 <80% predicted on the screening CPET per the core laboratory. (10) Patients on beta-blockers, verapamil, or diltiazem should have been on a stable regimen for >6 weeks prior to randomization and anticipate remaining on the same medication regimen during the trial. (11) Male patients are eligible to participate if they agree to the following during the trial and for at least 4 weeks after the last dose of aficamten: (a) Refrain from donating sperm, plus (b) either (i) be abstinent from heterosexual intercourse as their preferred and usual lifestyle (abstinent on a long term and persistent basis) and agree to remain abstinent, or (ii) must agree to use a male condom and, when his female partner is a woman of childbearing potential, have his female partner use a highly effective method of contraception. (12) a female patient is eligible to participate if she is not pregnant, breastfeeding or planning to donate eggs, and at least one of the following conditions applies: (a) is not a woman of childbearing potential (WOCBP), or is a WOCBP and using a highly effective method of contraception and male partner agrees to use a condom, during the trial and for at least 4 weeks after the last dose of aficamten, and (b) a WOCBP must have a negative pregnancy test (urine or serum as required by local regulations) at Day 1, prior to the first dose of study aficamten. (13) Able to complete all screening procedures.

Patients will be excluded from the trial if any of the following criteria apply: (1) Significant valvular heart disease (per investigator judgment), including moderate-severe valvular aortic stenosis and/or regurgitation, or moderate-severe mitral regurgitation not due to systolic anterior motion of the mitral valve. (2) Documented history of current obstructive coronary artery disease (>70% stenosis in one or more epicardial coronary arteries) or documented history of myocardial infarction. (3) Known or suspected infiltrative, genetic or storage disorder causing cardiac hypertrophy that mimics oHCM (e.g., Noonan syndrome, Fabry disease, amyloidosis). (4) Prior treatment with cardiotoxic agents such as doxorubicin or similar. (5) History of LV systolic dysfunction (LVEF <45%) or stress cardiomyopathy at any time during their clinical course. (6) Has any ECG abnormality considered by the investigator to pose a risk to patient safety (e.g., second degree atrioventricular block type II). (7) Documented paroxysmal atrial fibrillation during the screening period. (8) Paroxysmal or permanent atrial fibrillation requiring rhythm restoring treatment (eg, direct-current cardioversion, atrial fibrillation ablation procedure, or antiarrhythmic therapy) <6 months prior to screening. (This exclusion does not apply if atrial fibrillation has been treated with anticoagulation and adequately rate-controlled for >6 months.) (9) History of syncope or sustained ventricular tachyarrhythmia with exercise within 6 months prior to screening. (10) ICD placement within 3 months prior to screening or planned ICD placement during the trial. (11) History of appropriate ICD discharge for life-threatening ventricular arrhythmia within 6 months prior to screening. (12) Has been treated with septal reduction therapy (surgical myectomy or percutaneous alcohol septal ablation) or has plans for either treatment during the trial period. (13) Inability to exercise on a treadmill or bicycle (e.g., orthopedic limitations). (14) Documented room air oxygen saturation reading <90% at screening. (15) Hepatic impairment defined by a total bilirubin (TBL) ≥1.5×the upper limit of normal (ULN), or alanine aminotransferase (ALT) or aspartate aminotransferase (AST) ≥3×ULN at screening. Patients with documented Gilbert syndrome and TBL ≥1.5×ULN due to unconjugated hyperbilirubinemia, without other hepatic impairment, are permitted. (16) Recipient of a major organ transplant (eg, heart, lung, liver, bone marrow, renal) or anticipated transplantation within 12 months from randomization. (17) History or evidence of any other clinically significant disorder, malignancy, active infection, other condition, or disease that, in the opinion of the investigator or the Medical Monitor, would pose a risk to patient safety or interfere with the trial evaluation, procedures, or completion. (18) Estimated glomerular filtration rate (eGFR) <30 mL/min/1.73 m2 (by the modified Modification of Diet in Renal Disease equation) at screening. (19) Currently participating in another investigational device or drug trial or received an investigational device or drug <1 month (or 5 half-lives for drugs, whichever is longer) prior to screening. Other investigational procedures while participating in this trial are not permitted. (20) Has received prior treatment with aficamten or mavacamten. (21) Any known hypersensitivity to excipients in study drug tablets (e.g., mannitol, microcrystalline cellulose, croscarmellose, hydroxypropyl cellulose, sodium lauryl sulfate, magnesium stearate, Opadry QX White 21A180025).

Exclusion Criteria for CMR sub-study include (1) inability to tolerate CMR, (2) has an ICD, or (3) has a cardiac pacemaker.

Dose modifications and scheduled dose titrations. Patients randomized to aficamten may receive up to four escalating doses of aficamten over the initial 6 weeks of the trial as outlined in Table 29 and FIG. 43. Patients receiving aficamten start at a dose of 5 mg once daily (Dose 1) and may escalate through doses of 10, 15, and 20 mg once daily if they continue to meet the escalation criteria or will stop at their current dose when escalation criteria are not met.

TABLE 29
Echocardiogram Criteria for Scheduled Dose Titrations

| | | Post-Valsalva | |
|---|---|---|---|
| Biplane LVEF | | LVOT-G | Action |
| <50% | | | Reduce Dose^{a} |
| ≥50%-55% | | | No Dose Change |
| ≥55% | and | <30 mmHg | No Dose Change |
| ≥55% | and | ≥30 mmHg | Increase Dose |

^{a}Once a patient's aficamten dose is down titrated, no further escalation is permitted. If LVEF <50% on 5 mg, the patient will receive placebo.

After randomization, each patient receives Dose 1 (5 mg) once daily for two weeks. At the Week 2 visit, the patient has an echocardiogram 2 hours following administration of their dose of aficamten. Patients will up-titrate to Dose 2 (10 mg) if the following conditions are met on echocardiography: Post-Valsalva LVOT-G ≥30 mmHg, and the biplane LVEF ≥55%. Otherwise, the patient will remain on Dose 1, except that if LVEF is <50% at Week 2, the IWRS will assign the patient to placebo.

After two more weeks on the assigned dose, at the Week 4 visit each patient has an echocardiogram 2 hours following administration of their dose of aficamten. Patients will up-titrate to the next higher dose if the following conditions are met on echocardiography: Post-Valsalva LVOT-G ≥30 mmHg, and the biplane LVEF ≥55%. Otherwise, the patient will remain on the same dose, except that if LVEF is <50% at Week 4, the IWRS will assign the patient to the prior dose level or to placebo if the patient was on Dose 1

After 2 more weeks on the assigned dose, at the Week 6 visit each patient has an echocardiogram 2 hours following administration of their dose of aficamten. Patients will up-titrate to the next higher dose if the following conditions are met on echocardiography: post-Valsalva LVOT-G ≥30 mmHg, and the biplane LVEF ≥55%. Otherwise, the patient will remain on the same dose, except that if LVEF is <50% at Week 6, the IWRS will assign the patient to the prior dose level or to placebo if the patient was on Dose 1.

After two additional weeks on the assigned dose, at the Week 8 visit each patient has an echocardiogram 2 hours following administration of their dose of aficamten to ensure the LVEF is ≥50%. If the LVEF is <50% at Week 8, the IWRS will assign the patient to the next lower dose or to placebo if the patient was on Dose 1.

After Week 6, no further dose escalations may occur. During the course of the study, for safety reasons, dose reductions may occur at scheduled or unscheduled visits. Dose reductions is determined by the IWRS system based on echocardiography results. After Week 8, dose reductions are based on echocardiogram results from the initial scheduled or unscheduled visits. If the LVEF is <50%, then the IWRS assigns the patient to the next lower dose or to placebo if the patient was on Dose 1. The IWRS will not further reduce the dose for at least seven days after the previous reduction.

Cardiopulmonary Exercise Testing (CPET). All patients undergo CPET with gas-exchange analysis and the methodology will be standardized across all participating sites, as described in the CPET manual. Testing includes continuous ECG monitoring by trained personnel and be performed in an area that is equipped for cardiopulmonary resuscitation. Treadmill is the preferred modality for exercise testing. For CPET laboratories that do not perform treadmill testing, cycle ergometry is an acceptable alternative. Exercise protocols for both modalities are provided in the CPET manual. Patients must use the same testing modality for all exercise tests during the trial. Whenever possible, CPET is administered by the same trial personnel using the same equipment and performed after the other trial procedures on that visit day (including echocardiogram, KCCQ, EQ-5D-5L, CGI, PGI-C, NYHA class, SAQ-7, vital signs, ECG, blood sampling, IP administration). Patients naïve to exercise protocols will be familiarized with the technique during screening.

All CPET testing is symptom-limited and patients will be strongly encouraged to achieve maximal exertion and an RER ≥1.05. The reason(s) for termination of sub-maximal exercise tests will be documented. A test is identified as being maximal effort if the RER is ≥1.05.

The Week 24 CPET should be performed at approximately the same time of day (eg, morning, mid-day, afternoon) as the baseline CPET at screening, at a consistent time after the last dose of beta-blocker and IP. Whenever possible, patients should perform exercise testing between three and ten hours after taking beta blocking agents.

If a life-threatening arrhythmia, early ischemia, severe hypotension or other serious finding is identified by the investigator during CPET, the patient will be asked to stop the exercise test, and his/her physicians will be notified of the results. If the patient is performing the screening test, s/he will not be randomized to the trial. Enrolled patients who have a non-life-threatening event or finding that stops the test can resume testing when it is safe to do so and after appropriate treatment, per the investigator.

Echocardiography. Echocardiography is done during screening and prior to dosing on Day 1. Echocardiography is also performed 2 hours after dosing in the clinic on Weeks 2, 4, 6, 8, 12, 16, 20, 24, and 28.

Certified sonographers will perform echocardiography using standard high-quality, high-fidelity machines. Whenever possible, the same sonographer will perform all studies for a single patient. Echocardiograms will be performed after the patient has been resting in a supine position for at least 10 minutes and in accordance with the echocardiography manual. Instructions for the performance of the Valsalva maneuver and imaging the LVOT-G will also be included in the echocardiography manual.

When echocardiograms are scheduled at the same time as blood draws, vital signs, and/or ECGs, the order of evaluation will be vital signs, ECGs, blood draw and echocardiogram. The blood draw should be obtained at the scheduled time point and the echocardiograms will follow.

Echocardiographic parameters to be measured at least include the left ventricular parameters (resting left ventricular outflow tract pressure gradient (LVOT-G), post-Valsalva LVOT-G, LVEF, LVFS, global longitudinal strain (GLS), left ventricular end diastolic diameter (LVEDD), left ventricular end diastolic volume (LVEDV), left ventricular end systolic diameter (LVESD), left ventricular end systolic volume (LVESV), left ventricular cardiac output (LVCO), LV Stroke Volume, LVOT velocity time integral (VTI), interventricular septum thickness (IVST), isovolumic contraction time (IVCT), IVRT, E/E, ratio (septal and lateral), and left atrial volume (LAV)) in addition to right heart function metrics detailed in the echocardiography protocol.

Unscheduled echocardiograms may be obtained when clinically indicated, for example to assess an AE or follow-up a clinically significant change in a prior echocardiogram, as determined by the investigator. Results will be interpreted by the unblinded Echo Cardiologist at the investigational site.

All echocardiograms (including unscheduled) will be sent to the core laboratory for interpretation. On-site interpretation of LVEF and LVOT-G will be used for dose escalation and reduction decisions via IWRS. The core laboratory quantification of the echocardiograms will be used for all statistical analyses.

Cardiac Magnetic Resonance. A CMR imaging sub-study will assess the effects of administration of aficamten dosing on cardiac morphology, function, and fibrosis in approximately 40 oHCM patients who are eligible and consent to participate. CMR will be performed during screening period and Week 24. Patients with eGFR <30 mL/min/1.73 ma or an allergy to gadolinium may have a non-contrast CMR.

Baseline Characteristics: 282 patients were enrolled. Patient characteristics at baseline are shown in Table 30. The mean (SD) age was 59.1 (12.94) years, 40.1% were female, and 22% were non-white. Baseline New York Heart Association functional class was Class II for 203 patients (72%), Class III for 67 (23.8%), and Class IV for 1 patient (0.4%). More than half the patients (172; 68%) were taking beta-blockers. The mean baseline pVO2 was 18.5 (SD 4.5) mL/kg/min or 57.1% of predicted maximum, and the mean Kansas City Cardiomyopathy Questionnaire Clinical Symptom Score was 74.7 (SD 18.1). Geometric mean high-sensitivity troponin I was 16.9 (7.7, 27.2) ng/L. Left ventricular ejection fraction, LVOT-G, and N-terminal pro brain natriuretic peptide were blinded.

TABLE 30
Baseline Characteristics

| | N (%) or Mean (SD) | |
|---|---|---|
| Baseline Characteristics | except where indicated | |
| Age, years | 59.1 | (13) |
| Female | 115 | (40.1) |
| Race/ethnicity: | | |
| White | 220 | (78) |
| Black | 3 | (1.1) |
| Asian | 53 | (18.8) |
| Hispanic | 9 | (3.2) |
| Region: | | |
| United States | 94 | (33.3) |
| China | 46 | (16.3) |
| Europe and Israel | 142 | (50.4) |
| Vital signs: | | |
| Weight (kg) | 82.0 | (15.7) |
| Body mass index (kg/m²) | 28.2 | (3.7) |
| Systolic blood pressure (mmHg) | 125.7 | (16.2) |
| Diastolic blood pressure (mmHg) | 74.4 | (10.8) |
| Heart rate (bpm) | 65.4 | (11.2) |
| HCM history: | | |
| History of known | 46 | (16.3) |
| HCM-causing gene mutation | | |
| Positive family history of HCM | 70 | (24.8) |
| Time since initial HCM | 5.9 | (IQR 1.5-8.5) |
| diagnosis (median), years | | |
| HCM medical therapies: | | |
| Beta-blocker (%) | 172 | (67.5) |
| Non-dihydropyridine | 67 | (23.8) |
| calcium channel blocker | | |
| (%) | | |
| Disopyramide (%) | 36 | (12.8) |
| HCM symptoms: | | |
| KCCQ-CSS | 74.6 | (18.1) |
| NYHA class II/III/IV (%) | 203 (72)/67 (23.8)/1 (0.4) | |
| | [11 (3.9) not reported] | |
| SRT guideline eligible* | 67 | (23.8) |
| Comorbidities: | | |
| Hypertension** | 130 | (46.1) |
| Diabetes^{†} | 24 | (8.5) |
| Permanent atrial fibrillation (%) | 1 | (0.4) |
| Paroxysmal atrial fibrillation (%) | 39 | (13.8) |
| CPET metrics: | | |
| Treadmill (%) | 154 | (54.6) |
| pVO₂ (mg/kg/min) | 18.5 | (4.5) |
| pVO₂ percent of predicted | 57.2 | (12.0) |
| maximum (%)^{††} | | |
| Total workload (Watts) | 122.8 | (41.2) |
| Biomarker: | | |
| hs-cTnI (ng/L) geometric mean | 16.9 | (7.7, 27.2) |

*NYHA Class III and any LVOTO ≥50 mmHg
**combines “hypertension” and “essential hypertension”
^{†}combines “type 2 diabetes mellitus”, “type 1 diabetes mellitus”, and “diabetes mellitus”
^{††}Fletcher et al, Circulation, 1995; 91: 580-615
bpm = beats per minute; KCCQ-CSS = Kansas City Cardiomyopathy Questionnaire Clinical Symptom Score; NYHA = New York Heart Association; SRT = septal reduction therapy; pVO₂ = peak oxygen uptake; hs-cTnI = high-sensitivity cardiac troponin-I

### Example 6

Polymorphic Form I, Form II, Form III, Form IV, Form V, and Form VI of aficamten were characterized by various analytical techniques, including XRPD, DSC, TGA, and DVS, as described in WO 2021/011807.

1. A method of treating symptomatic obstructive hypertrophic cardiomyopathy (oHCM) in a patient in need thereof, comprising administering to the patient a therapeutically effective amount of aficamten 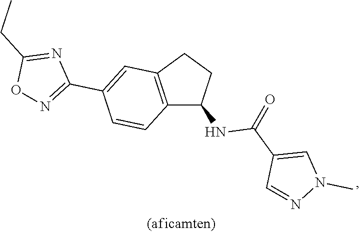 [SMILES: CCc1nc(-c2ccc3c(c2)CC[C@H]3NC(=O)c2cnn(C)c2)no1]
or a pharmaceutically acceptable salt thereof, wherein the patient has been diagnosed with oHCM within 12 months prior to administering aficamten, or a pharmaceutically acceptable salt thereof.

2. A method of treating symptomatic obstructive hypertrophic cardiomyopathy (oHCM) in a patient in need thereof, comprising administering to the patient a therapeutically effective amount of aficamten 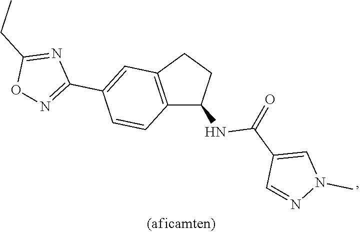 [SMILES: CCc1nc(-c2ccc3c(c2)CC[C@H]3NC(=O)c2cnn(C)c2)no1]
or a pharmaceutically acceptable salt thereof, wherein the patient is treatment naïve for oHCM.

3. The method of claim 1, wherein the method comprises administering aficamten, or a pharmaceutically acceptable salt thereof, as a monotherapy for oHCM.

4. A method of treating symptomatic obstructive hypertrophic cardiomyopathy (oHCM) in a patient in need thereof, comprising administering to the patient a therapeutically effective amount of aficamten 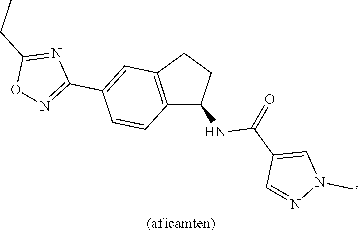 [SMILES: CCc1nc(-c2ccc3c(c2)CC[C@H]3NC(=O)c2cnn(C)c2)no1]
or a pharmaceutically acceptable salt thereof, as a monotherapy for oHCM, wherein the patient has chronic oHCM and has previously received standard of care medical therapy for oHCM and has discontinued said therapy prior to administering aficamten, or a pharmaceutically acceptable salt thereof.

5. The method of claim 4, wherein the standard of care medical therapy comprises treatment with one or more therapeutics selected from a beta-blocker, a calcium channel blocker, and disopyramide.

6. The method of claim 1, wherein the patient has an LVEF ≥60% prior to administering aficamten, or a pharmaceutically acceptable salt thereof.

7-69. (canceled)

70. A method of treating symptomatic obstructive hypertrophic cardiomyopathy (oHCM) in a patient in need thereof, wherein the patient is receiving one or more background therapies for oHCM, comprising (1) administering to the patient a therapeutically effective amount of aficamten 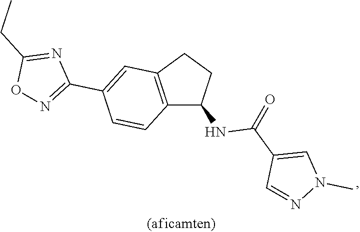 [SMILES: CCc1nc(-c2ccc3c(c2)CC[C@H]3NC(=O)c2cnn(C)c2)no1]
or a pharmaceutically acceptable salt thereof, and (2) reducing and/or stopping at least one background therapy.

71. The method of claim 70, wherein the method comprises reducing at least one background therapy.

72-90. (canceled)

91. A method of treating symptomatic obstructive hypertrophic cardiomyopathy (oHCM) in a patient in need thereof, comprising:
(1) administering to the patient a therapeutically effective amount of aficamten 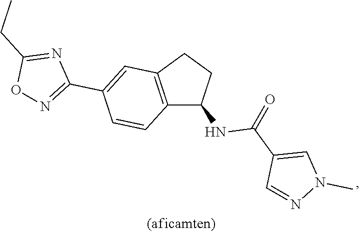 [SMILES: CCc1nc(-c2ccc3c(c2)CC[C@H]3NC(=O)c2cnn(C)c2)no1]
or a pharmaceutically acceptable salt thereof, in combination with a first dose of a standard-of-care therapeutic for oHCM, for a first time period; and
(2) administering to the patient a therapeutically effective amount of aficamten, or a pharmaceutically acceptable salt thereof, in combination with a second dose of the standard-of-care therapeutic for oHCM, for a second time period,
wherein the second dose of the standard-of-care therapeutic for oHCM is lower than the first dose of the standard-of-care therapeutic.

92. The method of claim 91, wherein the second dose of the standard-of-care therapeutic for oHCM is 50% of the first dose of the standard-of-care therapeutic, or lower.

93. A method of treating symptomatic obstructive hypertrophic cardiomyopathy (oHCM) in a patient in need thereof, comprising:
(1) administering to the patient a therapeutically effective amount of aficamten 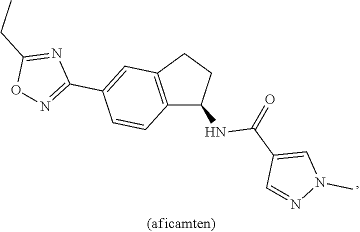 [SMILES: CCc1nc(-c2ccc3c(c2)CC[C@H]3NC(=O)c2cnn(C)c2)no1]
or a pharmaceutically acceptable salt thereof, in combination with a standard-of-care therapeutic for oHCM, for a first time period; and
(2) administering to the patient a therapeutically effective amount of aficamten, or a pharmaceutically acceptable salt thereof, as a monotherapy, for a second time period.

94. The method of claim 91, wherein the patient in need thereof has been receiving the standard-of-care therapeutic for oHCM prior to administering aficamten.

95. A method of treating symptomatic obstructive hypertrophic cardiomyopathy (oHCM) in a patient in need thereof, comprising:
(1) administering to the patient a background therapy comprising at least one standard-of-care therapeutic selected from the group consisting of beta blockers, non-dihydropyridine calcium channel blockers, and disopyramide;
(2) administering to the patient a therapeutically effective amount of aficamten 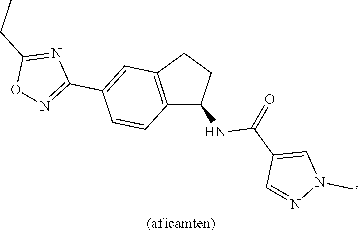 [SMILES: CCc1nc(-c2ccc3c(c2)CC[C@H]3NC(=O)c2cnn(C)c2)no1]
or a pharmaceutically acceptable salt thereof, in combination with the background therapy, for a first time period; and
(3) administering to the patient a therapeutically effective amount of aficamten, or a pharmaceutically acceptable salt thereof, in combination with a second dose of the standard-of-care therapeutic for oHCM, for a second time period,
wherein the second dose of the standard-of-care therapeutic for oHCM is lower than the first dose of the standard-of-care therapeutic.

96. The method of claim 95, wherein the second dose of the standard-of-care therapeutic for oHCM is 50% of the first dose of the standard-of-care therapeutic, or lower.

97. A method of treating symptomatic obstructive hypertrophic cardiomyopathy (oHCM) in a patient in need thereof, comprising:
(1) administering to the patient a background therapy comprising at least one standard-of-care therapeutic selected from the group consisting of beta blockers, non-dihydropyridine calcium channel blockers, and disopyramide;
(2) administering to the patient a therapeutically effective amount of aficamten 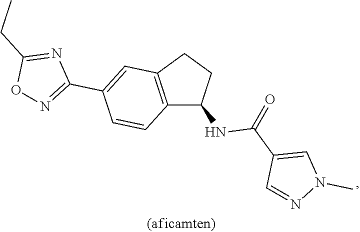 [SMILES: CCc1nc(-c2ccc3c(c2)CC[C@H]3NC(=O)c2cnn(C)c2)no1]
or a pharmaceutically acceptable salt thereof, in combination with the background therapy, for a first time period; and
(3) administering to the patient a therapeutically effective amount of aficamten, or a pharmaceutically acceptable salt thereof, as a monotherapy, for a second time period.

98. The method of claim 91, wherein the first time period is at least 12 weeks.

99. The method of claim 91, wherein the therapeutically effective amount of aficamten, or a pharmaceutically acceptable salt thereof, is selected by titrating a daily dose of aficamten, or a pharmaceutically acceptable salt thereof, administered to the patient.

100-105. (canceled)

106. A method of improving myocardial mechanics or global longitudinal strain (GLS) in a patient in need thereof, comprising administering to the patient a therapeutically effective amount of aficamten 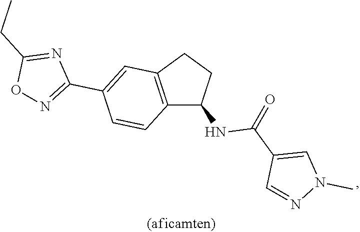 [SMILES: CCc1nc(-c2ccc3c(c2)CC[C@H]3NC(=O)c2cnn(C)c2)no1]
or a pharmaceutically acceptable salt thereof, wherein the patient has obstructive hypertrophic cardiomyopathy (oHCM).

107. (canceled)

108. The method of claim 106, wherein the therapeutically effective amount of aficamten, or a pharmaceutically acceptable salt thereof, is selected by titrating a daily dose of aficamten, or a pharmaceutically acceptable salt thereof, administered to the patient.

109-125. (canceled)

126. The method of claim 1, wherein the aficamten or pharmaceutically acceptable salt thereof comprises one or more of polymorphic Form I, Form II, Form III, Form IV, Form V, and Form VI of aficamten.
